(19) United States
(12) Patent Application Publication
(10) Pub. No.: US 2011/0178107 A1
(43) Pub. Date: Jul. 21, 2011

(54) HCV PROTEASE INHIBITORS

(75) Inventors: Ching-Cheng Wang, Taipei City (TW); Chen-Fu Liu, Taipei City (TW); Kuang-Yuan Lee, Hsinchu City (TW); Mei-Chun Lin, Banciao City (TW); Shu-Huei Wang, Taipei City (TW); Chi-Feng Yen, Taipei County (TW); Yu-Hsuan Wang, Taipei City (TW); Chi-Hsin Richard King, Holladay, UT (US)

(73) Assignee: TaiGen Biotechnology Co., Ltd., Taipei (TW)

(21) Appl. No.: 13/009,213

(22) Filed: Jan. 19, 2011

Related U.S. Application Data
(60) Provisional application No. 61/296,676, filed on Jan. 20, 2010.

(51) Int. Cl. A61K 31/519; C07D 487/04; C07D 471/14; A61P 31/14

(52) U.S. Cl. 514/267; 540/460; 544/250

(57) ABSTRACT

This invention relates to compounds of Formula (I), (II), or (III) shown in the specification. These compounds can be used to treat hepatitis C virus infection.

### CROSS REFERENCE

This application claims priority to U.S. Provisional Application Ser. No. 61/296,676, filed Jan. 20, 2010, the content of which is incorporated herein by reference.

### BACKGROUND

Hepatitis C virus (HCV), a (+)-sense single-stranded RNA virus, is the major causative agent for most cases of non-A, non-B hepatitis. Infection by HCV is a compelling human health problem. See, e.g., WO 05/007681; WO 89/04669; EP 381216; Alberti et al., J. Hepatology, 31 (Suppl. 1), 17-24 (1999); Alter, J. Hepatology, 31 (Suppl. 1), 88-91 (1999); and Lavanchy, J. Viral Hepatitis, 6, 35-47 (1999).

Hepatitis caused by HCV infection is difficult to treat since the virus can quickly mutate and escape the natural immune response. The only anti-HCV therapies currently available are interferon-α, interferon-α/ribavirin combination, and pegylated interferon-α. However, sustained response rates for interferon-α or interferon-α/ribavirin combination were found to be <50% and patients suffer greatly from side effects of these therapeutic agents. See, e.g., Walker, DDT, 4, 518-529 (1999); Weiland, FEMS Microbial. Rev., 14, 279-288 (1994); and WO 02/18369. Thus, there remains a need for developing more effective and better-tolerated therapeutic drugs.

The HCV genome contains a single polyprotein of about 3000 amino acids. It includes a nucleocapsid protein (C), envelope proteins (E1 and E2), and several non-structural proteins (p7, NS2, NS3, NS4a, NS5a, and NS5b). Of which the NS3 protein possesses serine protease activity and is considered essential for viral replication and infectivity. The essentiality of the NS3 protease was inferred from the fact that mutations in the yellow fever virus NS3 protease decreased viral infectivity. See, e.g., Chamber et al., Proc. Natl. Acad. Sci. USA 87, 8898-8902 (1990). It was also demonstrated that mutations at the active site of the HCV NS3 protease completely inhibited the HCV infection in chimpanzee model. See, e.g., Rice et al., J. Virol. 74 (4) 2046-51 (2000). Further, the HCV NS3 protease was found to facilitate proteolysis at the NS3/NS4a, NS4a/NS4b, NS4b/NS5a, NS5a/NS5b junctions and was thus responsible for generating four viral proteins during viral replication. See, e.g., US 2003/0207861.

Consequently, the HCV NS3 protease enzyme is an attractive target in treating HCV infection. Potential NS3 HCV protease inhibitors can be found in Current Opinion in Investigational Drugs, 10, 821-837 (2009), WO 2008/095058, WO2008/057995, WO 2009/055335, WO 02/18369, WO 00/09558, WO 00/09543, WO 99/64442, WO 99/07733, WO 99/07734, WO 99/50230, WO 98/46630, WO 98/17679, WO 97/43310, U.S. Pat. No. 5,990,276, Dunsdon et al., Biorg. Med. Chem. Lett, 10, 1571-1579 (2000); Llinas-Brunet et al., Biorg. Med. Chem. Lett. 10, 2267-2270 (2000); and S. LaPlante et al., Biorg. Med. Chem. Lett. 10, 2271-2274 (2000).

### SUMMARY

This invention is based on unexpected discoveries that certain macrocyclic compounds block activity of NS3-4A proteases, decrease HCV RNA levels, inhibit HCV protease mutants resistant to other inhibitors, and show a prolonged half-life in the blood system.

In one aspect, this invention relates to compounds of Formula (I):

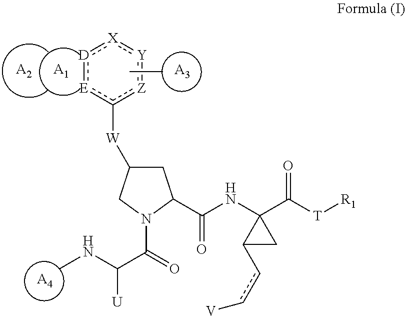

wherein R₁ is H, or is a moiety selected from C₁₋₆ alkyl, C₃₋₁₀ cycloalkyl, C₁₋₁₀ heterocycloalkyl, aryl, and heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl; T is —O—, —NH—, —NH(CO)—, —NHS(O)—, or —NHSO₂—; each of U and V, independently, is H, C₁₋₆ alkyl, or C₃₋₁₀ cycloalkyl; W is —O—, —S—, —NH—, —OCH₂—, or a bond; each of D and E, independently, is C or N; one of X, Y, and Z is C and bonded to A₃, and each of the others, independently, is CH, N, or deleted; each of A₁, A₂, and A₃, independently, is a moiety selected from C₄₋₁₀ cycloalkyl, C₁₋₁₀ heterocycloalkyl, aryl, and heteroaryl, each of which is optionally mono-, di-, or tri-substituted with halo, nitro, cyano, amino, amideo, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; A₄ is a moiety selected from C₁₋₁₀ heterocycloalkyl, aryl, and heteroaryl, each of which is optionally mono-, di-, or tri-substituted with halo, cyano, amino, amido, carbonyloxy, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; each of C₁₋₆ alkyl, C₁₋₆ alkoxyl, C₂₋₆ alkenyl, and C₂₋₆ alkynyl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, and each of C₃₋₁₀ cycloalkyl, C₁₋₁₀ heterocycloalkyl, aryl, and heteroaryl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, or optionally fused with C₃₋₁₀ cycloalkyl, C₁₋₁₀ heterocycloalkyl, aryl, or heteroaryl; and [symbol] is a single bond or a double bond.

Referring to formula (I), the compounds described above may features that

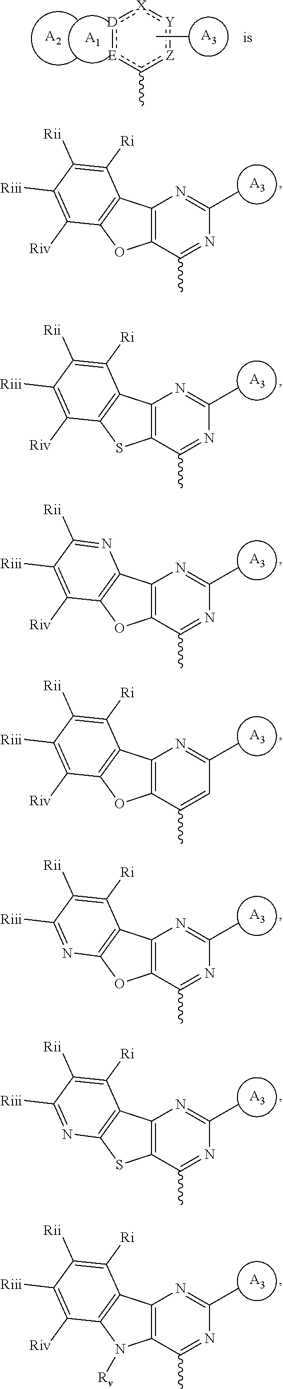

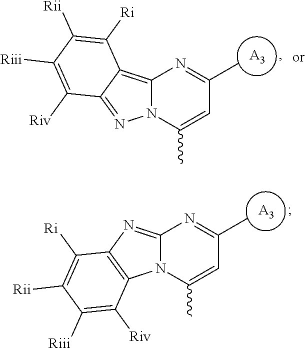

in which A₃ is aryl or heteroaryl optionally substituted with halo, amino, amido, C₁₋₆ alkyl, or C₁₋₆ alkoxyl; and each of Rᵢ, Rᵢᵢ, Rᵢᵢᵢ, Rᵢᵥ, and Rᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

The compounds described above may also feature that A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl.

The compounds described above may also have one or more of the following features: R₁ is

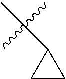

or

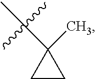

W is O, T is —NHSO₂—, U is C₁₋₆ alkyl, V is H, and [symbol] is a double bond.

In another aspect, this invention relates to compounds of formula (II):

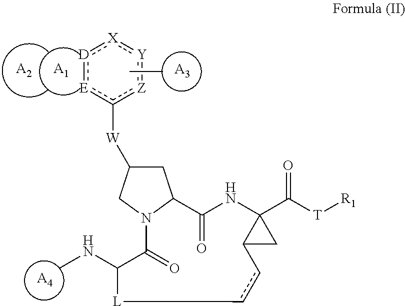

wherein R₁ is H, or is a moiety selected from C₁₋₆ alkyl, C₃₋₁₀ cycloalkyl, C₁₋₁₀ heterocycloalkyl, aryl, and heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl; T is —O—, —NH—, —NH(CO)—, —NHS(O)—, or —NHSO₂—; L is C₁₋₆ alkylene, C₂₋₆ alkenylene, or C₃₋₁₀ cycloalkylene; W is —O—, —S—, —NH—, —OCH₂—, or a bond; each of D and E is C or N; one of X, Y, and Z is C and bonded to A₃, and each of the others, independently, is CH, N, or deleted; each of A₁, A₂, and A₃, independently, is a moiety selected from C₄₋₁₀ cycloalkyl, C₁₋₁₀ heterocycloalkyl, aryl, and heteroaryl, each of which is optionally mono-, di-, or tri-substituted with halo, nitro, cyano, amino, amideo, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; A₄ is a moiety selected from C₁₋₁₀ heterocycloalkyl, aryl, and heteroaryl, each of which is optionally mono-, di-, or tri-substituted with halo, cyano, amino, amido, carbonyloxy, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; each of C₁₋₆ alkyl, C₁₋₆ alkoxyl, C₂₋₆ alkenyl, and C₂₋₆ alkynyl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, and each of C₃₋₁₀ cycloalkyl, C₁₋₁₀ heterocycloalkyl, aryl, and heteroaryl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, or optionally fused with C₃₋₁₀ cycloalkyl, C₁₋₁₀ heterocycloalkyl, aryl, or heteroaryl; and [symbol] is a single bond or a double bond.

Referring to formula (II), the compounds described above may features that

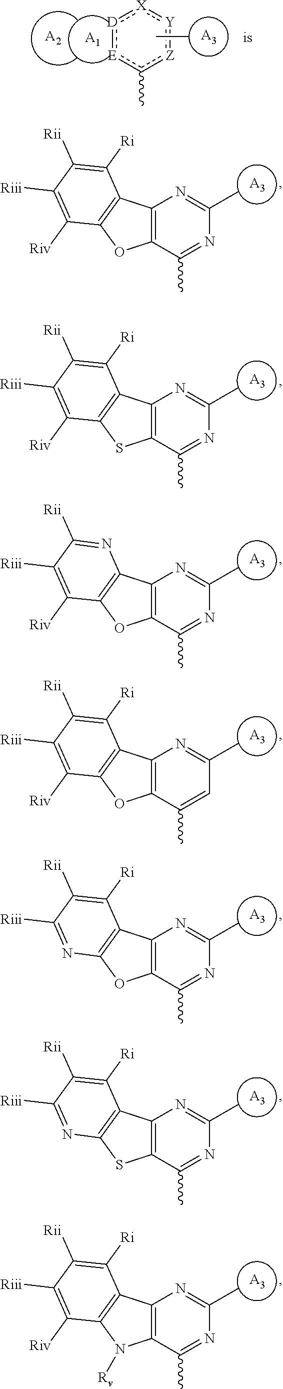

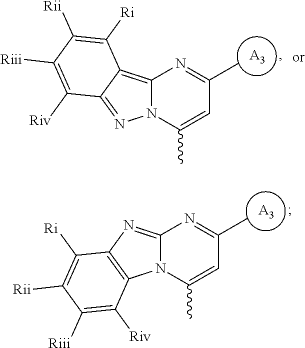

in which A₃ is aryl or heteroaryl optionally substituted with halo, amino, amido, C₁₋₆ alkyl, or C₁₋₆ alkoxyl; and each of Rᵢ, Rᵢᵢ, Rᵢᵢᵢ, Rᵢᵥ, and Rᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

The compounds described above may also feature that A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl.

The compounds described above may also have one or more of the following features: R₁ is

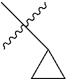

or

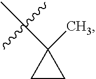

W is O, T is —NHSO₂—, [symbol] is a double bond, and L is —(CH₂)ₘ—, m being 3, 4, or 5.

In still another aspect, this invention relates to compounds of formula (III):

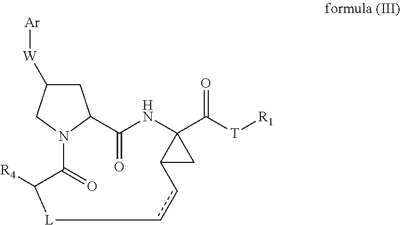

wherein R₁ is H, OH, halo, nitro, cyano, amino, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, or heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; T is —O—, —NH—, —NH(CO)—, —NHS(O)—, or —NHSO₂—; L is —(CH₂)ₘ—, m being 3, 4, or 5; W is —O—, —S—, —NH—, —OCH₂—, or a bond; R₄ is H, NHR, or NHZR; in which R is C₁₋₆ alkyl, C₃₋₁₀ cycloalkyl, C₁₋₁₀ heterocycloalkyl, aryl, or heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl; Z is —(C═O)—, —(C═O)O—, —(C═O)(C═O)O—, —(C═O)(C═O)NH—, —(C═O)NHR′—, —(C═O)S—, (C═S)NHR, ORC(NH)O, R′ being H, C₁₋₆ alkyl, C₃₋₁₀ cycloalkyl, C₁₋₁₀ heterocycloalkyl, aryl, or heteroaryl; and Ar is

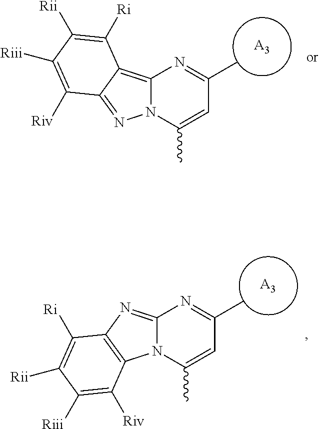

in which A₃ is C₁₋₆ alkyl, C₁₋₆ alkoxy, C₃₋₈ cycloalkyl, C₁₋₈ heterocycloalkyl, aryl, or heteroaryl optionally substituted with halo, amino, amido, C₁₋₆ alkyl, or C₁₋₆ alkoxyl; and each of Rᵢ, Rᵢᵢ, Rᵢᵢᵢ, and Rᵢᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

Referring to formula (III), the compounds described above may have one or more of the following features: wherein R₁ is

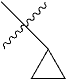

or

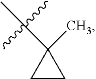

T is —NHSO₂—, [symbol] is a double bond, and L is —(CH₂)ₘ—, m being 3, 4, or 5.

The term “alkyl” refers to a saturated, linear or branched hydrocarbon moiety, such as —CH₃, —CF₃, or —CH(CH₃)₂. The term “alkoxy” refers to an —O—(C₁₋₆ alkyl) radical. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl. The term “amino” refers to a radical of —NH₂, —NH—(C₁₋₆ alkyl), or —N(C₁₋₆ alkyl)₂.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

Shown below are 305 exemplary compounds of this invention.

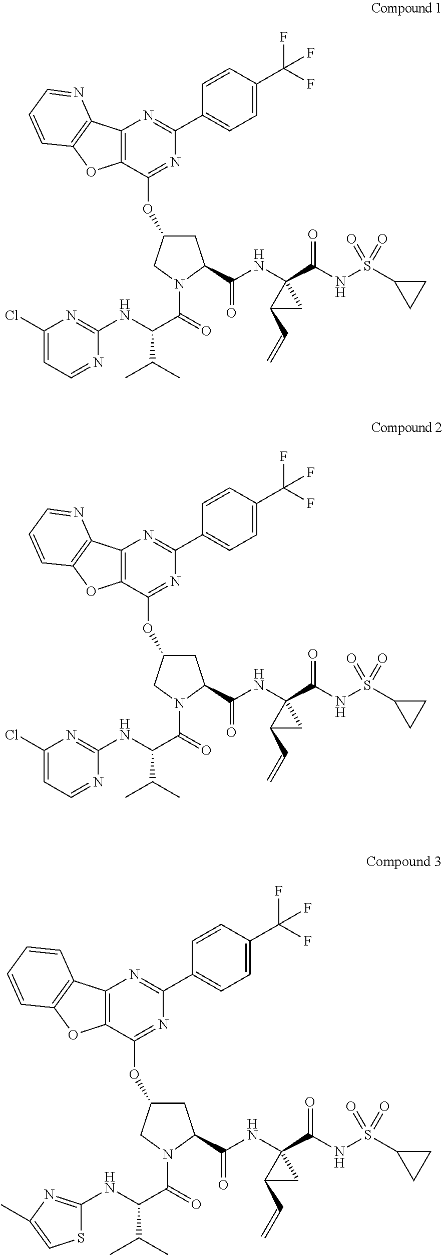

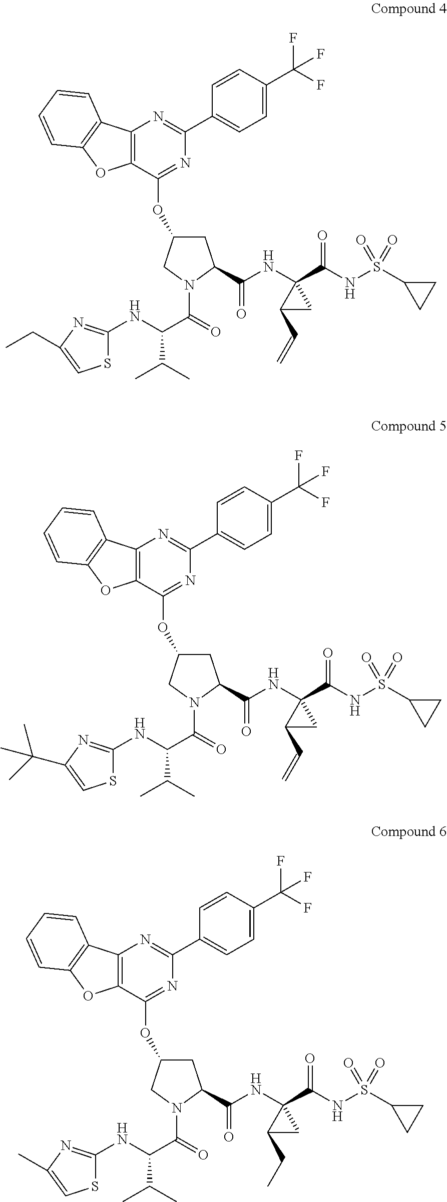

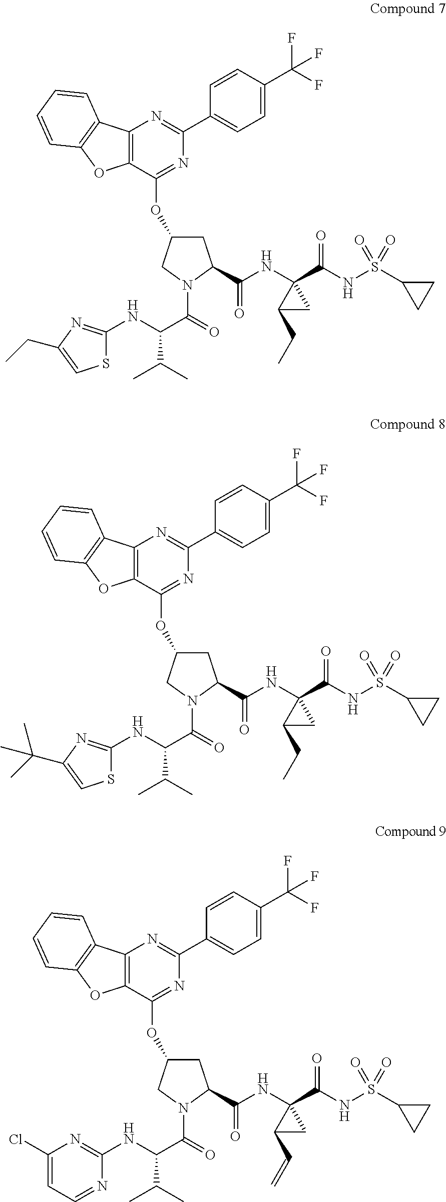

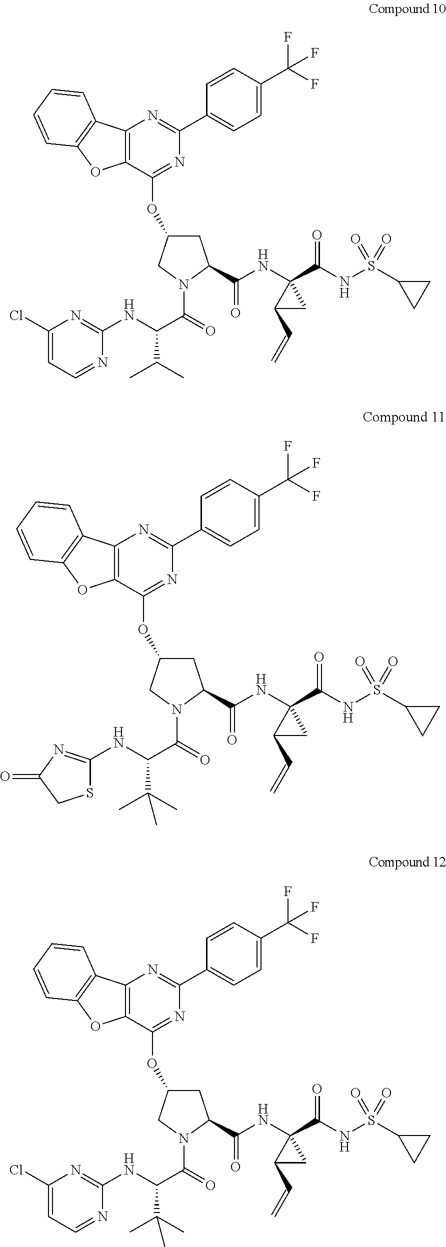

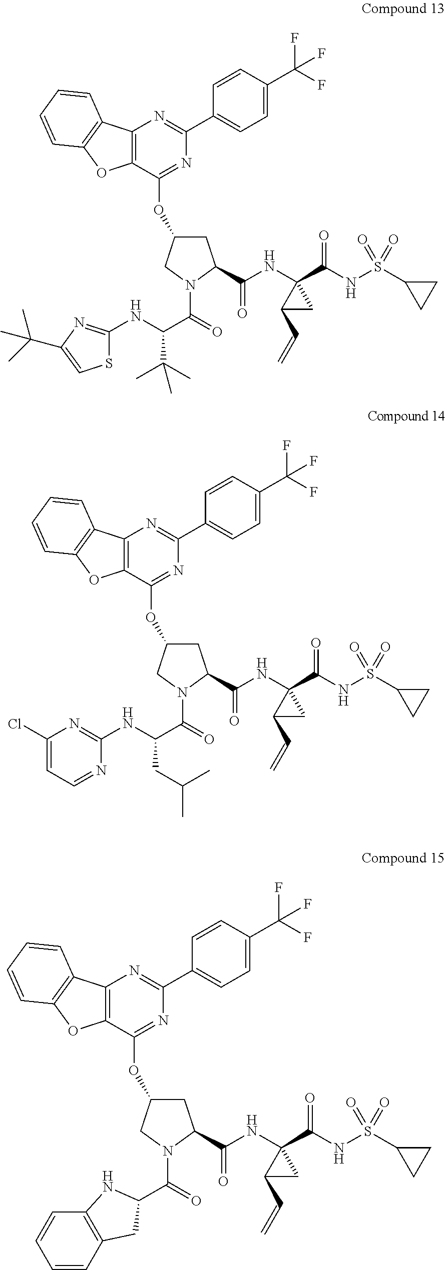

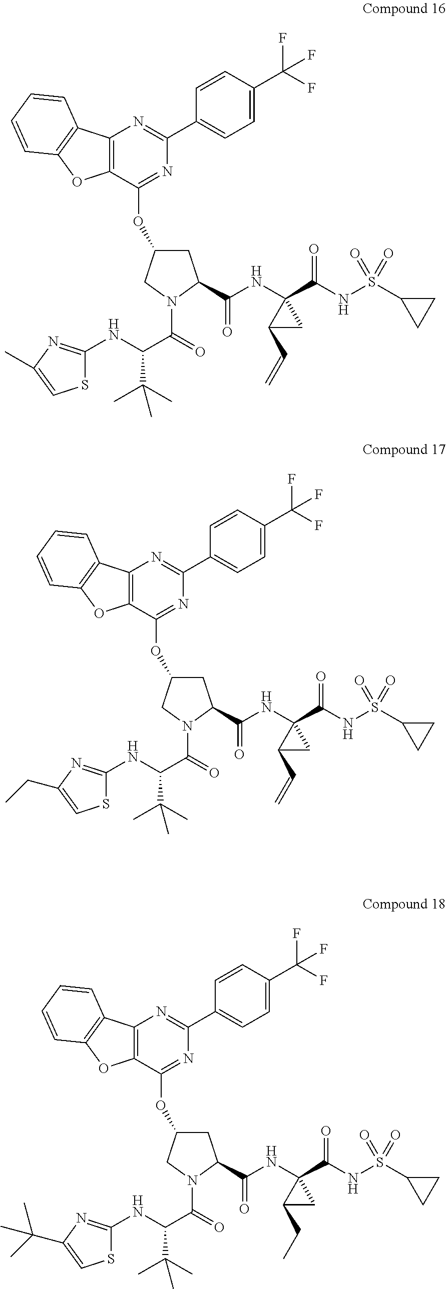

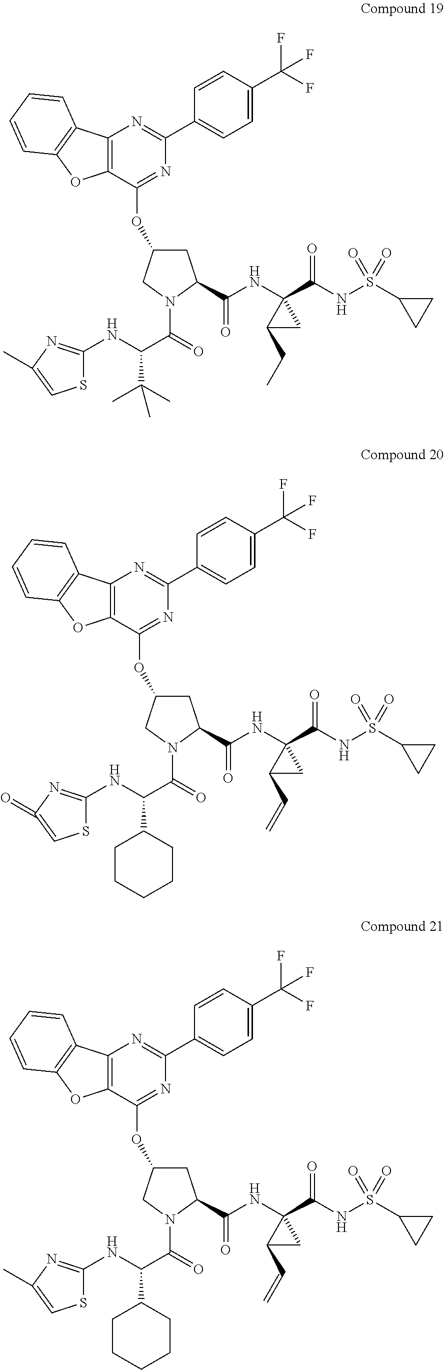

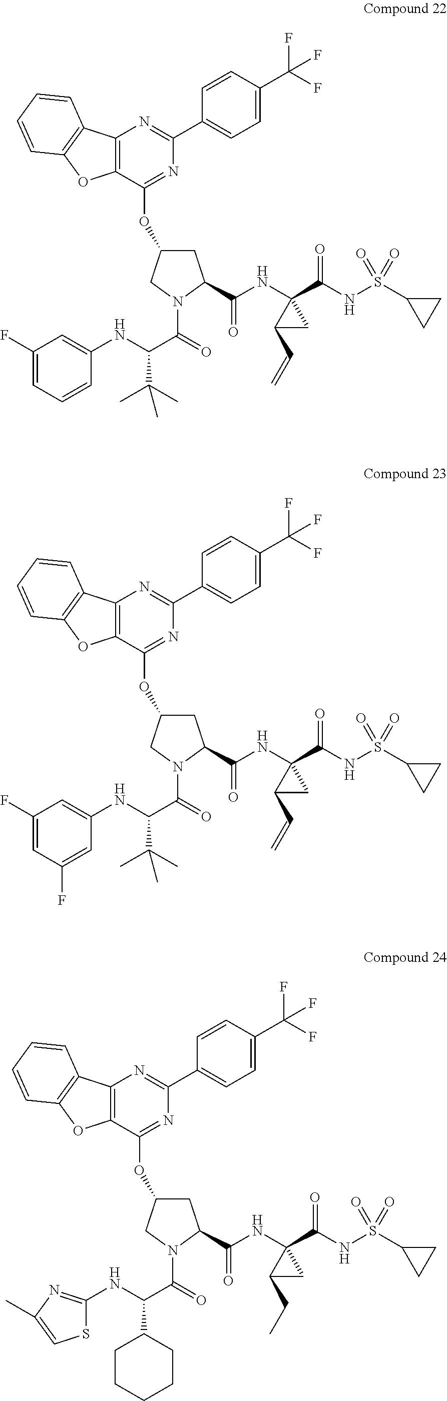

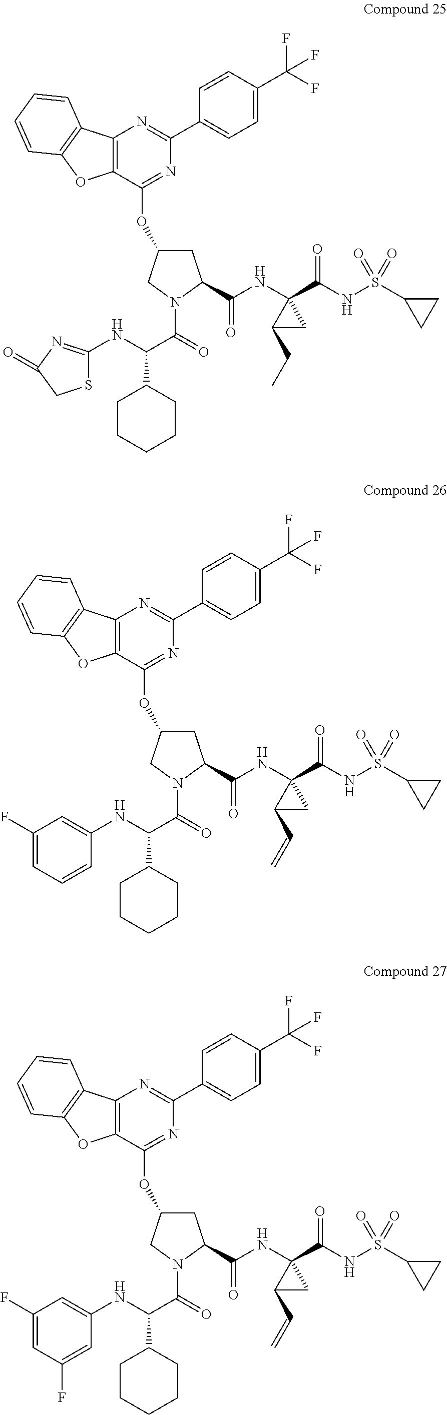

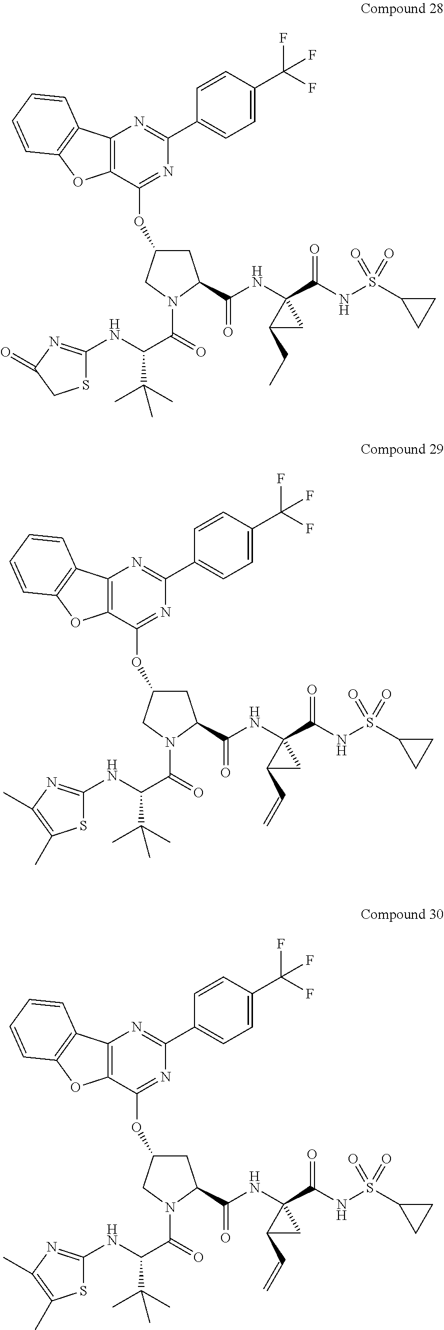

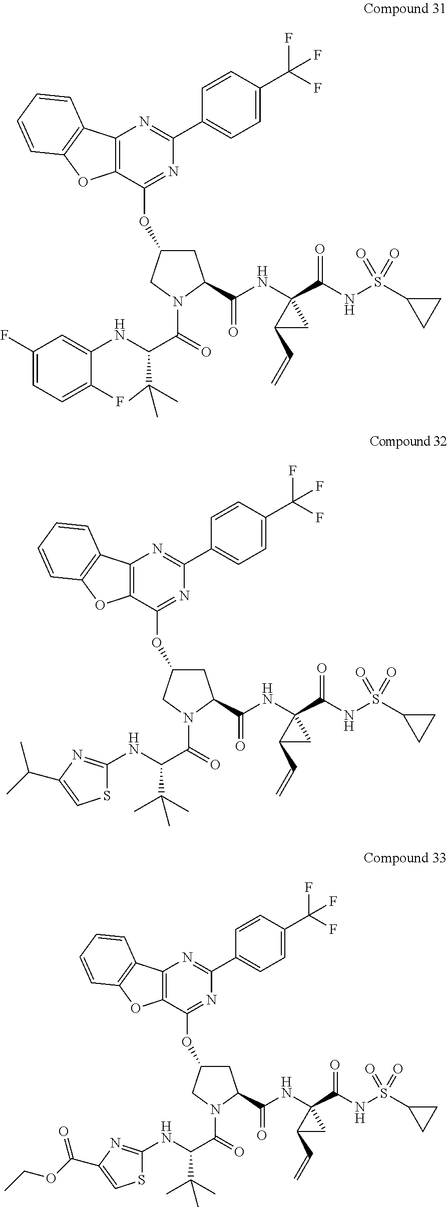

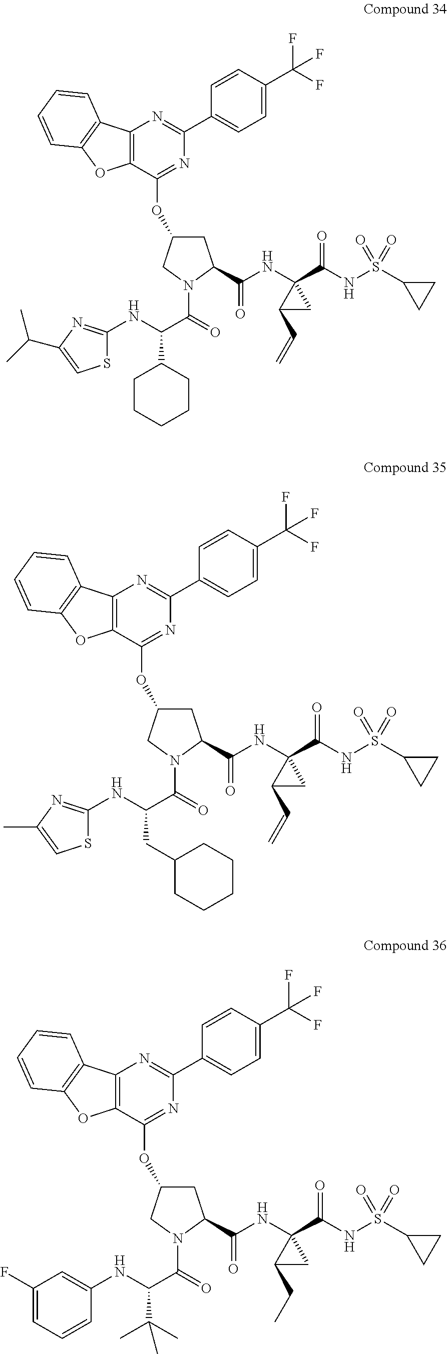

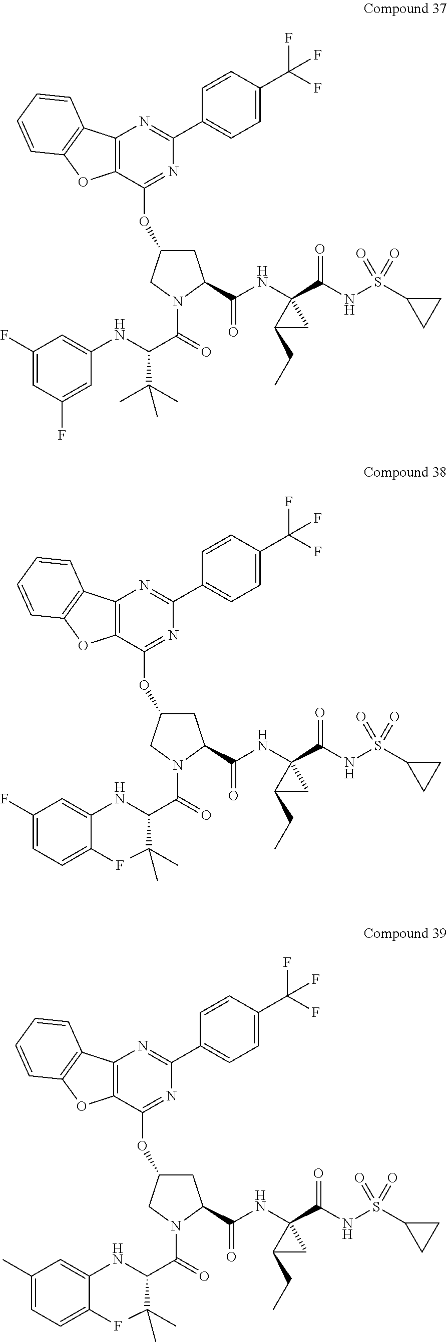

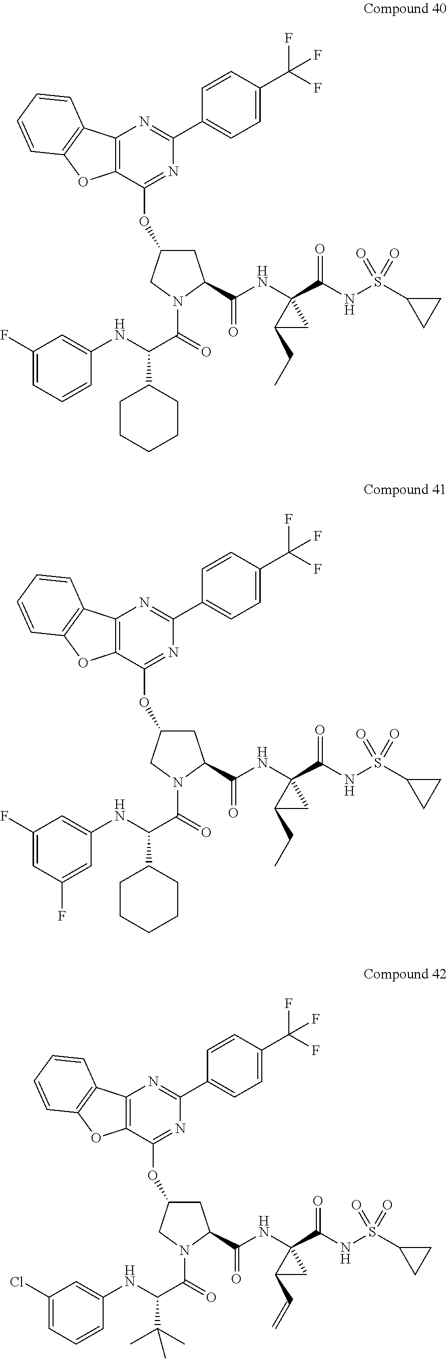

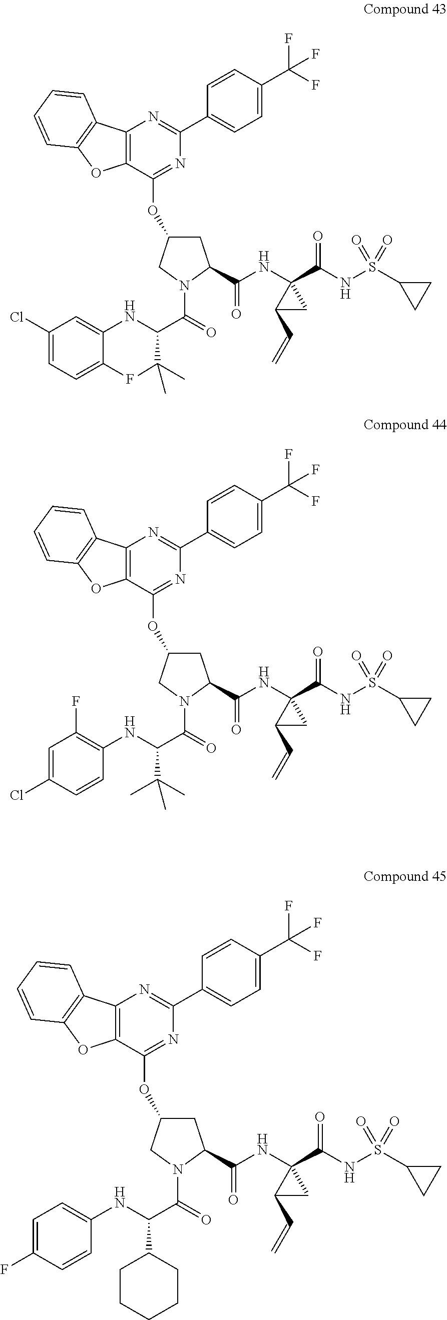

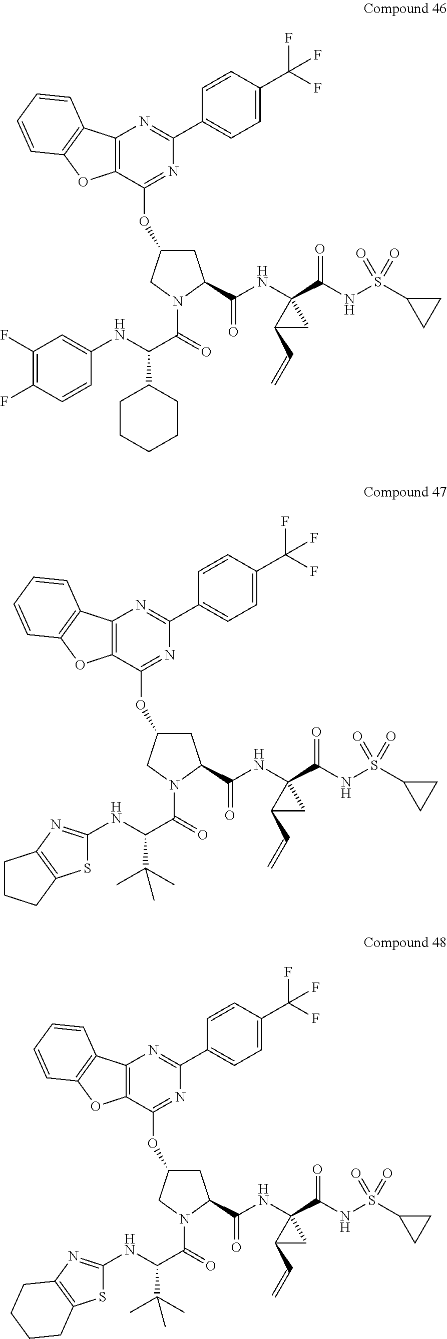

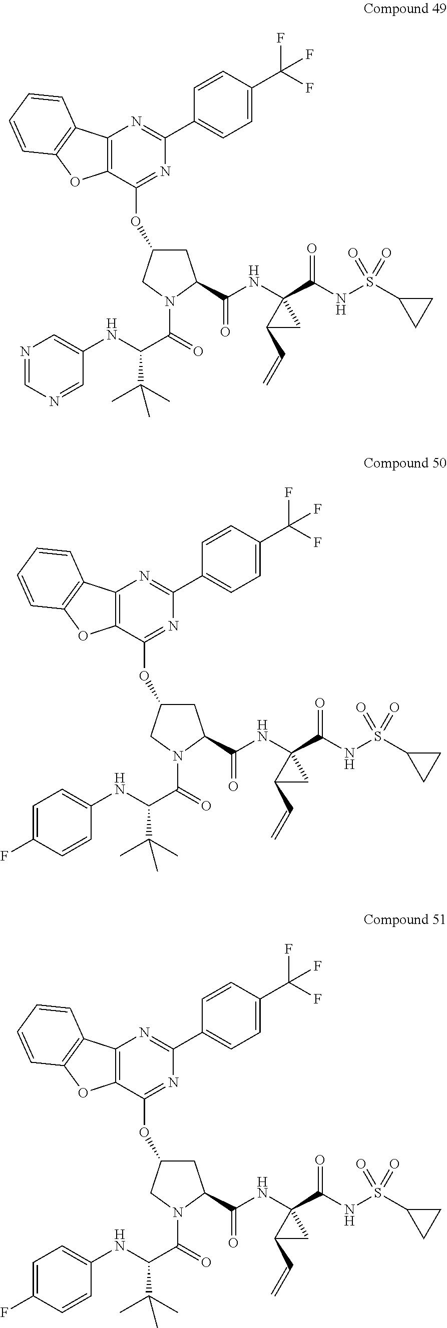

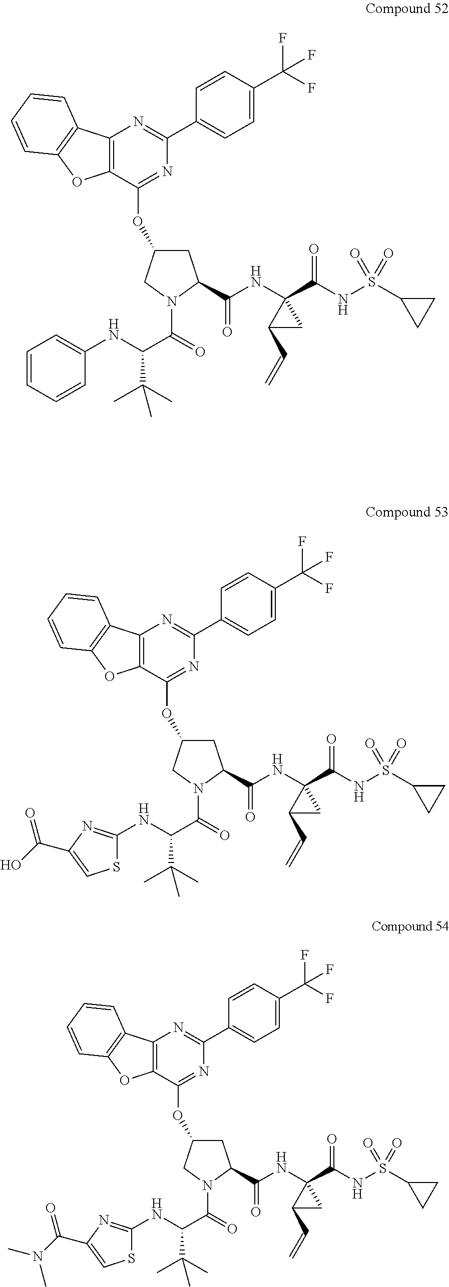

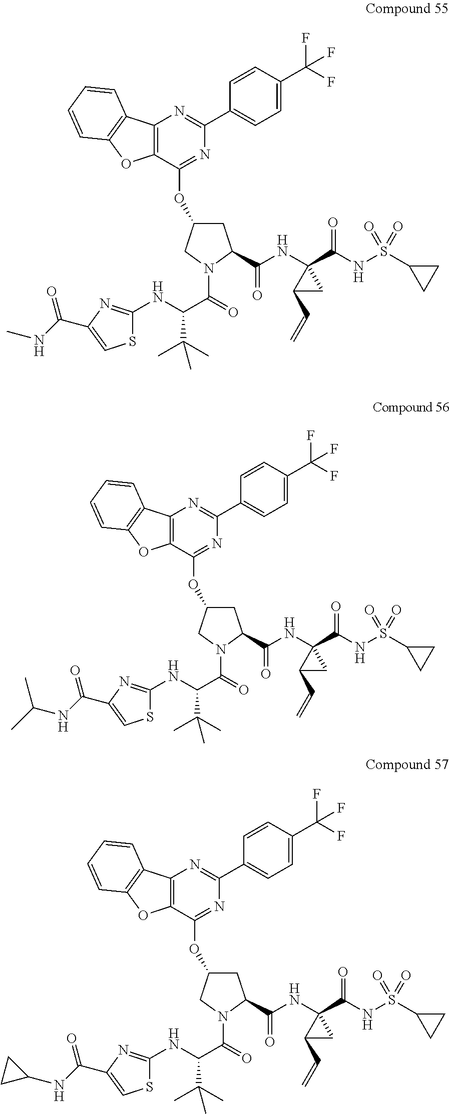

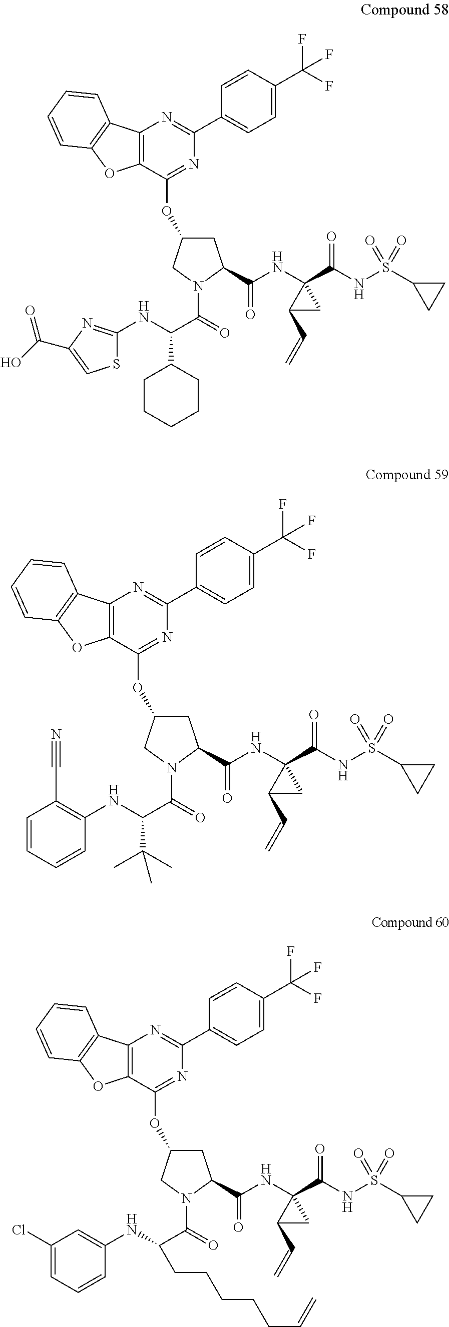

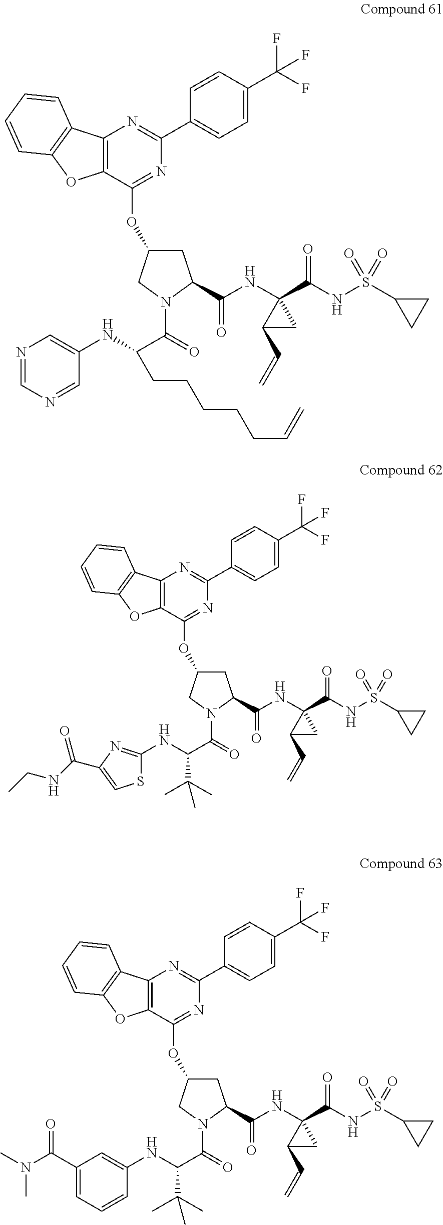

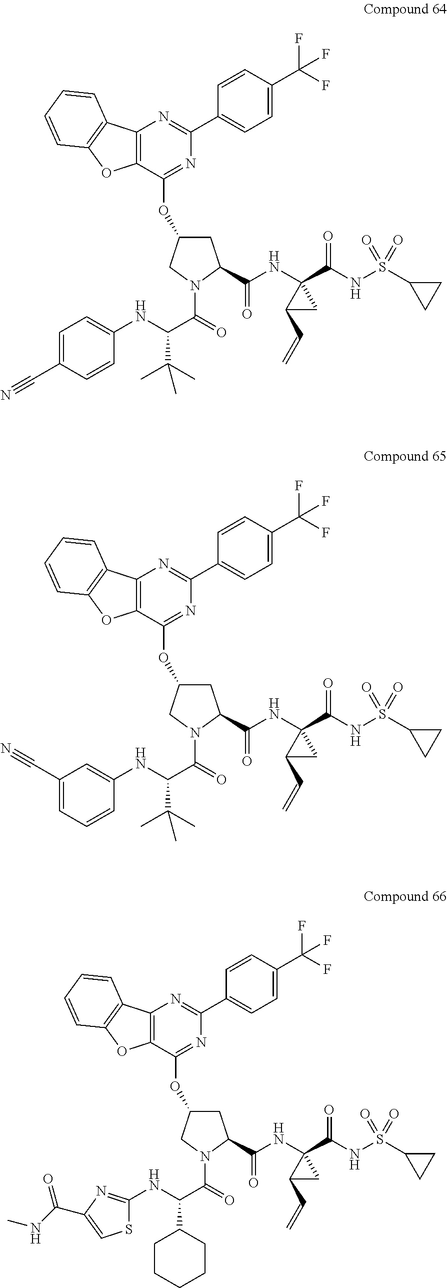

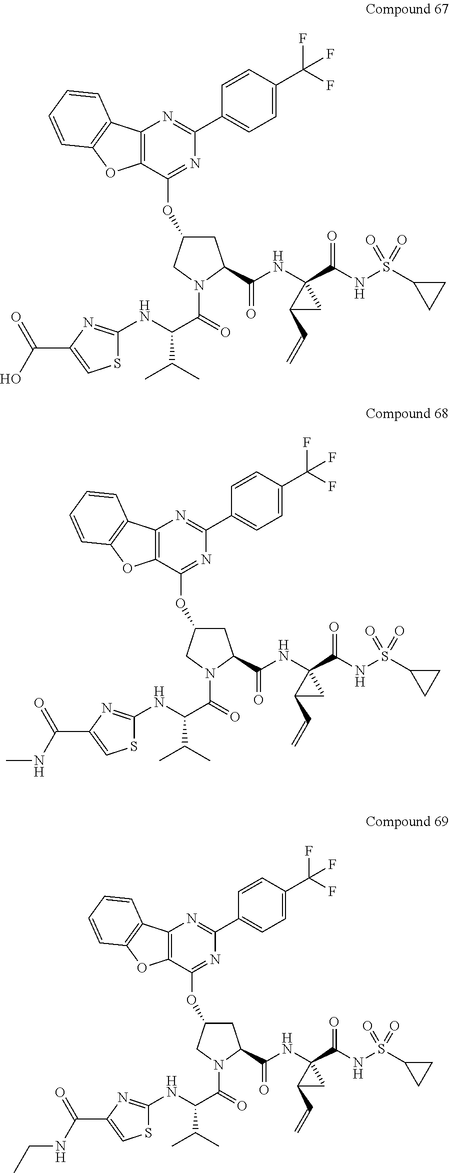

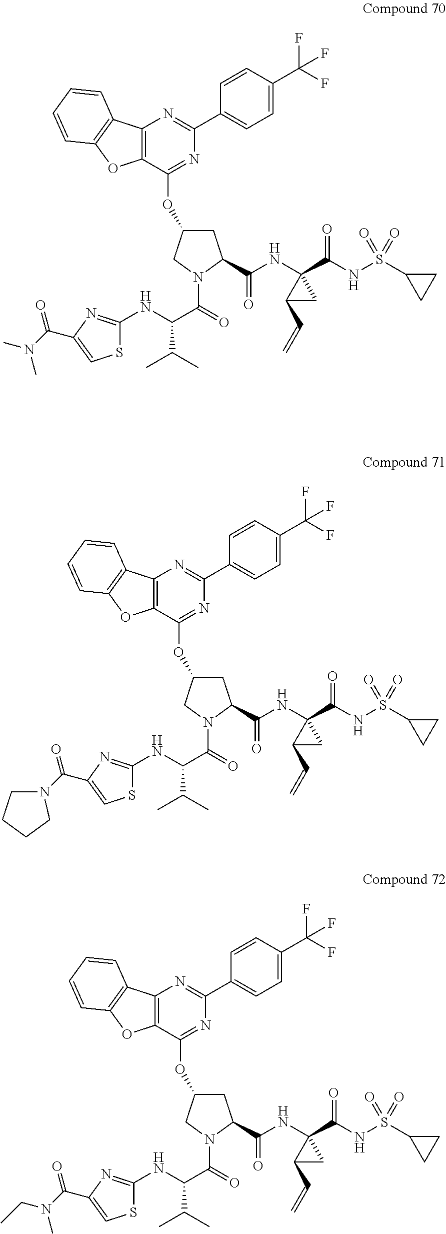

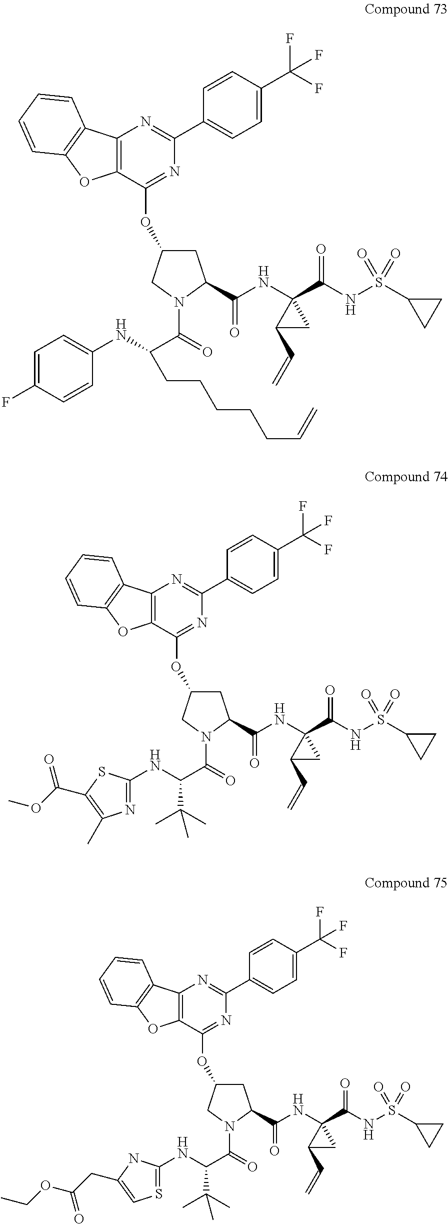

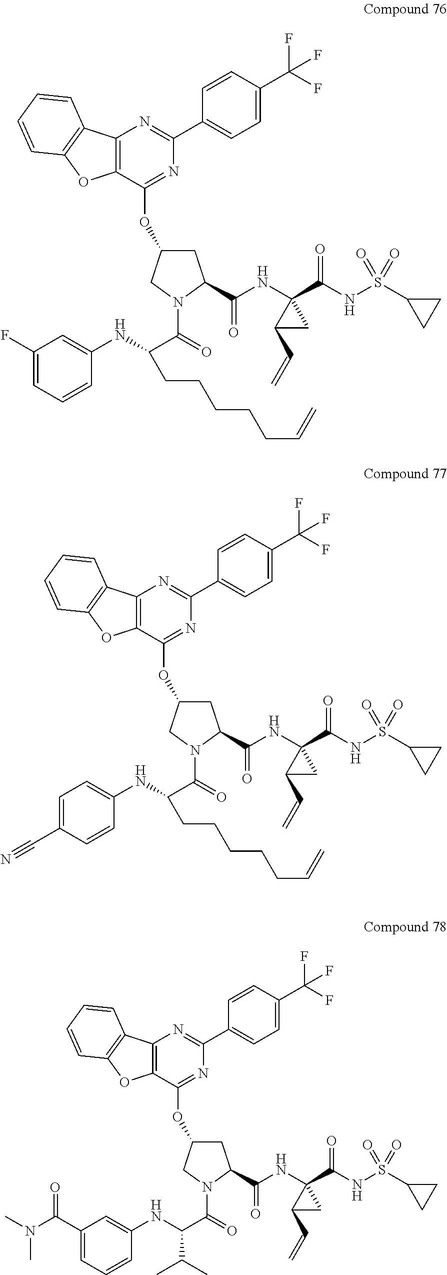

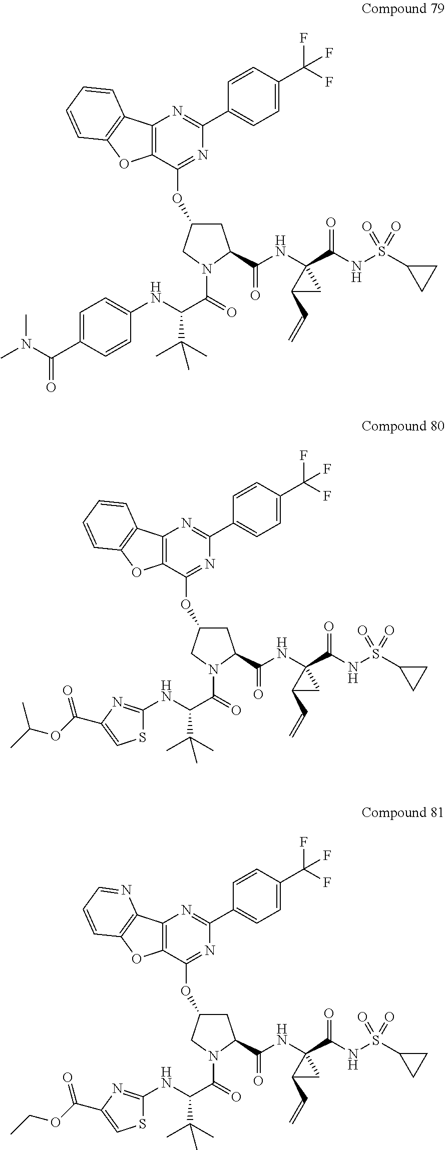

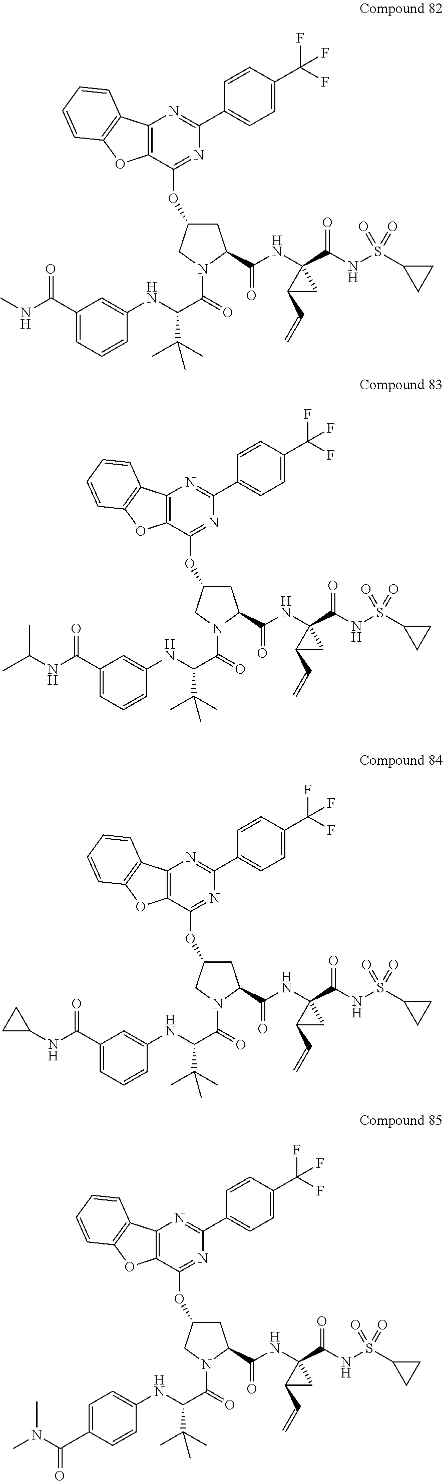

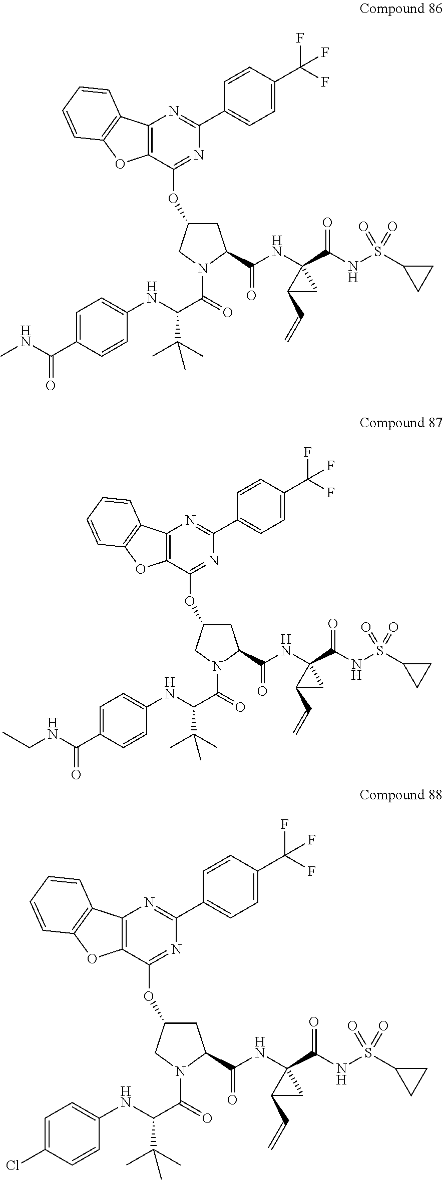

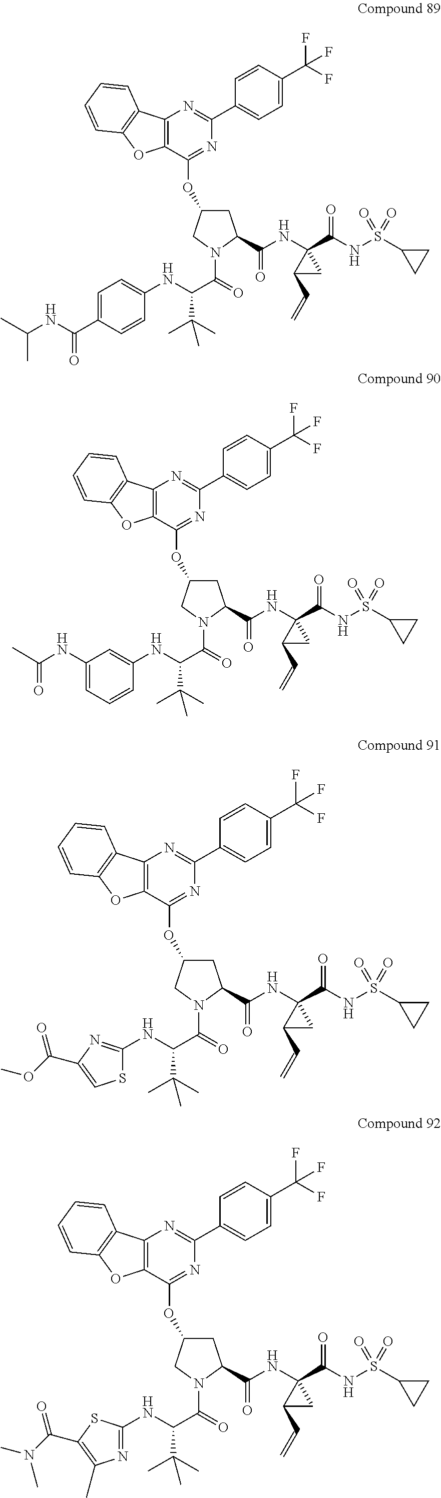

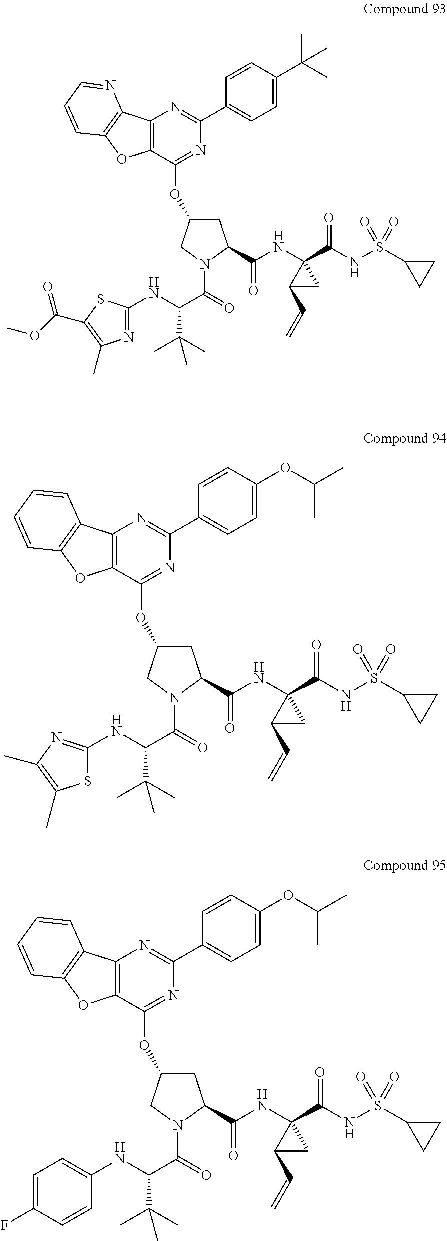

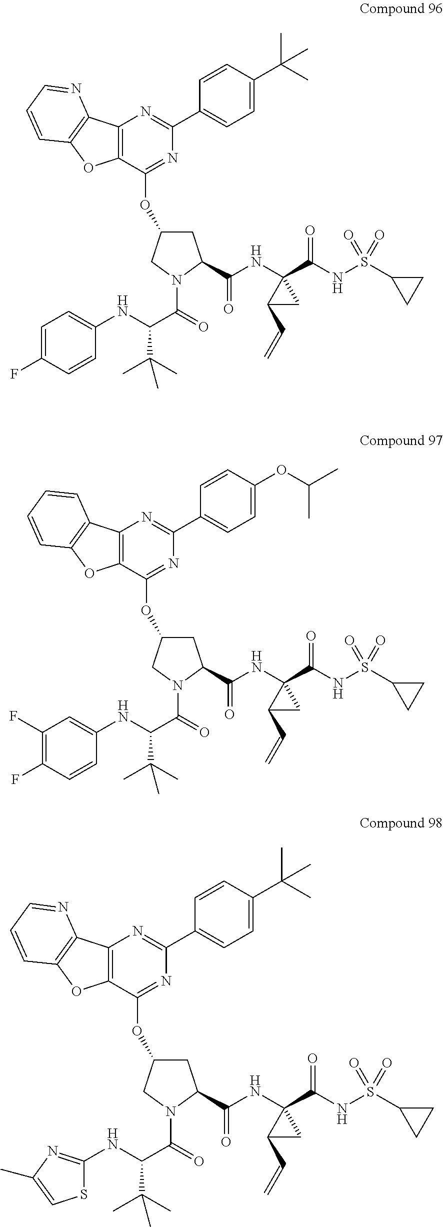

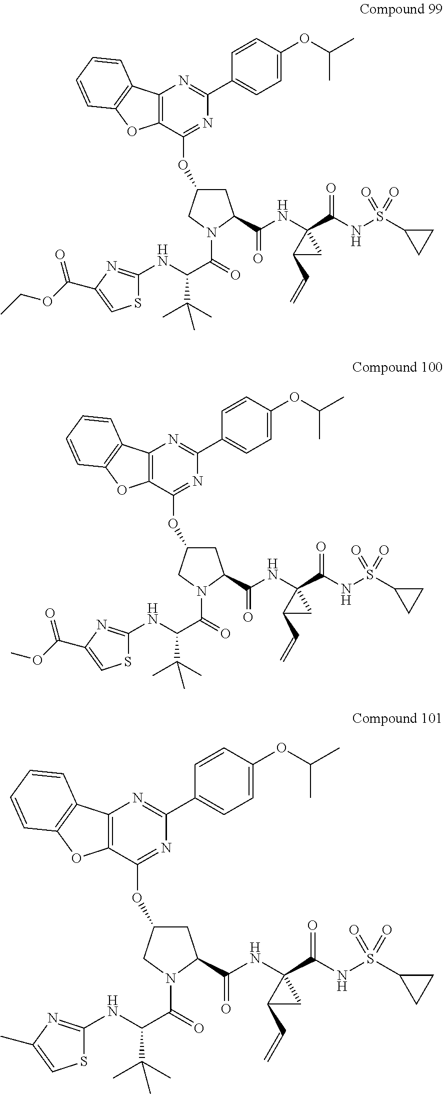

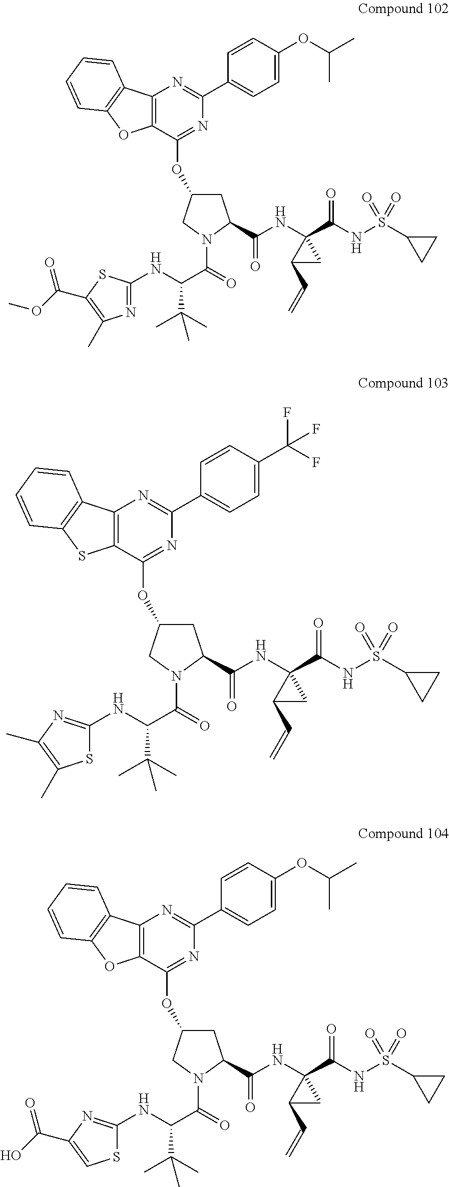

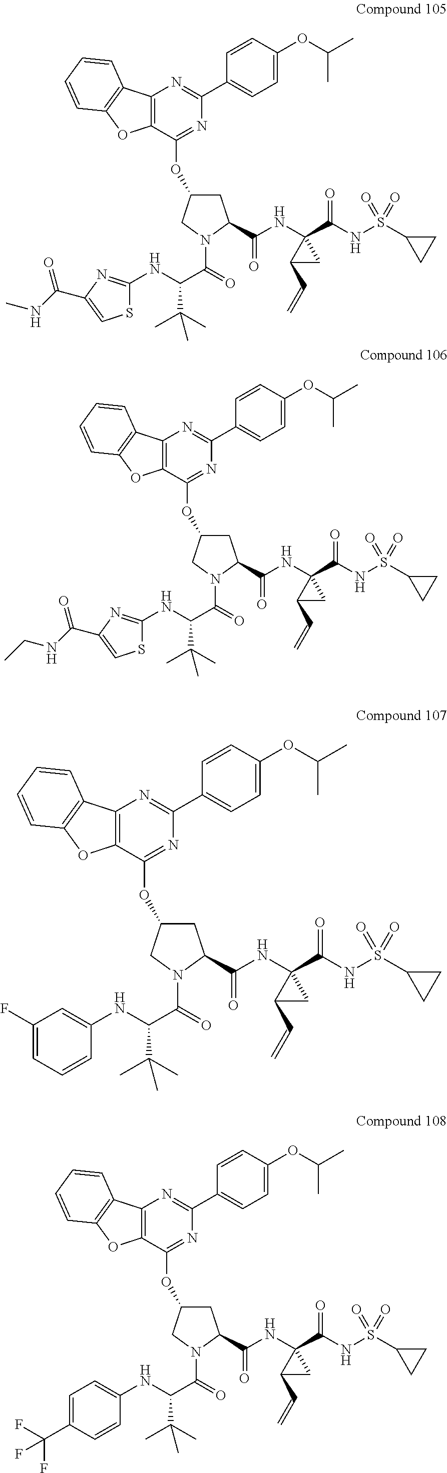

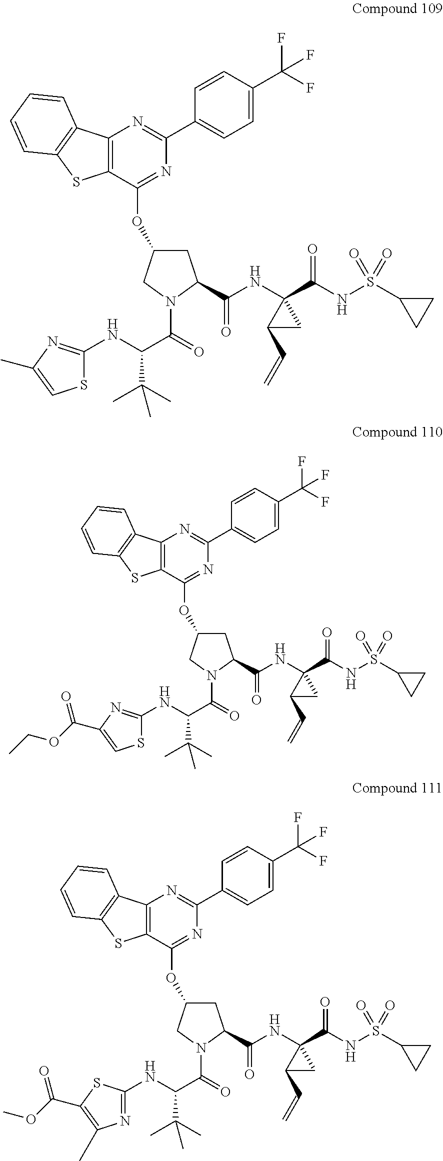

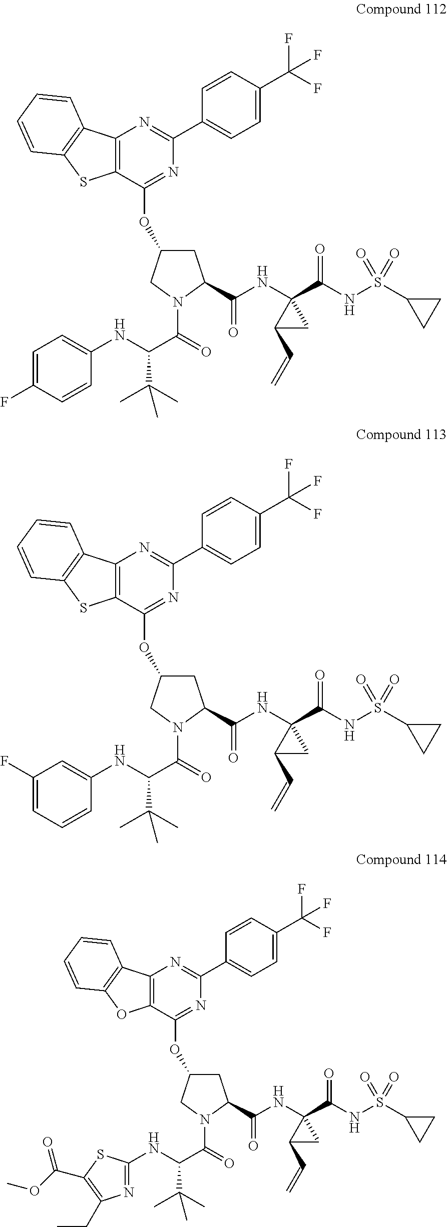

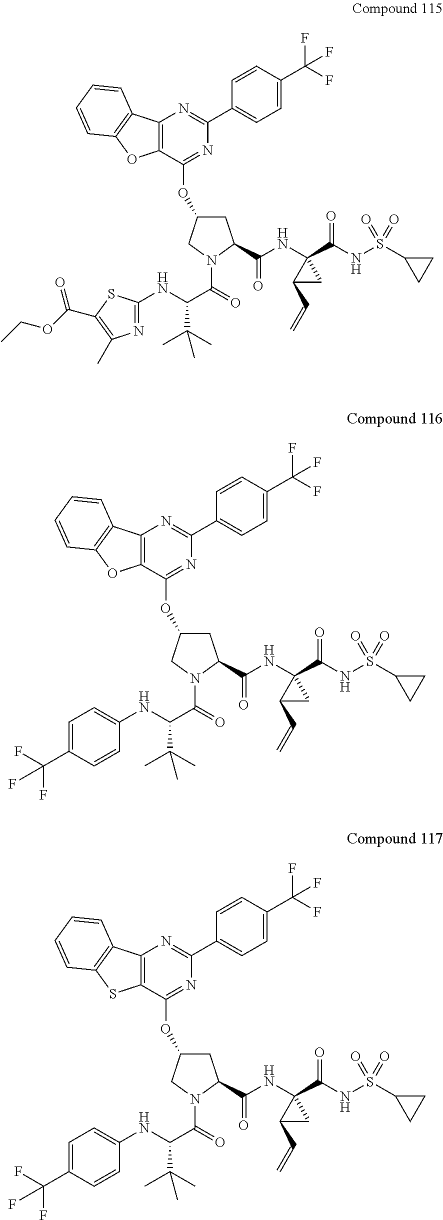

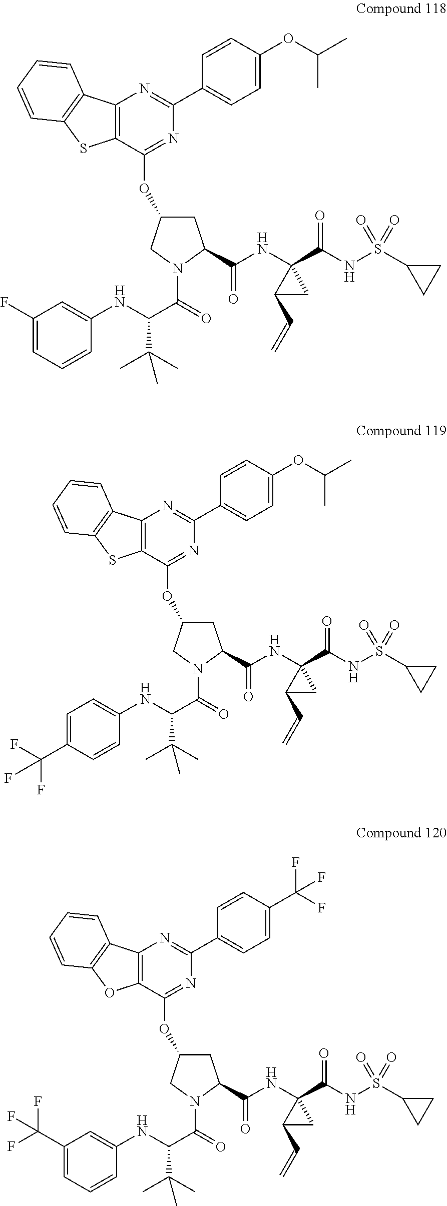

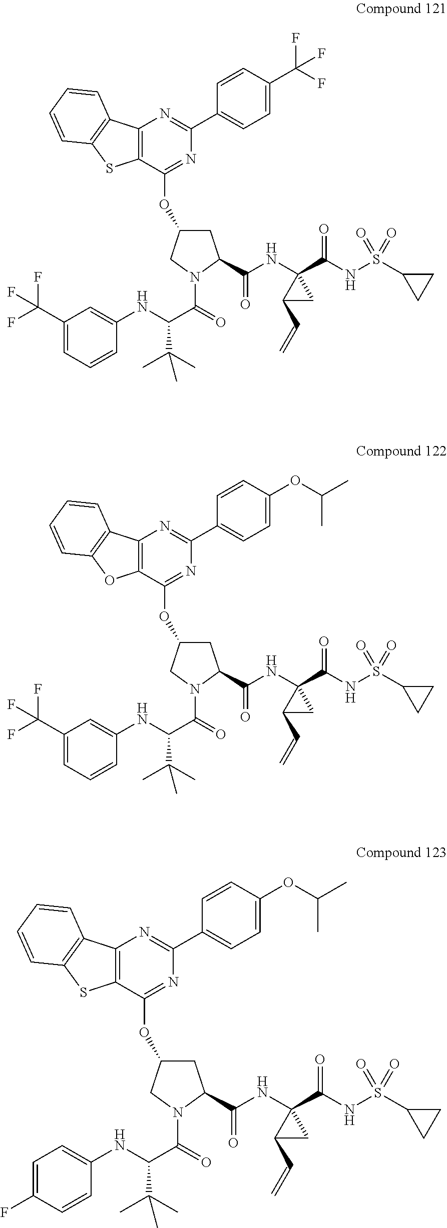

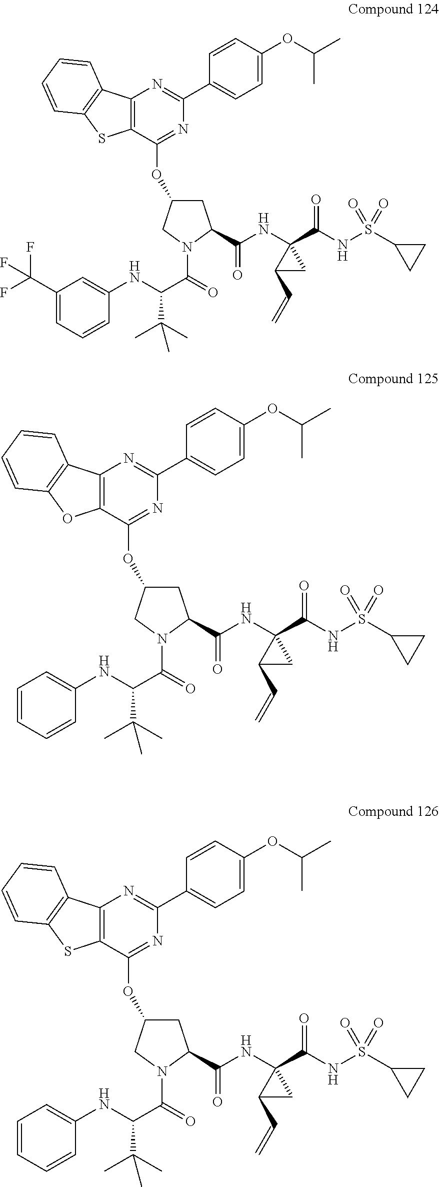

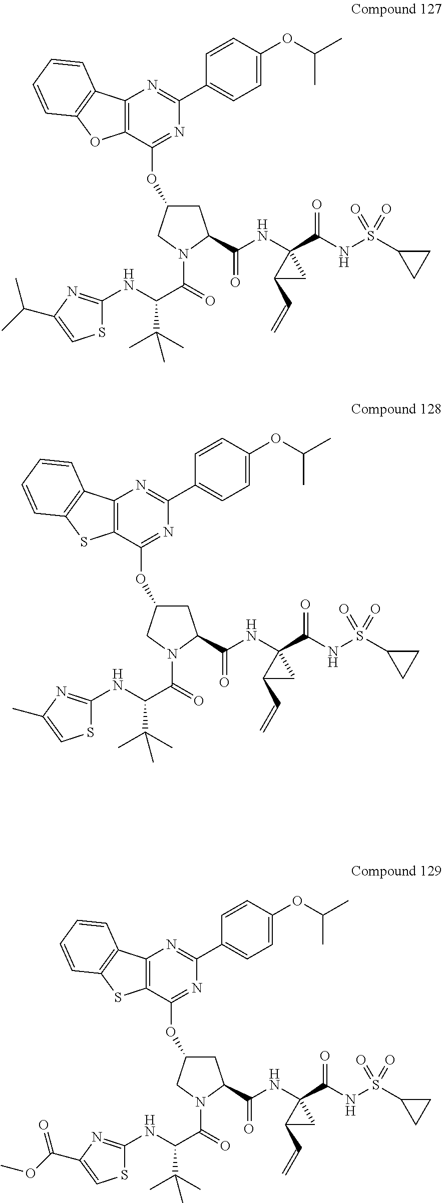

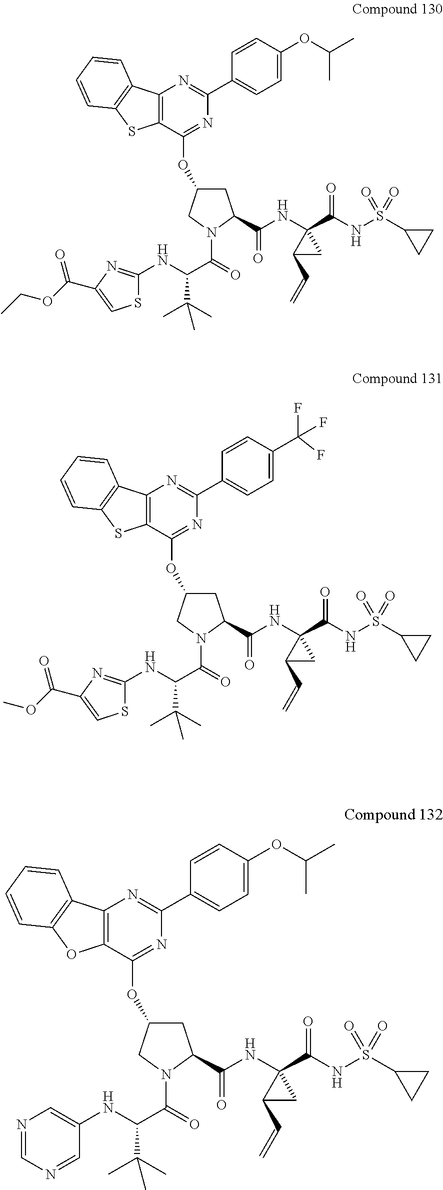

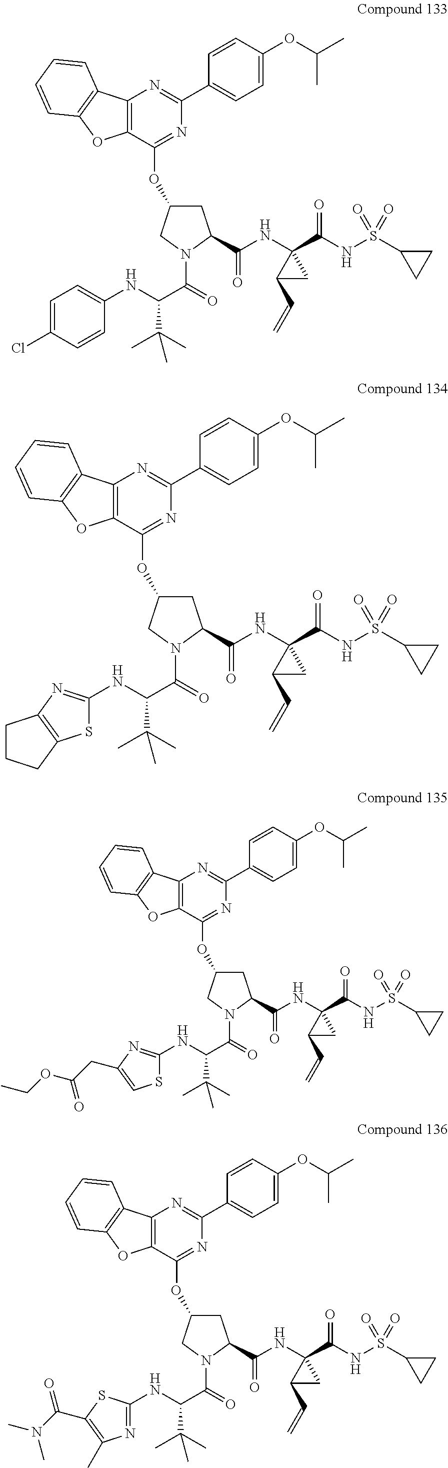

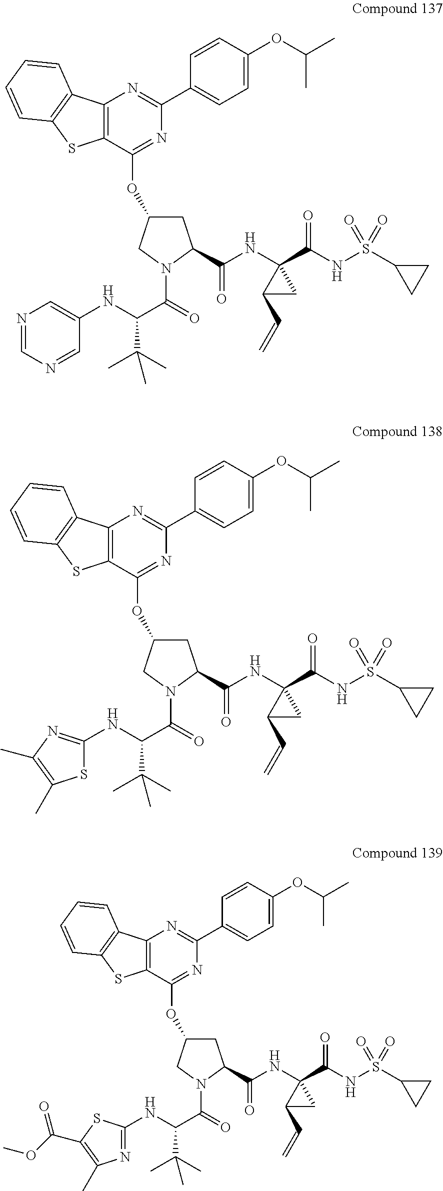

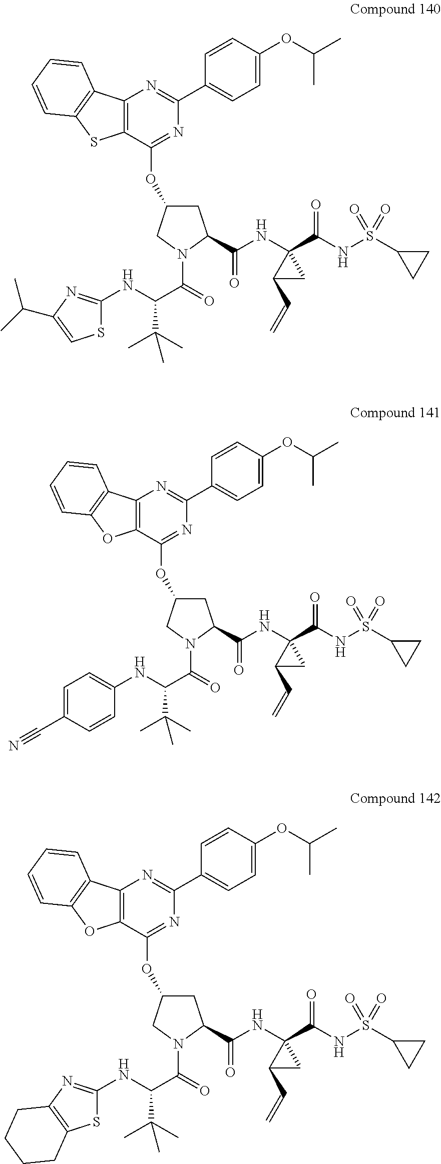

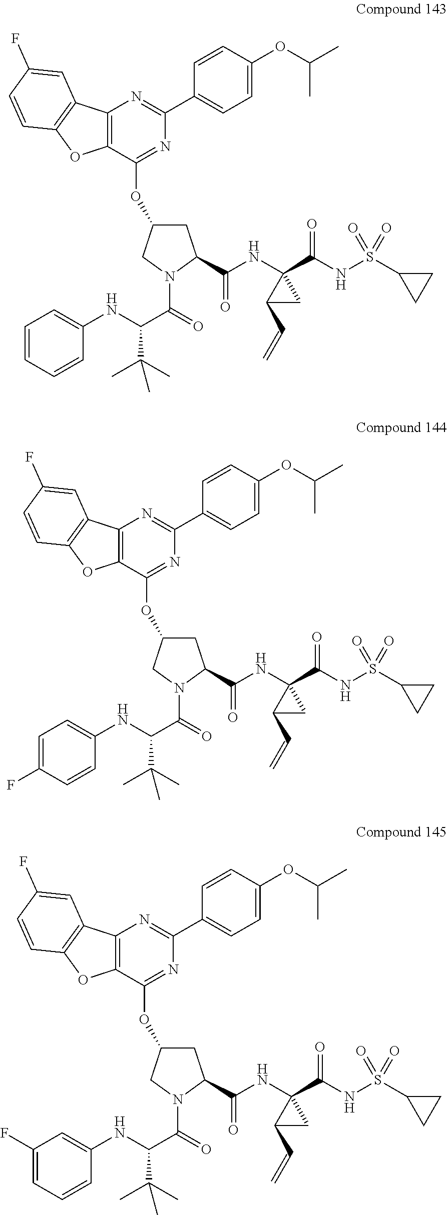

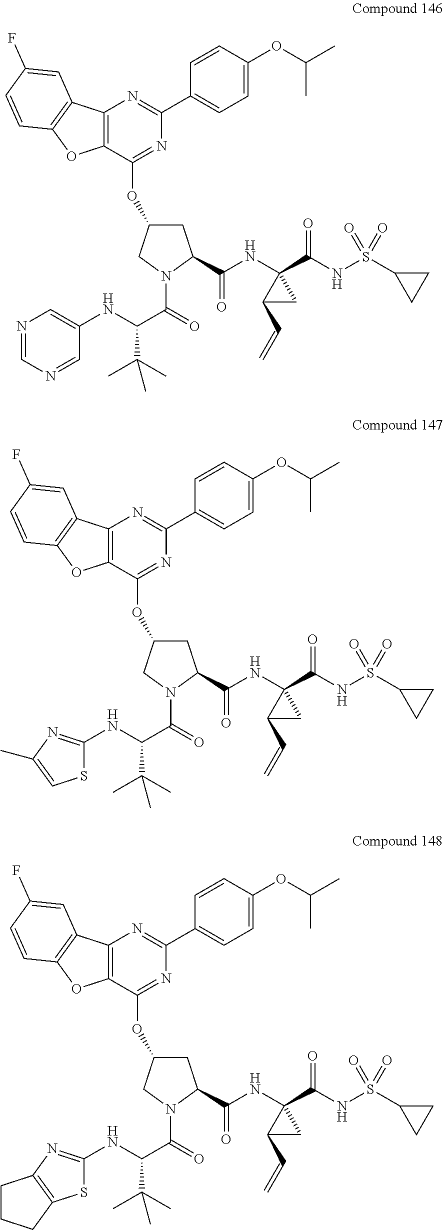

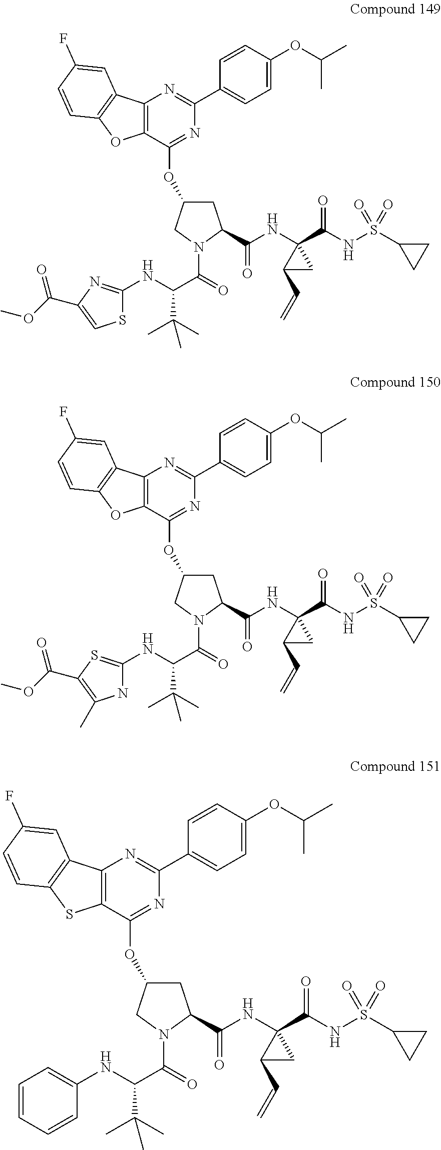

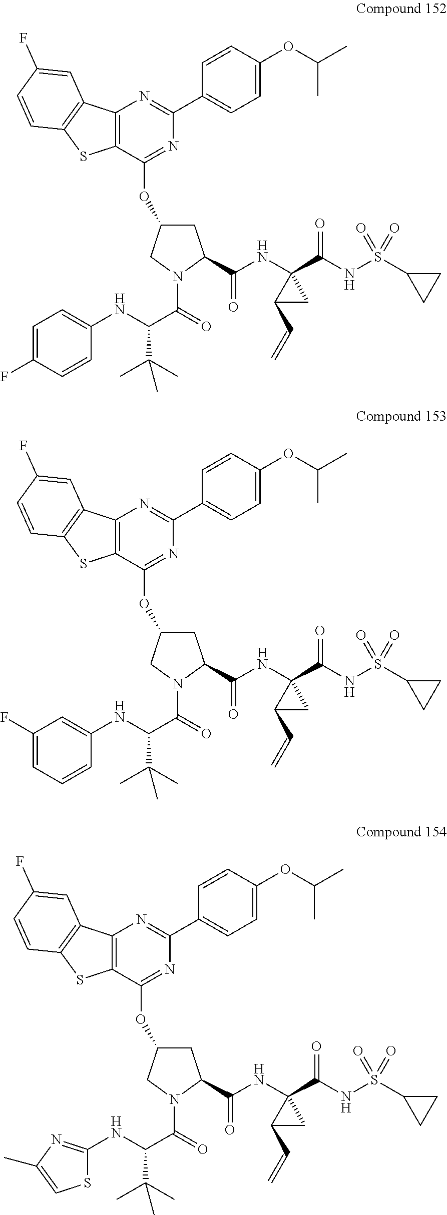

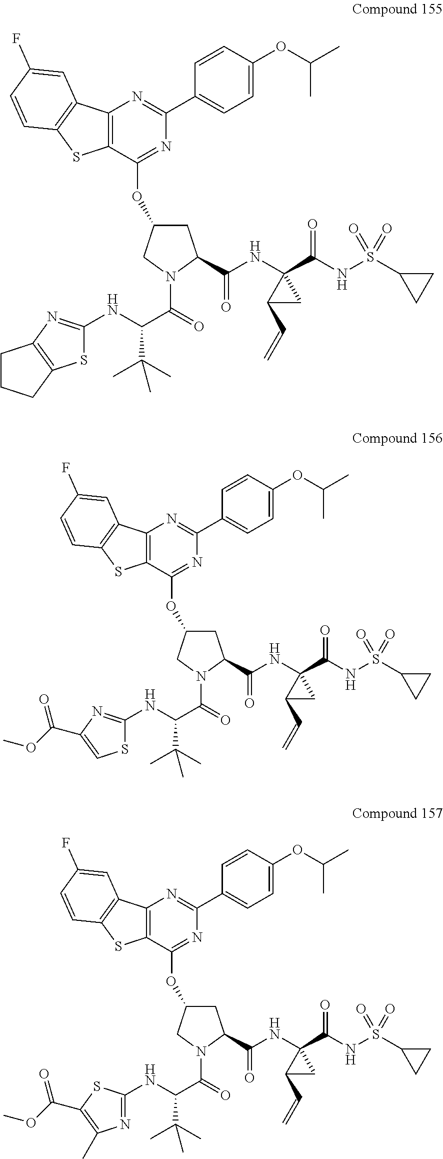

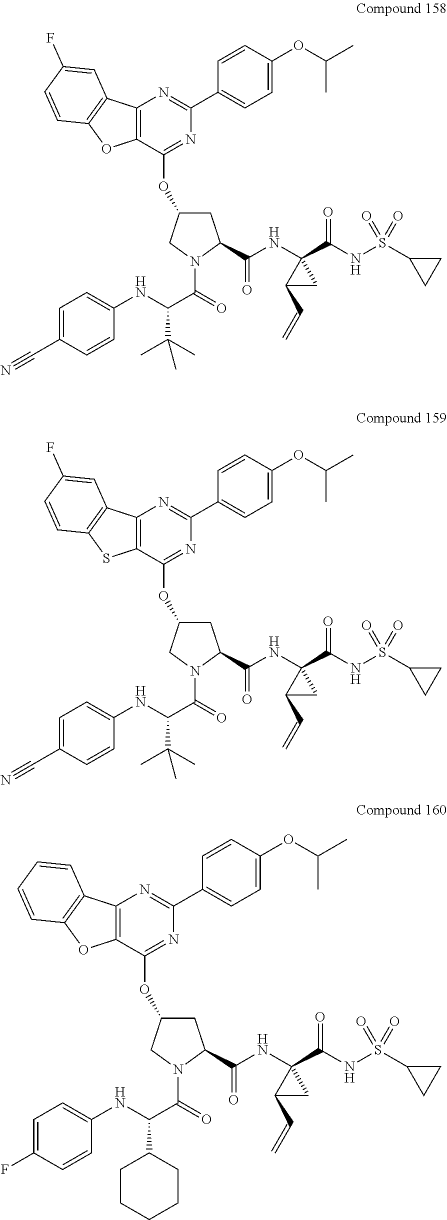

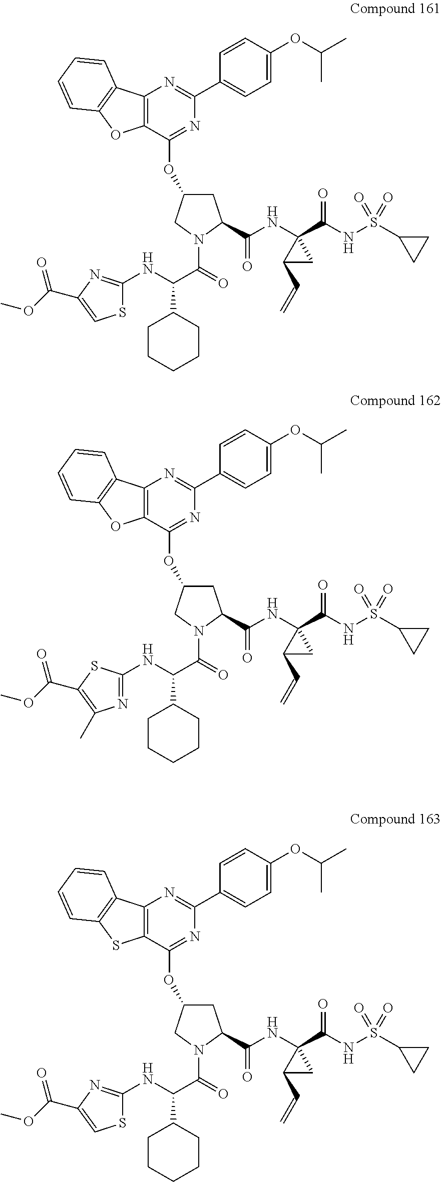

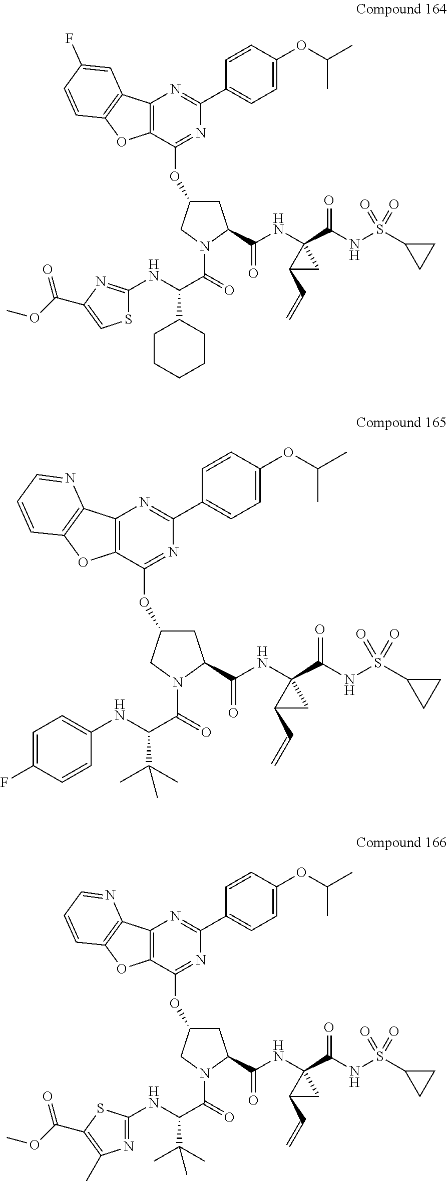

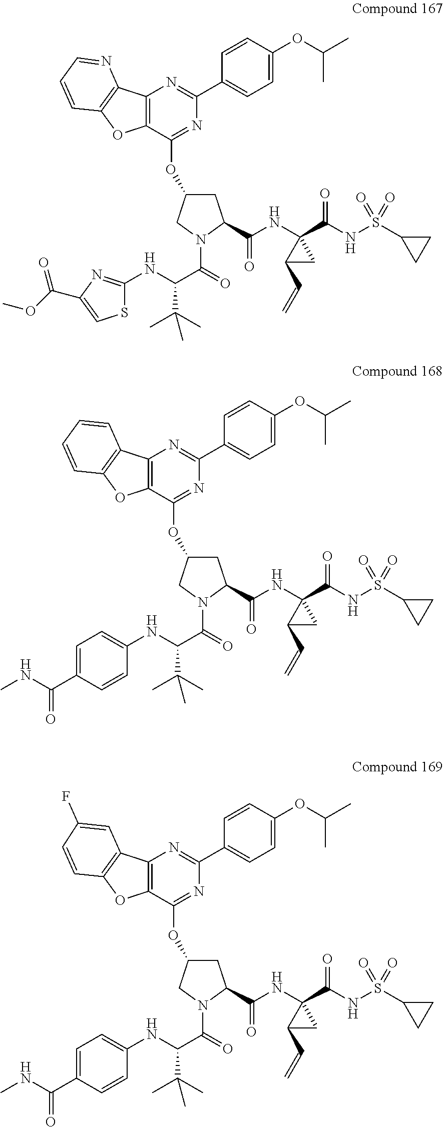

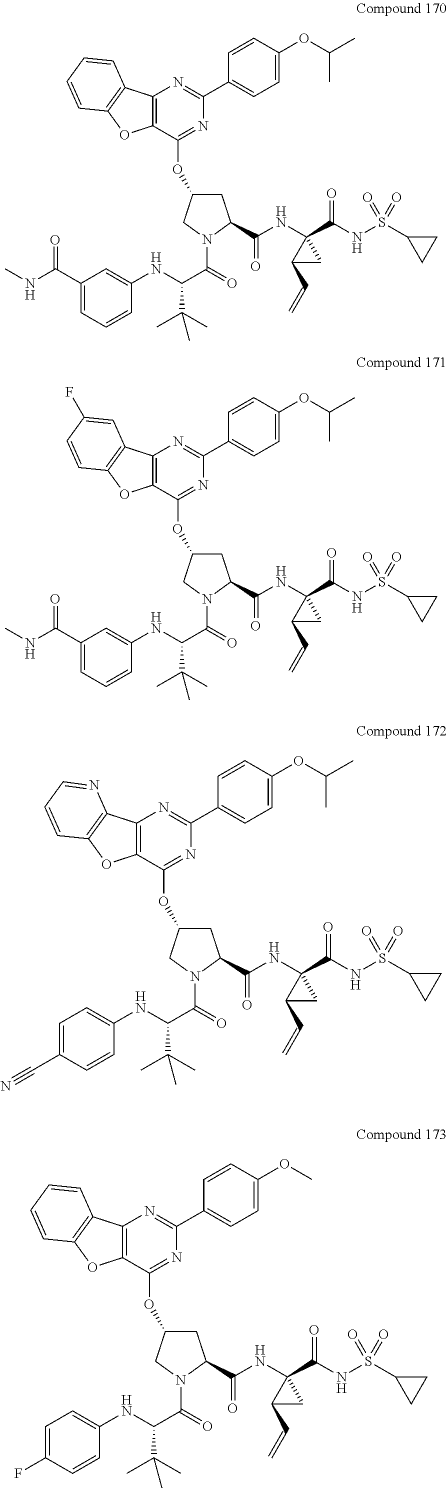

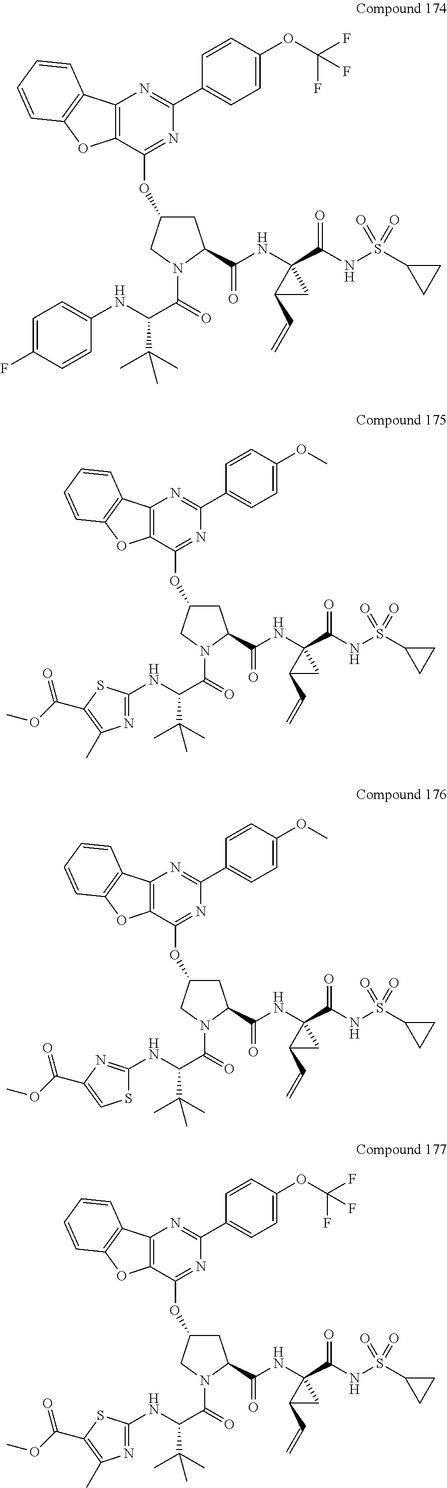

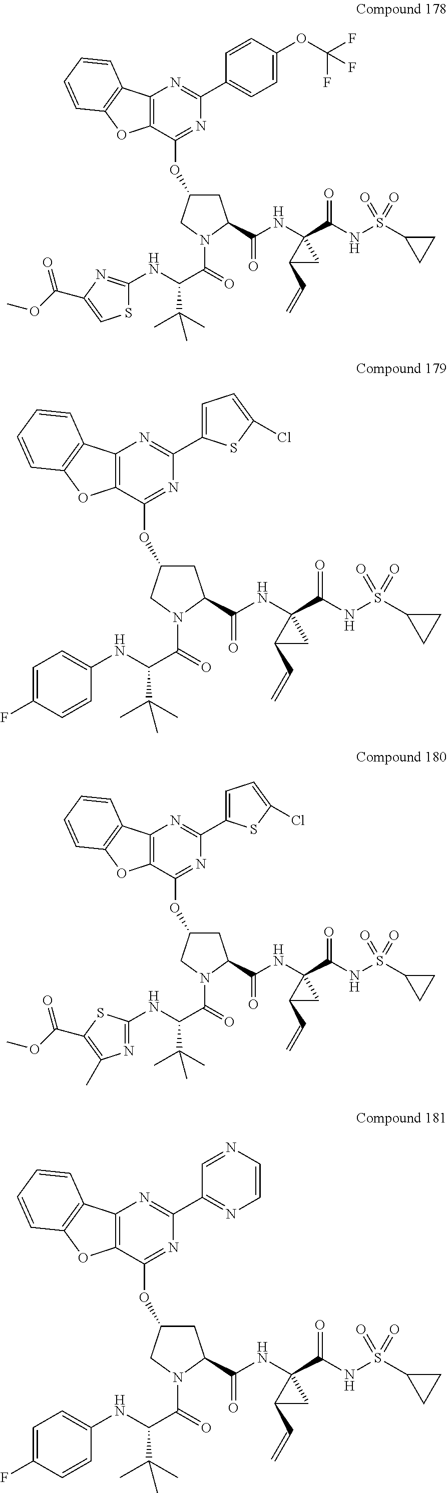

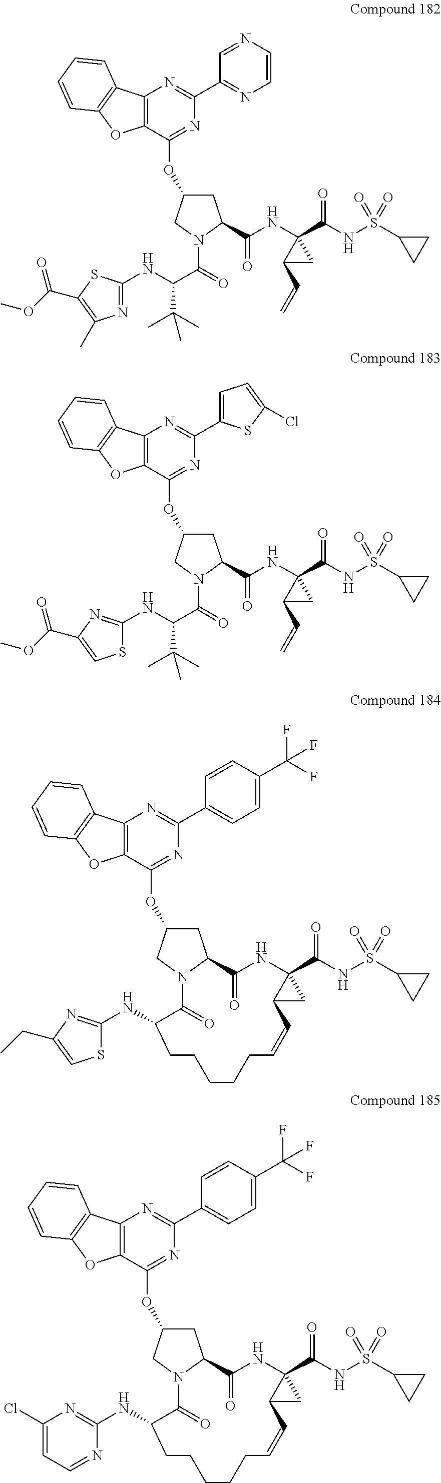

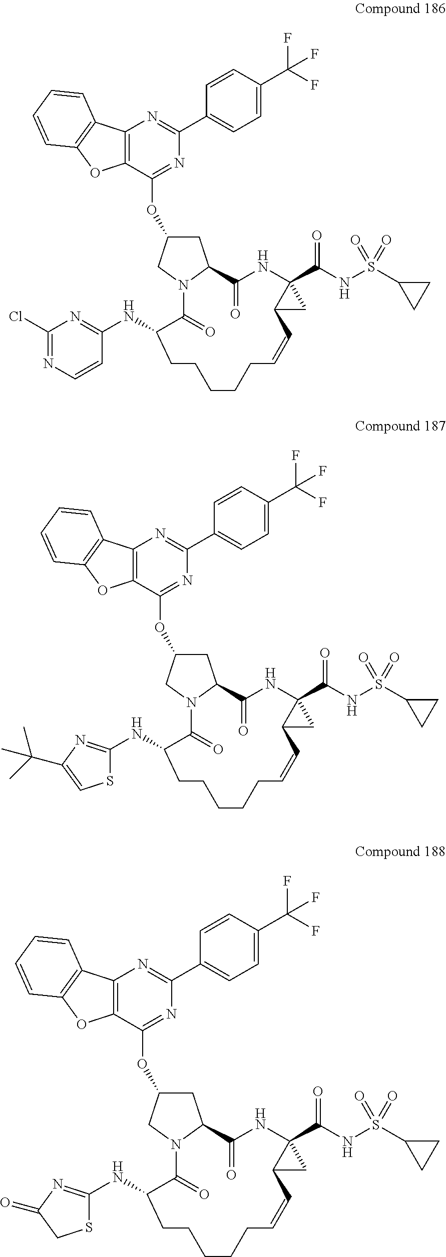

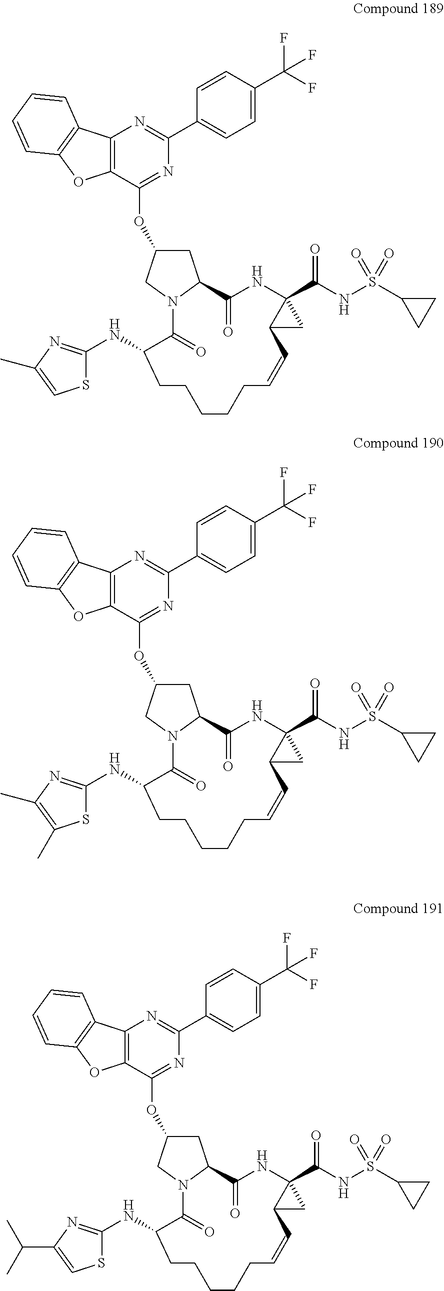

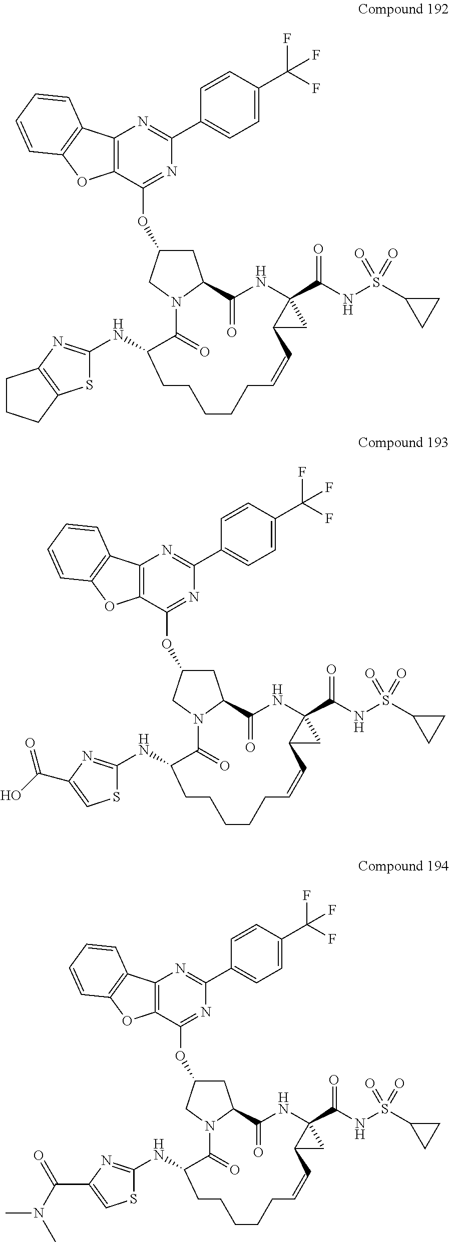

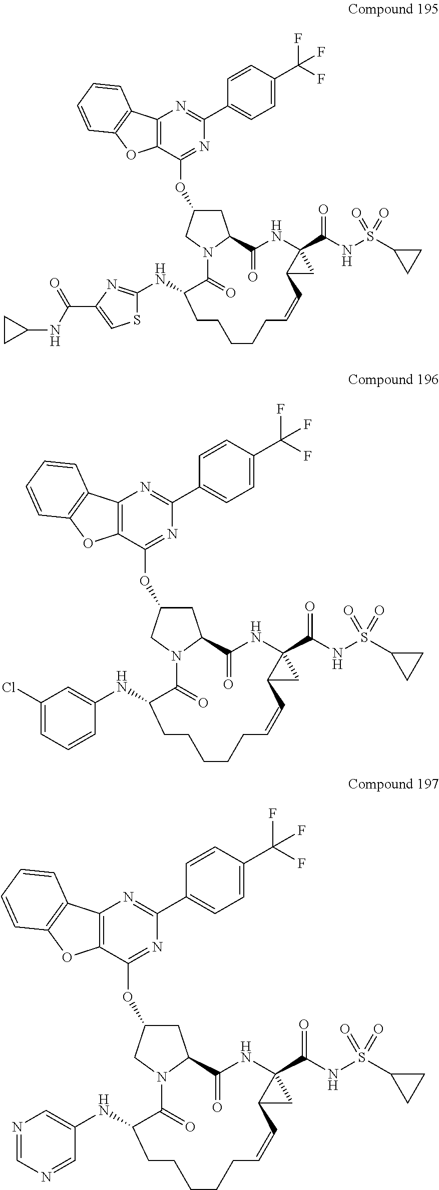

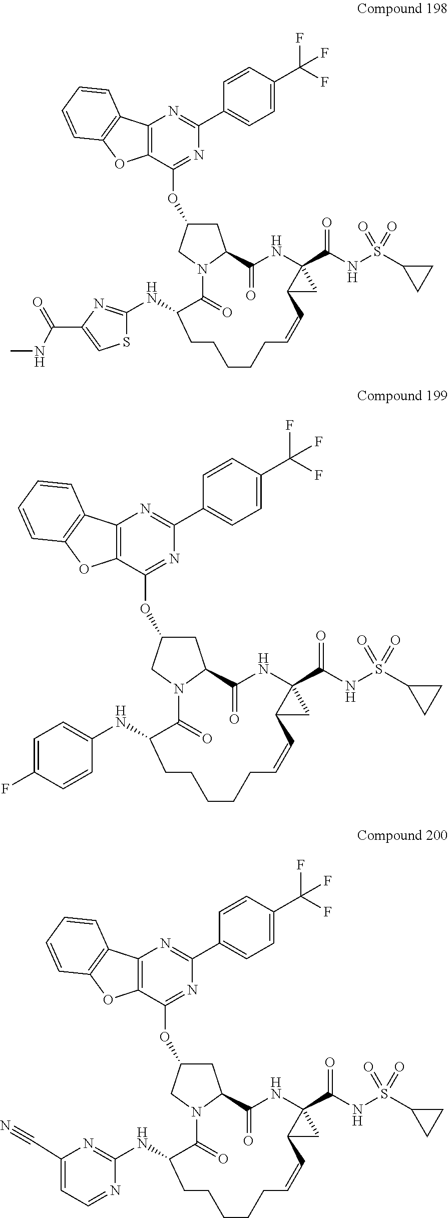

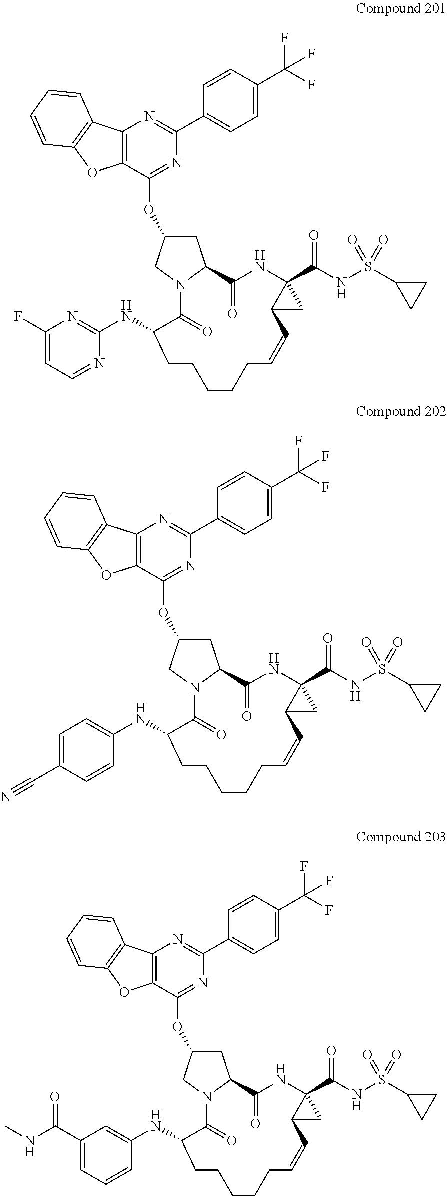

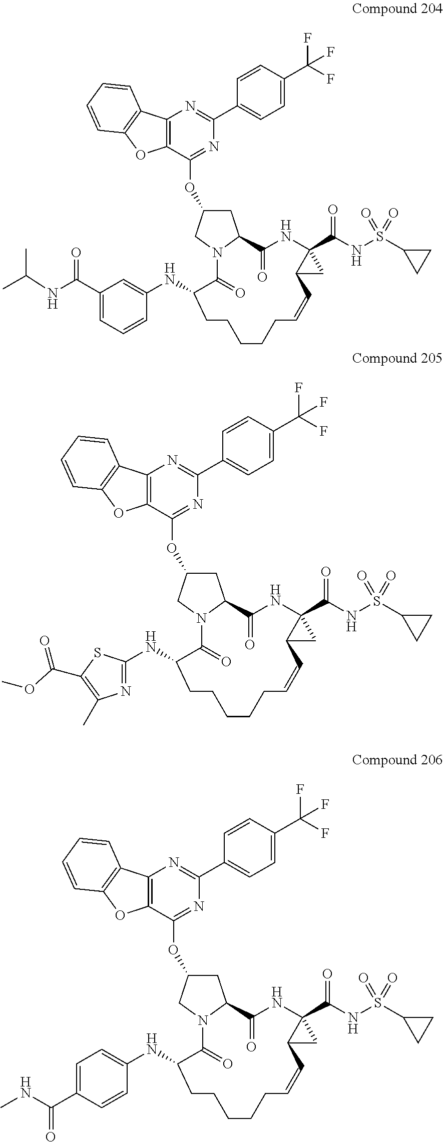

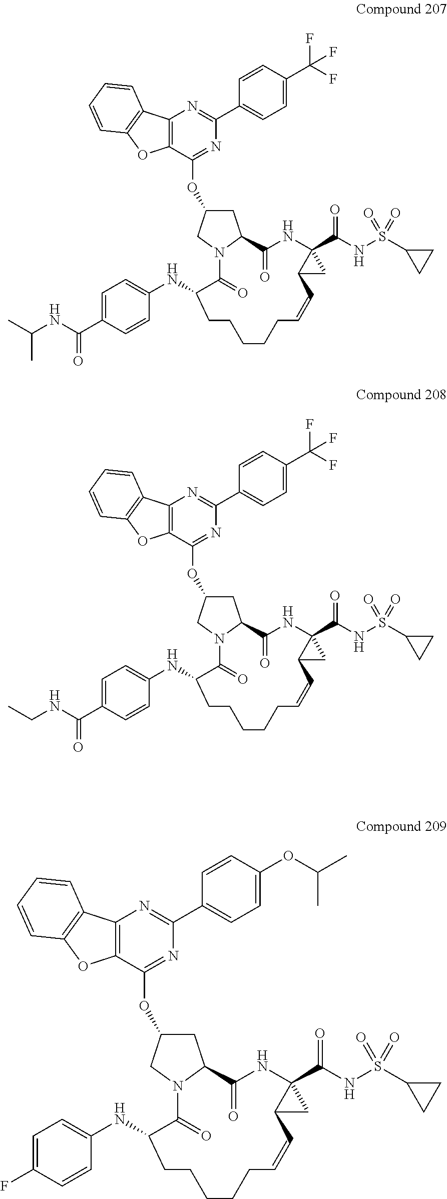

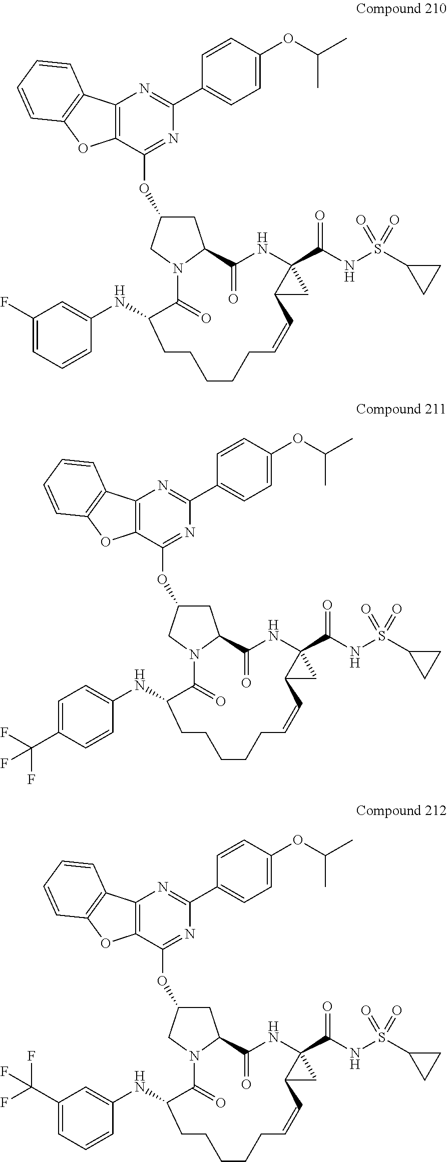

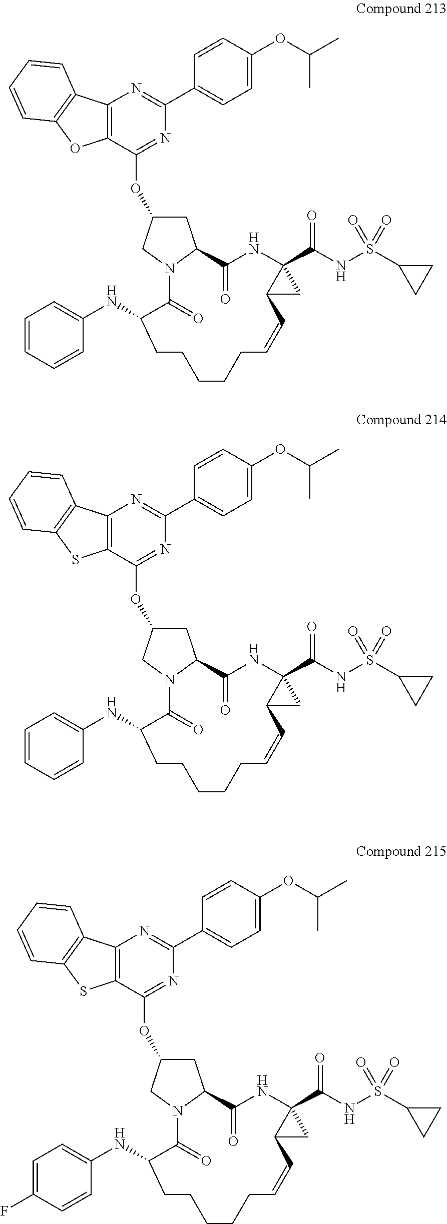

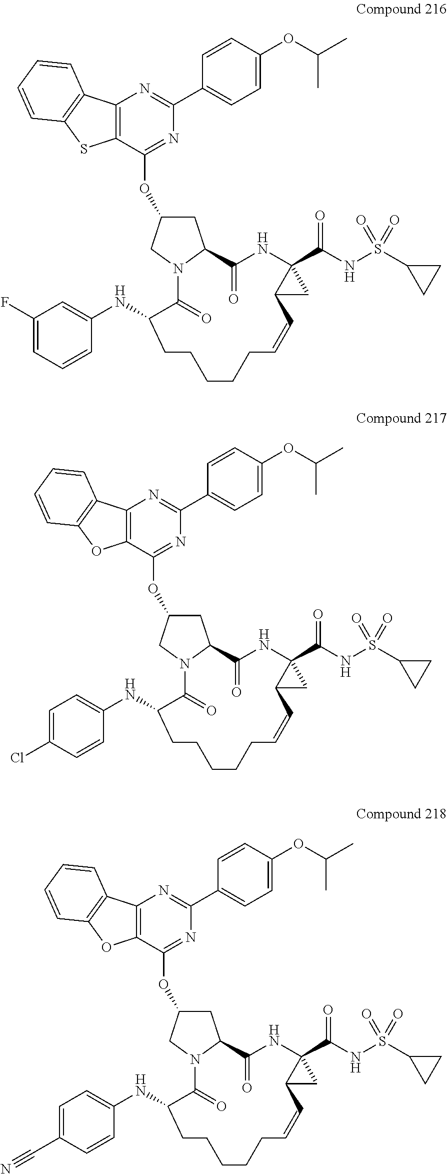

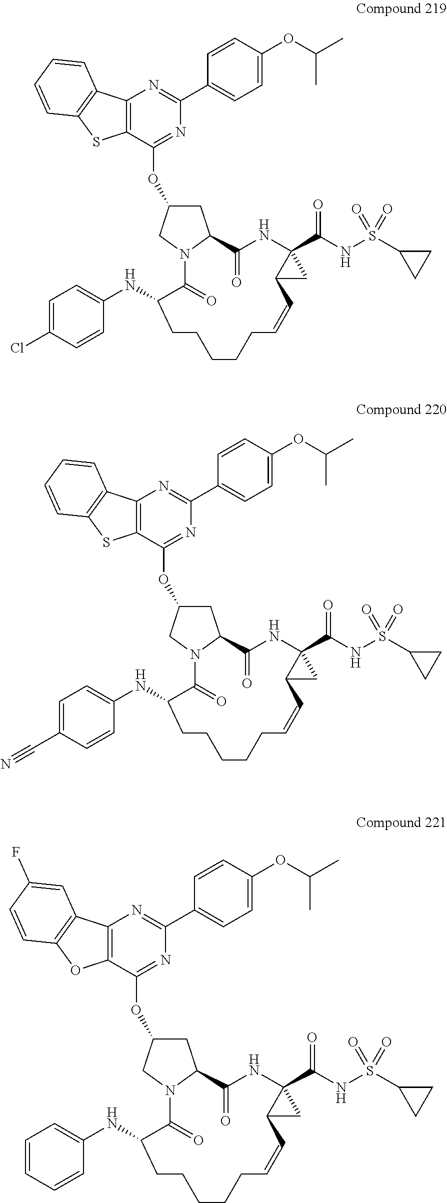

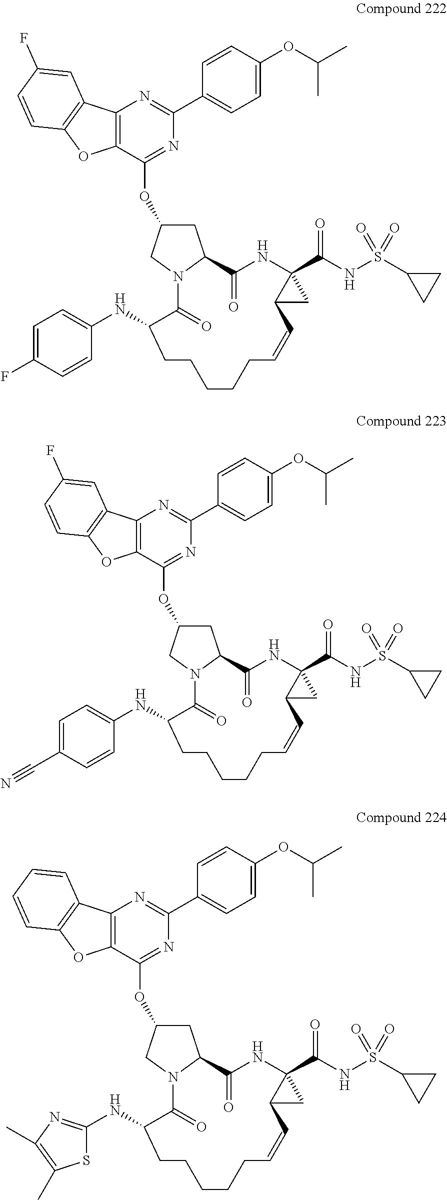

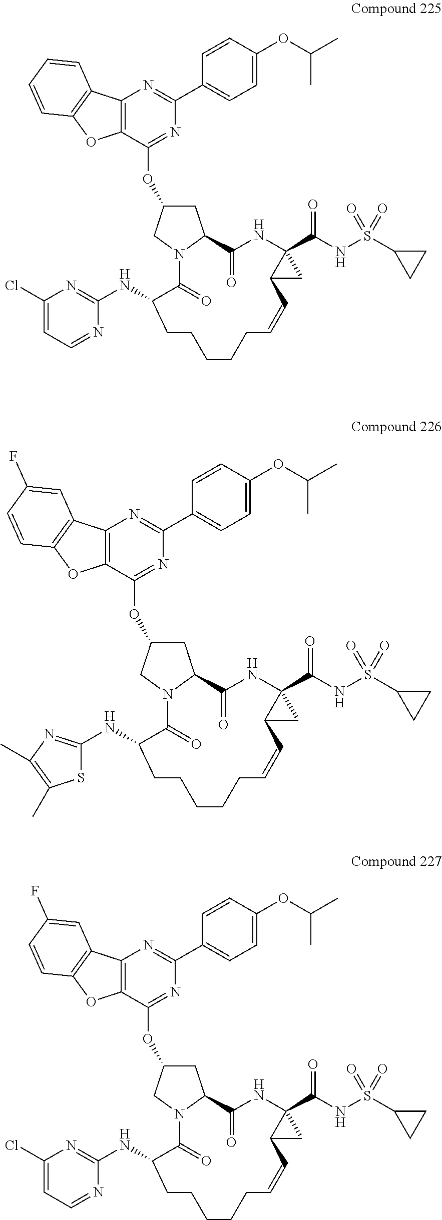

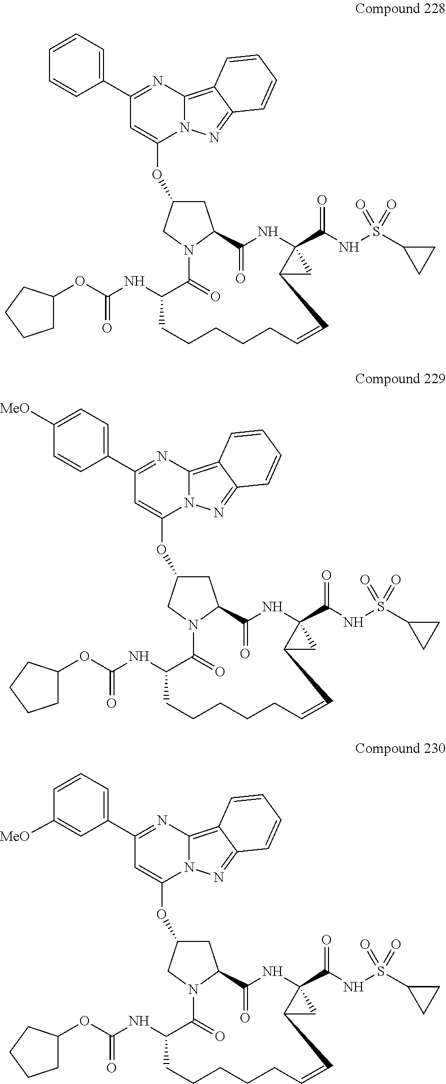

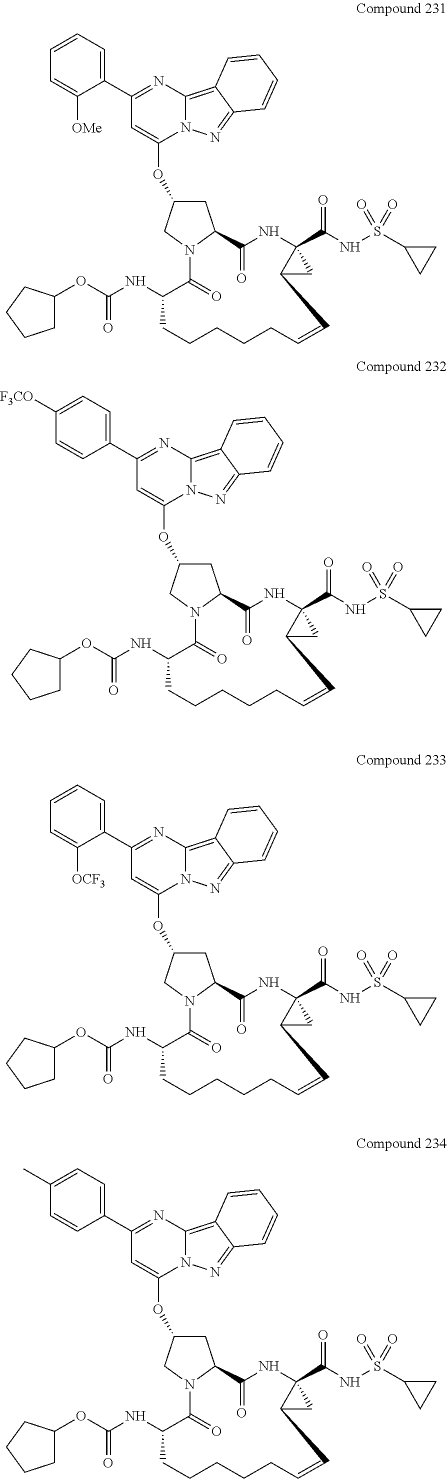

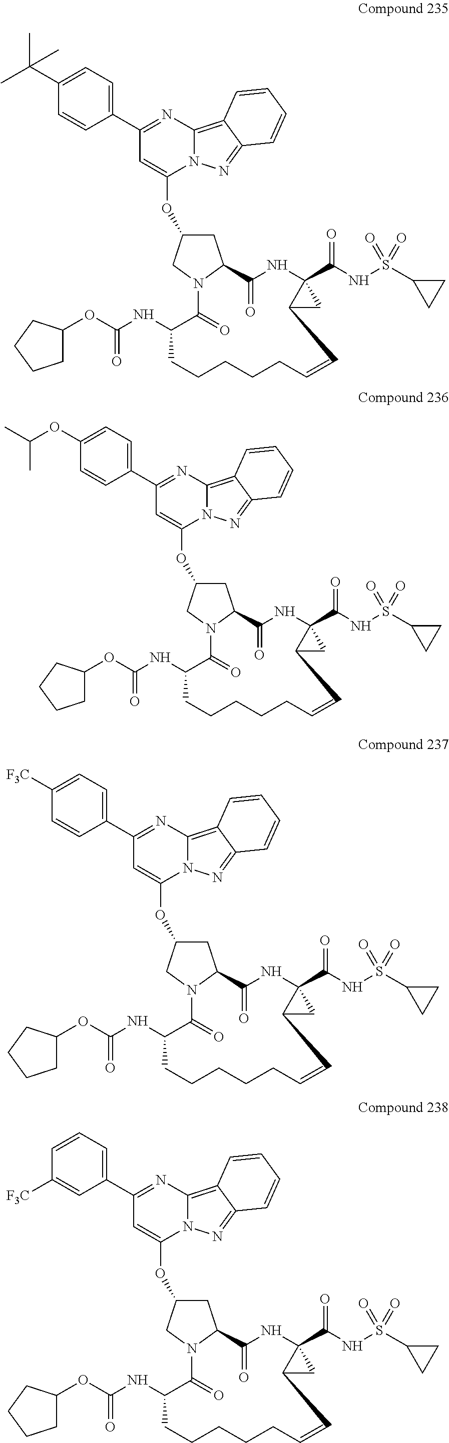

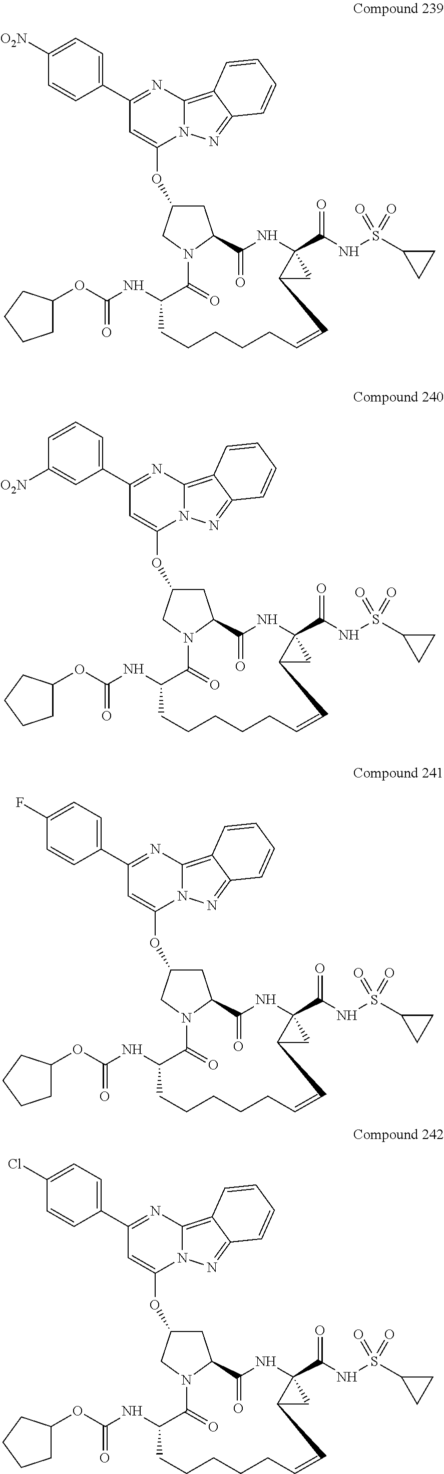

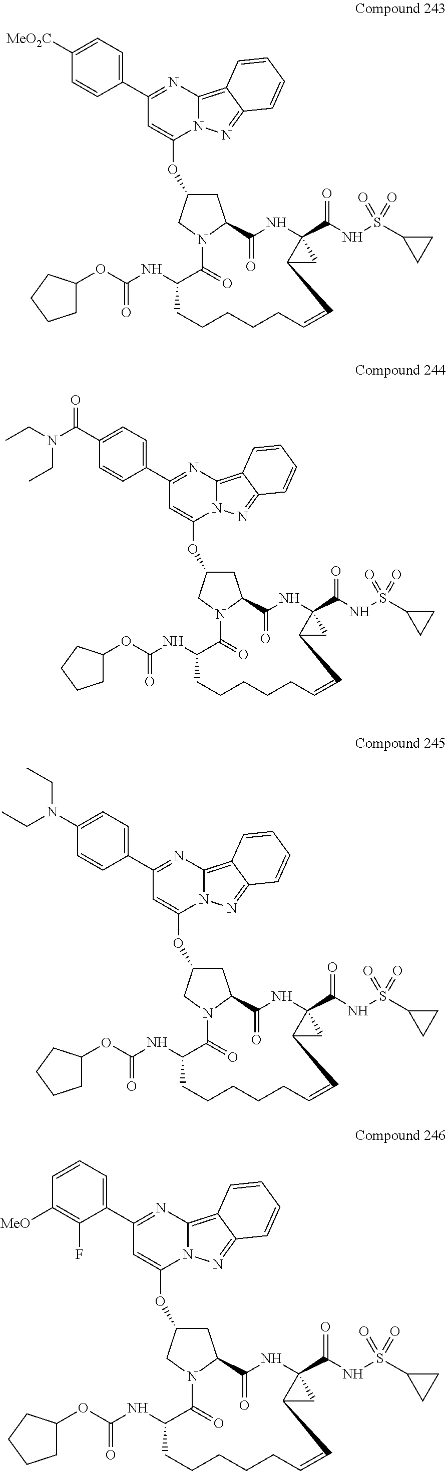

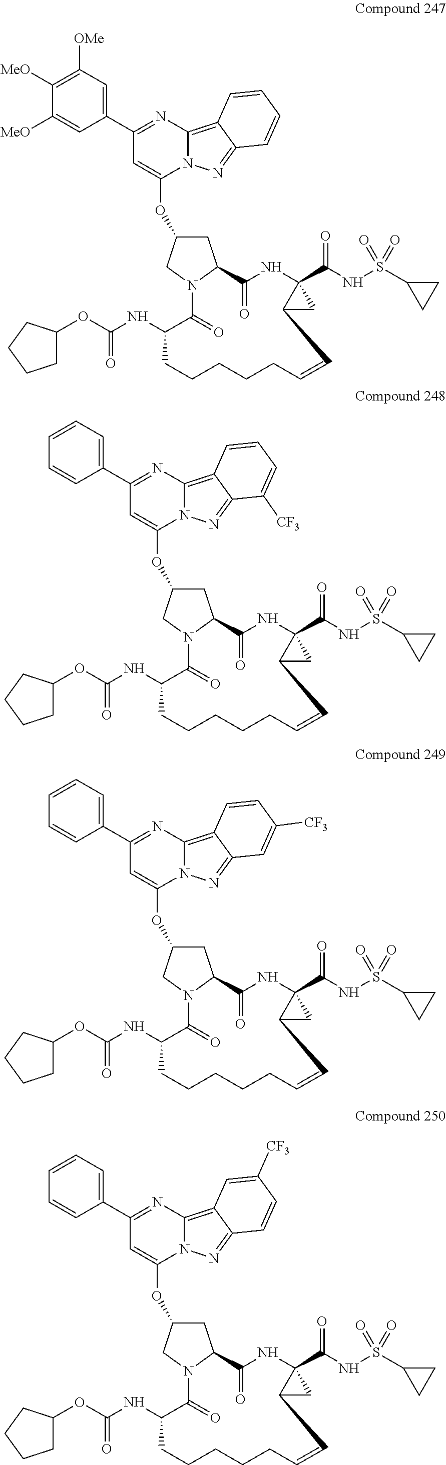

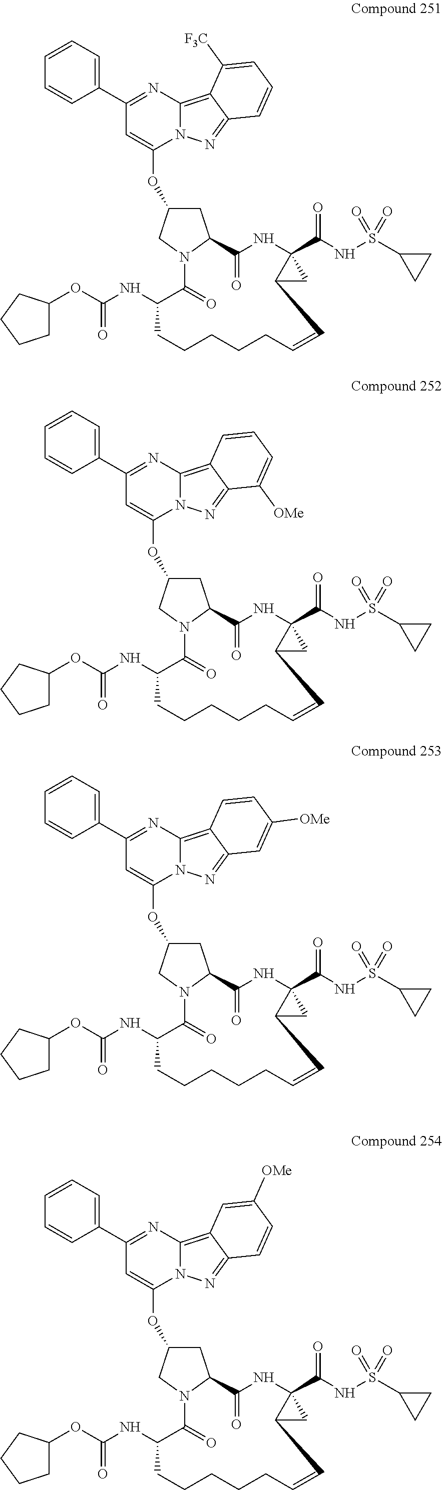

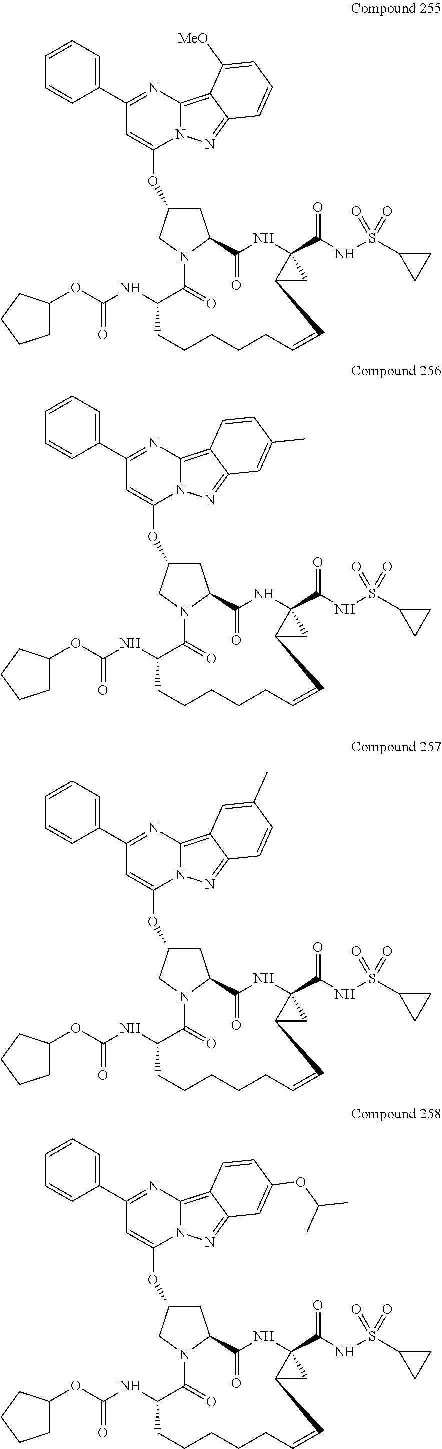

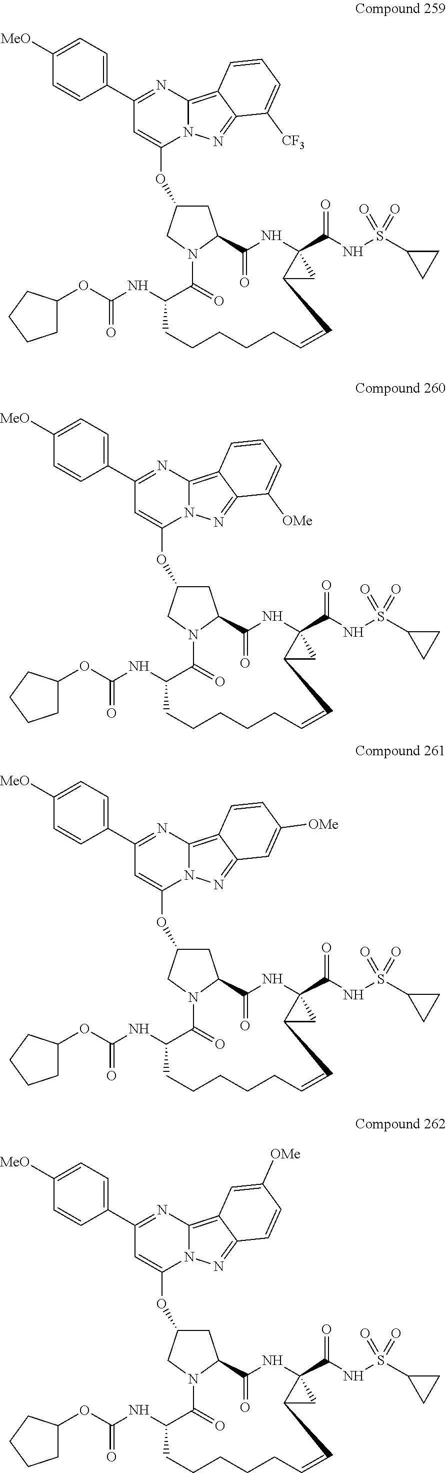

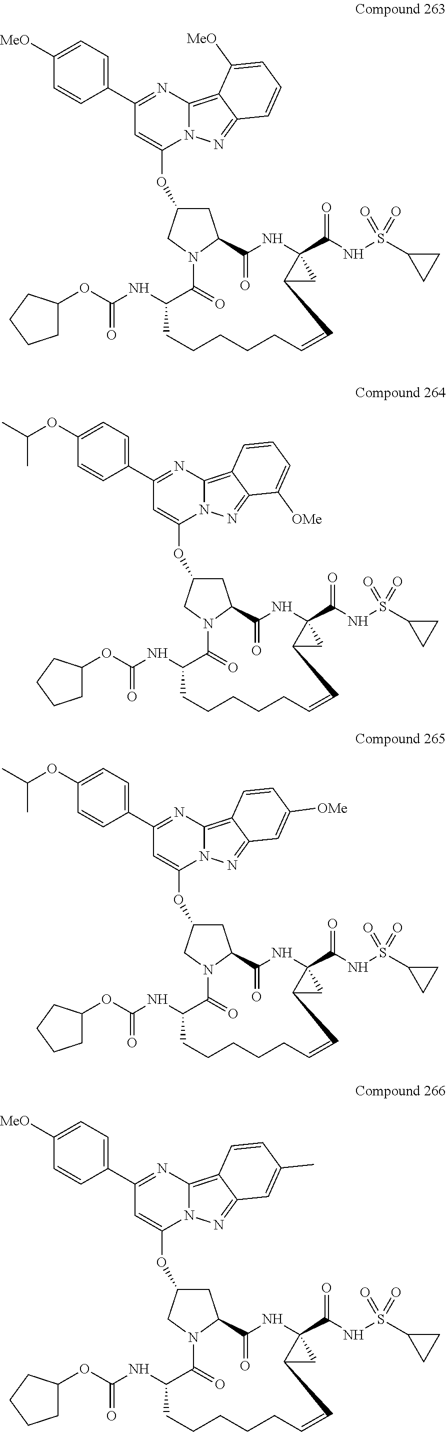

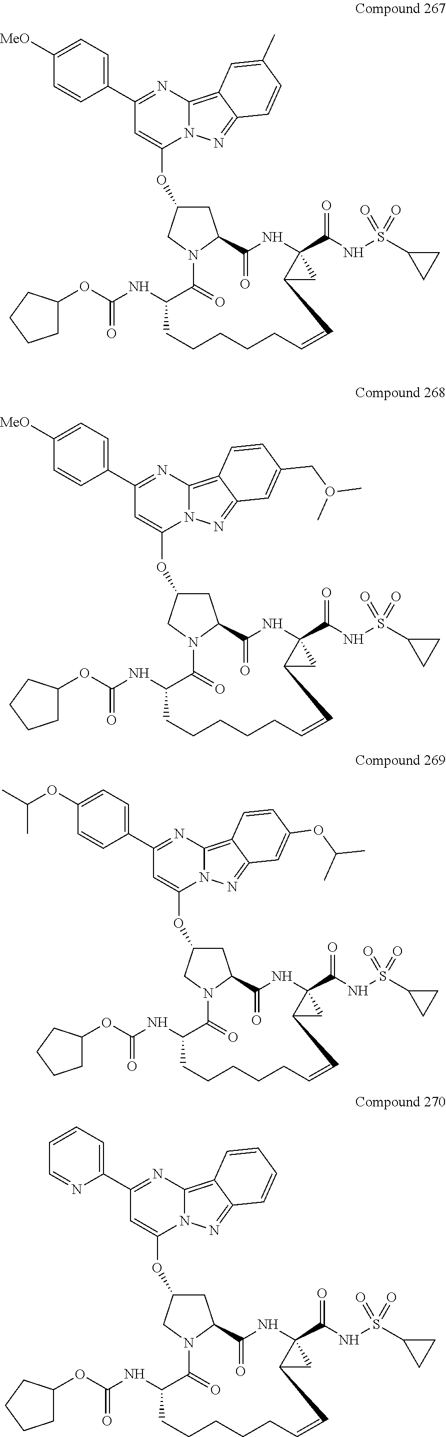

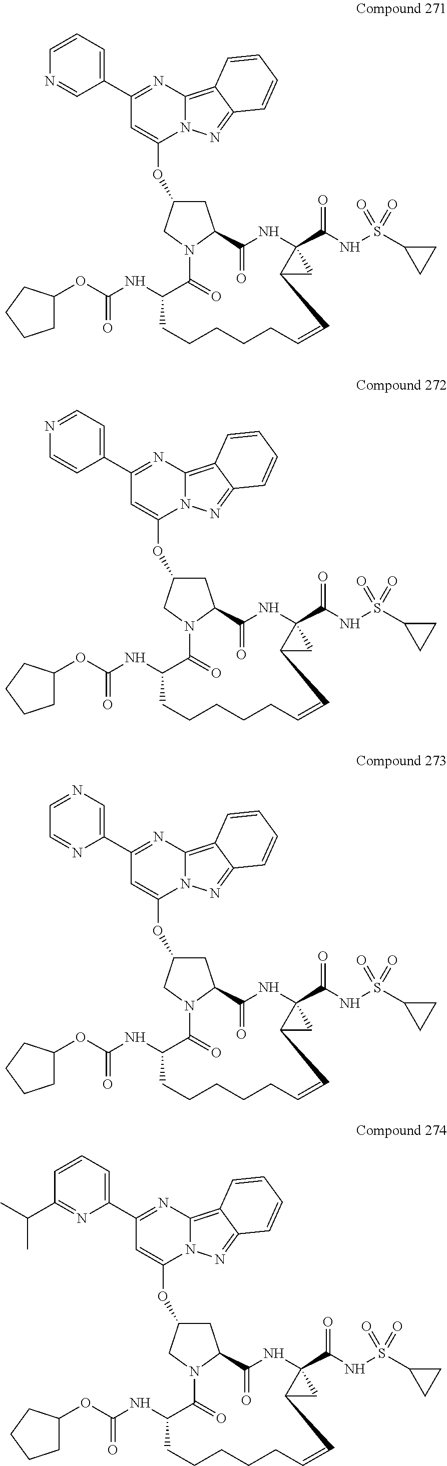

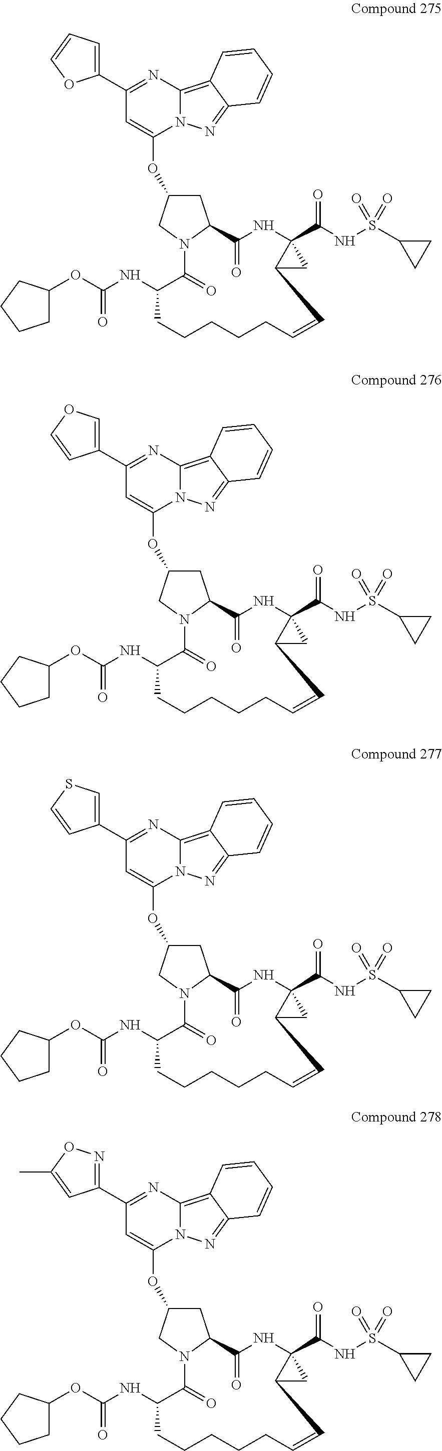

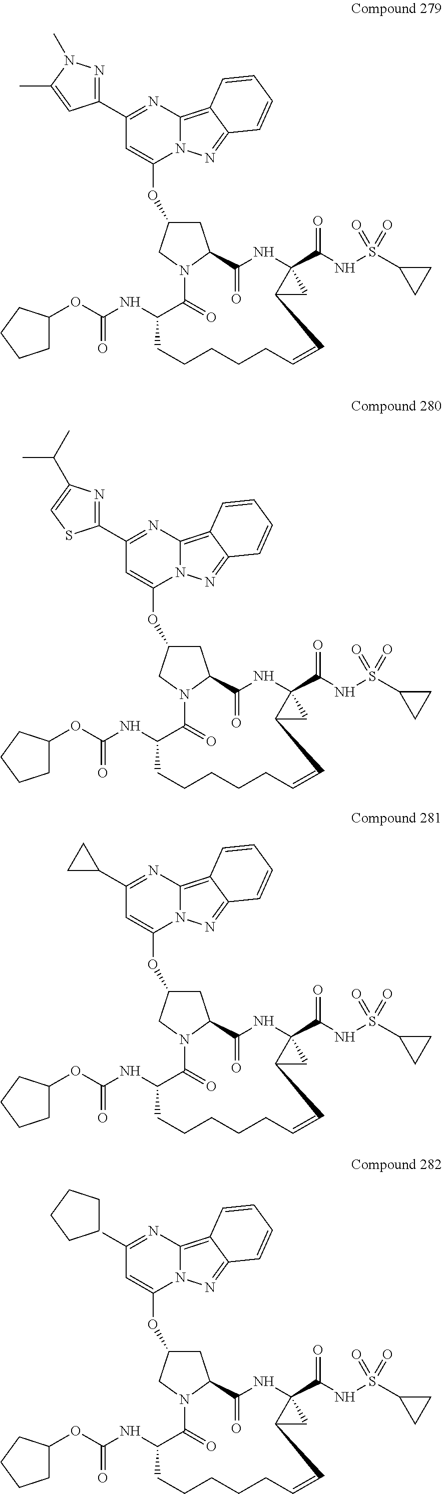

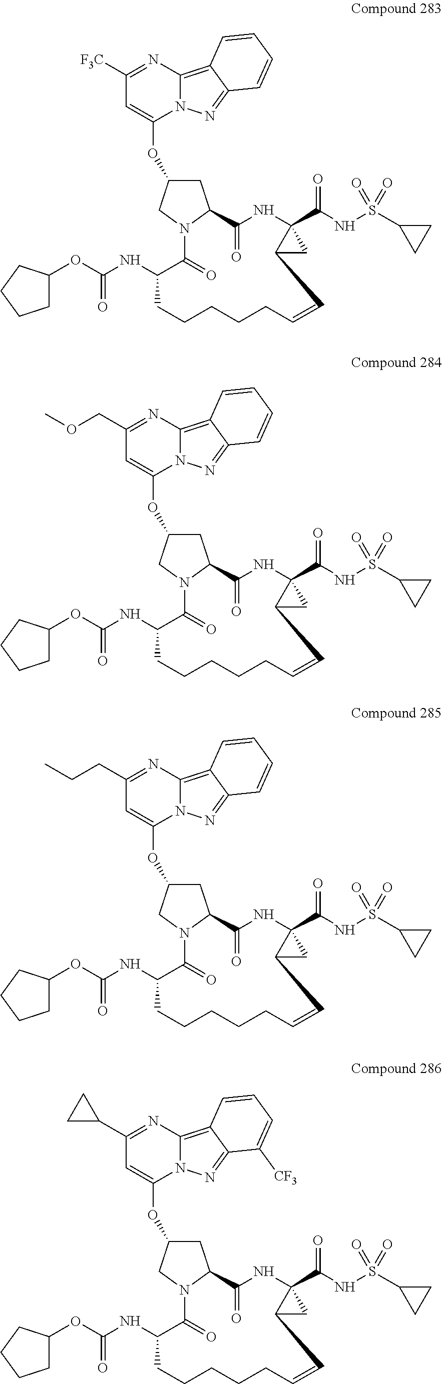

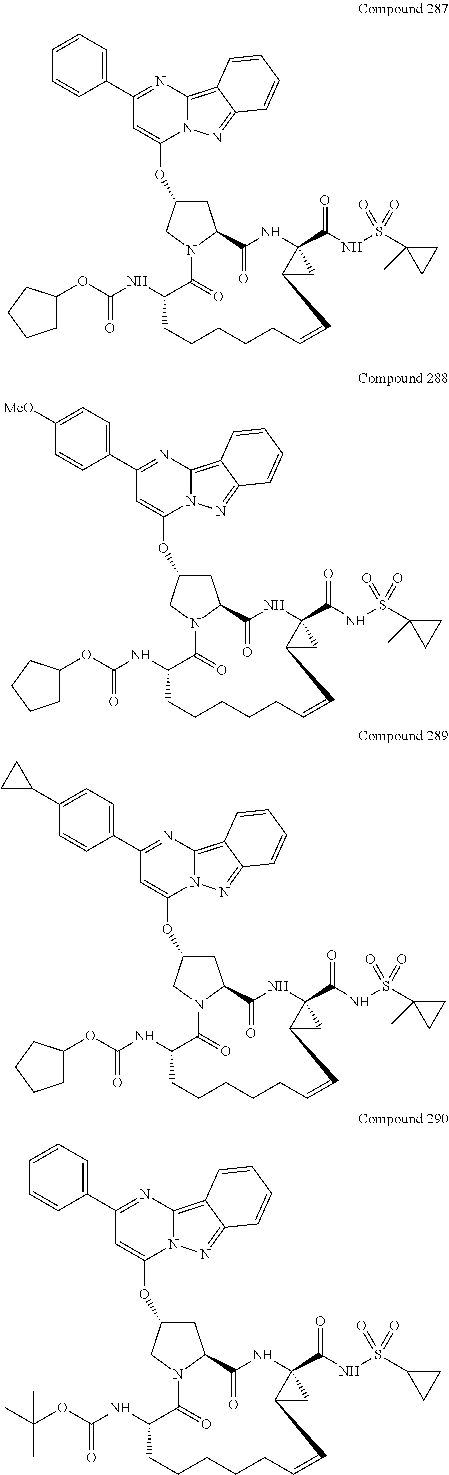

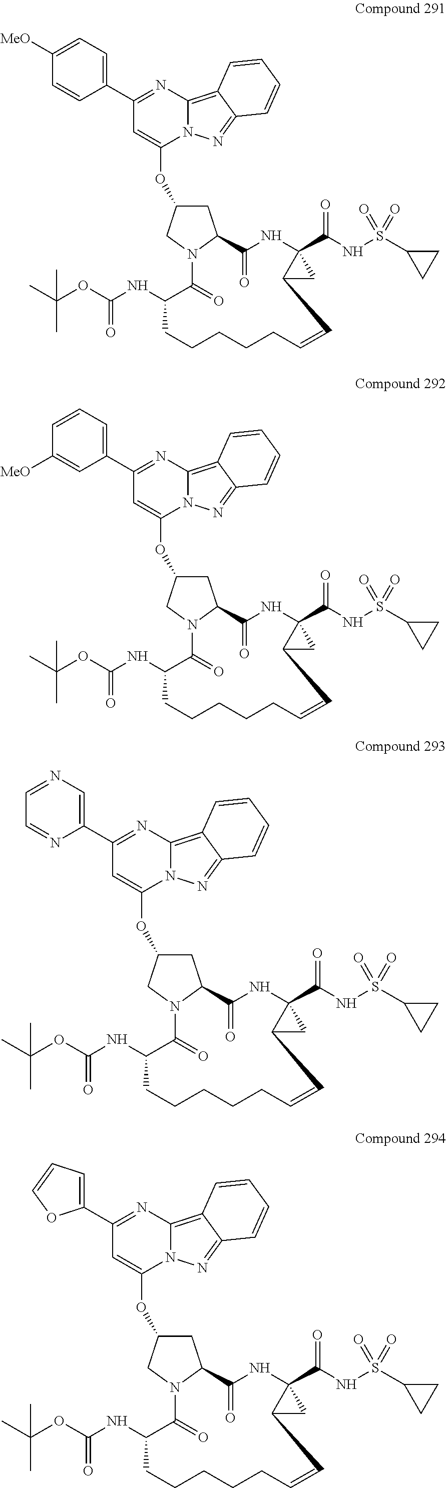

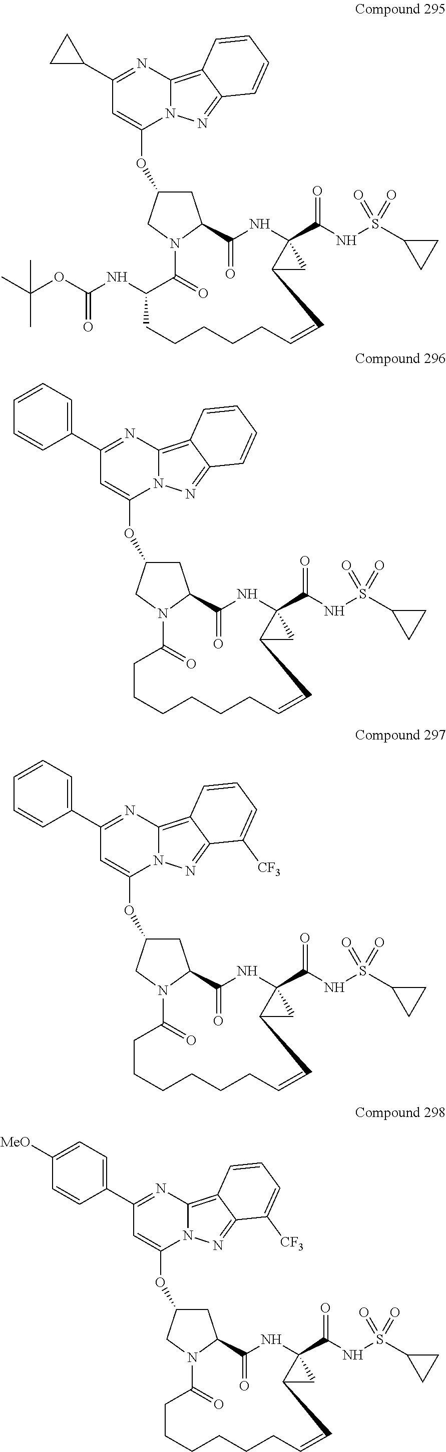

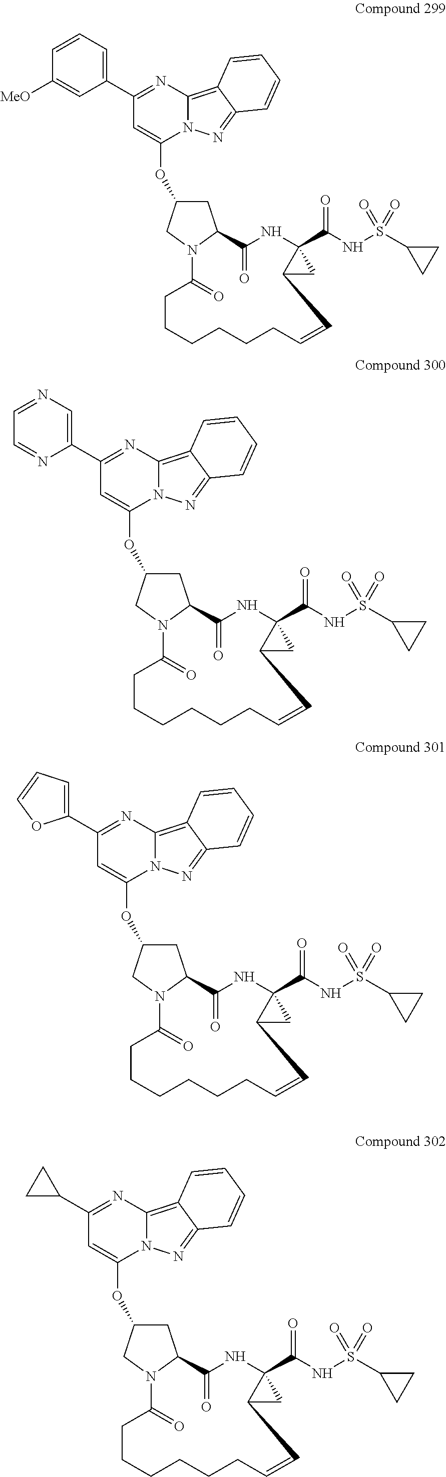

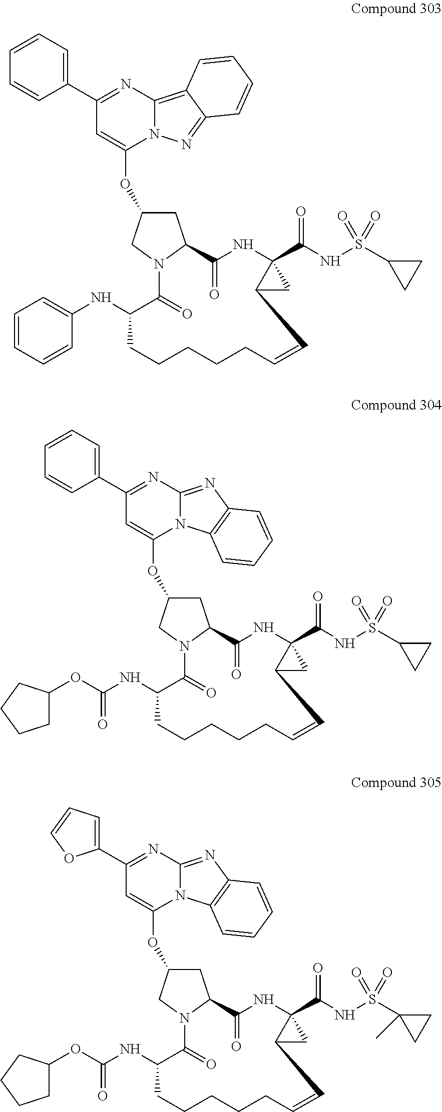

In yet another aspect, this invention relates to a method for treating hepatitis C virus infection. The method includes administering to a subject in need thereof an effective amount of compound of formula (I), (II), or (III) shown above.

In still another aspect, this invention relates to a pharmaceutical composition for use in treating HCV infection. The composition contains an effective amount of at least one of the compounds of formula (I), (II), or (III) and a pharmaceutically acceptable carrier. It may include an inhibitor of a target other than HCV NS3 protease in the HCV life cycle, e.g., NS5B polymerase, NS5A, NS4B, or p7.

Examples of such agents include, but are not limited to, N-[3-(1-cyclobutylmethyl-4-hydroxy-2-oxo-1,2-dihydro-quinolin-3-yl)-1,1-dioxo-1,4-dihydro-116-benzo[1,2,4]thiadiazin-7-yl]-methanesulfonamide (WO04041818), trans-1,2-di-4-[(phenylacetyl-pyrrolidine-2-(S)-carbonyl)amino]-phenylethylene (WO0401413), and 1-aminoadamantane (Amentadine, Griffin, 2004, J. Gen. Virol. 85: p451). The pharmaceutical composition may also contain an immunomodulatory agent or a second antiviral agent. An immunomodulatory agent refers to an active agent that mediates the immune response. Examples of immunomodulatory agents include, but are not limited to, Nov-205 (Novelos Therapeutics Inc., WO02076490) and IMO-2125 (Idera Pharmaceuticals Inc., WO05001055). An antiviral agent refers to an active agent that kills a virus or suppresses its replication. Examples of antiviral agents include, but are not limited to, ribavirin, ribamidin, interferon-α, pegylated interferon, and HCV protease inhibitors, such as 2-(2-{2-cyclohexyl-2-[(pyrazine-2-carbonyl)-amino]-acetylamino}-3,3-dimethyl-butyryl)-octahydro-cyclopenta[c]pyrrole-1-carboxylic acid (1-cyclopropylaminooxalyl-butyl)-amide (Telaprevir, Vertex Pharmaceuticals Inc., WO02018369), 3-[2-(3-tert-butyl-ureido)-3,3-dimethyl-butyryl]-6,6-dimethyl-3-azabicyclo[3.1.0]hexane-2-carboxylic acid (2-carbamoyl-1-cyclobutylmethyl-2-oxo-ethyl)-amide (Boceprevir, Schering-Plough Research Institute, WO03062265), and 4-fluoro-1,3-dihydro-isoindole-2-carboxylic acid 14-tert-butoxycarbonylamino-4-cyclopropanesulfonylaminocarbonyl-2,15-dioxo-3,16-diaza-tricyclo[14.3.0.04,6]nonadec-7-en-18-yl ester (ITMN-191, InterMune Inc., US2005/0267018).

Also within the scope of this invention is the use of the above-described composition for treating HCV infection or for the manufacture of a medicament for the treatment.

The details of one or more embodiments of the invention are set forth in the description below. Other features, objects, and advantages of the invention will be apparent from the description and from the claims.

### DETAILED DESCRIPTION

The compounds of this invention can be synthesized from commercially available starting materials by methods well known in the art. For example, one can prepare the compounds of Formula (I) via the route shown in Scheme 1 below:

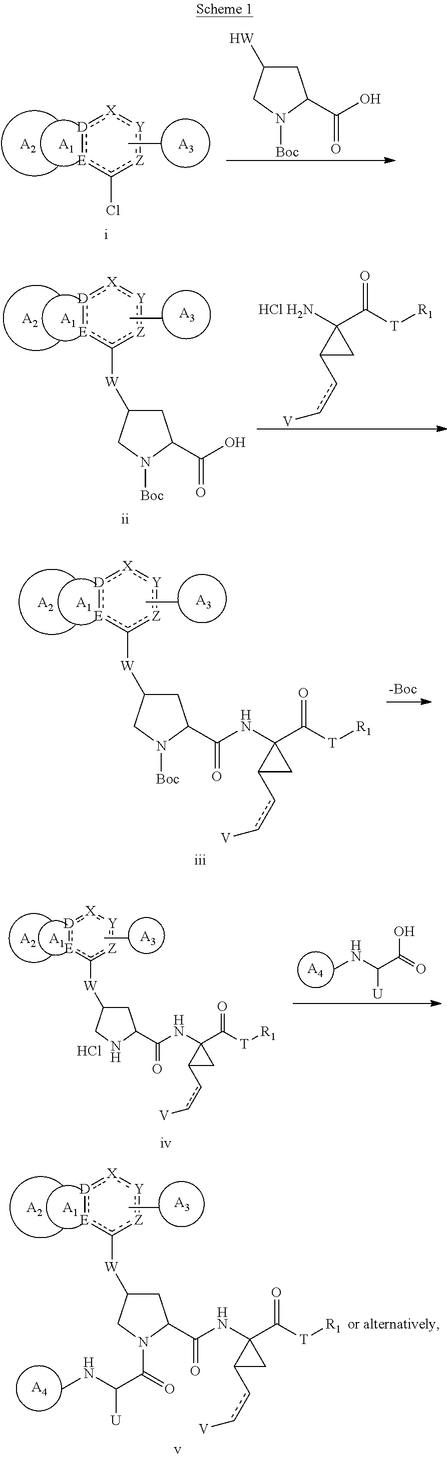

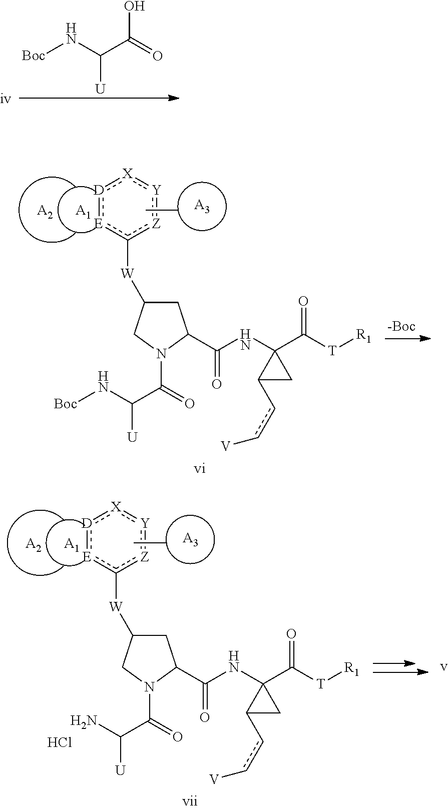

One can prepare the compounds of Formula (II) via the route shown in Scheme 2 below:

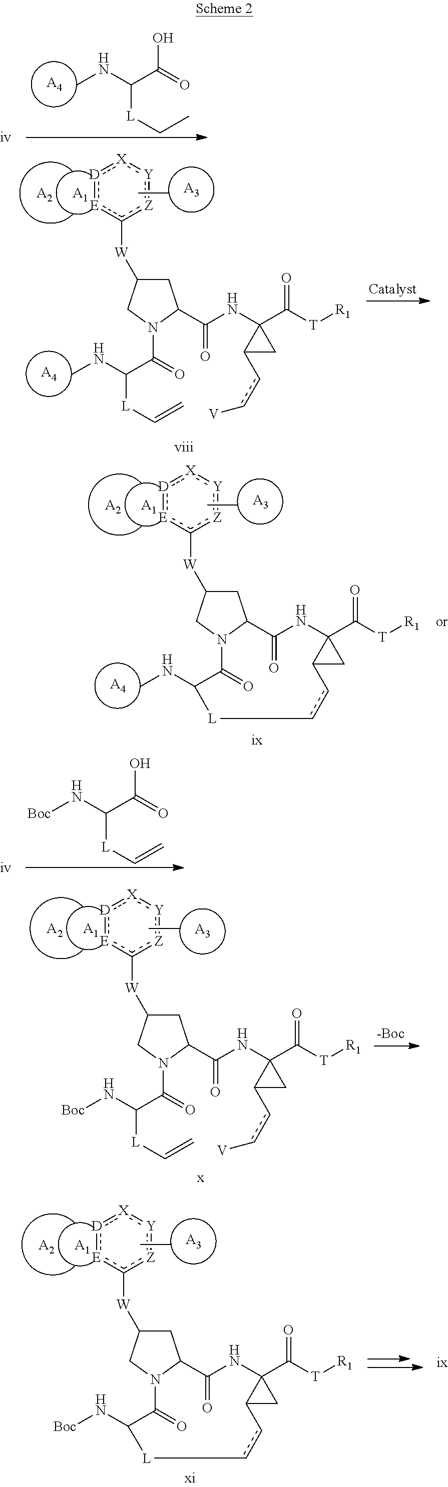

One can prepare the compounds of Formula (III) via the route shown in Scheme 3 below:

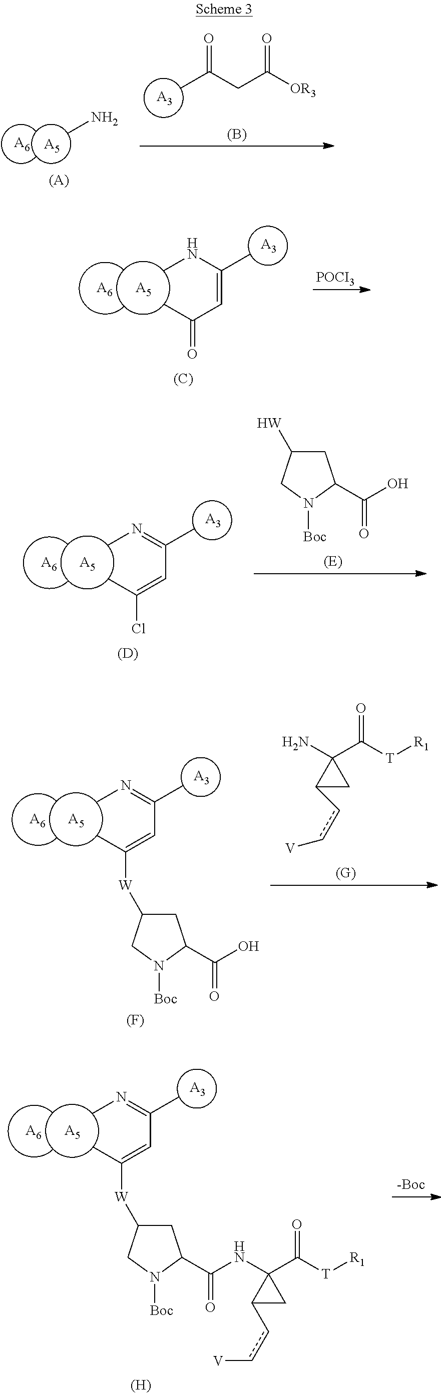

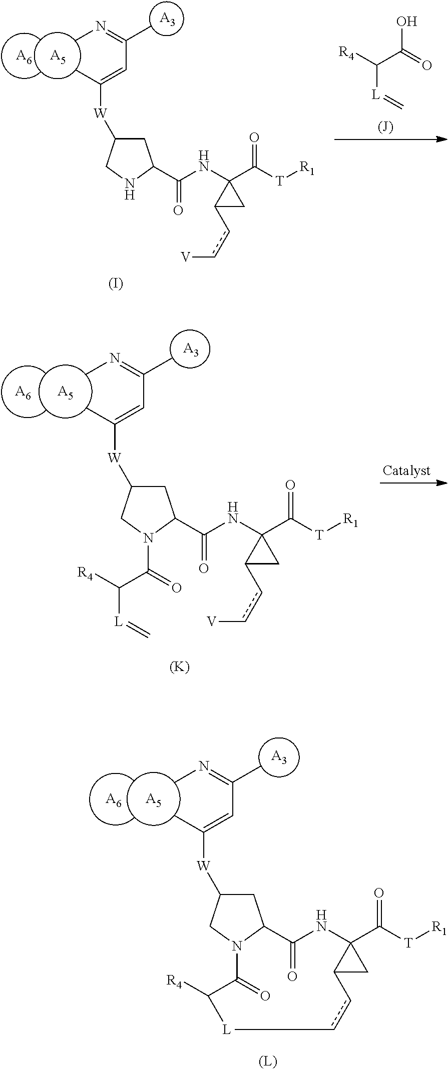

The methods described above may also additionally include steps, either before or after the steps described specifically in Schemes 1-3, to add or remove suitable protecting groups in order to ultimately allow synthesis of the desired compounds. In addition, various synthetic steps may be performed in an alternate sequence or order to give the desired compounds. Synthetic chemistry transformations and protecting group methodologies (protection and deprotection) useful in synthesizing applicable compounds of formula (I) are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The compounds mentioned herein contain a non-aromatic double bond and asymmetric centers. Thus, they can occur as racemates and racemic mixtures, single enantiomers, individual diastereomers, diastereomeric mixtures, tautomers, and cis- or trans-isomeric forms. All such isomeric forms are contemplated. For example, the compounds of Formulas (I), (II), and (III) shown above may possess the following stereochemical configurations as in Formulas (IV), (V), and (VI), respectively:

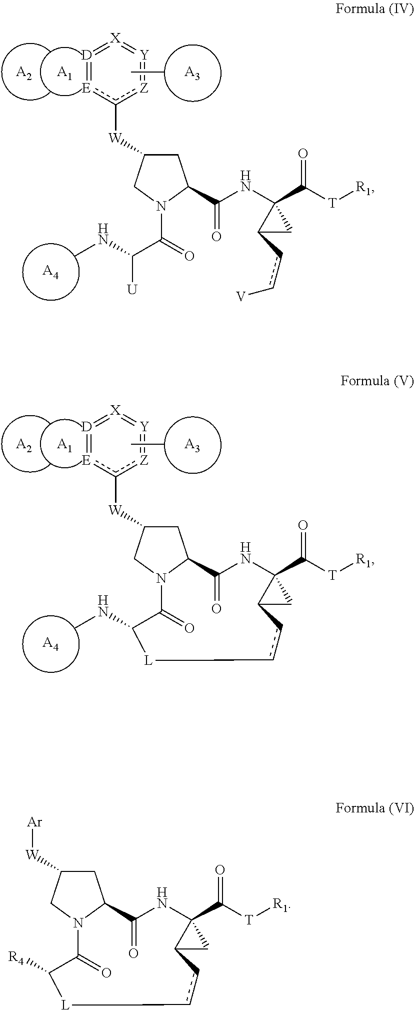

The compounds described above include the compounds themselves, as well as their salts, prodrugs, and solvates, if applicable. A salt, for example, can be formed between an anion and a positively charged group (e.g., amino) on a compound of this invention. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of this invention. Suitable cations include sodium ion, potassium ion, magnesium ion, calcium ion, and an ammonium cation such as tetramethylammonium ion. The compounds of this invention also include salts containing quaternary nitrogen atoms. Examples of prodrugs include esters and other pharmaceutically acceptable derivatives, which, upon administration to a subject, are capable of providing active compounds of this invention. A solvate refers to a complex formed between an active compound of this invention and a pharmaceutically acceptable solvent. Examples of pharmaceutically acceptable solvents include water, ethanol, isopropanol, ethyl acetate, acetic acid, and ethanolamine.

Also within the scope of this invention is a method of treating HCV infection by administering an effective amount of one or more of the compounds of this invention to a patient. The term “treating” or “treatment” refers to administering the compounds to a subject, who has HCV infection, a symptom of it, or a predisposition toward it, with the purpose to confer a therapeutic effect, e.g., to cure, relieve, alter, affect, ameliorate, or prevent the HCV infection, the symptom of it, or the predisposition toward it. The term “an effective amount” refers to the amount of an active compound of this invention that is required to confer a therapeutic effect on the treated subject. Effective doses will vary, as recognized by those skilled in the art, depending on the types of diseases treated, route of administration, excipient usage, and the possibility of co-usage with other therapeutic treatment.

The compounds of this invention can remain in the blood system at an effective level for a prolonged period. Thus, these compounds can be administered at an effective amount once a day to confer the therapeutic effect.

To practice the method of the present invention, a composition having one or more compounds of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active compounds of this invention can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound of this invention. Examples of other carriers include colloidal silicon oxide, magnesium stearate, cellulose, sodium lauryl sulfate, and D&C Yellow # 10.

The compounds of this invention can be used to treat HCV along with a second anti-HCV agent, such as an inhibitor of a target other than HCV NS3 protease in the HCV life cycle, another antiviral agent and an immunomodulatory agent. One of the compounds of this invention and the second anti-HCV agent can be administered concurrently or at different times. For concurrent administration, these two agents they may be admixed to form a single dose, or prepared as two separate doses. They are each used at such an amount that their total amount is effective for treating HCV.

The compounds of this invention described above can be preliminarily screened for their efficacy in treating HCV infection by an in vitro assay (see Examples 17 and 18) and then confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Listed below are abbreviations of compound names used herein and the terms they stand for:

DMF N,N-dimethylformamide

t-Bu tert-butyl

THF Tetrahydrofuran

Boc tert-butoxycarbonyl

DMSO Dimethyl sulfoxide

HATU O-(7-Azabenzotriazol-1-yl)-N,N,N′,N′-tetramethyluronium hexafluorophosphate

HOBt N-Hydroxybenzotriazole

NMM N-methylmorpholine

DCM dichloromethane or methylene chloride

TEA Triethylamine

HOAc Acetic acid

TFA Trifluoroacetic acid

### Example 1

### Preparation of Compound 3

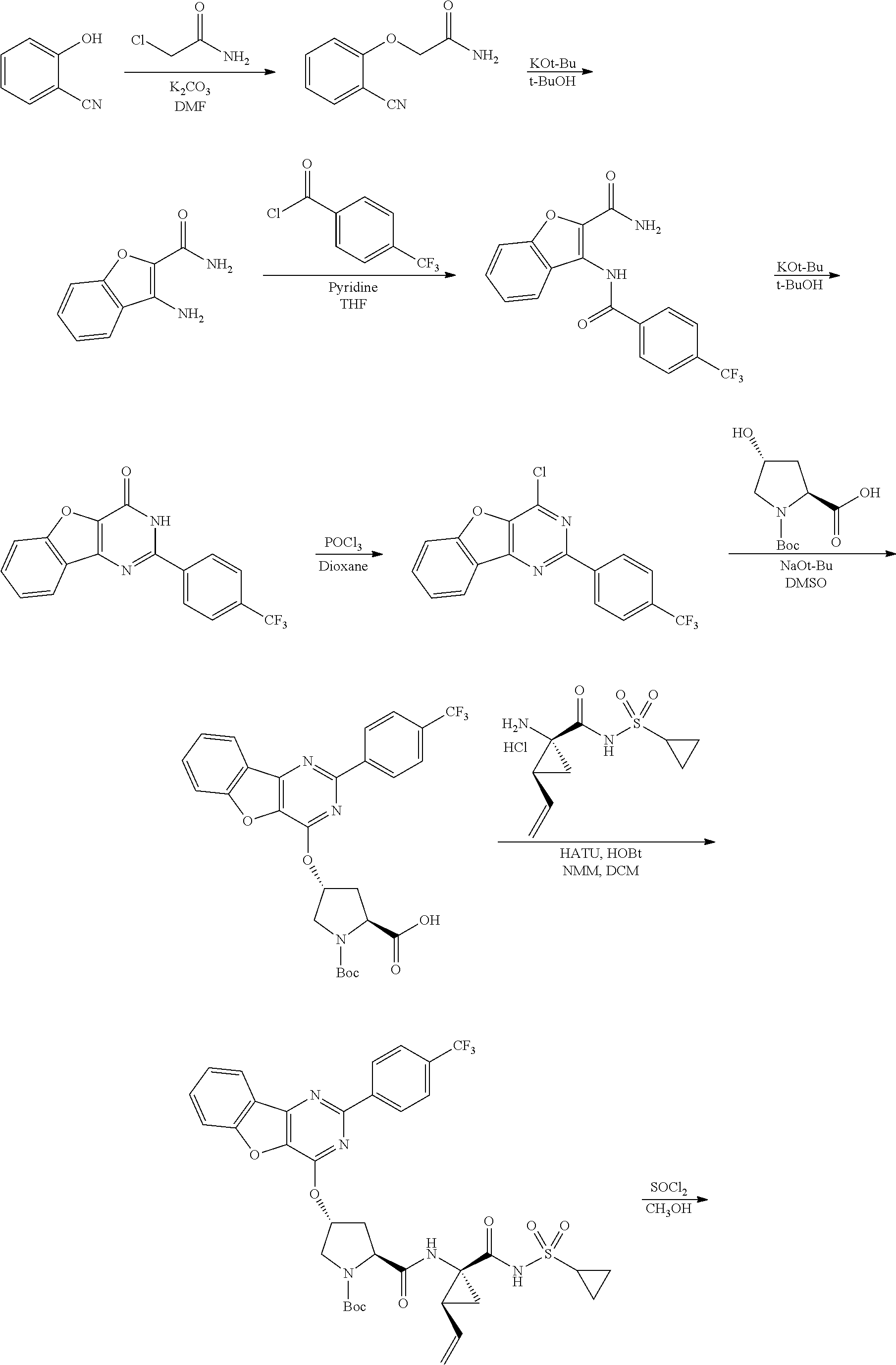

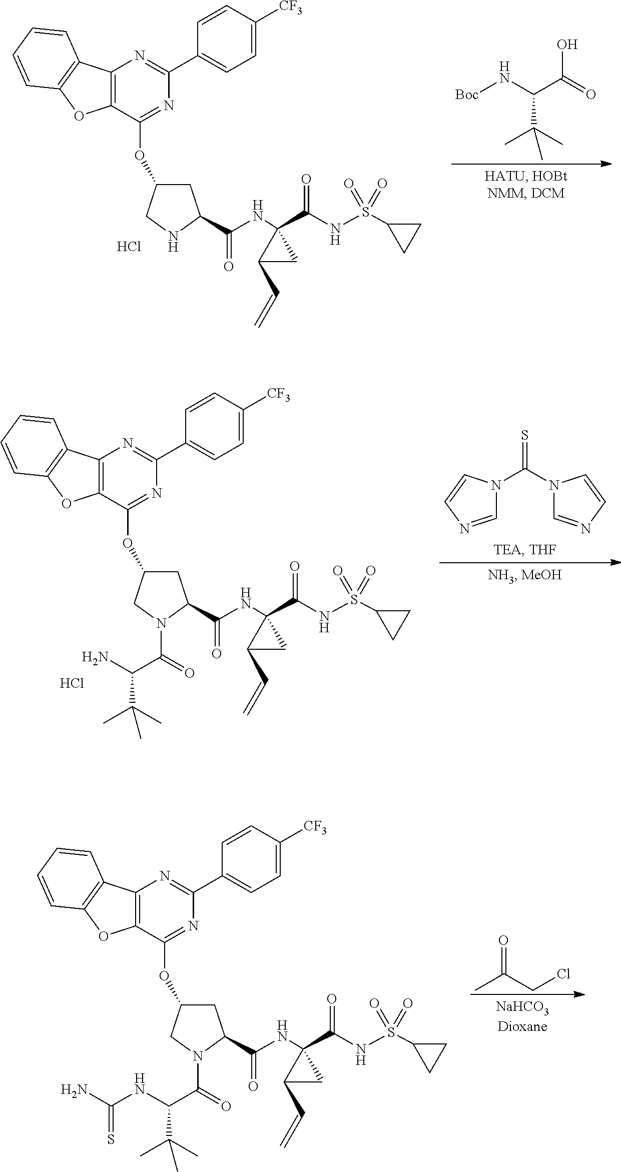

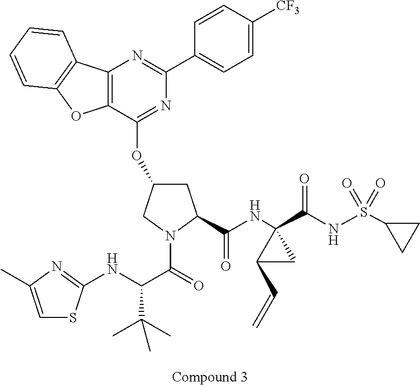

A mixture of 2-hydroxybenzonitrile (50.0 g, 0.42 mol) and potassium carbonate (174.0 g, 1.26 mol) in 0.84 L of dry DMF was stirred at 60° C. for 1 h. To the mixture was added 2-chloroacetamide (43.2 g, 0.46 mmol). After stirred at 80° C. for 3 h, the mixture was poured into ice water and stirred for 1 h. The precipitate was collected by filtration and dried under reduced pressure to give 61.2 g of 2-(2-cyanophenoxy)acetamide. LC-MS (M+H): 177.1.

To a stirred solution of 2-(2-cyanophenoxy)acetamide (121.3 g, 0.69 mol) in 1.27 L of t-BuOH was added KOt-Bu (92.7 g, 0.83 mol) and the mixture was stirred at 80° C. for 3 h. The solvent was removed by evaporation and the residue was dissolved in methylene chloride. The methylene chloride solution was washed with saturated NaCl aqueous solution, dried over MgSO₄, and concentrated in vacuo to give 106 g of 3-aminobenzo-furan-2-carboxamide. LC-MS (M+H): 177.0.

To a stirred solution of 3-aminobenzofuran-2-carboxamide (70.0 g, 0.40 mol) in 1.12 L of THF was added pyridine (64.1 mL, 0.79 mol) and 4-(trifluoromethyl)benzoyl chloride (67.9 mL, 0.46 mol) at 0° C. and the mixture was stirred at room temperature overnight. The solvent was removed by evaporation and the residue was poured into 1 L of water and 0.3 L of saturated NaHCO₃ aqueous solution. The mixture was stirred for 2 h and the precipitate was collected by filtration, washed with 0.5 L of ether, and dried under reduced pressure to give 115.2 g of 3-(4-(trifluoromethyl)benzamido)benzofuran-2-carboxamide. LC-MS M+H): 349.0.

To a stirred solution of 3-(4-(trifluoromethyl)benzamido)benzofuran-2-carboxamide (57.6 g, 165.4 mmol) in 414 mL of t-BuOH and 83 mL of water was added 1N KOt-Bu aqueous solution (330.8 mL, 330.8 mmol) and the mixture was stirred at 70° C. overnight. The solvent was removed by evaporation and the residue was poured into 1 L of water. The pH value was adjusted to about 2 using 6N HCl aqueous solution. The precipitate was collected by filtration, washed with 2 L of water and dried under reduced pressure to give 50.8 g of 2-(4-(trifluoromethyl)phenyl)benzofuro[3,2-d]pyrimidin-4(3H)-one. LC-MS (M+H): 331.0, ¹H-NMR (DMSO, 300 Hz): δ7.49 (t, J=7.5 Hz, 1H), 7.67 (t, 7.5 Hz, 1H), 7.83 (d, J=8.4 Hz, 1H), 7.90 (d, J=8.1 Hz, 2H), 8.09 (d, J=7.2 Hz, 1H), 8.35 (d, J=8.1 Hz, 2H).

To a stirred solution of 2-(4-(trifluoromethyl)phenyl)benzofuro[3,2-d]pyrimidin-4(3H)-one (50.8 g, 154.0 mmol) in 308 mL of 1,4-dioxane was added POCl₃ (72 mL, 770 mmol) at 0° C. The mixture was stirred at room temperature for 30 min and stirred at 110° C. for 6 h. The solvent was removed by evaporation and ice water was added to the residue. The pH value was adjusted to about 8 using saturated Na₂CO₃ aqueous solution. The precipitate was collected by filtration, washed with 2 L of water and dissolved in methylene chloride. The methylene chloride layer was dried over MgSO₄ and concentrated in vacuo to give 42.9 g of 4-chloro-2-(4-(trifluoromethyl)phenyl)benzofuro[3,2-d]pyrimidine. LC-MS (M+H): 349.0.

To a stirred solution of (2S,4R)-1-(tert-butoxycarbonyl)-4-hydroxypyrrolidine-2-carboxylic acid (34.1 g, 147.6 mmol) in 520 mL DMSO was added NaOt-Bu (27.2 g, 282.9 mmol) at 10° C. and the mixture was stirred at room temperature for 2 h. To the mixture was added 4-chloro-2-(4-(trifluoromethyl)phenyl)-benzofuro[3,2-d]pyrimidine (42.9 g, 123 mmol) and 116 mL THF. The mixture was stirred at room temperature for 1 h and then poured into ice water. The pH value was adjusted to about 3 using 6N HCl aqueous solution and the solution was extracted with ethyl acetate. The ethyl acetate layer was washed thrice with water and then brine, dried over MgSO₄ and concentrated in vacuo. The resulting residue was washed with hexane and dried under high vacuum to give 59.3 g of (2S,4R)-1-(tert-butoxycarbonyl)-4-(2-(4-(trifluoromethyl)phenyl)benzofuro[3,2-d]pyrimidin-4-yloxy)pyrrolidine-2-carboxylic acid. LC-MS (M+H): 544.1.

A mixture of (2S,4R)-1-(tert-butoxycarbonyl)-4-(2-(4-(trifluoromethyl)-phenyl)benzofuro[3,2-d]pyrimidin-4-yloxy)pyrrolidine-2-carboxylic acid (59.3 g, 109.2 mmol), NMM (42.0 mL, 382.2 mmol), HATU (83.0 g, 218.4 mmol), HOBt (4.5 g, 33.0 mmol) and hydrochloride salt of (1R,2S)-1-amino-N-(cyclopropylsulfonyl)-2-vinylcyclopropanecarboxamide (37.7 g, 163.8 mmol) in 546 mL of dry methylene chloride was stirred at room temperature for 3 h. To the mixture was added 600 mL of methylene chloride. The mixture was stirred for 1 h and filtered to move the solid. The methylene chloride solution was extracted thrice with saturated NH₄Cl aqueous solution, dried over MgSO₄ and concentrated in vacuo. To the crude compound was added 800 mL of methylene chloride. The mixture was filtered to give 46.8 g of the Boc-protected compound. LC-MS (M+H): 756.2.

To the Boc-protected compound (46.8 g, 62.0 mmol) in 644 mL of methanol was added thionyl chloride (9.1 mL, 124.0 mmol) at 0° C. and the mixture was stirred at 45° C. for 5 h. The organic solvent was removed by evaporation, and the resulting residue washed with 1 L of ether, washed with 500 mL of ethyl acetate/ether(1/4) and dried under high vacuum to give 45.0 g of the hydrochloride salt of (2S,4R)—N-((1R,2S)-1-(cyclopropylsulfonylcarbamoyl)-2-vinylcyclopropyl)-4-(2-(4-(trifluoromethyl)phenyl)benzofuro[3,2-d]pyrimidin-4-yloxy)pyrrolidine-2-carboxamide. LC-MS (M+H): 656.1, ¹H-NMR (CD₃OD, 300 Hz): δ1.12 (m, 4H), 1.37 (m, 1H), 1.93 (m, 1H), 2.28 (m, 1H), 2.52 (m, 1H), 2.96 (m, 2H), 3.92 (d, J=3.6 Hz, 2H), 4.67 (dd, J₁=10.4 Hz, J₂=7.5 Hz, 1H), 5.11 (dd, J₁=11.1 Hz, J₂=1.7 Hz, 1H), 5.28 (dd, J₁=16.2 Hz, J₂=1.7 Hz, 1H), 5.63 (m, 1H), 6.25 (m, 1H), 7.55 (m, 1H), 7.73 (m, 2H), 7.80 (d, J=8.1 Hz, 2H), 8.27 (d, J=7.5 Hz, 1H), 8.68 (d, J=8.1 Hz, 2H), 9.10 (s, 1H).

A mixture of Boc-L-tert-Leucine (1.27 g, 5.49 mmol), NMM (2 mL, 18.30 mmol), HATU (3.48 g, 9.15 mmol), HOBt (0.19 g, 1.37 mmol) and the hydrochloride salt of (2S,4R)—N-((1R,2S)-1-(cyclopropylsulfonylcarbamoyl)-2-vinylcyclopropyl)-4-(2-(4-(trifluoromethyl)phenyl)benzofuro[3,2-d]pyrimidin-4-yloxy)pyrrolidine-2-carboxamide (3 g, 4.57 mmol) in 23 mL of dry methylene chloride was stirred at room temperature overnight. The solid of the reaction solution was removed by filtration and the methylene chloride layer was extracted twice with saturated NH₄Cl aqueous solution, dried over MgSO₄ and concentrated in vacuo. The crude material was purified by silica gel column with 6%˜10% ethyl acetate/methylene chloride to give 3.48 g of the Boc-protected compound. LC-MS (M+H): 869.3

To the Boc-protected compound (2.0 g, 2.3 mmol) in 23 mL of methylene chloride was added 11.5 mL of 4N HCl in 1,4-dioxane at 0° C. The mixture was stayed at 4° C. overnight and the organic solvent was removed by evaporation. The resulting residue was washed with ether and dried under high vacuum to give 1.9 g of the hydrochloride salt of (2S,4R)-1-((S)-2-amino-3,3-dimethylbutanoyl)-N-((1R,2S)-1-(cyclopropylsulfonylcarbamoyl)-2-vinylcyclopropyl)-4-(2-(4-(trifluoromethyl)phenyl)benzofuro[3,2-d]pyrimidin-4-yloxy)pyrrolidine-2-carboxamide. LC-MS (M+H): 769.2.

To a stirred solution of the hydrochloride salt of (2S,4R)-1-((S)-2-amino-3,3-dimethylbutanoyl)-N-((1R,2S)-1-(cyclopropylsulfonylcarbamoyl)-2-vinylcyclopropyl)-4-(2-(4-(trifluoromethyl)phenyl)benzofuro[3,2-d]pyrimidin-4-yloxy)pyrrolidine-2-carboxamide (0.6 g, 0.78 mmol) in 3.9 mL of THF was added triethylamine (0.54 mL, 3.90 mmol) and di(1H-imidazol-1-yl)methanethione (0.14 g, 1.56 mmol) at 0° C. After the mixture was stirred at room temperature for 1 h, ammonia ca. 7N solution in methanol (1.1 mL, 7.80 mmol) was added. The mixture was stirred at room temperature for additional 3 h. The solvent was removed by evaporation to give 0.8 g of 1-((S)-1-((2S,4R)-2-(((1R,2S)-1-(cyclo-propylsulfonylcarbamoyl)-2-vinylcyclopropyl)carbamoyl)-4-(2-(4-(trifluoromethyl)-phenyl)benzofuro[3,2-d]pyrimidin-4-yloxy)pyrrolidin-1-yl)-3,3-dimethyl-1-oxobutan-2-yl)thiourea. LC-MS (M+H): 828.2.

A mixture of 1-((S)-1-((2S,4R)-2-(((1R,2S)-1-(cyclopropylsulfonyl-carbamoyl)-2-vinylcyclopropyl)carbamoyl)-4-(2-(4-(trifluoromethyl)phenyl)-benzofuro[3,2-d]pyrimidin-4-yloxy)pyrrolidin-1-yl)-3,3-dimethyl-1-oxobutan-2-yl)thiourea (0.22 g, 0.26 mmol), sodium bicarbonate (0.22 g, 2.62 mmol) and chloroacetone (0.073 mL, 0.47 mmol) in 2.6 mL of 1,4-dioxane at room temperature was stirred at 60° C. for 4.5 h. The solvent was removed by evaporation and the residue was dissolved in methylene chloride. The methylene chloride solution was washed with water, dried over MgSO₄, and concentrated in vacuo. The crude material was purified by silica gel column with 20% ethyl acetate/1% methanol/methylene chloride to give 27 mg of (2S,4R)—N-((1R,2S)-1-(cyclopropyl-sulfonylcarbamoyl)-2-vinylcyclopropyl)-1-((S)-3,3-dimethyl-2-(4-methylthiazol-2-ylamino)butanoyl)-4-(2-(4-(trifluoromethyl)phenyl)-benzofuro[3,2-d]pyrimidin-4-yloxy)pyrrolidine-2-carboxamide (Compound 3). LC-MS (M+H): 866.2 ¹H-NMR (CDCl₃, 300 Hz): δ1.05 (m, 2H), 1.10 (s, 9H), 1.32 (m, 2H), 1.51 (m, 1H), 1.80 (s, 3H), 1.93 (m, 1H), 2.12 (m, 1H), 2.71 (m, 2H), 2.91 (m, 1H), 4.18 (m, 1H), 4.27 (dd, J₁=12.0 Hz, J₂=4.4 Hz, 1H), 4.61 (d, J=11.7 Hz, 1H), 4.75 (t, J=7.8 Hz, 1H), 5.15 (d, J=10.8 Hz, 1H), 5.26 (d, J=16.8 Hz, 1H), 5.65 (s, 1H), 5.71 (m, 1H), 6.24 (m, 1H), 7.52 (t, J=7.5 Hz, 1H), 7.66 (m, 2H), 7.80 (d, J=8.1 Hz, 2H), 8.28 (d, J=8.1 Hz, 1H), 8.66 (d, J=8.1 Hz, 2H), 10.16 (s, 1H).

### Example 2

### Preparation of Compound 64

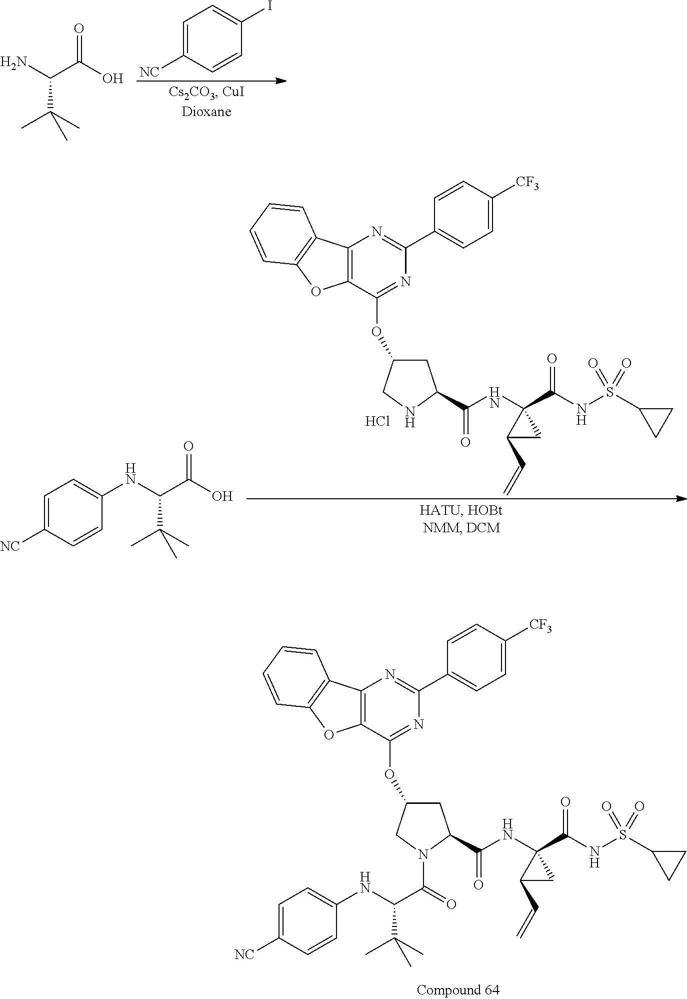

A mixture of L-tert-Leucine (0.50 g, 3.81 mmol), cesium carbonate (3.73 g, 11.44 mmol), copper(I) iodide (0.36 g, 1.91 mmol) and 4-iodobenzonitrile (2.18 g, 9.53 mmol) in 7.6 mL 1,4-dioxane was stirred at 90° C. for 74 h. The organic solvent was removed by evaporation and the residue was dissolved in 100 mL of water. The solution was washed twice with ether. The aqueous solution was treated with 6 N HCl aqueous solution to adjust pH to ˜3 and then extracted with ethyl acetate. The ethyl acetate layer was washed with water, brine, dried over MgSO₄ and concentrated in vacuo to give 72 mg of (S)-2-(4-cyanophenylamino)-3,3-dimethylbutanoic acid. LC-MS (M+H): 233.1, ¹H-NMR (CD₃OD, 300 Hz): δ1.12 (s, 9H), 3.86 (s, 1H), 6.78 (d, J=9.0 Hz, 2H), 7.43 (d, J=8.7 Hz, 2H).

A mixture of (S)-2-(4-cyanophenylamino)-3,3-dimethylbutanoic acid (72 mg, 0.31 mmol), NMM (0.07 mL, 0.61 mmol), HATU (116 mg, 0.31 mmol), HOBt (6.2 mg, 0.05 mmol) and hydrochloride salt of (2S,4R)—N-((1R,2S)-1-(cyclopropylsulfonylcarbamoyl)-2-vinylcyclopropyl)-4-(2-(4-(trifluoromethyl)phenyl)benzofuro[3,2-d]-pyrimidin-4-yloxy)pyrrolidine-2-carboxamide (100 mg, 0.15 mmol) in 0.8 mL of dry methylene chloride was stirred at room temperature for 4 h. The solid in the reaction mixture was removed by filtration and the methylene chloride solution was extracted twice with saturated NH₄Cl aqueous solution, dried over MgSO₄ and concentrated in vacuo. The crude material was purified by silica gel column with 10% ethyl acetate/methylene chloride to give 28 mg of (2S,4R)-1-((S)-2-(4-cyanophenylamino)-3,3-dimethylbutanoyl)-N-((1R,2S)-1-(cyclopropylsulfonylcarbamoyl)-2-vinylcyclopropyl)-4-(2-(4-(trifluoromethyl)phenyl)benzofuro[3,2-d]pyrimidin-4-yloxy)pyrrolidine-2-carboxamide (Compound 64). LC-MS (M+H): 870.3, ¹H-NMR (CDCl₃, 300 Hz): δ1.06 (m, 2H), 1.11 (s, 9H), 1.40 (m, 2H), 2.54 (m, 2H), 2.70 (m, 2H), 2.91 (m, 1H), 3.97 (d, J=9.9 Hz, 1H), 4.22 (m, 2H), 4.57 (t, J=8.3 Hz, 1H), 4.87 (d, J=9.3 Hz, 1H), 5.16 (d, J=10.5 Hz, 1H), 5.25 (d, J=16.8 Hz, 1H), 5.76 (m, 1H), 6.22 (m, 1H), 6.47 (d, J=8.7 Hz, 2H), 6.98 (s, 1H), 7.24 (d, J=9.6 Hz, 2H), 7.54 (m, 1H), 7.74 (d, J=3.9 Hz, 2H), 7.80 (d, J=8.4 Hz, 2H), 8.30 (d, J=7.8 Hz, 1H), 8.65 (d, J=8.4 Hz, 2H), 10.11 (s, 1H).

### Example 3

### Preparation of Compound 50

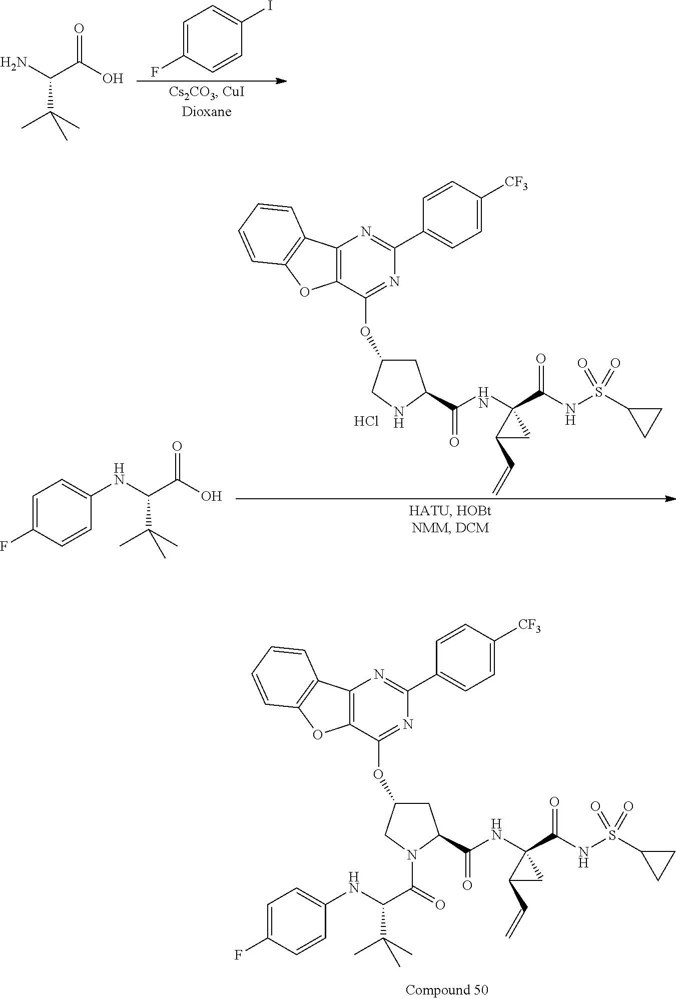

To a stirred solution of (S)-2-(4-fluorophenylamino)-3,3-dimethylbutanoic acid (0.052 g, 0.23 mmol) (prepared similarly to the procedure for the synthesis of (S)-2-(4-cyanophenylamino)-3,3-dimethylbutanoic acid described above) in 0.75 mL of methylene chloride at 0° C. were added N-methyl-morpholine (NMM, 0.082 mL, 0.75 mmol), O-(7-Azabenzotriazol-1-yl)-N,N,N′,N′-tetramethyluronium hexafluorophosphate (HATU, 0.11 g, 0.30 mmol), N-hydroxybenzotriazole (HOBt, 0.006 g, 0.045 mmol) and (2S,4R)—N-((2S)-1-(cyclopropyl-sulfonylcarbamoyl)-2-vinylcyclopropyl)-4-(2-(4-(trifluoromethyl)-phenyl)benzofuro[3,2-d]pyrimidin-4-yloxy)pyrrolidine-2-carboxamide hydrochloride salt (0.1 g, 0.15 mmol). The mixture was stirred at room temperature for 24 hours. The solvent was removed by evaporation and the residue was dissolved in methylene chloride. The solution was washed with saturated ammonium chloride aqueous solution, brine, dried over anhydrous MgSO₄, and concentrated in vacuum. The residue was purified by silica gel column chromatography with 4% ethyl acetate/methylene chloride to give 0.07 6 g of (2S,4R)—N-((2S)-1-(cyclopropylsulfonylcarbamoyl)-2-vinylcyclopropyl)-1-((S)-2-(4-fluorophenylamino)-3,3-dimethylbutanoyl)-4-(2-(4-(trifluoromethyl)phenyl)-benzofuro[3,2-d]pyrimidin-4-yloxy)pyrrolidine-2-carboxamide (Compound 50). ¹H NMR (300 MHz, CDCl₃): δ 10.18 (s, 1H), 8.63 (d, J=8.1 Hz, 2H), 8.26 (d, J=7.8, 1H), 7.79 (d, J=9.0 Hz, 2H), 7.68 (d, J=2.7 Hz, 2H), 7.49-7.54 (m, 1H), 7.21 (s, 1H), 6.65 (t, J=8.7 Hz, 2H), 6.47 (dd, J=8.7, 4.5 Hz, 2H), 6.14 (s, 1H), 5.67-5.79 (m, 1H), 5.22 (d, J=31.8 Hz, 1H), 5.17 (d, J=25.8 Hz, 1H), 4.55 (t, J=8.1 Hz, 1H), 4.10-4.24 (m, 2H), 3.81 (d, J=10.5 Hz, 1H), 2.87-2.94 (m, 1H), 2.62-2.67 (m, 2H), 1.95-2.10 (m, 2H), 1.46-1.51 (m, 1H), 1.32-1.42 (m, 3H), 1.11 (s, 9H), 1.02-1.07 (m, 2H). LC-MS (M+H): 863.1

### Example 4

### Preparation of Compound 91

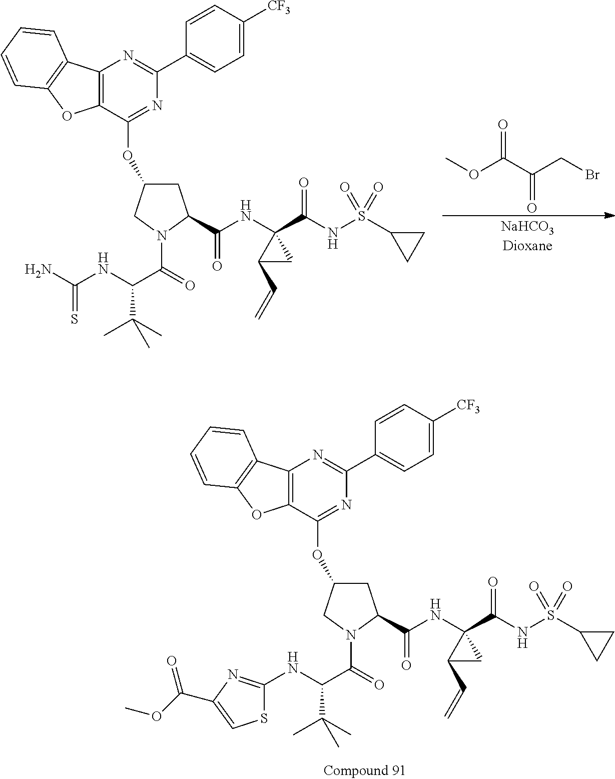

To the solution of (2S,4R)—N-((2S)-1-(cyclopropylsulfonylcarbamoyl)-2-vinylcyclopropyl)-1-((S)-3,3-dimethyl-2-thioureidobutanoyl)-4-(2-(4-(trifluoro-methyl)-phenyl)benzofuro[3,2-d]pyrimidin-4-yloxy)pyrrolidine-2-carboxamide (0.157 g, 0.19 mmol) in 1.9 mL of dry dioxane were added sodium bicarbonate (NaHCO₃, 0.80 g, 9.5 mmol) and methyl 3-bromo-2-oxopropanoate (0.062 g, 0.34 mmol). The reaction mixture was stirred at 60° C. for 2 hours. The solvent was removed by evaporation and the residue was dissolved in methylene chloride. The solution was washed with water brine, dried over anhydrous MgSO₄, and concentrated in vacuum. The residue was purified by silica gel column chromatography with 20% ethyl acetate/methylene chloride to give 0.12 g of methyl 2-((2S)-1-((2S,4R)-2-((2S)-1-(cyclopropylsulfonylcarbamoyl)-2-vinylcyclopropyl-carbamoyl)-4-(2-(4-(trifluoromethyl)phenyl)benzofuro[3,2-d]pyrimidin-4-yloxy-)pyrrolidin-1-yl)-3,3-dimethyl-1-oxobutan-2-ylamino)thiazole-4-carboxylate (Compound 91). 1H NMR (300 MHz, CDCl₃): δ 10.16 (s, 1H), 8.68 (d, J=7.8 Hz, 2H), 8.28 (d, J=7.8 Hz, 1H), 7.81 (d, J=8.4 Hz, 2H), 7.49-7.69 (m, 3H), 7.04 (s, 1H), 6.28 (m, 1H), 5.65-5.77 (m, 1H), 5.26 (d, J=16.8 Hz, 1H), 5.15 (d, J=10.5 Hz, 1H), 4.94-5.10 (m, 1H), 4.42-4.57 (m, 1H), 4.30-4.42 (m, 1H), 3.95-4.17 (m, 1H), 3.66-3.95 (m, 1H), 3.56 (s, 3H), 2.86-2.96 (m, 1H), 2.64-2.82 (m, 2H), 2.12-2.22 (m, 1H), 1.78-1.90 (m, 1H), 1.54-1.56 (m, 1H), 1.36-1.48 (m, 2H), 1.21-1.36 (m, 2H), 1.14 (s, 9H), 0.98-1.09 (m, 1H). LC-MS (M+H): 910.2

### Example 5

### Preparation of Compound 95

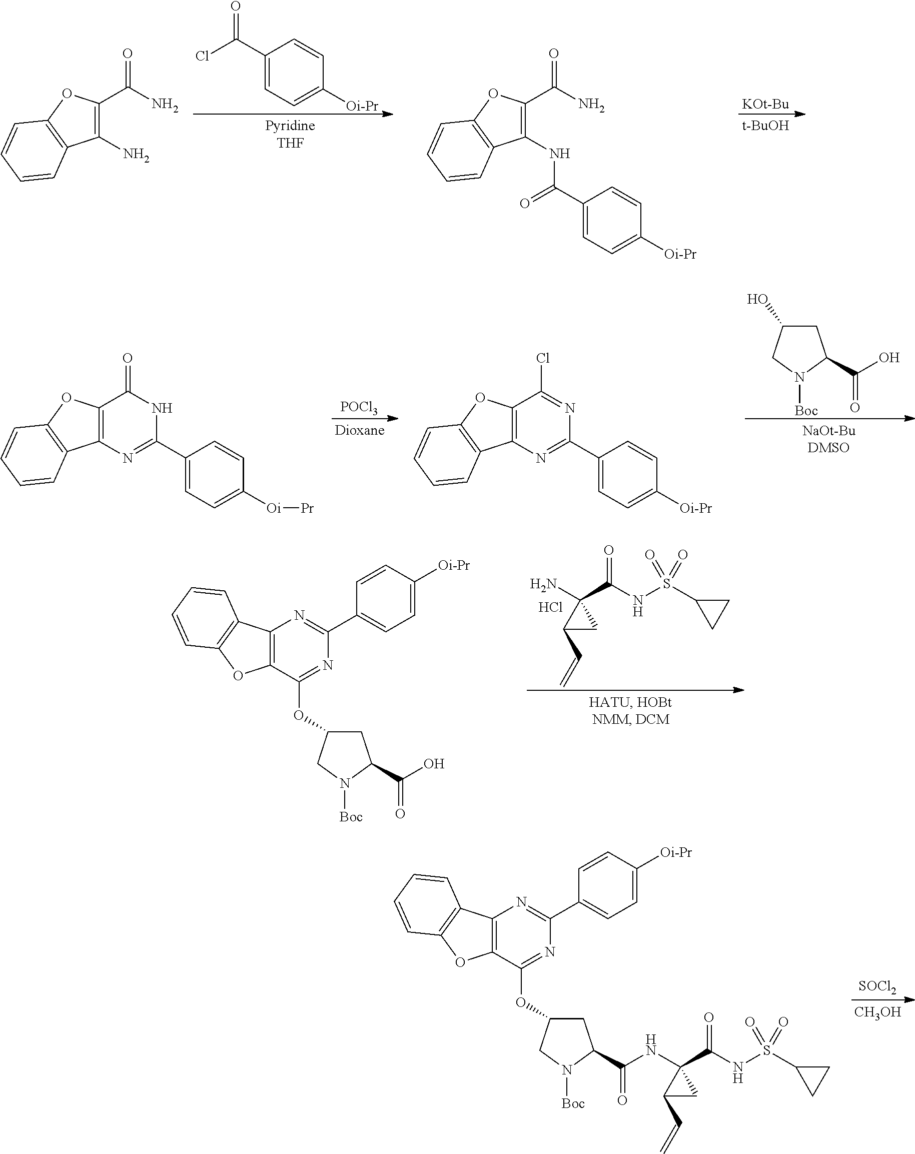

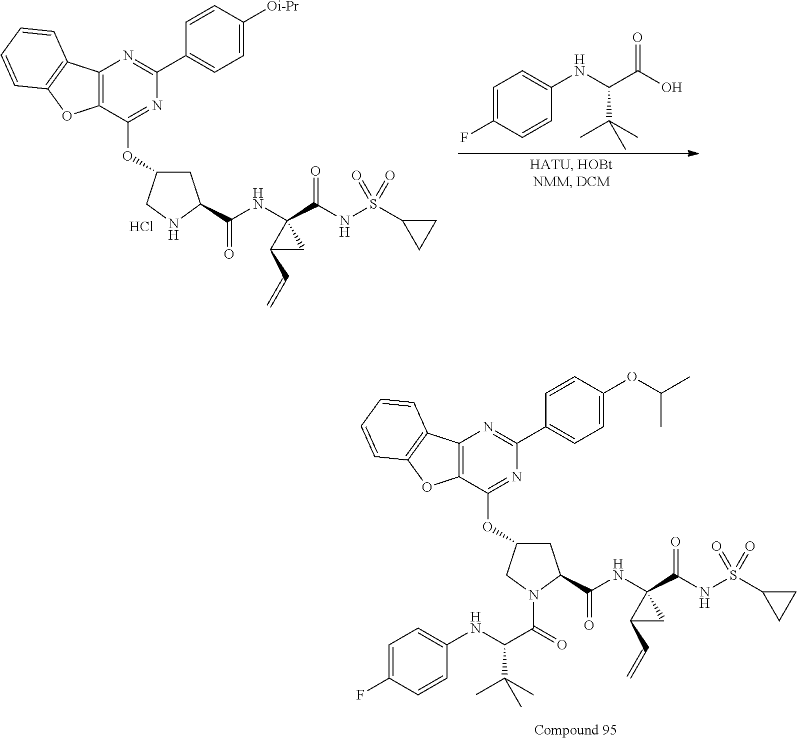

To a stirred solution of 3-aminobenzofuran-2-carboxamide (3.00 g, 17.03 mmol) in 85 mL of THF were added pyridine (4.10 mL, 51.09 mmol) and 4-isopropoxybenzoyl chloride (4.06 g, 20.44 mmol) at 0° C. and the mixture was stirred at room temperature overnight. The solvent was removed by evaporation and the residue was poured into 200 ml, of water. The mixture was stirred for 1 h. The precipitate was collected by filtration and washed with 200 mL of hexane, washed with 400 mL of methylene chloride/hexane (1/50) and dried under reduced pressure to give 5.34 g of 3-(4-isopropoxybenzamido)benzofuran-2-carboxamide. LC-MS (M+H): 339.1.

To a stirred solution of 3-(4-isopropoxybenzamido)benzofuran-2-carboxamide (5.34 g, 15.79 mmol) in 39.5 mL of tBuOH and 7.9 mL of water was added 1N KOtBu aqueous solution (31.6 mL, 31.58 mmol) and the mixture was stirred at 70° C. overnight. The solvent was removed by evaporation and the residue was poured into ice water. The aqueous solution was treated with 6N HCl aqueous solution to adjust pH to ˜2. The precipitate was collected by filtration, washed with hexane and then methylene chloride/hexane, and dried under reduced pressure to give 4.92 g of 2-(4-isopropoxyphenyl)benzofuro[3,2-d]pyrimidin-4(3H)-one. LC-MS (M+H): 321.1, ¹H-NMR (CDCl₃, 300 Hz): δ1.41 (d, J=6.0 Hz, 6H), 4.70 (m, 1H), 7.09 (d, J=8.7 Hz, 2H), 7.46 (t, 7.2 Hz, 1H), 7.62 (t, J=8.0 Hz, 1H), 7.69 (d, J=8.1 Hz, 1H), 8.18 (d, J=7.5 Hz, 1H), 8.24 (d, J=9.0 Hz, 2H), 11.45 (s, 1H).

To a stirred solution of 2-(4-isopropoxyphenyl)benzofuro[3,2-d]-pyrimidin-4(3H)-one (4.91 g, 15.33 mmol) in 31.0 mL of 1,4-dioxane was added POCl₃ (7.1 mL, 76.66 mmol) at 0° C. and the reaction mixture was stirred at 110° C. for 2 h. The solvent was removed by evaporation, ice water was added, and saturated NaHCO₃ aqueous solution was used to adjust pH to ˜9. The precipitate was collected by filtration, washed with water, and then dissolved in methylene chloride. The methylene chloride solution was dried over MgSO₄ and concentrated in vacuo to give 4.38 g of 4-chloro-2-(4-isopropoxyphenyl)benzofuro[3,2-d]pyrimidine. LC-MS (M+H): 339.0.

To a stirred solution of (2S,4R)-1-(tert-butoxycarbonyl)-4-hydroxy-pyrrolidine-2-carboxylic acid (3.59 g, 15.52 mmol) in 65 mL of DMSO was added NaOtBu (2.86 g, 29.75 mmol) at 0° C. and the mixture was stirred at room temperature for 2 h. To the mixture was added 4-chloro-2-(4-isopropoxyphenyl)benzofuro[3,2-d]pyrimidine (4.38 g, 12.93 mmol) and 65 mL DMSO. The mixture was stirred at room temperature for 1 h. The mixture was poured into ice water, treated with 6N HCl aqueous solution to adjust pH to ˜3, and extracted with ethyl acetate. The ethyl acetate layer was washed with water and then brine, dried over MgSO₄ and concentrated in vacuo to give 6.91 g of (2S,4R)-1-(tert-butoxycarbonyl)-4-(2-(4-isopropoxyphenyl)benzofuro[3,2-d]pyrimidin-4-yloxy)pyrrolidine-2-carboxylic acid. LC-MS (M+H): 534.2.

A mixture of (2S,4R)-1-(tert-butoxycarbonyl)-4-(2-(4-isopropoxyphenyl)-benzofuro[3,2-d]pyrimidin-4-yloxy)pyrrolidine-2-carboxylic acid (6.91 g, 12.95 mmol), NMM (7.1 mL, 64.77 mmol), HATU (9.85 g, 25.91 mmol), HOBt (0.53 g, 3.89 mmol) and hydrochloride salt of (1R,2S)-1-amino-N-(cyclopropylsulfonyl)-2-vinylcyclopropanecarboxamide (4.48 g, 19.43 mmol) in 65 mL of dry methylene chloride at 0° C. was stirred at room temperature for 6 h. The solid in the reaction solution was removed by filtration and the methylene chloride solution was extracted twice with saturated NH₄Cl aqueous solution, dried over MgSO₄, and concentrated in vacuo. The crude material was purified by flash column with 25% ethyl acetate/25% methylene chloride/0.5% methanol/49.5% hexane to give 6.58 g of the Boc-protected compound. LC-MS (M+H): 746.3, ¹H-NMR (CDCl₃, 300 Hz): δ1.06 (m, 2H), 1.28 (m, 3H), 1.39 (d, J=6.0 Hz, 6H), 1.48 (s, 9H), 2.03 (m, 1H), 2.12 (m, 1H), 2.63 (m, 2H), 2.96 (m, 1H), 3.95 (m, 2H), 4.39 (t, J=8.0 Hz, 1H), 4.67 (m, 1H), 5.16 (d, J=10.2 Hz, 1H), 5.30 (d, J=17.1 Hz, 1H), 5.80 (m, 1H), 6.07 (m, 1H), 7.01 (d, J=9.0 Hz, 2H), 7.05 (s, 1H), 7.48 (m, 1H), 7.66 (d, J=3.6 Hz, 2H), 8.26 (d, J=7.8 Hz, 1H), 8.44 (d, J=9.0 Hz, 2H), 10.11 (s, 1H).

The Boc-protected compound (6.58 g, 8.82 mmol) in 88 mL of methanol was added thionyl chloride (1.93 mL, 26.46 mmol) at 0° C. and the reaction mixture was then stirred at 45° C. for 2 h. The organic solvent was removed by evaporation to give 6.08 g of the hydrochloride salt of (2S,4R)—N-((1R,2S)-1-(cyclopropylsulfonylcarbamoyl)-2-vinylcyclopropyl)-4-(2-(4-isopropoxyphenyl)benzofuro[3,2-d]pyrimidin-4-yloxy)pyrrolidine-2-carboxamide. LC-MS (M+H): 646.2.

To a stirred solution of (S)-2-(4-fluorophenylamino)-3,3-dimethylbutanoic acid (0.12 g, 0.46 mmol) in 1.55 mL of methylene chloride at 0° C. were added N-methyl-morpholine (NMM, 0.17 mL, 1.55 mmol), O-(7-Azabenzotriazol-1-yl)-N,N,N′,N′-tetramethyluronium hexafluorophosphate (HATU, 0.24 g, 0.62 mmol), N-hydroxybenzotriazole (HOBt, 0.013 g, 0.093 mmol), and (2S,4R)—N-((2S)-1-(cyclopropyl-sulfonylcarbamoyl)-2-vinylcyclopropyl)-4-(2-(4-isopropoxyphenyl)benzofuro[3,2-d]pyrimidin-4-yloxy)pyrrolidine-2-carboxamide hydrochloride salt (0.2 g, 0.31 mmol). The mixture was stirred at room temperature for 22 hours. The solvent was removed by evaporation and the residue was dissolved in methylene chloride. The solution was washed with a saturated ammonium chloride aqueous solution, and then brine, dried over anhydrous MgSO₄, and concentrated in vacuum. The residue was purified by silica gel column chromatography with 10% ethyl acetate/methylene chloride to give 0.14 g of (2S,4R)—N-((2S)-1-(cyclopropylsulfonylcarbamoyl)-2-vinylcyclopropyl)-1-((S)-2-(4-fluorophenylamino)-3,3-dimethylbutanoyl)-4-(2-(4-isopropoxyphenyl)benzofuro[3,2-d]pyrimidin-4-yloxy)pyrrolidine-2-carboxamide (Compound 95). ¹H NMR (300 MHz, CDCl₃): δ 10.19 (s, 1H), 8.44 (d, J=9.0 Hz, 2H), 8.25 (d, J=7.8 Hz, 1H), 7.66 (d, J=3.0 Hz, 2H), 7.45-7.50 (m, 1H), 7.10 (s, 1H), 7.03 (d, J=9.0 Hz, 2H), 6.65 (t, J=8.7 Hz, 2H), 6.44-6.48 (m, 2H), 6.11 (s, 1H), 5.68-5.80 (m, 1H), 5.22 (d, J=32.7 Hz, 1H), 5.18 (d, J=28.3 Hz, 1H), 4.65-4.73 (m, 1H), 4.52 (t, J=8.4 Hz, 1H), 4.09-4.24 (m, 2H), 3.81 (s, 1H), 2.88-2.96 (m, 1H), 2.62 (d, J=8.4 Hz, 2H), 1.95-2.17 (m, 2H), 1.47-1.52 (m, 1H), 1.41 (d, J=5.7 Hz, 6H), 1.37-1.38 (m, 3H), 1.13 (s, 9H), 1.00-1.09 (m, 2H). LC-MS (M+H): 853.3

### Example 6

### Preparation of Compound 100

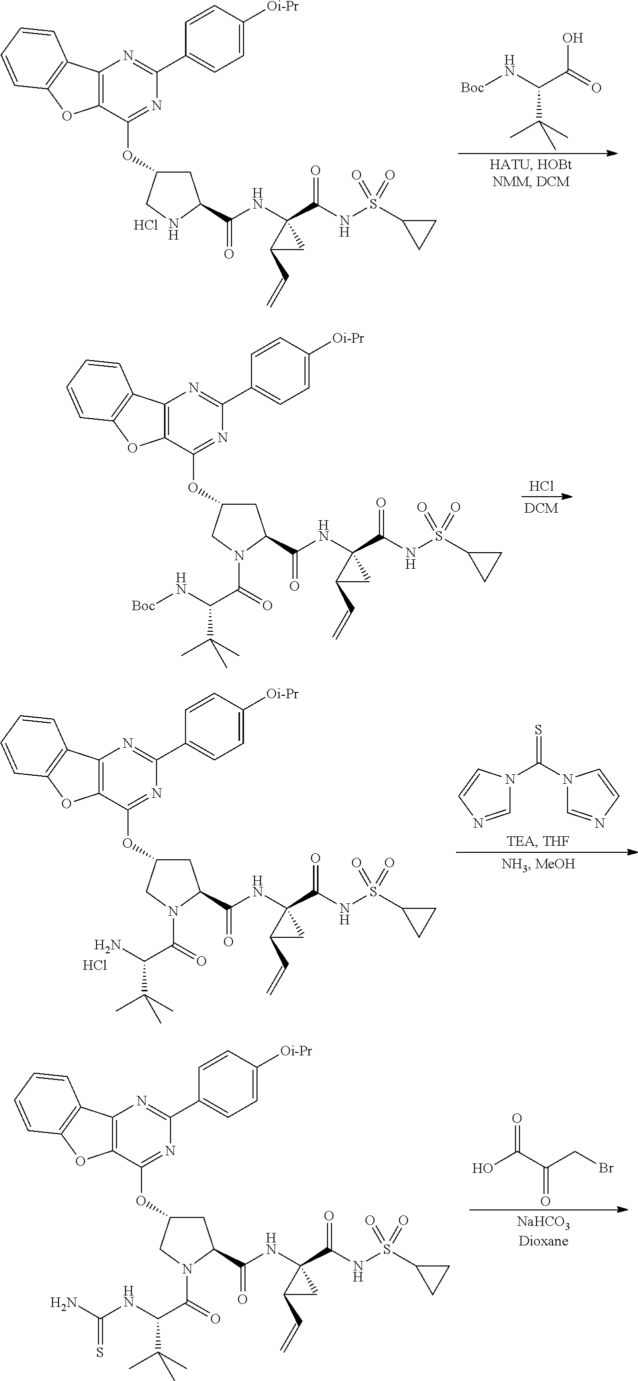

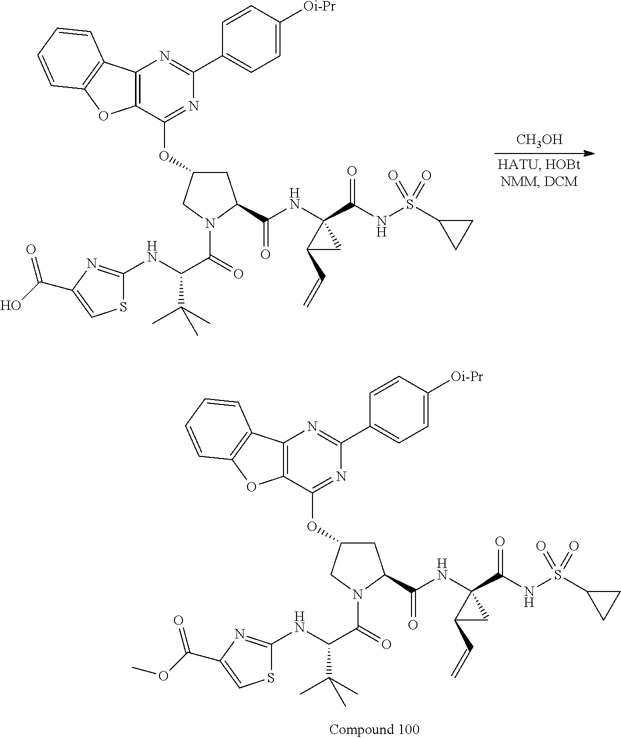

To a stirred solution of (S)-2-(tert-butoxycarbonylamino)-3,3-dimethylbutanoic acid (0.59 g, 2.41 mmol) in 10 mL of methylene chloride at 0° C. were added N-methylmorpholine (NMM, 0.90 mL, 8.04 mmol), O-(7-Azabenzotriazol-1-yl)-N,N,N′,N′-tetramethyluronium hexafluorophosphate (HATU, 1.53 g, 4.02 mmol), N-hydroxybenzotriazole (HOBt, 0.081 g, 0.6 mmol) and (2S,4R)—N-((2S)-1-(cyclopropylsulfonylcarbamoyl)-2-vinylcyclopropyl)-4-(2-(4-isopropoxyphenyl)benzofuro[3,2-d]-pyrimidin-4-yloxy)pyrrolidine-2-carboxamide(S)-2-(tert-butoxycarbonylamino)-3,3-dimethylbutanoate hydrochloride salt (1.3 g, 2.01 mmol). The mixture was stirred at room temperature for 5 hours. The solvent was removed by evaporation and the residue was dissolved in methylene chloride. The solution was washed with a saturated ammonium chloride aqueous solution, and then brine, dried over anhydrous MgSO₄, and concentrated in vacuum. The residue was purified by silica gel column chromatography with 18% ethyl acetate/methylene chloride to give 1.37 g of tert-butyl (2S)-1-((2S,4R)-2-((2S)-1-(cyclopropyl-sulfonylcarbamoyl)-2-vinylcyclopropylcarbamoyl)-4-(2-(4-isopropoxyphenyl)-benzofuro[3,2-d]pyrimidin-4-yloxy)pyrrolidin-1-yl)-3,3-dimethyl-1-oxobutan-2-ylcarbamate. LC-MS (M+H): 859.3.

To a stirred solution of tert-butyl (2S)-1-((2S,4R)-2-((2S)-1-(cyclopropyl-sulfonylcarbamoyl-)-2-vinylcyclopropylcarbamoyl)-4-(2-(4-isopropoxyphenyl)benzofuro-[3,2-d]pyrimidin-4-yloxy)pyrrolidin-1-yl)-3,3-dimethyl-1-oxobutan-2-ylcarbamate (1.37 g, 1.59 mmol) in 16.0 mL of dichloromethane at 0° C. was added 4M HCl in dioxane (8.0 mL, 7.95 mmol). The mixture was stirred at 0° C. for 5.5 hours. The solvent was removed by concentrated in vacuum to give 1.33 g of (2S,4R)-1-((S)-2-amino-3,3-dimethylbutanoyl)-N-((2S)-1-(cyclopropylsulfonylcarbamoyl)-2-vinylcyclopropyl)-4-(2-(4-isopropoxy-phenyl)benzofuro[3,2-d]pyrimidin-4-yloxy)pyrrolidine-2-carboxamide hydrochloride salt. LC-MS (M+H): 759.3.

To the solution of (2S,4R)-1-((S)-2-amino-3,3-dimethylbutanoyl)-N-((2S)-1-(cyclopropylsulfonylcarbamoyl)-2-vinylcyclopropyl)-4-(2-(4-isopropoxyphenyl)-benzofuro[3,2-d]pyrimidin-4-yloxy)pyrrolidine-2-carboxamide hydrochloride salt (1.33 g, 1.75 mmol) in 9.0 mL of dry tetrahydrofuran at 0° C. were added triethylamine (Et₃N, 1.20 mL, 8.75 mmol) and di(1H-imidazol-1-yl)methanethione (0.62 g, 3.5 mmol). The mixture was stirred at room temperature for 2 hours. Then 7N NH₃/MeOH (2.5 mL, 17.5 mmol) was added at 0° C. and the mixture was stirred at room temperature for 3 h. The solvent was removed by concentrated in vacuum to give 1.54 g of (2S,4R)—N-((2S)-1-(cyclopropylsulfonyl-carbamoyl)-2-vinylcyclopropyl)-1-((S)-3,3-dimethyl-2-thioureidobutanoyl)-4-(2-(4-isopropoxyphenyl)benzofuro[3,2-d]pyrimidin-4-yloxy)pyrrolidine-2-carboxamide. LC-MS (M+H): 818.3.

To the solution of (2S,4R)—N-((2S)-1-(cyclopropylsulfonylcarbamoyl)-2-vinylcyclopropyl)-1-((S)-3,3-dimethyl-2-thioureidobutanoyl)-4-(2-(4-isopropoxyphenyl)-benzofuro[3,2-d]pyrimidin-4-yloxy)pyrrolidine-2-carboxamide (0.78 g, 0.95 mmol) in 9.5 mL of dry dioxane were added sodium bicarbonate (NaHCO₃, 0.80 g, 9.5 mmol) and 3-bromo-2-oxopropanoic acid (0.47 g, 2.84 mmol). The mixture was stirred at 60° C. for 1.5 hours. The solvent was removed by evaporation and the residue was dissolved in methylene chloride. The solution was washed with water, and then brine, dried over anhydrous MgSO₄, and concentrated in vacuum. 0.41 g of 2-((2S)-1-((2S,4R)-2-((2S)-1-(cyclopropyl-sulfonylcarbamoyl)-2-vinylcyclopropylcarbamoyl)-4-(2-(4-isopropoxyphenyl)-benzofuro[3,2-d]pyrimidin-4-yloxy)pyrrolidin-1-yl)-3,3-dimethyl-1-oxobutan-2-ylamino)thiazole-4-carboxylic acid. LC-MS (M+H): 886.3.

To a stirred solution of 2-((2S)-1-((2S,4R)-2-((2S)-1-(cyclopropylsulfonylcarbamoyl)-2-vinylcyclopropylcarbamoyl)-4-(2-(4-isopropoxyphenyl)benzofuro[3,2-d]pyrimidin-4-yloxy)pyrrolidin-1-yl)-3,3-dimethyl-1-oxobutan-2-ylamino)thiazole-4-carboxylic acid (0.1 g, 0.11 mmol) in 0.56 mL of methylene chloride at 0° C. were added N-methylmorpholine (NMM, 0.05 mL, 0.45 mmol), O-(7-Azabenzotriazol-1-yl)-N,N,N′,N′-tetramethyluronium hexafluorophosphate (HATU, 0.086 g, 0.22 mmol), N-hydroxybenzotriazole (HOBt, 0.005 g, 0.034 mmol), and MeOH (0.05 mL, 1.1 mmol). The mixture was stirred at room temperature for 4 hours. The solvent was removed by evaporation and the residue was dissolved in methylene chloride. The solution was washed with a saturated ammonium chloride aqueous solution, and then brine, dried over anhydrous MgSO₄, and concentrated in vacuum. The residue was purified by silica gel column chromatography with 20% ethyl acetate/methylene chloride to give 0.021 g of methyl 2-((2S)-1-((2S,4R)-2-((2S)-1-(cyclopropylsulfonylcarbamoyl)-2-vinylcyclopropyl-carbamoyl)-4-(2-(4-isopropoxyphenyl)benzofuro[3,2-d]pyrimidin-4-yloxy)pyrrolidin-1-yl)-3,3-dimethyl-1-oxobutan-2-ylamino)thiazole-4-carboxylate (Compound 100). ¹H NMR (300 MHz, CDCl₃): δ 10.21 (s, 1H), 8.49 (d, J=9.0 Hz, 2H), 8.26 (d, J=7.8 Hz, 1H), 7.45-7.64 (m, 3H), 7.04 (d, J=8.7 Hz, 2H), 6.98 (s, 1H), 6.18-6.28 (m, 1H), 5.65-5.77 (m, 1H), 5.25 (d, J=16.5 Hz, 1H), 5.14 (d, J=11.7 Hz, 1H), 5.0-5.10 (m, 1H), 4.66-4.74 (m, 1H), 4.30-4.48 (m, 2H), 3.89-4.04 (m, 1H), 3.60 (s, 3H), 2.87-2.96 (m, 1H), 2.63-2.77 (m, 2H), 2.14-2.22 (m, 1H), 1.76-1.84 (m, 1H), 1.44-1.47 (m, 3H), 1.41 (d, J=5.7 Hz, 6H), 1.21-1.29 (m, 2H), 1.13 (s, 9H), 0.97-1.08 (m, 2H). LC-MS (M+H): 900.3.

### Example 7

### Preparation of Compound 101

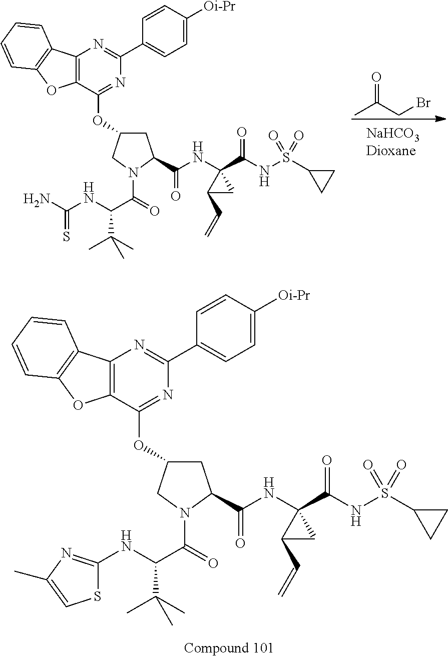

A mixture of 1-((S)-1-((2S,4R)-2-(((1R,2S)-1-(cyclopropylsulfonylcarbamoyl)-2-vinylcyclopropyl)carbamoyl)-4-(2-(4-isopropoxyphenyl)benzofuro[3,2-d]pyrimidin-4-yloxy)pyrrolidin-1-yl)-3,3-dimethyl-1-oxobutan-2-yl)thiourea (0.20 g, 0.24 mmol), sodium bicarbonate (0.20 g, 2.4 mmol) and bromoacetone (0.04 mL, 0.48 mmol) in 2.4 mL of 1,4-dioxane was stirred at 60° C. for 3 h. The solvent was removed by evaporation and the residue was dissolved in methylene chloride. The methylene chloride solution was washed with water, dried over MgSO₄ and concentrated in vacuo. The crude material was purified by silica gel column with 10% ethyl acetate/1% methanol/methylene chloride to give 38 mg of (2S,4R)—N-((1R,2S)-1-(cyclopropylsulfonylcarbamoyl-)-2-vinylcyclopropyl)-1-((S)-3,3-dimethyl-2-(4-methylthiazol-2-ylamino)butanoyl)-4-(2-(4-isopropoxyphenyl)benzofuro[3,2-d]pyrimidin-4-yloxy)pyrrolidine-2-carboxamide (Compound 101). LC-MS (M+H): 856.3, ¹H-NMR (CDCl₃, 300 Hz): δ1.06 (m, 2H), 1.10 (s, 9H), 1.31 (m, 2H), 1.41 (d, J=6.0 Hz, 6H), 1.53 (m, 1H), 1.82 (s, 3H), 1.92 (m, 1H), 2.12 (m, 1H), 2.67 (m, 2H), 2.92 (m, 1H), 4.14 (m, 1H), 4.27 (dd, J₁=11.7 Hz, J₂=4.4 Hz, 1H), 4.55 (d, J=11.7 Hz, 1H), 4.69 (m, 1H), 4.76 (t, J=8.1 Hz, 1H), 5.14 (d, J=11.1 Hz, 1H), 5.26 (d, J=16.8 Hz, 1H), 5.61 (s, 1H), 5.69 (m, 1H), 6.22 (m, 1H), 7.04 (d, J=8.7 Hz, 2H), 7.48 (t, J=7.4 Hz, 1H), 7.63 (m, 2H), 8.26 (d, J=7.8 Hz, 1H), 8.47 (d, J=8.7 Hz, 2H), 10.27 (s, 1H).

### Example 8

### Preparation of Compound 141

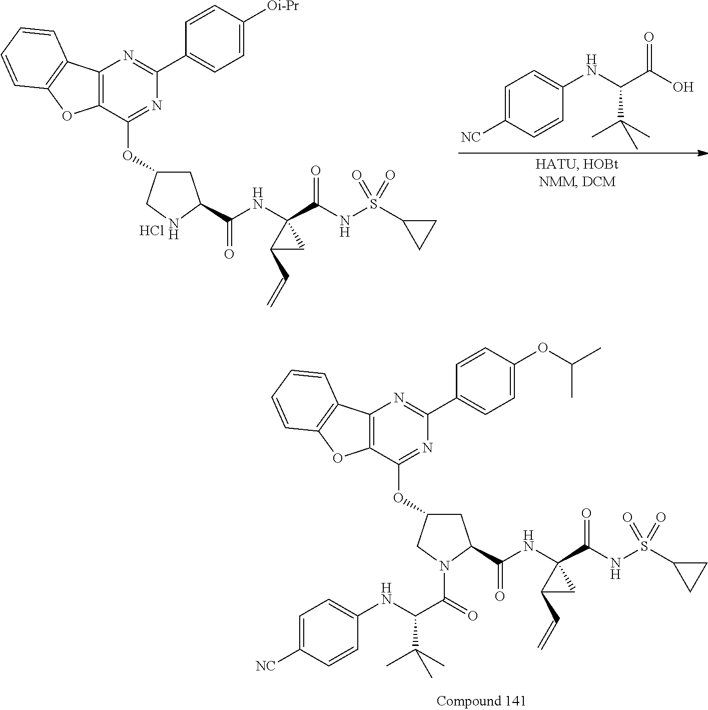

A mixture of (S)-2-(4-cyanophenylamino)-3,3-dimethylbutanoic acid (93 mg, 0.40 mmol), NMM (0.15 mL, 1.34 mmol), HATU (254 mg, 0.67 mmol), HOBt (14 mg, 0.10 mmol) and hydrochloride salt of (2S,4R)—N-((1R,2S)-1-(cyclopropylsulfonylcarbamoyl)-2-vinylcyclopropyl)-4-(2-(4-isopropoxyphenyl)benzofuro[3,2-d]pyrimidin-4-yloxy)pyrrolidine-2-carboxamide (216 mg, 0.33 mmol) in 1.7 mL of dry methylene chloride was stirred at room temperature overnight. The solid in the reaction solution was removed by filtration and the methylene chloride solution was extracted twice with saturated NH₄Cl aqueous solution, dried over MgSO₄ and concentrated in vacuo. The crude material was purified by a silica gel column with 12% ethyl acetate/10% hexane/methylene chloride to give 109 mg of (2S,4R)-1-((S)-2-(4-cyanophenylamino)-3,3-dimethylbutanoyl)-N-((1R,2S)-1-(cyclopropylsulfonylcarbamoyl)-2-vinylcyclopropyl)-4-(2-(4-isopropoxyphenyl)benzofuro[3,2-d]pyrimidin-4-yloxy)pyrrolidine-2-carboxamide (Compound 141). LC-MS (M+H): 860.3, ¹H-NMR (CDCl₃, 300 Hz): δ1.06 (m, 2H), 1.11 (s, 9H), 1.37 (m, 1H), 1.41 (d, J=6.0 Hz, 6H), 1.52 (m, 1H), 2.05 (m, 2H), 2.67 (m, 2H), 2.92 (m, 1H), 3.96 (d, J=9.6 Hz, 1H), 4.21 (m, 2H), 4.56 (t, J=8.6 Hz, 1H), 4.69 (m, 1H), 4.88 (d, J=9.9 Hz, 1H), 5.15 (d, J=11.4 Hz, 1H), 5.25 (d, J=17.4 Hz, 1H), 5.78 (m, 1H), 6.19 (m, 1H), 6.46 (d, J=8.7 Hz, 2H), 6.99 (s, 1H), 7.03 (d, J=9.0 Hz, 2H), 7.23 (d, J=8.7 Hz, 2H), 7.39 (m, 1H), 7.70 (d, J=3.6 Hz, 2H), 8.28 (d, J=8.1 Hz, 1H), 8.46 (d, J=8.7 Hz, 2H), 10.15 (s, 1H).

### Example 9

### Preparation of Compound 165

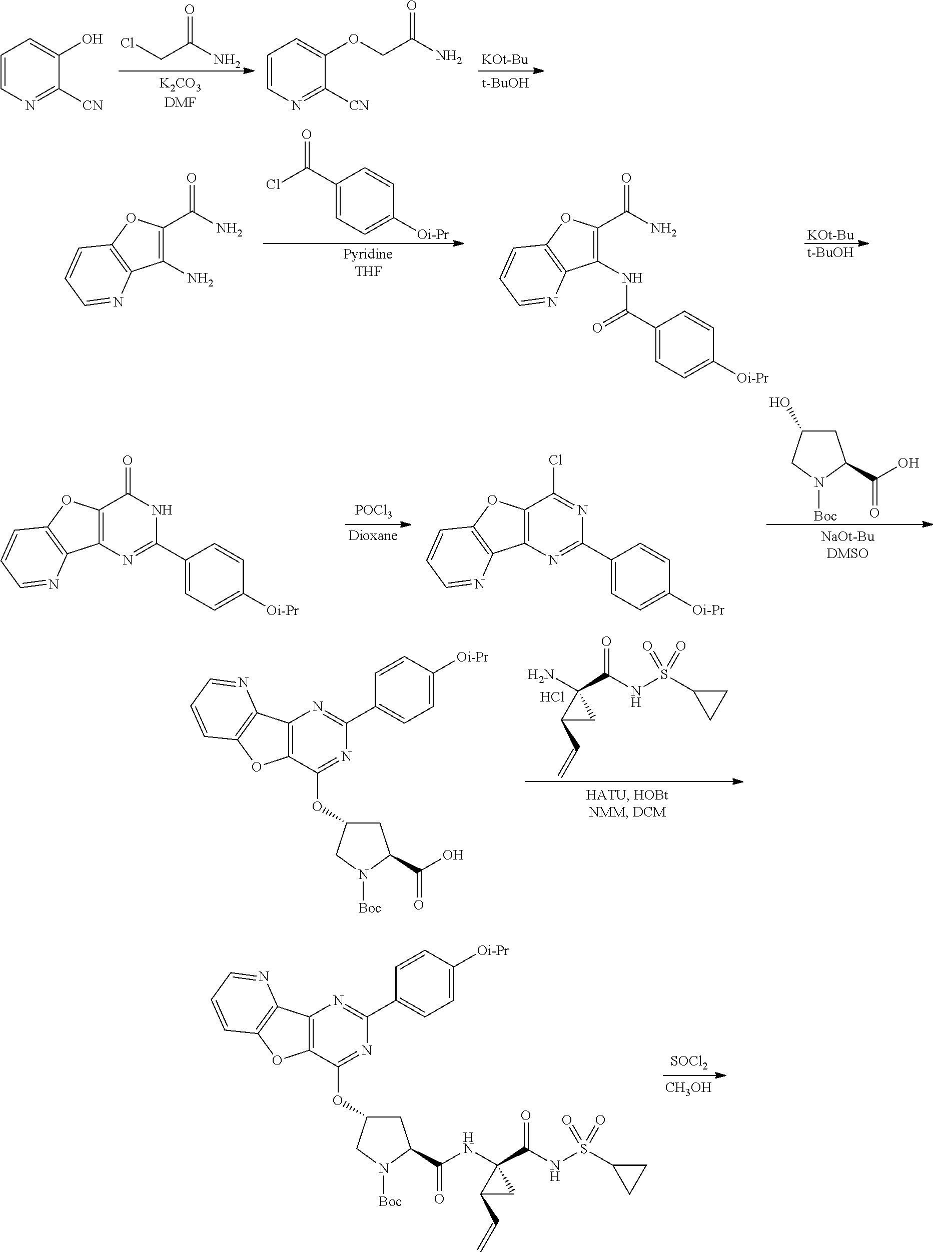

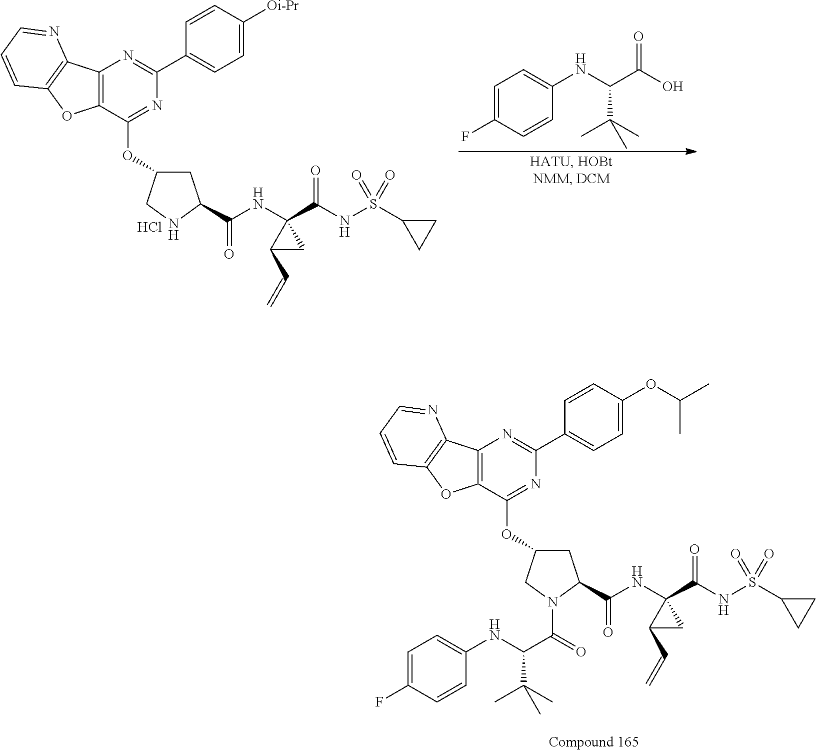

To a stirred solution of 3-hydroxypicolinonitrile (3.90 g, 32.47 mmol) in 65 mL of dried N,N-Dimethylformamide were added potassium carbonate (K₂CO₃, 1.46 g, 97.41 mmol) and 2-chloroacetamide (4.55 g, 48.71 mmol) at 80° C. for 3 hours. The mixture was poured into 40 mL water and extracted by ethyl acetate, and then brine, dried over anhydrous MgSO₄, and concentrated in vacuum. The solid was combined to give 5.39 g of 2-(2-cyanopyridin-3-yloxy)acetamide. LC-MS (M+H): 178.0.

To a stirred solution of 2-(2-cyanopyridin-3-yloxy)acetamide (5.39 g, 30.4 mmol) in 60.8 mL of tert-butanol was added potassium tert-butanoxide (KOt-Bu, 4.10 g, 36.5 mmol). The mixture was stirred at 80° C. for 2 hours. The solvent was removed by evaporation. The residue was poured into 50 mL water and extracted by methylene chloride, and then brine, dried over anhydrous MgSO₄, and concentrated in vacuum. The solid was combined to give 2.64 g of 3-aminofuro[3,2-b]pyridine-2-carboxamide. LC-MS (M+H): 178.0.

To the solution of 3-aminofuro[3,2-b]pyridine-2-carboxamide (2.0 g, 11.29 mmol) in 56 mL of dry tetrahydrofuran were added pyridine (2.73 mL, 33.87 mmol) and 4-isopropoxybenzoyl chloride (4.46 mL, 22.58 mmol) at 0° C. The mixture was stirred at 60° C. for 18 hours. The solvent was removed by evaporation. The residue was poured into of water and extracted by methylene chloride. The organic layer was separated, washed with brine, dried over MgSO₄, and concentrated in vacuo. The solid was combined to give 3.55 g of 3-(4-isopropoxybenzamido)furo[3,2-b]pyridine-2-carboxamide. LC-MS (M+H): 340.1.

To a stirred solution of 3-(4-isopropoxybenzamido)furo[3,2-b]pyridine-2-carboxamide (3.55 g, 10.46 mmol) in 26.2 mL of tert-butanol were added 1N potassium tert-butanoxide (KOtBu, 20.9 mL, 29.9 mmol) and H₂O (5.3 mL). The mixture was stirred at 75° C. for 23 hours. The solvent was removed by evaporation. The residue was poured into 100 mL of water and acidified with 2N HCl aqueous solution to pH 2. The precipitate was collected by filtration and dried under reduced pressure to give 3.3 g of 2-(4-isopropoxy-phenyl)furo[3,2-b]pyridin-[3,2-d]pyrimidin-4(3H)-one. ¹H NMR (300 MHz, CDCl3): δ 10.98 (s, 1H), 8.85 (dd, J=4.5, 1.5 Hz, 1H), 8.20 (d, J=9.0 Hz, 2H), 8.01 (dd, J=8.4, 1.5 Hz, 1H), 7.56 (dd, J=8.4, 4.5 Hz, 1H), 7.05 (d, J=6.9 Hz, 2H), 4.67-4.73 (m, 1H), 1.38 (d, J=4.8 Hz, 6H). LC-MS (M+H): 322.1.

To a stirred solution of 2-(4-isopropoxyphenyl)furo[3,2-b]pyridin[3,2-d]pyrimidin-4(3H)-one (3.20 g, 9.97 mmol) in 30.0 mL of dioxane was added phosphoryl trichloride (4.63 mL, 49.6 mmol) at 0° C. The mixture was stirred at 110° C. for 2.5 hours. The solvent was removed by evaporation. The residue was poured into 100 mL of ice water and basified with sodium bicarbonate aqueous solution to pH 8. The precipitate was collected by filtration, dried over MgSO₄, and concentrated in vacuo to give 1.13 g of 4-chloro-2-(4-isopropoxy-phenyl)furo[3,2-b]pyridi[3,2-d]pyrimidine. ¹H NMR (300 MHz, CD3OD): δ 8.85 (d, J=4.5 Hz, 1H), 8.46 (d, J=8.1 Hz, 2H), 8.30 (d, J=8.1 Hz, 1H), 7.80-7.84 (m, 1H), 6.97-7.05 (m, 2H), 4.68-4.76 (m, 1H) 1.37 (d, J=5.1 Hz, 6H). LC-MS (M+1): 340.0.

To a stirred solution of (2S,4R)-1-(tert-butoxycarbonyl)-4-hydroxypyrrolidine-2-carboxylic acid (0.92 g, 3.98 mmol) in 17.0 mL of dimethyl sulfoxide was added sodium tert-butanoxide (0.73 g, 7.64 mmol). The mixture was stirred at room temperature for 2 hours, then 4-chloro-2-(4-isopropoxy-phenyl)furo[3,2-b]pyridi[3,2-d]pyrimidine (1.13 g, 3.32 mmol) was added. The mixture was stirred at room temperature for 2 hours. The mixture was added into 100 mL of water and acidified with 2N HCl aqueous solution to pH 3. The residue was poured into water and extracted by ethyl acetate. The organic layer was separated, washed with brine, dried over MgSO₄, and concentrated in vacuum to give 1.06 g of (2S,4R)-1-(tert-butoxycarbonyl)-4-(2-(4-isopropoxyphenyl)furo[3,2-b]pyridine[3,2-d]pyrimidin-4-yloxy)pyrrolidine-2-carboxylic acid. LC-MS (M+H): 534.2.

To a stirred solution of (2S,4R)-1-(tert-butoxycarbonyl)-4-(2-(4-isopropoxyphenyl)furo[3,2-b]pyridin[3,2-d]pyrimidin-4-yloxy)pyrrolidine-2-carboxylic acid (1.06 g, 1.95 mmol) in 9.75 mL of methylene chloride at 0° C. were added N-methylmorpholine (NMM, 0.75 mL, 6.83 mmol), O-(7-Azabenzotriazol-1-yl)-N,N,N′,N′-tetramethyluronium hexafluorophosphate (HATU, 1.48 g, 3.9 mmol), N-hydroxybenzotriazole (HOBt, 0.08 g, 0.59 mmol) and (2S)-1-amino-N-(cyclopropylsulfonyl)-2-vinylcyclopropanecarboxamide hydrochloride salt (0.66 g, 2.87 mmol). The mixture was stirred at room temperature for 2 hours. The solvent was removed by evaporation and the residue was dissolved in methylene chloride. The solution was washed with a saturated ammonium chloride aqueous solution, and then brine, dried over anhydrous MgSO₄, and concentrated in vacuum. The residue was purified by silica gel column chromatography with 5% ethyl acetate/methylene chloride to give 1.19 g of (2S,4R)-tert-butyl 2-((2S)-1-(cyclopropyl-sulfonylcarbamoyl)-2-vinylcyclopropylcarbamoyl)-4-(2-(4-isopropoxyphenyl)furo[3,2-b]pyridin[3,2-d]pyrimidin-4-yloxy)pyrrolidine-1-carboxylate. ¹H NMR (300 MHz, CDCl3): δ 10.31 (s, 1H), 8.97 (d, J=4.5 Hz, 1H), 8.46 (d, J=8.7 Hz, 2H), 8.04 (d, J=8.7 Hz, 1H), 7.90 (s, 1H), 7.69 (dd, J=8.7, 4.5 Hz, 1H), 7.03 (d, J=9.0 Hz, 2H), 6.07 (s, 1H), 5.83-5.95 (m, 1H), 5.20 (t, J=10.5 Hz, 1H), 4.76 (q, J=6.0 Hz, 1H), 4.54-4.58 (m, 1H), 3.94-4.07 (m, 2H), 3.04-3.11 (m, 1H), 2.66-2.75 (m, 2H), 2.21-2.27 (m, 1H), 2.10-2.14 (m, 1H), 1.57 (s, 9H), 1.48 (d, J=6.0 Hz, 6H), 1.34-1.35 (m, 2H), 1.14-1.17 (m, 2H), 0.98-1.05 (m, 2H). LC-MS (M+H): 747.3.

To a stirred solution of (2S,4R)-tert-butyl 2-((2S)-1-(cyclopropylsulfonylcarbamoyl)-2-vinylcyclopropylcarbamoyl)-4-(2-(4-isopropoxyphenyl)furo[3,2-b]pyridin[3,2-d]pyrimidin-4-yloxy)pyrrolidine-1-carboxylate (1.09 g, 1.46 mmol) in 14.6 mL of methanol at 0° C. was added thionyl chloride (0.32 mL, 4.38 mmol). The mixture was stirred at 45° C. for 4 hours. The solvent was removed by concentrated in vacuum to give 0.98 g of (2S,4R)—N-((2S)-1-(cyclopropyl-sulfonylcarbamoyl)-2-vinylcyclopropyl)-4-(2-(4-isopropoxyphenyl)furo[3,2-b]pyridin[3,2-d]pyrimidin-4-yloxy)pyrrolidine-2-carboxamide hydrochloride salt. LC-MS (M+H): 647.2.

A mixture of (S)-2-(4-fluorophenylamino)-3,3-dimethylbutanoic acid (54 mg, 0.24 mmol), NMM (0.11 mL, 0.99 mmol), HATU (151 mg, 0.40 mmol), HOBt (8 mg, 0.06 mmol) and hydrochloride salt of amino starting material (150 mg, 0.20 mmol) in 1.0 mL of dry methylene chloride was stirred at room temperature overnight. The solid in the reaction solution was removed by filtration and the methylene chloride solution was extracted twice with saturated NH₄Cl aqueous solution, dried over MgSO₄ and concentrated in vacuo. The crude material was purified by silica gel column with 20% ethyl acetate/2% methanol/methylene chloride to give 93 mg of the desired product (Compound 165). LC-MS (M+H): 854.3, ¹H-NMR (CDCl₃, 300 Hz): δ1.06 (m, 2H), 1.17 (s, 9H), 1.25 (m, 1H), 1.41 (d, J=6.0 Hz, 6H), 1.65 (m, 2H), 2.02 (m, 1H), 2.13 (m, 1H), 2.50 (m, 1H), 2.95 (m, 1H), 3.83 (d, J=10.5 Hz, 1H), 4.10 (m, 2H), 4.33 (d, J=11.1 Hz, 1H), 4.66 (m, 1H), 4.97 (m, 3H), 5.77 (m, 1H), 5.88 (m, 1H), 6.55 (dd, J₁=8.6 Hz, J₂=4.2 Hz, 2H), 6.80 (dd, J₁=9.3 Hz, J₂=9.3 Hz, 2H), 6.84 (d, J=9.0 Hz, 2H), 7.47 (dd, J₁=8.4 Hz, J₂=4.5 Hz, 1H), 7.84 (d, J=8.1 Hz, 1H), 8.11 (d, J=8.7 Hz, 2H), 8.49 (s, 1H), 8.60 (d, J=4.5 Hz, 1H).

### Example 10

### Preparation of Compound 167

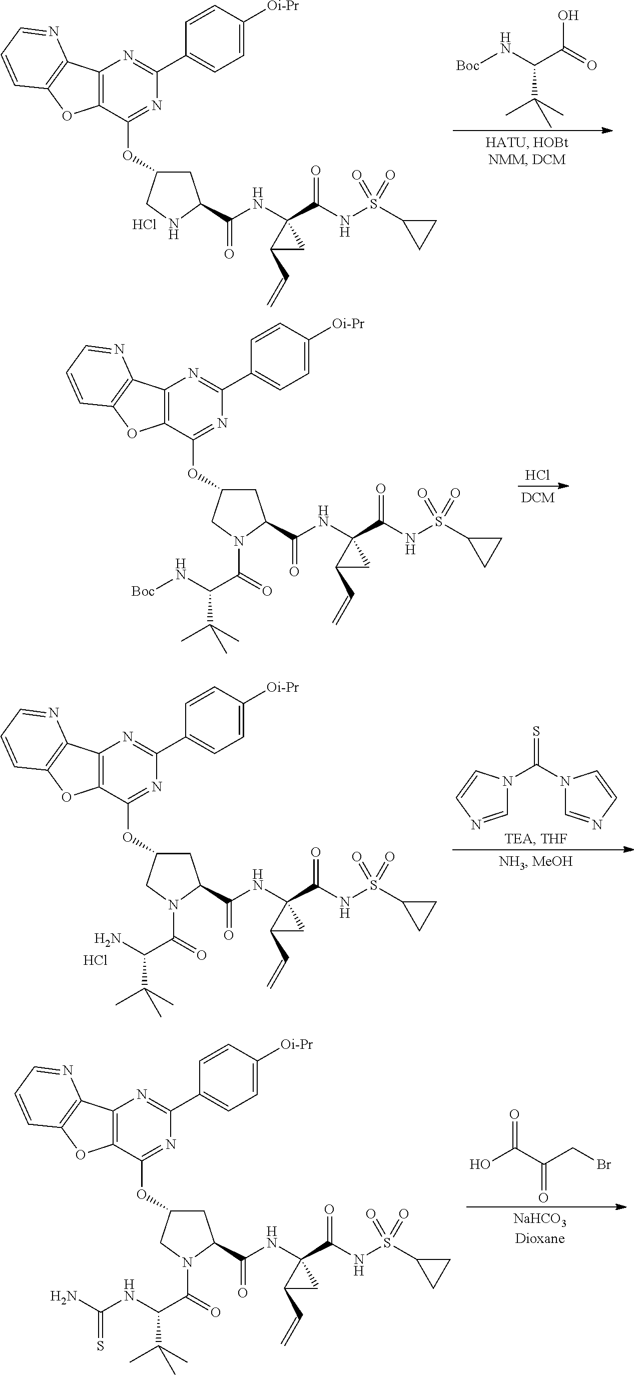

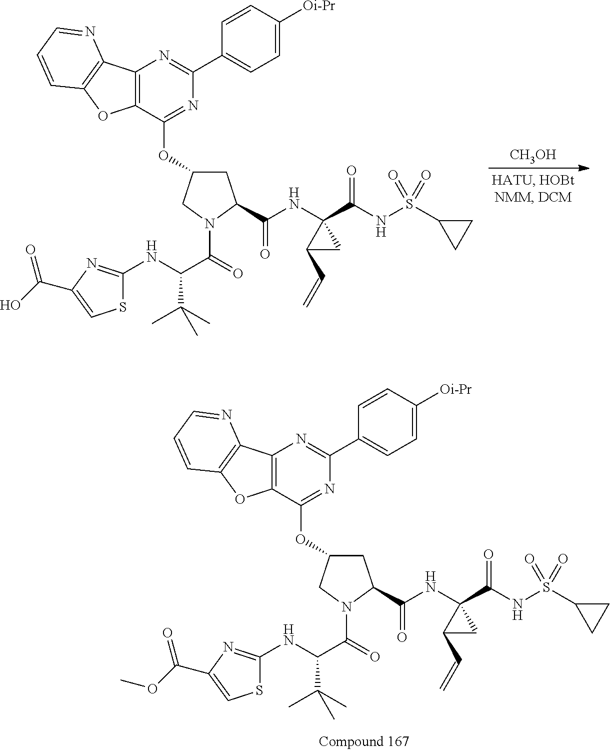

To a stirred solution of (S)-2-(tert-butoxycarbonylamino)-3,3-dimethylbutanoic acid (0.22 g, 0.95 mmol) in 3.95 mL of methylene chloride were added N-methyl-1-morpholine (NMM, 0.44 mL, 3.97 mmol), O-(7-Azabenzotriazol-1-yl)-N,N,N′,N′-tetramethyluronium hexafluorophosphate (HATU, 0.60 g, 1.58 mmol), N-hydroxybenzotriazole (HOBt, 0.032 g, 0.24 mmol) and (2S,4R)—N-((2S)-1-(cyclopropylsulfonylcarbamoyl)-2-vinylcyclopropyl)-4-(2-(4-isopropoxyphenyl)furo[3,2-b]pyridin[3,2-d]pyrimidin-4-yloxy)pyrrolidine-2-carboxamide hydrochloride salt (0.6 g, 0.79 mmol). The mixture was stirred at room temperature overnight. The solvent was removed by evaporation and residue was dissolved in methylene chloride. The solution was washed with a saturated ammonium chloride aqueous solution, and then brine, dried over anhydrous MgSO₄, and concentrated in vacuum. The residue was purified by silica gel column chromatography with 35/35/30 ethyl acetate/methylene chloride/hexane to give 0.52 g of tert-butyl (2S)-1-((2S,4R)-2-((2S)-1-(cyclopropylsulfonyl-carbamoyl)-2-vinylcyclopropylcarbamoyl)-4-(2-(4-isopropoxyphenyl)furo[3,2-b]pyridine[3,2-d]pyrimidin-4-yloxy)pyrrolidin-1-yl)-3,3-dimethyl-1-oxobutan-2-ylcarbamate. LC-MS (M+H): 860.4.

To a stirred solution of tert-butyl(2S)-1-((2S,4R)-2-((2S)-1-(cyclopropylsulfonylcarbamoyl)-2-vinylcyclopropylcarbamoyl)-4-(2-(4-isopropoxyphenyl)furo[3,2-b]pyridine[3,2-d]pyrimidin-4-yloxy)pyrrolidin-1-yl)-3,3-dimethyl-1-oxobutan-2-ylcarbamate (0.51 g, 0.6 mmol) in 6.0 mL of methylene chloride at 0° C. was added 4M HCl in dioxane (3.0 mL, 3.0 mmol). The mixture was stirred at 0° C. overnight. The solvent was removed by concentrated in vacuum to give 0.52 g of (2S,4R)-1-((S)-2-amino-3,3-dimethylbutanoyl)-N-((2S)-1-(cyclopropylsulfonyl-carbamoyl)-2-vinylcyclopropyl)-4-(2-(4-isopropoxyphenyl)furo[3,2-b]pyridine[3,2-d]pyrimidin-4-yloxy)pyrrolidine-2-carboxamide hydrochloride salt. ¹H NMR (300 MHz, CD₃OD): δ 8.41 (d, J=8.7 Hz, 2H), 8.33 (d, J=9.0 Hz, 1H), 7.00 (d, J=8.4 Hz, 2H), 6.14-6.24 (m, 1H), 5.68-5.80 (m, 1H), 5.32 (d, J=17.4 Hz, 1H), 5.15 (d, J=10.5 Hz, 1H), 4.67-4.75 (m, 1H), 4.46 (d, J=12.0 Hz, 1H), 4.23-4.37 (m, 2H), 2.92-3.05 (m, 1H), 2.70-2.80 (m, 1H), 2.37-2.51 (m, 1H), 2.24-2.37 (m, 1H), 1.88-1.98 (m, 1H), 1.43-1.48 (m, 1H), 1.38 (d, J=5.7 Hz, 6H), 1.25-1.27 (m, 2H), 1.20 (s, 9H), 1.09-1.15 (m, 2H) LC-MS (M+H): 760.3.

To a solution of (2S,4R)-1-((S)-2-amino-3,3-dimethylbutanoyl)-N-((2S)-1-(cyclopropylsulfonylcarbamoyl)-2-vinylcyclopropyl)-4-(2-(4-isopropoxyphenyl)furo[3,2-b]pyridine[3,2-d]pyrimidin-4-yloxy)pyrrolidine-2-carboxamide hydrochloride salt (0.51 g, 0.59 mmol) in 3.0 mL of dry tetrahydrofuran at 0° C. were added triethylamine (Et₃N, 0.41 mL, 2.99 mmol) and di(1H-imidazol-1-yl)methanethione (0.21 g, 1.19 mmol). The mixture was stirred at room temperature for 2 hours and then 7N NH₃/MeOH (0.85 mL, 5.9 mmol) was added at 0° C. The mixture was stirred at room temperature for 3 hours. The solvent was removed by concentrated in vacuum to give 0.85 g of (2S,4R)—N-((2S)-1-(cyclopropylsulfonyl-carbamoyl)-2-vinyl-cyclopropyl)-1-((S)-3,3-dimethyl-2-thioureidobutanoyl)-4-(2-(4-isopropoxyphenyl)furo[3,2-b]pyridine[3,2-d]pyrimidin-4-yloxy)pyrrolidine-2-carboxamide. LC-MS (M+H): 819.3.

A solution of (2S,4R)—N-((2S)-1-(cyclopropylsulfonylcarbamoyl)-2-vinylcyclopropyl)-1-((S)-3,3-dimethyl-2-thioureidobutanoyl)-4-(2-(4-isopropoxyphenyl)-furo[3,2-b]pyridine[3,2-d]pyrimidin-4-yloxy)pyrrolidine-2-carboxamide (0.424 g, 0.52 mmol) in 5.2 mL of dry dioxane added sodium bicarbonate (NaHCO₃, 0.44 g, 5.2 mmol) and 3-bromo-2-oxopropanoic acid (0.13 g, 0.78 mmol) was stirred at 60° C. for 2.5 hours. The solvent was removed by evaporation and the residue was dissolved in methylene chloride. The solution was washed with water, and then brine, dried over anhydrous MgSO₄, and concentrated in vacuum. 0.1 g of 2-((2S)-1-((2S,4R)-2-((2S)-1-(cyclopropylsulfonyl-carbamoyl)-2-vinyl-cyclopropylcarbamoyl)-4-(2-(4-isopropoxyphenyl)furo[3,2-b]pyridine[3,2-d]pyrimidin-4-yloxy)pyrrolidin-1-yl)-3,3-dimethyl-1-oxobutan-2-ylamino)thiazole-4-carboxylic acid. LC-MS (M+H): 887.3

To a stirred solution of 2-((2S)-1-((2S,4R)-2-((2S)-1-(cyclopropylsulfonyl carbamoyl)-2-vinylcyclopropylcarbamoyl)-4-(2-(4-isopropoxyphenyl)furo[3,2-b]pyridine[3,2-d]pyrimidin-4-yloxy)pyrrolidin-1-yl)-3,3-dimethyl-1-oxobutan-2-ylamino)thiazole-4-carboxylic acid (0.1 g, 0.11 mmol) in 0.55 mL of methylene chloride at 0° C. were added N-Methylmorpholine (NMM, 0.048 mL, 0.44 mmol), O-(7-azabenzotriazol-1-yl)-N,N,N′,N′-tetramethyluronium hexafluorophosphate (HATU, 0.084 g, 0.22 mmol), N-hydroxybenzotriazole (HOBt, 0.004 g, 0.033 mmol) and MeOH (0.045 mL, 1.1 mmol). The mixture was stirred at room temperature for 2 hours. The solvent was removed by evaporation and the residue was dissolved in methylene chloride. The solution was washed with a saturated ammonium chloride aqueous solution, and then brine, dried over anhydrous MgSO₄, and concentrated in vacuum. The residue was purified by silica gel column chromatography with 10% ethyl acetate/methylene chloride to give 0.025 g of methyl 2-((2S)-1-((2S,4R)-2-((2S)-1-(cyclopropylsulfonylcarbamoyl)-2-vinylcyclopropylcarbamoyl)-4-(2-(4-isopropoxyphenyl)furo[3,2-b]pyridine[3,2-d]pyrimidin-4-yloxy)-pyrrolidin-1-yl)-3,3-dimethyl-1-oxobutan-2-ylamino)thiazole-4-carboxylate (Compound 167). LC-MS (M+H): 901.3.

### Example 11

### Preparation of Compound 190

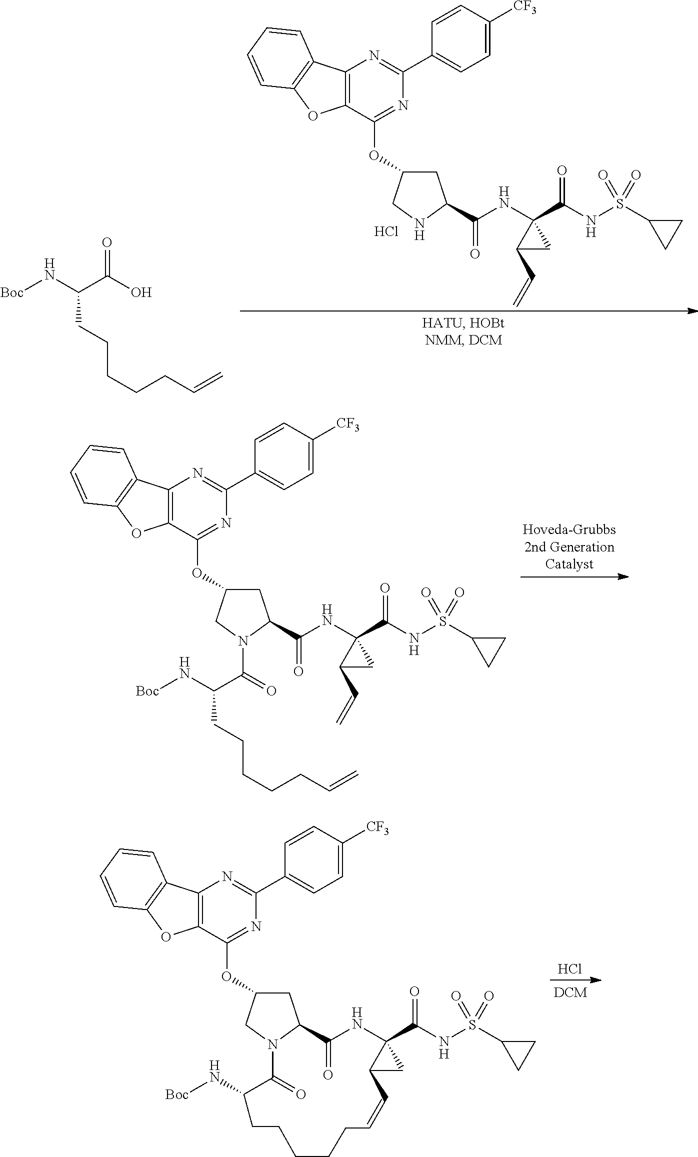

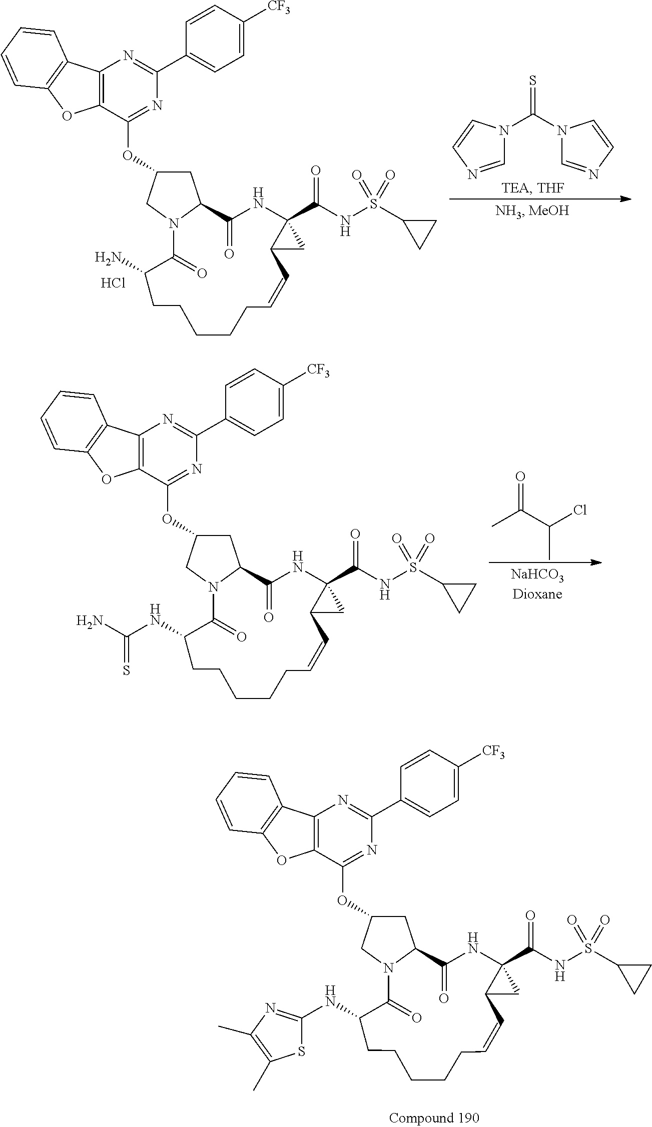

A mixture of (S)-2-(tert-butoxycarbonyl)non-8-enoic acid (1.50 g, 5.52 mmol), NMM (1.52 mL, 13.80 mmol), HATU (3.50 g, 9.20 mmol), HOBt (0.19 g, 1.38 mmol) and hydrochloride salt of (2S,4R)—N-((1R,2S)-1-(cyclopropylsulfonylcarbamoyl)-2-vinylcyclopropyl)-4-(2-(4-(trifluoromethyl)phenyl)benzofuro[3,2-d]pyrimidin-4-yloxy)-pyrrolidine-2-carboxamide (3.02 g, 4.60 mmol) in 23 mL of dry methylene chloride was stirred at room temperature for 2 h. The solid in the reaction solution was removed by filtration and the methylene chloride solution was extracted twice with saturated NH₄Cl aqueous solution, washed with brine, dried over MgSO₄ and concentrated in vacuo. The crude material was purified by silica gel column with 10% ethyl acetate/2% methanol/methylene chloride to give 3.99 g of tert-butyl(S)-1-((2S,4R)-2-(((1R,2S)-1-(cyclopropylsulfonyl-carbamoyl)-2-vinylcyclopropyl)carbamoyl)-4-(2-(4-(trifluoromethyl)phenyl)benzofuro-[3,2-d]pyrimidin-4-yloxy)pyrrolidin-1-yl)-1-oxonon-8-en-2-ylcarbamate. LC-MS (M+H): 909.3.

A mixture of tert-butyl (S)-1-((2S,4R)-2-(((1R,2S)-1-(cyclopropylsulfonylcarbamoyl)-2-vinylcyclopropyl)carbamoyl)-4-(2-(4-(trifluoromethyl)phenyl)benzofuro[3,2-d]pyrimidin-4-yloxy)pyrrolidin-1-yl)-1-oxonon-8-en-2-ylcarbamate (2.18 g, 2.40 mmol) and Hoveyda-Grubbs catalyst 2^{nd} generation (0.30 g, 0.48 mmol) in 480 mL of bubbling methylene chloride was stirred at 40° C. for 36 h. The organic solvent was removed by evaporation and the crude material was purified by silica gel column with 10% ethyl acetate/0.5% methanol/methylene chloride to give 1.39 g of the Boc-protected compound. LC-MS (M+H): 881.3. To the Boc-protected compound (0.20 g, 0.23 mmol) in 2.3 mL of methylene chloride was added 1.2 mL of 4N HCl in 1,4-dioxane at 0° C. The mixture was stirred at room temperature for 7 h and the organic solvent was removed by evaporation to give 0.22 g of the hydrochloride salt of (2R,6S,13aS,14aR,16aS,Z)-6-amino-N-(cyclopropylsulfonyl)-5,16-dioxo-2-(2-(4-(trifluoromethyl)phenyl)-benzofuro[3,2-d]pyrimidin-4-yloxy)-1,2,3,5,6,7,8,9,10,11,13a,14,14a,15,16,16a-hexadecahydrocyclopropa[e]pyrrol-e[1,2-a][1,4]diazacyclopentadecine-14a-carboxamide. LC-MS (M+H): 780.6.

To a stirred solution of the hydrochloride salt of (2R,6S,13aS,14aR,16aS,Z)-6-amino-N-(cyclopropylsulfonyl)-5,16-dioxo-2-(2-(4-(trifluoromethyl)phenyl)benzofuro-[3,2-d]pyrimidin-4-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide (0.26 g, 0.31 mmol) in 1.5 mL of THF were added triethylamine (0.21 mL, 1.53 mmol) and di(1H-imidazol-1-yl)methanethione (0.11 g, 0.61 mmol) at 0° C. The mixture was stirred at room temperature for 1.5 h, and ca. 7N ammonia solution in methanol (0.44 mL, 3.05 mmol) was added. The mixture was stirred at room temperature for 3.5 h. The solvent was removed by evaporation to give 0.42 g of (2R,6S,13aS,14aR,16aS,Z)—N-(cyclopropylsulfonyl)-5,16-dioxo-6-thioureido-2-(2-(4-(trifluoromethyl)phenyl)benzofuro[3,2-d]pyrimidin-4-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. LC-MS (M+H): 840.2.

A mixture of (2R,6S,13aS,14aR,16aS,Z)—N-(cyclopropylsulfonyl)-5,16-dioxo-6-thioureido-2-(2-(4-(trifluoromethyl)phenyl)benzofuro[3,2-d]pyrimidin-4-yloxy)-1,2,3,5,-6,7,8,9,10,11,13a,14,14a,15,16,16a-hexadecahydrocyclopropa[e]pyrrolo[1,2-a][1,4]-diazacyclopentadecine-14a-carboxamide (0.20 g, 0.145 mmol), sodium bicarbonate (0.12 g, 1.45 mmol) and 3-chloro-2-butanone (0.02 6 mL, 0.26 mmol) in 1.5 mL of 1,4-dioxane was stirred at 60° C. for 2 h. To the mixture was added twice 3-chloro-2-butanone (0.026 mL, 0.26 mmol). The mixture was then stirred at 60° C. overnight. The solvent was removed by evaporation and the residue was dissolved in methylene chloride. The methylene chloride solution was washed with water, dried over MgSO₄ and concentrated in vacuo. The crude material was purified by silica gel column with 20% ethyl acetate/1% methanol/methylene chloride to give 14 mg of (2R,6S,13aS,14aR,16aS,Z)—N-(cyclo-propylsulfonyl)-6-(4,5-dimethylthiazol-2-ylamino)-5,16-dioxo-2-(2-(4-(trifluoromethyl-)phenyl)benzofuro[3,2-d]pyrimidin-4-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide (Compound 190). LC-MS (M+H): 892.3, ¹H-NMR (CDCl₃, 300 Hz): δ0.93 (m, 1H), 1.14 (m, 2H), 1.33 (m, 3H), 1.51 (m, 6H), 1.79 (s, 3H), 1.85 (m, 1H), 1.95 (s, 3H), 2.17 (m, 1H), 2.29 (m, 2H), 2.60 (m, 1H), 2.69 (m, 1H), 2.82 (m, 1H), 2.94 (m, 1H), 4.29 (m, 2H), 4.59 (d, J=10.5 Hz, 1H), 4.86 (t, J=7.5 Hz, 1H), 5.00 (dd, J₁=9.3 Hz, J₂=9.3 Hz, 1H), 5.74 (m, 1H), 6.24 (m, 1H), 7.50 (t, J=8.1 Hz, 1H), 7.65 (m, 2H), 7.80 (d, J=8.4 Hz, 2H), 8.26 (d, J=8.1 Hz, 1H), 8.63 (d, J=8.1 Hz, 2H).

### Example 12

### Preparation of Compound 209

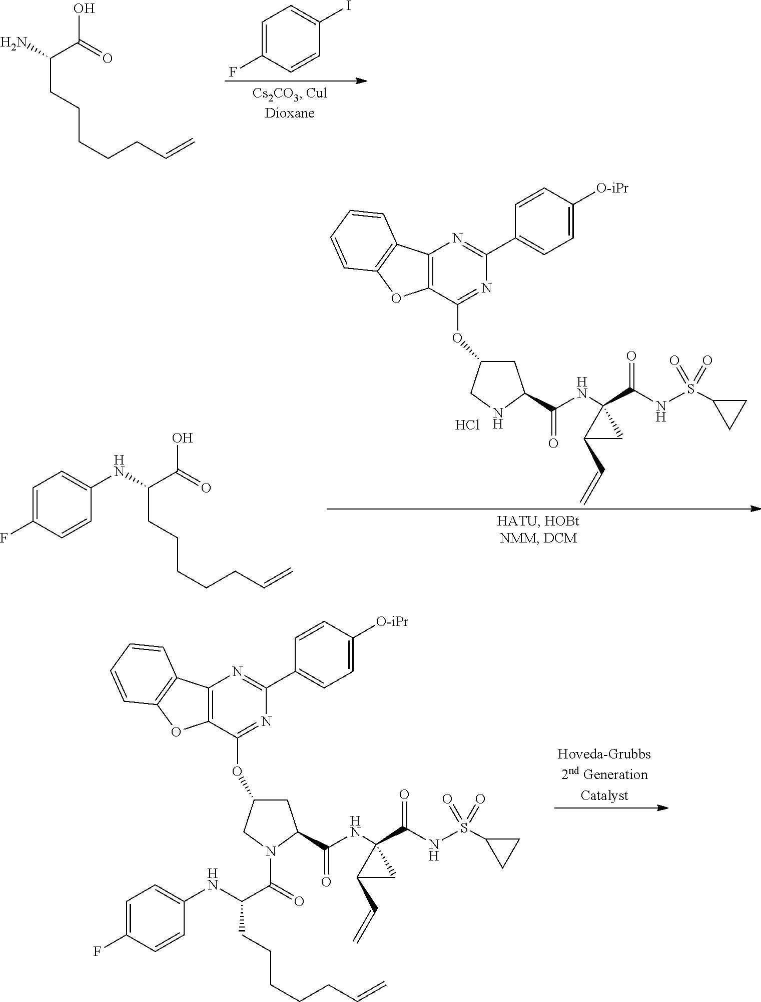

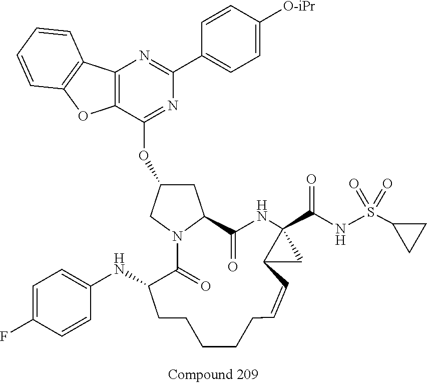

A mixture of (S)-2-aminonon-8-enoic acid (0.40 g, 2.34 mmol), cesium carbonate (2.28 g, 6.78 mmol), copper(I) iodide (0.22 g, 1.17 mmol) and 4-fluoroiodobenzene (0.54 mL, 4.67 mmol) in 4.7 mL 1,4-dioxane was stirred at 90° C. for 14 h. The organic solvent was removed by evaporation and the residue was dissolved in water and washed with hexane. 6N HCl aqueous solution was added to adjust pH to ˜2. The solution was extracted with ether. The ether layer was dried over MgSO₄ and concentrated in vacuo to give 0.246 g of (S)-2-(4-fluorophenylamino)non-8-enoic acid. LC-MS (M+H): 266.0.

A mixture of (S)-2-(4-fluorophenylamino)non-8-enoic acid (0.11 g, 0.42 mmol), NMM (0.15 mL, 1.39 mmol), HATU (0.21 g, 0.56 mmol), HOBt (0.011 g, 0.08 mmol) and hydrochloride salt of (2S,4R)—N-((1R,2S)-1-(cyclopropylsulfonylcarbamoyl)-2-vinylcyclopropyl)-4-(2-(4-isopropoxyphenyl)benzofuro[3,2-d]pyrimidin-4-yloxy)pyrrolidine-2-carboxamide (0.2 g, 0.28 mmol) in 1.4 mL of dry methylene chloride was stirred at room temperature for 5 h. The solid in the reaction solution was removed by filtration and the methylene chloride solution was extracted twice with saturated NH₄Cl aqueous solution, dried over MgSO₄ and concentrated in vacuo. The crude material was purified by silica gel column with 30% ethyl acetate/0.2% methanol/hexane to give 0.12 g of (2S,4R)—N-((1R,2S)-1-(cyclopropylsulfonylcarbamoyl)-2-vinylcyclopropyl)-1-((S)-2-(4-fluorophenylamino)non-8-enoyl)-4-(2-(4-isopropoxyphenyl)benzofuro[3,2-d]pyrimidin-4-yloxy)pyrrolidine-2-carboxamide. LC-MS (M+H): 893.3.

A mixture of (2S,4R)—N-((1R,2S)-1-(cyclopropylsulfonylcarbamoyl)-2-vinylcyclopropyl)-1-((S)-2-(4-fluorophenylamino)non-8-enoyl)-4-(2-(4-isopropoxyphenyl)-1-benzofuro[3,2-d]pyrimidin-4-yloxy)pyrrolidine-2-carboxamide (0.12 g, 0.13 mmol) and Hoveyda-Grubbs catalyst 2^{nd} generation (0.012 g, 0.02 mmol) in 26 mL of bubbling methylene chloride was stirred at 40° C. overnight. The organic solvent was removed by evaporation and the crude material was purified by silica gel column with 5% ethyl acetate/0.5% methanol/methylene chloride to give 51 mg of (2R,6S,13aS,14aR,16aS,Z)—N-(cyclopropylsulfonyl)-6-(4-fluorophenylamino)-2-(2-(4-isopropoxyphenyl)benzofuro[3,2-d]pyrimidin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide (Compound 209). LC-MS (M+H): 865.1, ¹H-NMR (CDCl₃, 300 Hz): δ0.91 (m, 2H), 1.16 (m, 2H), 1.42 (d, J=6.3 Hz, 6H), 1.51 (m, 6H), 1.78 (m, 2H), 1.93 (m, 2H), 2.14 (m, 1H), 2.58 (m, 1H), 2.73 (m, 2H), 2.92 (m, 1H), 4.12 (m, 3H), 4.25 (d, J=12.0 Hz, 1H), 4.60 (t, J=7.2 Hz, 1H), 4.70 (m, 1H), 5.01 (dd, J₁=9.3 Hz, J₂=9.3 Hz, 1H), 5.75 (m, 1H), 6.24 (m, 1H), 6.45 (dd, J₁=9.0 Hz, J₂=4.2 Hz, 2H), 6.83 (dd, J₁=8.7 Hz, J₂=8.7 Hz, 2H), 7.03 (d, J=8.7 Hz, 2H), 7.23 (s, 1H), 7.38 (t, J=7.2 Hz, 1H), 7.56 (m, 2H), 8.15 (d, J=7.5 Hz, 1H), 8.39 (d, J=8.7 Hz, 2H), 10.41 (s, 1H).

### Example 13

### Preparation of Compound 224

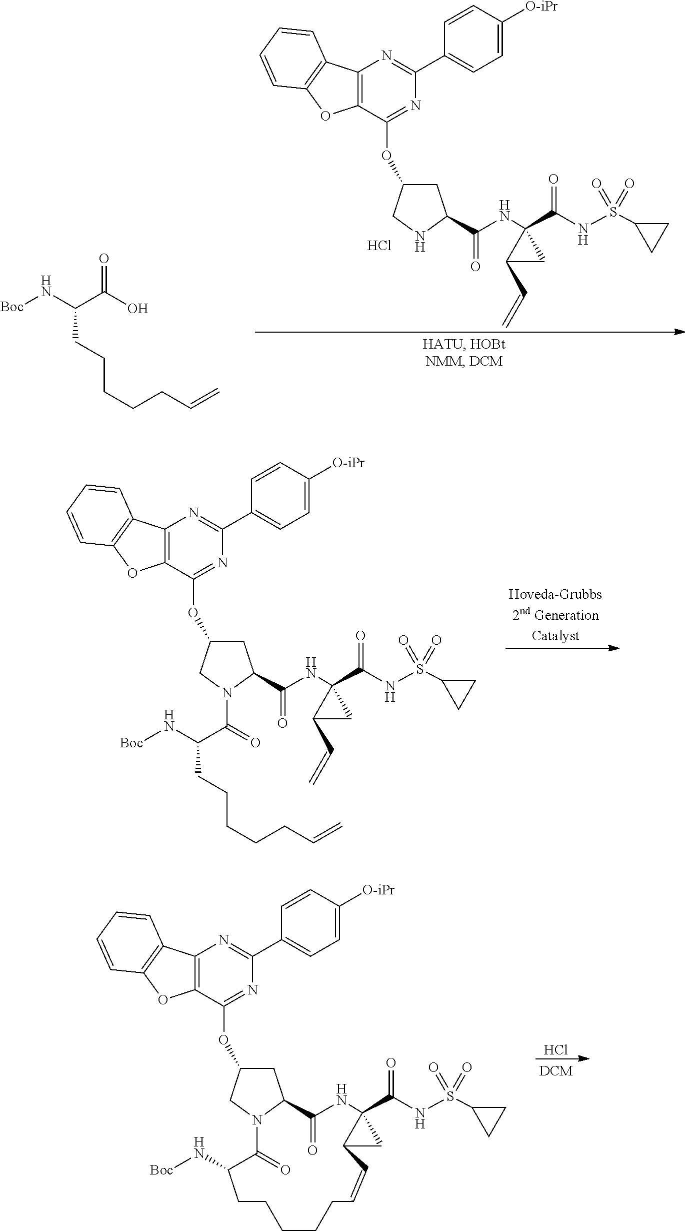

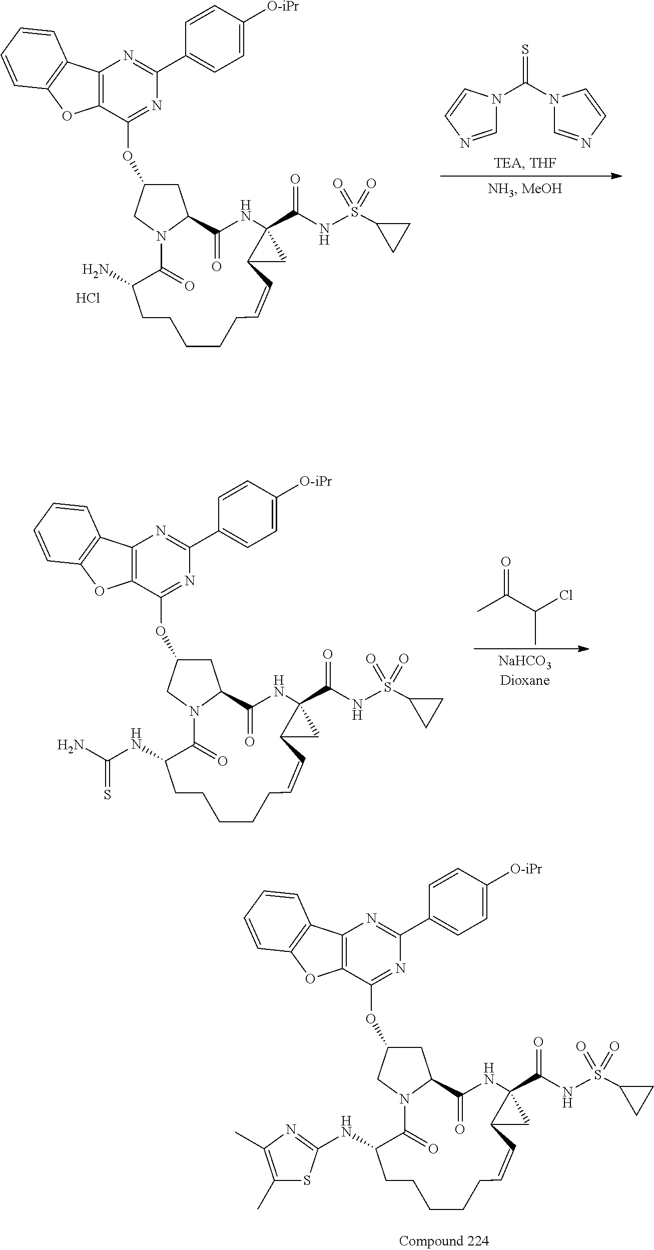

A mixture of (S)-2-(tert-butoxycarbonyl)non-8-enoic acid (0.38 g, 1.42 mmol), NMM (0.52 mL, 4.72 mmol), HATU (0.72 g, 1.89 mmol), HOBt (0.038 g, 0.28 mmol) and hydrochloride salt of (2S,4R)—N-((1R,2S)-1-(cyclopropylsulfonylcarbamoyl)-2-vinylcyclopropyl)-4-(2-(4-isopropoxyphenyl)benzofuro[3,2-d]pyrimidin-4-yloxy)pyrrolidine-2-carboxamide dihydrochloride (0.61 g, 0.94 mmol) in 4.7 mL of dry methylene chloride was stirred at room temperature overnight. The solid in the reaction solution was removed by filtration and the methylene chloride solution was extracted twice with saturated NH₄Cl aqueous solution, dried over MgSO₄ and concentrated in vacuo. The crude material was purified by silica gel column with 7% ethyl acetate/0.2% methanol/methylene chloride to give 0.76 g of tert-butyl (S)-1-((2S,4R)-2-(((1R,2S)-1-(cyclopropylsulfonylcarbamoyl)-2-vinylcyclopropyl)carbamoyl)-4-(2-(4-isopropoxyphenyl)benzo-furo[3,2-d]pyrimidin-4-yloxy)pyrrolidin-1-yl)-1-oxonon-8-en-2-ylcarbamate. LC-MS (M+H): 899.4.

A mixture of tert-butyl (S)-1-((2S,4R)-2-(((1R,2S)-1-(cyclopropylsulfonylcarbamoyl)-2-vinylcyclopropyl)carbamoyl)-4-(2-(4-isopropoxyphenyl)benzofuro[3,2-d]pyrimidin-4-yloxy)pyrrolidin-1-yl)-1-oxonon-8-en-2-ylcarbamate (0.76 g, 0.84 mmol) and Hoveyda-Grubbs catalyst 2^{nd} generation (0.08 g, 0.13 mmol) in 169 mL of bubbling methylene chloride was stirred at 40° C. overnight. The organic solvent was removed by evaporation and the crude material was purified by silica gel column with 10% ethyl acetate/1% methanol/methylene chloride to give 0.15 g of the Boc-protected compound. LC-MS (M+H): 871.3.

To the Boc-protected compound (0.15 g, 0.18 mmol) in 1.8 mL of methylene chloride was added 0.9 mL of 4N HCl in 1,4-dioxane at 0° C. The mixture was stirred at room temperature for 8 h and the organic solvent was removed by evaporation to give 0.15 g of the hydrochloride salt of (2R,6S,13aS,14aR,16aS,Z)-6-amino-N-(cyclopropylsulfonyl)-2-(2-(4-isopropoxyphenyl)benzofuro[3,2-d]pyrimidin-4-yloxy)-5,16-dioxo-1,2,3,5,6,7,8,9,10,11,13a,14,14a,15,16,16a-hexadecahydrocyclopropa[e]pyrrole-[1,2-a][1,4]diazacyclopentadecine-14a-carboxamide. LC-MS (M+H): 771.3.

To a stirred solution of the hydrochloride salt of (2R,6S,13aS,14aR,16aS,Z)-6-amino-N-(cyclopropylsulfonyl)-2-(2-(4-isopropoxyphenyl)benzofuro[3,2-d]pyrimidin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide (0.15 g, 0.18 mmol) in 0.9 mL of THF were added triethylamine (0.12 mL, 0.89 mmol) and di(1H-imidazol-1-yl)methanethione (0.063 g, 0.35 mmol) at 0° C. The mixture was stirred at room temperature for 1 h, and ca. 7N ammonia solution in methanol (0.25 mL, 1.77 mmol) was added. The mixture was stirred at room temperature for 3 h. The solvent was removed by evaporation to give 0.28 g of (2R,6S,13aS,14aR,16aS,Z)—N-(cyclopropylsulfonyl)-2-(2-(4-isopropoxy-phenyl)benzofuro[3,2-d]pyrimidin-4-yloxy)-5,16-dioxo-6-thioureido-1,2,3,5,6,7,8,9,10,11,13a,14,14a,15,16,16a-hexadecahydrocyclopropa[e]pyrrolo[1,2-a][1,4]diazacyclo-pentadecine-14a-carboxamide. LC-MS (M+H): 830.3.

A mixture of (2R,6S,13aS,14aR,16aS,Z)—N-(cyclopropylsulfonyl)-2-(2-(4-isopropoxyphenyl)benzofuro[3,2-d]pyrimidin-4-yloxy)-5,16-dioxo-6-thioureido-1,2,3,5,6,7,8,9,10,11,13a,14,14a,15,16,16a-hexadecahydrocyclopropa[e]pyrrolo[1,2-a][1,4]diazacyclopentadecine-14a-carboxamide (0.28 g, 0.34 mmol), sodium bicarbonate (0.29 g, 3.42 mmol) and 3-chloro-2-butanone (0.14 mL, 1.37 mmol) in 1.7 mL of 1,4-dioxane at room temperature was stirred at 60° C. for 17 h. To the mixture was added twice 3-chloro-2-butanone (0.07 mL, 0.69 mmol). The mixture was stirred at 60° C. overnight. The solvent was removed by evaporation and the residue was dissolved in methylene chloride. The methylene chloride solution was washed with water, dried over MgSO₄ and concentrated in vacuo. The crude material was purified by silica gel column with 40% ethyl acetate/2% methanol/hexane to give 41 mg of (2R,6S,13aS,14aR,16aS,Z)—N-(cyclopropylsulfonyl)-6-(4,5-dimethylthiazol-2-ylamino)-2-(2-(4-isopropoxyphenyl)benzofuro[3,2-d]pyrimidin-4-yloxy)-5,16-dioxo-1,2,3,5,6,7,8,9,10,11,13a,14,14a,15,16,16a-hexadecahydrocyclopropa[e]pyrrolo[1,2-a][1,4]diazacyclopentadecine-14a-carboxamide (Compound 224). LC-MS (M+H): 882.3, ¹H-NMR (CDCl₃, 300 Hz): δ0.90 (m, 2H), 1.13 (m, 3H), 1.40 (d, J=5.7 Hz, 6H), 1.51 (m, 6H), 1.73 (m, 2H), 1.83 (s, 3H), 1.94 (s, 3H), 2.20 (m, 3H), 2.62 (m, 2H), 2.82 (m, 1H), 2.93 (m, 1H), 4.20 (m, 1H), 4.30 (dd, J₁=11.7 Hz, J₂=4.5 Hz, 1H), 4.48 (d, J=10.8 Hz, 1H), 4.69 (m, 1H), 4.91 (t, J=7.7 Hz, 1H), 5.00 (dd, J₁=9.6 Hz, J₂=9.6 Hz, 1H), 5.71 (m, 1H), 6.23 (m, 1H), 7.04 (d, J=8.7 Hz, 2H), 7.47 (t, J=7.4 Hz, 1H), 7.60 (m, 2H), 8.25 (d, J=7.5 Hz, 1H), 8.45 (d, J=9.0 Hz, 2H).

Compounds 1, 2, 4-49, 51-63, 65-90, 92-94, 96-99, 102-140, 142-164, 166, 168-189, 191-223, and 225-227 were prepared in the manners similar to those described above. Their analytical data are summarized below.

Compound 1: LC-MS (M+H): 868.2.

Compound 2: LC-MS (M+H): 867.3.

Compound 3: LC-MS (M+H): 852.2.

Compound 4: LC-MS (M+H): 866.2.

Compound 5: LC-MS (M+H): 894.2.

Compound 6: LC-MS (M+H): 854.2.

Compound 7: LC-MS (M+H): 868.2

Compound 8: LC-MS (M+H): 896.3.

Compound 9: LC-MS (M+H): 867.2.

Compound 10: LC-MS (M+H): 869.2.

Compound 11: LC-MS (M+H): 868.2.

Compound 12: LC-MS (M+H): 881.2.

Compound 13: LC-MS (M+H): 908.2.

Compound 14: LC-MS (M+H): 881.2.

Compound 15: LC-MS (M+H): 801.2.

Compound 16: LC-MS (M+H): 866.2.

Compound 17: LC-MS (M+H): 880.2.

Compound 18: LC-MS (M+H): 910.4.

Compound 19: LC-MS (M+H): 868.2.

Compound 20: LC-MS (M+H): 894.1.

Compound 21: LC-MS (M+H): 892.2.

Compound 22: LC-MS (M+H): 863.2.

Compound 23: LC-MS (M+H): 881.3.

Compound 24: LC-MS (M+H): 894.3.

Compound 25: LC-MS (M+H): 896.3.

Compound 26: LC-MS (M+H): 889.2.

Compound 27: LC-MS (M+H): 907.3.

Compound 28: LC-MS (M+H): 870.3.

Compound 29: LC-MS (M+H): 880.3.

Compound 30: LC-MS (M+H): 877.3.

Compound 31: LC-MS (M+H): 881.3.

Compound 32: LC-MS (M+H): 894.4.

Compound 33: LC-MS (M+H): 924.

Compound 34: LC-MS (M+H): 920.3.

Compound 35: LC-MS (M+H): 906.3.

Compound 36: LC-MS (M+H): 865.4.

Compound 37: LC-MS (M+H): 883.4.

Compound 38: LC-MS (M+H): 883.3.

Compound 39: LC-MS (M+H): 879.4.

Compound 40: LC-MS (M+H): 891.4.

Compound 41: LC-MS (M+H): 909.4.

Compound 42: LC-MS (M+H): 879.3.

Compound 43: LC-MS (M+H): 897.2.

Compound 44: LC-MS (M+H): 897.3.

Compound 45: LC-MS (M+H): 889.4.

Compound 46: LC-MS (M+H): 907.3.

Compound 47: LC-MS (M+H): 892.3.

Compound 48: LC-MS (M+H): 906.3.

Compound 49: LC-MS (M+H): 847.3.

Compound 50: LC-MS (M+H): 863.1.

Compound 51: LC-MS (M+H): 881.1.

Compound 52: LC-MS (M+H): 845.1.

Compound 53: LC-MS (M+H): 896.1.

Compound 54: LC-MS (M+H): 923.1.

Compound 55: LC-MS (M+H): 909.1.

Compound 56: LC-MS (M+H): 937.1.

Compound 57: LC-MS (M+H): 935.1.

Compound 58: LC-MS (M+H): 922.1.

Compound 59: LC-MS (M+H): 870.1.

Compound 60: LC-MS (M+H): 919.3.

Compound 61: LC-MS (M+H): 887.2.

Compound 62: LC-MS (M+H): 923.3.

Compound 63: LC-MS (M+H): 916.3.

Compound 64: LC-MS (M+H): 870.3.

Compound 65: LC-MS (M+H): 870.3.

Compound 66: LC-MS (M+H): 935.3.

Compound 67: LC-MS (M+H): 882.2.

Compound 68: LC-MS (M+H): 895.2.

Compound 69: LC-MS (M+H): 909.3

Compound 70: LC-MS (M+H): 909.3.

Compound 71: LC-MS (M+H): 935.3.

Compound 72: LC-MS (M+H): 923.3.

Compound 73: LC-MS (M+H): 903.3.

Compound 74: LC-MS (M+H): 923.7.

Compound 75: LC-MS (M+H): 938.3.

Compound 76: LC-MS (M+H): 902.7.

Compound 77: LC-MS (M+H): 909.8.

Compound 78: LC-MS (M+H): 902.2.

Compound 79: LC-MS (M+H): 902.2.

Compound 80: LC-MS (M+H): 938.3.

Compound 81: LC-MS (M+H): 925.2.

Compound 82: LC-MS (M+H): 902.2.

Compound 83: LC-MS (M+H): 930.2.

Compound 84: LC-MS (M+H): 928.3.

Compound 85: LC-MS (M+H): 916.2.

Compound 86: LC-MS (M+H): 902.2.

Compound 87: LC-MS (M+H): 916.2.

Compound 88: LC-MS (M+H): 899.1.

Compound 89: LC-MS (M+H): 930.3.

Compound 90: LC-MS (M+H): 902.3.

Compound 91: LC-MS (M+H): 910.2.

Compound 92: LC-MS (M+H): 937.2.

Compound 93: LC-MS (M+H): 913.3.

Compound 94: LC-MS (M+H): 870.3.

Compound 95: LC-MS (M+H): 853.3.

Compound 96: LC-MS (M+H): 852.4.

Compound 97: LC-MS (M+H): 871.3.

Compound 98: LC-MS (M+H): 855.3.

Compound 99: LC-MS (M+H): 914.3.

Compound 100: LC-MS (M+H): 900.3.

Compound 101: LC-MS (M+H): 856.3.

Compound 102: LC-MS (M+H): 914.3.

Compound 103: LC-MS (M+H): 896.3.

Compound 104: LC-MS (M+H): 886.3.

Compound 105: LC-MS (M+H): 899.4.

Compound 106: LC-MS (M+H): 913.4.

Compound 107: LC-MS (M+H): 853.4.

Compound 108: LC-MS (M+H): 903.4.

Compound 109: LC-MS (M+H): 882.3.

Compound 110: LC-MS (M+H): 940.3.

Compound 111: LC-MS (M+H): 940.3.

Compound 112: LC-MS (M+H): 879.3.

Compound 113: LC-MS (M+H): 879.3.

Compound 114: LC-MS (M+H): 938.3.

Compound 115: LC-MS (M+H): 938.3.

Compound 116: LC-MS (M+H): 913.3.

Compound 117: LC-MS (M+H): 929.2.

Compound 118: LC-MS (M+H): 869.3.

Compound 119: LC-MS (M+H): 919.3.

Compound 120: LC-MS (M+H): 913.3.

Compound 121: LC-MS (M+H): 929.3.

Compound 122: LC-MS (M+H): 903.4.

Compound 123: LC-MS (M+H): 869.3.

Compound 124: LC-MS (M+H): 919.3.

Compound 125: LC-MS (M+H): 835.3.

Compound 126: LC-MS (M+H): 851.3.

Compound 127: LC-MS (M+H): 884.1.

Compound 128: LC-MS (M+H): 872.3.

Compound 129: LC-MS (M+H): 916.0.

Compound 130: LC-MS (M+H): 930.0.

Compound 131: LC-MS (M+H): 926.2.

Compound 132: LC-MS (M+H): 837.1.

Compound 133: LC-MS (M+H): 869.3.

Compound 134: LC-MS (M+H): 882.3

Compound 135: LC-MS (M+H): 928.3.

Compound 136: LC-MS (M+H): 927.3.

Compound 137: LC-MS (M+H): 853.3.

Compound 138: LC-MS (M+H): 886.3.

Compound 139: LC-MS (M+H): 930.3.

Compound 140: LC-MS (M+H): 900.3.

Compound 141: LC-MS (M+H): 860.3.

Compound 142: LC-MS (M+H): 896.4.

Compound 143: LC-MS (M+H): 853.4.

Compound 144: LC-MS (M+H): 871.3.

Compound 145: LC-MS (M+H): 871.3.

Compound 146: LC-MS (M+H): 855.3.

Compound 147: LC-MS (M+H): 874.3.

Compound 148: LC-MS (M+H): 900.3.

Compound 149: LC-MS (M+H): 918.3.

Compound 150: LC-MS (M+H): 932.3.

Compound 151: LC-MS (M+H): 869.3.

Compound 152: LC-MS (M+H): 887.3.

Compound 153: LC-MS (M+H): 887.3.

Compound 154: LC-MS (M+H): 890.3

Compound 155: LC-MS (M+H): 916.3.

Compound 156: LC-MS (M+H): 934.2.

Compound 157: LC-MS (M+H): 948.3.

Compound 158: LC-MS (M+H): 878.3.

Compound 159: LC-MS (M+H): 894.3.

Compound 160: LC-MS (M+H): 879.3.

Compound 161: LC-MS (M+H): 926.3.

Compound 162: LC-MS (M+H): 940.3.

Compound 163: LC-MS (M+H): 942.3.

Compound 164: LC-MS (M+H): 944.3.

Compound 165: LC-MS (M+H): 854.3.

Compound 166: LC-MS (M+H): 915.3.

Compound 167: LC-MS (M+H): 901.3.

Compound 168: LC-MS (M+H): 892.4.

Compound 169: LC-MS (M+H): 910.3.

Compound 170: LC-MS (M+H): 892.4.

Compound 171: LC-MS (M+H): 910.4.

Compound 172: LC-MS (M+H): 861.3.

Compound 173: LC-MS (M+H): 825.3.

Compound 174: LC-MS (M+H): 879.3

Compound 175: LC-MS (M+H): 886.3.

Compound 176: LC-MS (M+H): 872.3.

Compound 177: LC-MS (M+H): 940.3.

Compound 178: LC-MS (M+H): 926.3.

Compound 179: LC-MS (M+H): 835.2.

Compound 180: LC-MS (M+H): 896.2.

Compound 181: LC-MS (M+H): 797.3.

Compound 182: LC-MS (M+H): 858.3.

Compound 183: LC-MS (M+H): 882.2.

Compound 184: LC-MS (M+H): 892.2.

Compound 185: LC-MS (M+H): 893.2.

Compound 186: LC-MS (M+H): 893.2.

Compound 187: LC-MS (M+H): 920.2.

Compound 188: LC-MS (M+H): 880.1.

Compound 189: LC-MS (M+H): 878.3.

Compound 190: LC-MS (M+H): 892.3.

Compound 191: LC-MS (M+H): 906.4.

Compound 192: LC-MS (M+H): 904.2.

Compound 193: LC-MS (M+H): 908.2.

Compound 194: LC-MS (M+H): 935.1.

Compound 195: LC-MS (M+H): 947.1.

Compound 196: LC-MS (M+H): 891.3.

Compound 197: LC-MS (M+H): 859.3.

Compound 198: LC-MS (M+H): 921.3.

Compound 199: LC-MS (M+H): 875.3.

Compound 200: LC-MS (M+H): 882.2.

Compound 201: LC-MS (M+H): 875.3.

Compound 202: LC-MS (M+H): 882.3.

Compound 203: LC-MS (M+H): 914.2.

Compound 204: LC-MS (M+H): 942.2.

Compound 205: LC-MS (M+H): 936.1.

Compound 206: LC-MS (M+H): 914.3.

Compound 207: LC-MS (M+H): 942.3.

Compound 208: LC-MS (M+H): 928.3.

Compound 209: LC-MS (M+H): 865.1.

Compound 210: LC-MS (M+H): 865.3.

Compound 211: LC-MS (M+H): 915.1.

Compound 212: LC-MS (M+H): 915.3.

Compound 213: LC-MS (M+H): 847.3.

Compound 214: LC-MS (M+H): 863.3.

Compound 215: LC-MS (M+H): 881.3.

Compound 216: LC-MS (M+H): 881.3.

Compound 217: LC-MS (M+H): 881.3.

Compound 218: LC-MS (M+H): 872.3.

Compound 219: LC-MS (M+H): 897.3.

Compound 220: LC-MS (M+H): 888.3.

Compound 221: LC-MS (M+H): 865.4.

Compound 222: LC-MS (M+H): 883.3.

Compound 223: LC-MS (M+H): 890.3.

Compound 224: LC-MS (M+H): 882.3.

Compound 225: LC-MS (M+H): 882.4.

Compound 226: LC-MS (M+H): 899.4.

Compound 227: LC-MS (M+H): 900.4.

### Example 14

### Synthesis of [4-Cyclopropanesulfonylaminocarbonyl-2,15-dioxo-18-(2-phenyl-pyrimido[1,2-b]indazol-4-yloxy)-3,16-diaza-tricyclo[14.3.0.04,6]nonadec-7-en-14-yl]-carbamic acid cyclopentyl ester (Compound 228)

Compound 228 was prepared by the route shown below:

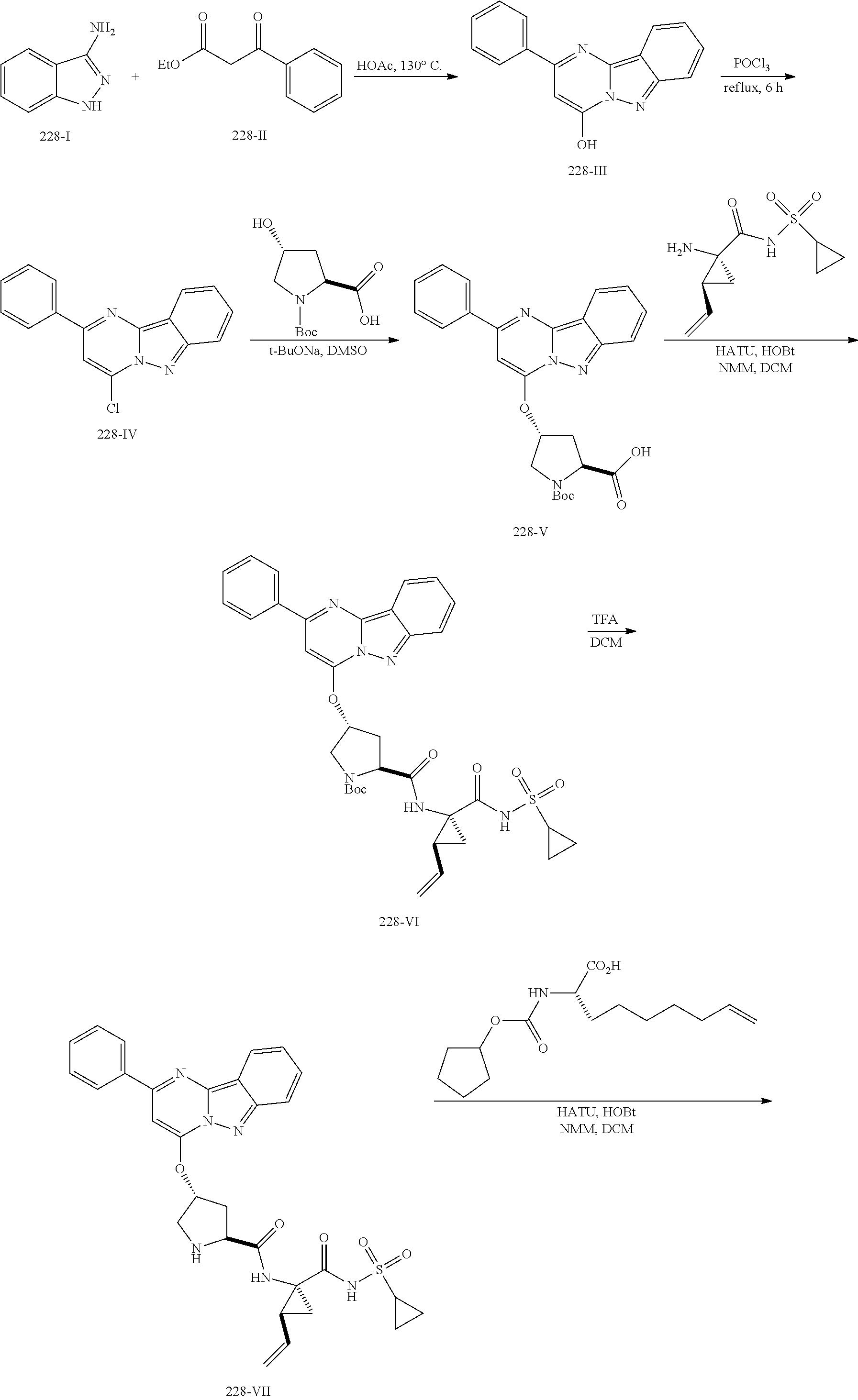

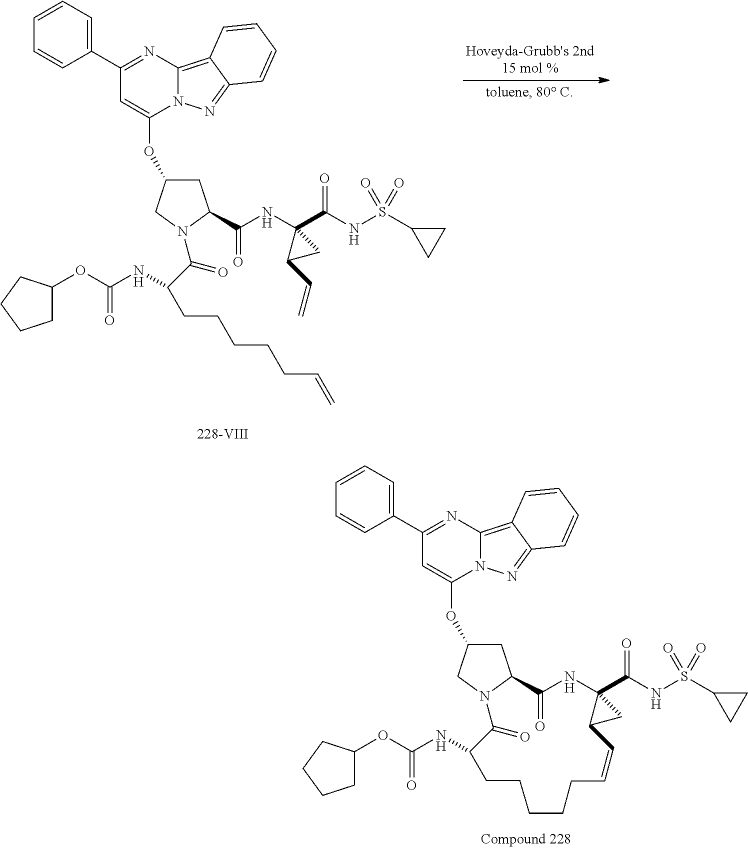

To a solution of ethyl benzoylacetate (228-II) (0.86 g, 4.5 mmol) in 10 mL of acetic acid was added 1H-indazole-3-ylamine (228-I) (0.60 g, 4.5 mmol) at room temperature. The reaction mixture was then heated to 130° C. and stirred for 18 hours. After reaction was completed, the mixture was concentrated under vacuum. The residue was washed with 20 mL of 50% ethyl acetate/hexanes solution to give white precipitate. The suspension was filtered to give compound 228-III as an off-white solid (0.74 g, 95%). MS m/z 262.0 (M⁺+1).

A solution of 228-III (1.71 g, 5.2 mmol) and excess phosphorus oxychloride (POCl₃) was refluxed for 2 hours. After cooled and thoroughly concentrated, the mixture was subjected to extraction with methylene chloride and 10% sodium hydroxide. The organic layer was dried over MgSO₄, concentrated, and crystallized from CH₂Cl₂ and n-hexane to give compound 228-IV (0.74 g, 82%). MS m/z 280.0 (M⁺+1).

To a suspension of boc-trans-4-hydroxy-L-proline (0.74 g, 3.2 mmol) in DMSO (30 mL) was added t-BuONa (0.54 g, 5.6 mmol) at 0° C. After the mixture was allowed to warm to room temperature and stirred for 30 min, compound 228-IV (0.74 g, 2.65 mmol) was added slowly at 10° C. After stirring in cold water bath for 1 hour, the reaction mixture was neutralized to pH 6˜7 by 10% NaHSO₄ aqueous solution and diluted by 50 mL of water. The suspension was then filtered and air dried to afford compound 228-V (0.90 g, 72%). MS m/z 475.2 (M⁺+1).

To a solution of HATU (1.4 g, 3.8 mmol), 1-hydroxybenzotriazole (HOBT, 0.38 g, 2.8 mmol), 228-V (0.90 g, 1.9 mmol) and 2-t-butoxycarbonylamino-non-8-enoic acid (0.69 g, 2.1 mmol) in CH₂Cl₂ (20 mL) at room temperature was added N-methylmorpholine (NMM, 1.1 g, 10.6 mmol). After 18 hour of stirring, the reaction mixture was washed with saturated NaHCO₃ aqueous solution. Extraction with CH₂Cl₂ followed by concentration gave the crude compound 228-VI. The residue was purified by silica gel column chromatography to give compound 228-VI (0.85 g, 1.24 mmol, 66%). MS m/z 687.3 (M⁺+1).

To a solution of compound 228-VI (0.85 g, 1.4 mmol) in CH₂Cl₂ (20 mL) was added 4 mL of TFA at room temperature. After 2 h of stirring, the reaction mixture was concentrated under vacuum to give compound 228-VII. The resulting residue was ready for the next step. MS: m/z 587.3 (M⁺+1).

NMM (0.12 g, 1.2 mmol) was added to a solution of 2-t-butoxycarbonyl aminonon-8-enoic acid (0.63 g, 1.4 mmol), HATU (0.94 g, 2.5 mmol), HOBT (0.25 g, 1.9 mmol) and crude compound 228-VII (0.73 g, 1.24 mmol) in CH₂Cl₂ (20 mL) at room temperature. After 18 h of stirring, the reaction mixture was washed with saturated NaHCO₃ aqueous solution. Extraction with CH₂Cl₂ followed by concentration gave the crude compound 228-VIII. The residue was purified by silica gel column chromatography (3% MeOH/CH₂Cl₂) to give compound 228-VIII (0.84 g, 0.98 mmol, 79%). MS m/z 852.4 (M⁺+1).

To a solution of compound 228-VIII (0.84 g, 0.98 mmol) in toluene (200 mL) was added Hoveyda-Grubbs 2^{nd} (100 mg, 0.159 mmol) at room temperature under N₂. Then, the reaction mixture was stirred at 80° C. for 18 h to carry out metathesis cyclization. The reaction was quenched and the reaction mixture was purified by silica gel column chromatography (3% MeOH/CH₂Cl₂) to give compound 228 (0.17 g, 21%). MS: m/z 824.4 (M⁺+1); ¹H NMR (CDCl₃) 10.50 (s, 1H), 8.16 (d, J=8.1 Hz, 1H), 8.11-8.09 (m, 2H), 7.74 (d, J=9.0 Hz, 1H), 7.58-7.51 (m, 3H), 7.14 (t, J=7.2 Hz, 1H), 6.8 (s, 1H), 5.69 (dt, 1H), 5.61 (brs, 1H), 5.1-4.95 (m, 3H), 4.74 (d, J=11.4 Hz, 1H), 4.18-4.12 (m, 2H), 4.03 (m, 1H), 2.97-2.89 (m, 2H), 2.71-2.56 (m, 2H), 2.38 (q, J=9.0 Hz, 1H), 1.87 (m, 9H), 1.54-1.08 (m, 14H), 0.96-0.90 (m, 2H).

### Example 15

### Syntheses of Compound 229-303

Each of Compounds 229-303 were prepared according to the procedure described in the above example.

Compound 229: MS: m/z 854.4 (M⁺+1); ¹H NMR (CDCl₃) 10.38 (s, 1H), 8.18 (d, 1H), 8.09 (d, 2H), 7.74 (d, 1H), 7.56 (s, 1H), 7.53 (t, 1H), 7.14 (t, 1H), 7.03 (d, 2H), 6.83 (s, 1H), 5.69 (dt, 1H), 5.64 (brs, 1H), 5.04-4.86 (m, 3H), 4.82 (dd, 1H), 4.23 (m, 1H), 4.15 (t, 1H), 4.02 (dd, 1H), 3.89 (s, 3H), 2.90 (m, 1H), 2.73 (m, 1H), 2.55 (m, 1H), 2.35 (dd, 1H), 1.98-1.84 (m, 2H), 1.82-1.62 (m, 3H), 1.56-1.20 (m, 16H), 1.09 (m, 2H), 0.93 (m, 1H).

Compound 230: MS: m/z 854.4 (M⁺+1); ¹H NMR (CDCl₃) 10.52 (s, 1H), 8.22 (s, 1H), 8.16 (d, 1H), 7.78-7.70 (m, 2H), 7.57-7.54 (m, 2H), 7.42 (t, 1H), 7.15 (t, 1H), 7.05 (d, 1H), 6.74 (s, 1H), 5.69 (dt, 1H), 5.56 (brs, 1H), 5.06-4.96 (m, 3H), 4.87 (d, 1H), 4.16-4.02 (m, 3H), 3.95 (s, 3H), 2.98-2.87 (m, 2H), 2.64-2.54 (m, 2H), 2.38 (q, 1H), 2.06 (m, 9H), 1.84 (m, 4H), 1.45-1.12 (m, 8H), 0.92 (m, 2H).

Compound 231: MS: m/z 854.3 (M⁺+1); ¹H NMR (CDCl₃) 10.38 (s, 1H), 8.20 (d, 1H), 8.14 (dd, 1H), 7.76 (d, 1H), 7.62 (brs, 1H), 7.54 (t, 1H), 7.46 (ddd, 1H), 7.16 (m, 2H), 7.03 (d, 1H), 5.66 (dt, 1H), 5.57 (brs, 1H), 5.03 (d, 1H), 4.96 (t, 1H), 4.91 (t, 1H), 7.81 (d, 1H), 4.18 (m, 2H), 7.03 (dd, 1H), 3.89 (s, 3H), 2.90 (m, 2H), 2.67 (m, 1H), 2.50 (m, 1H), 2.32 (dd, 1H), 1.96-1.76 (m, 7H), 1.58-1.18 (m, 14H), 1.06 (m, 2H), 0.91 (m, 1H).

Compound 232: MS: m/z 908.3 (M⁺+1); ¹H NMR (CDCl₃) 10.49 (s, 1H), 8.11-8.02 (m, 3H), 7.75 (d, 1H), 7.57 (d, 1H), 7.37 (d, 2H), 7.14 (t, 1H), 6.73 (s, 1H), 5.70 (dt, 1H), 5.59 (s, 1H), 5.02-4.84 (m, 3H), 4.18 (m, 2H), 4.03 (d, 1H), 2.94 (m, 2H), 2.68 (m, 1H), 2.55 (m, 1H), 2.36 (m, 1H), 1.89-1.80 (m, 6H), 1.47-1.11 (m, 16H), 0.94-0.90 (m, 2H).

Compound 233: MS: m/z 908.3 (M⁺+1); ¹H NMR (CDCl₃) 10.50 (s, 1H), 8.19 (d, 1H), 8.15-8.12 (m, 1H), 8.05-8.01 (m, 1H), 7.78 (d, 1H), 7.60-7.52 (m, 3H), 7.43 (br s, 1H), 7.17 (t, 1H), 6.87 (s, 1H), 5.68 (dt, 1H), 5.55 (br s, 1H), 5.05-4.95 (m, 4H), 4.37-4.31 (m, 1H), 4.03-3.99 (m, 2H), 3.84 (s, 1H), 2.98-2.90 (m, 2H), 2.70-0.47 (m, 3H), 2.39-2.25 (m, 2H), 2.00-1.76 (m, 6H), 1.54-1.01 (m, 12H), 0.97-0.89 (m, 2H).

Compound 234: MS: m/z 838.3 (M⁺+1); ¹H NMR (CDCl₃) 10.44 (s, 1H), 7.15 (d, 1H), 7.79 (d, 2H), 7.84 (brs, 1H), 7.72 (d, 1H), 7.53 (t, 1H), 7.31 (d, 2H), 7.12 (t, 1H), 6.80 (s, 1H), 5.70 (dt, 1H), 5.60 (brs, 1H), 5.06-4.88 (m, 3H), 4.82 (d, 1H), 4.19 (m, 1H), 4.13 (t, 1H), 3.99 (dd, 1H), 2.91 (m, 2H), 2.66 (m, 1H), 2.51 (m, 1H), 2.43 (s, 3H), 2.35 (dd, 1H), 1.98-1.68 (m, 6H), 1.58-1.16 (m, 14H), 1.07 (m, 2H), 0.91 (m, 1H).

Compound 235: MS: m/z 880.4 (M⁺+1); ¹H NMR (CDCl₃) 10.46 (s, 1H), 8.20 (d, 1H), 8.11-8.05 (m, 2H), 7.87-7.69 (m, 2H), 7.58-7.55 (m, 3H), 7.15 (t, 1H), 6.86 (s, 1H), 5.70-5.64 (m, 2H), 5.39-5.33 (m, 2H), 5.01-4.84 (m, 4H), 4.13-4.00 (m, 3H), 2.93 (m, 2H), 2.70-2.54 (m, 2H), 1.89-0.83 (m, 31H).

Compound 237: MS: m/z 892.4 (M⁺+1); ¹H NMR (CDCl₃) 10.43 (s, 1H), 8.21 (d, 2H), 8.15 (d, 1H), 7.88 (brs, 1H), 7.77 (d, 2H), 7.56 (t, 1H), 7.16 (t, 1H), 6.83 (s, 1H), 5.70 (dt, 1H), 5.64 (brs, 1H), 4.96 (m, 3H), 4.85 (d, 1H), 4.11 (m, 2H), 4.01 (d, 1H), 2.94 (m, 2H), 2.70 (m, 1H), 2.54 (m, 1H), 2.36 (dd, 1H), 1.86 (t, 2H), 1.74-1.60 (m, 3H), 1.58-1.18 (m, 16H), 1.10 (m, 2H), 0.90 (m, 1H).

Compound 236: MS: m/z 882.1 (M⁺+1).

Compound 238: MS: m/z 892.3 (M⁺+1); ¹H NMR (CDCl₃) 10.57 (s, 1H), 8.31 (s, 2H), 8.14 (d, 1H), 8.02 (d, 1H), 7.74 (t, 2H), 7.63 (t, 1H), 7.56 (t, 1H), 7.08 (t, 1H), 6.55 (s, 1H), 5.69 (dt, 1H). 5.49 (br s, 1H), 5.01-4.88 (m, 4H), 4.19-4.00 (m, 3H), 2.96-2.89 (m, 2H), 2.63-2.41 (m, 3H), 2.16 (m, 4H), 1.89-1.79 (m, 3H), 1.49-0.95 (m, 14H), 0.95-0.83 (m, 2H).

Compound 239: MS: m/z 869.9 (M⁺+1); ¹H NMR (CDCl₃) 10.42 (s, 1H), 8.36 (dd, 4H), 8.22 (d, 1H), 7.81 (d, 1H), 7.75 (s, 1H), 7.61 (t, 1H), 7.00 (s, 1H), 5.73-5.70 (m, 2H), 5.40-5.34 (m, 1H). 5.03-4.96 (m, 2H), 4.87 (d, 2H), 4.15-4.05 (m, 3H), 3.75-3.64 (m, 2H), 2.96-2.91 (m, 2H), 2.80-2.76 (m, 1H), 2.58-2.55 (m, 1H), 2.37-2.34 (m, 1H), 1.89-1.86 (m, 1H), 1.68-1.10 (m, 19H), 0.95-0.83 (m, 2H).

Compound 240: MS: m/z 869.3 (M⁺+1); ¹H NMR (CDCl₃) 10.29 (s, 1H), 9.04 (s, 1H), 8.58 (d, 1H), 8.37-8.30 (m, 4H), 7.86 (d, 1H), 7.75 (t, 1H), 7.64-7.59 (m, 2H), 7.20-7.07 (m, 4H). 5.81-5.73 (m, 2H), 5.42-5.30 (m, 2H), 5.10 (m, 4H), 4.21-4.06 (m, 3H), 3.83-3.63 (m, 6H), 2.90-2.18 (m, 4H), 1.68-0.95 (m, 13H).

Compound 241: MS: m/z 842.3 (M⁺+1); ¹H NMR (CDCl₃) 10.53 (s, 1H), 8.24-8.18 (m, 2H), 8.07-8.05 (m, 2H), 7.88 (d, 1H), 7.71 (d, 1H), 7.58-7.53 (m, 2H), 7.30-7.09 (m, 4H), 6.54 (s, 1H), 5.67 (dt, 1H). 5.55 (brs, 1H), 5.08 (d, 1H), 5.06-4.93 (m, 2H), 4.83 (d, 1H), 4.24-4.03 (m, 3H), 3.29-3.17 (m, 2H), 2.97-2.93 (m, 2H), 2.64-2.41 (m, 2H), 2.39 (q, 1H), 2.10-1.95 (m, 6H), 1.86-0.90 (m, 10H), 0.95-0.83 (m, 2H).

Compound 242: MS: m/z 858.3 (M⁺+1); ¹H NMR (CDCl₃) 10.58 (s, 1H), 8.45 (s, 1H), 8.06 (d, 1H), 7.98 (d, 2H), 7.69 (d, 1H), 7.56-7.41 (m, 3H), 7.11 (t, 1H), 6.64 (s, 1H). 5.65 (dt, 1H), 5.52 (brs, 1H), 5.12 (d, 1H), 4.97 4.91 (m, 3H), 4.80 (d, 1H), 4.23-4.13 (m, 2H), 4.02 (d, 1H), 2.91-2.89 (m, 2H), 2.60-2.37 (m, 4H), 2.34-1.79 (m, 4H), 1.66-1.95 (m, 6H), 1.86-0.89 (m, 11H).

Compound 243: MS: m/z 882.3 (M⁺+1); ¹H NMR (CDCl₃) 10.41 (s, 1H), 8.22 (q, 4H), 7.82 (d, 1H), 7.70 (m, 1H), 7.62-7.54 (m, 1H), 7.19-7.12 (m, 2H), 7.03 (s, 1H), 5.74-5.68 (m, 2H), 5.39-5.29 (m, 2H), 5.07-4.87 (m, 4H), 4.14-4.02 (m, 3H), 3.98 (s, 3H), 2.92 (m, 2H), 2.20-0.83 (m, 24H).

Compound 244: MS: m/z 923.4 (M⁺+1); ¹H NMR (CDCl₃) 10.35 (s, 1H), 8.26 (d, 2H), 7.82 (d, 1H), 7.59 (t, 1H), 7.45 (d, 2H), 7.39 (m, 1H), 7.16 (t, 1H), 7.13 (s, 1H), 6.81 (brs, 1H), 5.80 (s, 1H), 5.70 (dt, 1H), 5.01-4.83 (m, 4H), 3.66-3.49 (m, 4H), 3.35-3.33 (m, 2H), 3.11-3.09 (m, 3H), 3.00-2.90 (m, 2H), 2.74-2.28 (m, 3H), 2.03-1.20 (m, 25H), 0.95-0.87 (m, 2H).

Compound 245: MS: m/z 895.4 (M⁺+1); ¹H NMR (CDCl₃) 10.46 (s, 1H), 8.17 (d, 1H), 8.03 (d, 2H), 7.80 (s, 1H), 7.72 (d, 2H), 7.52 (t, 1H), 7.11 (t, 1H), 6.79-6.76 (m, 3H), 5.68 (dt, 1H), 5.61 (brs, 1H), 5.07 (dd, 1H), 5.00-4.90 (m, 3H), 4.81 (d, 1H), 4.30 (s, 1H), 4.19 (t, 1H), 4.02 (dd, 1H), 3.45 (q, 4H), 2.93-2.84 (m, 1H), 2.70-2.34 (m, 3H), 1.89-1.75 (m, 6H), 1.48-1.10 (m, 20H), 0.92-0.85 (m, 2H).

Compound 246: MS: m/z 872.3 (M⁺+1); ¹H NMR (CDCl₃) 8.24 (d, 1H), 7.81 (d, 2H), 7.57 (t, 2H), 7.25-7.06 (m, 4H), 5.61 (s, 2H), 5.35-5.29 (m, 1H), 4.99-4.97 (m, 3H), 4.14 (m, 2H), 3.96 (s, 3H), 3.92 (s, 2H), 2.95-2.88 (m, 3H), 2.29-2.16 (m, 2H), 2.01-1.09 (m, 21H), 0.87 (m, 2H).

Compound 247: MS: m/z 914.4 (M⁺+1); ¹H NMR (CDCl₃) 10.46 (s, 1H), 8.22 (d, 1H), 8.00 (s, 1H), 7.75 (d, 1H), 7.57 (t, 1H), 7.34 (s, 2H), 7.18 (t, 1H), 6.78 (s, 1H), 5.70-5.65 (m, 2H), 5.03 (d, 1H), 4.96 (dd, 2H), 4.81 (dd, 1H), 4.18-4.13 (m, 2H), 4.03 (s, 6H), 3.95 (s, 3H), 3.10-3.09 (m, 1H), 2.96-2.89 (m, 2H), 2.71-2.31 (m, 2H), 2.05-1.79 (m, 6H), 1.62-1.08 (m, 16H), 0.96-0.91 (m, 2H).

Compound 248: MS: m/z 892.3 (M⁺+1); ¹H NMR (CDCl₃) 10.50 (s, 1H), 8.39-8.33 (m, 2H), 8.08-8.06 (m, 2H), 7.78 (d, 1H), 7.51-7.50 (m, 3H), 7.13 (t, 1H), 6.98 (s, 1H), 5.76 (s, 1H), 5.65 (dt, 1H), 5.26 (d, 1H), 4.98-4.96 (m, 2H), 4.82 (d, 1H), 4.52-4.49 (m, 1H), 4.34 (t, 2H), 4.24-4.19 (m, 1H), 4.06-4.03 (m, 1H), 3.74-3.69 (m, 1H), 2.94-2.88 (m, 2H), 2.65-2.46 (m, 6H), 2.35-2.22 (m, 2H), 1.91-1.78 (m, 5H), 1.52-1.05 (m, 8H), 0.92-0.90 (m, 2H).

Compound 249: MS: m/z 892.3 (M⁺+1); ¹H NMR (CDCl₃) 10.45 (s, 1H), 8.35 (d, 1H), 8.18-8.16 (m, 2H), 8.09 (s, 1H), 7.93 (d, 1H), 7.56-7.54 (m, 3H), 7.35, (d, 1H), 7.06 (s, 1H), 5.74 (s, 1H), 5.65 (dt, 1H), 5.11 (d, 1H), 4.93-4.82 (m, 3H), 4.15-4.03 (m, 3H), 2.97-2.91 (m, 2H), 2.80-2.75 (m, 1H), 2.54 (m, 1H), 2.31 (m, 1H), 1.85-1.81 (m, 5H), 1.46-1.10 (m, 16H), 0.92-0.90 (m, 2H).

Compound 250: MS: m/z 892.3 (M⁺+1); ¹H NMR (CDCl₃) 10.55 (s, 1H), 8.45-8.35 (d, 2H), 8.09 (m, 2H), 7.77 (dd, 2H), 7.54 (m, 3H), 6.83 (s, 1H), 5.67-5.60 (m, 2H), 5.11-5.09 (m, 1H), 4.99-4.85 (m, 3H), 4.10-4.01 (m, 3H), 2.92 (m, 2H), 2.67-2.53 (m, 2H), 2.36-2.24 (m, 7H), 1.84-1.81 (m, 5H), 1.45-1.10 (m, 10H), 0.95-0.90 (m, 2H).

Compound 251: MS: m/z 892.3 (M⁺+1); ¹H NMR (CDCl₃) 10.38 (s, 1H), 8.28 (d, 2H), 7.97 (d, 1H), 7.59-7.49 (d, 4H), 7.13 (s, 1H), 5.75-5.69 (m, 2H), 5.02-4.93 (m, 3H), 4.11-4.03 (m, 3H), 2.97-2.93 (m, 2H), 2.80-2.72 (m, 1H), 2.57 (m, 1H), 2.34 (q, 1H), 2.02-1.72 (m, 8H), 1.50-1.12 (m, 16H), 0.98-0.92 (m, 2H).

Compound 252: MS: m/z 854.4 (M⁺+1); ¹H NMR (CDCl₃) 10.47 (s, 1H), 8.12 (m, 3H), 7.77 (d, 1H), 7.45 (m, 3H), 7.04 (t, 1H), 6.83 (d, 1H), 5.57 (s, 1H), 5.34-5.29 (m, 1H), 4.99 (m, 2H), 4.35-4.21 (m, 1H), 4.04 (s, 3H), 3.50 (m, 1H), 2.97-2.61 (m, 2H), 2.23-2.13 (m, 1H), 2.01-1.13 (m, 27H), 0.87 (m, 2H).

Compound 253: MS: m/z 854.4 (M⁺+1); ¹H NMR (CDCl₃) 10.47 (s, 1H), 8.11 (d, 2H), 8.01 (d, 1H), 7.90 (d, 1H), 7.54-7.52 (m, 2H), 7.00 (s, 1H), 6.82 (d, 1H), 6.80 (s, 1H), 5.71-5.83 (m, 2H), 5.08-4.94 (m, 2H), 4.83 (d, 1H), 4.30 (s, 1H), 4.17 (s, 1H), 4.03 (s, 1H), 3.91 (s, 3H), 2.93-2.91 (m, 1H), 2.69-2.68 (m, 1H), 2.58 (m, 1H), 2.36 (m, 1H), 1.89-1.82 (m, 6H), 1.48-1.25 (m, 15H), 1.13-1.11 (m, 3H), 0.94-0.90 (m, 2H).

Compound 254: MS: m/z 854.4 (M⁺+1); ¹H NMR (CDCl₃) 10.45 (s, 1H), 8.09 (d, 2H), 7.83 (s, 1H), 7.68 (d, 1H), 7.55-7.50 (m, 3H), 7.42 (s, 1H), 6.74 (s, 1H), 5.71 (dt, 1H), 5.61 (s, 1H), 5.03-4.94 (m, 4H), 4.85 (d, 1H), 4.29 (brs, 1H), 4.17 (t, 1H), 4.04 (dd, 1H), 3.85 (s, 3H), 2.95-2.94 (m, 2H), 2.74-2.70 (m, 1H), 2.60-2.54 (m, 1H), 2.37 (q, 1H), 2.02-1.70 (m, 5H), 1.51-1.25 (m, 14H), 1.21-1.09 (m, 2H), 0.96-0.92 (m, 2H).

Compound 255: MS: m/z 854.4 (M⁺+1); ¹H NMR (CDCl₃) 10.44 (s, 1H), 8.16-8.14 (m, 3H), 7.77 (s, 1H), 7.50-7.44 (m, 4H), 7.33 (d, 1H), 6.88 (s, 1H), 6.44 (d, 1H), 5.65 (m, 2H), 5.07-4.85 (m, 5H), 4.32-3.97 (m, 5H), 4.02 (s, 3H), 2.92-2.90 (m, 2H), 2.69-2.33 (m, 3H), 2.03-1.68 (m, 6H), 1.49-0.69 (m, 13H).

Compound 256: MS: m/z 838.4 (M⁺+1); ¹H NMR (CDCl₃) 10.60 (s, 1H), 8.65 (d, 1H), 8.05 (d, 2H), 7.98 (d, 1H), 7.53-7.51 (m, 3H), 7.41 (s, 1H), 6.96-6.90 (m, 2H), 6.69 (s, 1H), 5.65 (dt, 1H), 5.55 (brs, 1H), 5.13-4.85 (m, 4H), 4.82 (d, 1H), 4.23-4.00 (m, 3H), 2.97-2.91 (m, 2H), 2.45 (s, 3H), 2.20 (m, 6H), 1.81 (t, 3H), 1.25 (m, 12H).

Compound 257: MS: m/z 838.4 (M⁺+1); ¹H NMR (CDCl₃) 10.50 (s, 1H), 8.08 (d, 3H), 7.86 (s, 1H), 7.64 (d, 1H), 7.53 (d, 3H), 7.39 (d, 1H), 6.68 (s, 1H), 5.72 (dt, 1H), 5.55 (brs, 1H), 5.02-4.96 (m, 3H), 4.88 (d, 1H), 4.24 (s, 1H), 4.15 (t, 1H), 4.03 (d, 1H), 3.63 (m, 1H), 2.93 (m, 2H), 2.75 (t, 1H), 2.53 (m, 1H), 2.40 (s, 3H), 2.01-1.01 (m, 20H), 0.89 (m, 3H).

Compound 258: MS: m/z 882.4 (M⁺+1); ¹H NMR (CDCl₃) 10.66 (s, 1H), 8.14 (dd, 2H), 8.05 (d, 1H), 7.56-7.44 (m, 4H), 7.00 (d, 1H), 6.84 (s, 1H), 6.80 (dd, 1H), 5.70 (m, 2H), 4.98 (m, 2H), 4.89 (t, 1H), 4.82 (d, 1H), 4.68 (ddd, 1H), 4.22 (m, 1H), 4.15 (t, 1H), 4.01 (dd, 1H), 2.91 (m, 1H), 2.71 (m, 1H), 2.53 (m, 1H), 2.33 (dd, 1H), 1.90 (t, 2H), 1.80 (m, 2H), 1.73-1.58 (m, 6H), 1.54-1.18 (m, 17H), 1.10 (m, 2H), 0.92 (m, 1H).

Compound 259: MS: m/z 922.3 (M⁺+1); ¹H NMR (CDCl₃) 10.43 (s, 1H), 8.38 (d, 1H), 8.08 (d, 2H), 7.94 (s, 1H), 7.82 (d, 1H), 7.17 (t, 1H), 7.02-7.00 (m, 3H), 5.81 (brs, 1H), 5.69 (dt, 1H), 5.15 (d, 1H), 5.02-4.91 (m, 2H), 4.80 (dd, 1H), 4.62 (brs, 1H), 4.24 (t, 1H), 4.06 (d, 1H), 3.90 (s, 3H), 2.94-2.91 (m, 2H), 2.73-2.70 (m, 1H), 2.61-2.56 (m, 1H), 2.35 (q, 1H), 1.95-1.80 (m, 5H), 1.56-1.25 (m, 14H), 1.16-1.08 (m, 3H), 0.97-0.89 (m, 2H).

Compound 260: MS: m/z 884.4 (M⁺+1); ¹H NMR (CDCl₃) 10.52 (s, 1H), 8.09 (d, 2H), 7.77 (d, 1H), 7.04 (d, 4H), 6.81 (s, 2H), 5.65 (m, 2H), 5.15 (d, 1H), 4.95 (t, 2H), 4.78 (d, 1H), 4.30 (s, 1H), 4.18 (t, 1H), 4.06 (s, 3H), 3.89 (s, 3H), 2.90 (m, 2H), 2.63-2.52 (m, 2H), 2.35 (q, 1H), 2.03-1.09 (m, 22H), 0.91 (m, 2H).

Compound 261: MS: m/z 868.4 (M⁺+1); ¹H NMR (CDCl₃) 10.52 (s, 1H), 8.13-8.11 (m, 1H), 8.04 (d, 2H), 8.94 (d, 1H), 7.02 (d, 2H), 6.96 (s, 1H), 6.77 (d, 1H), 6.62 (s, 1H), 5.67 (dt, 1H), 5.54 (brs, 1H), 5.16 (dd, 1H), 5.00-4.93 (m, 2H), 4.76 (d, 1H), 4.38 (s, 1H), 4.19 (t, 1H), 4.00 (dd, 1H), 3.90 (s, 6H), 2.92-2.88 (m, 2H), 2.68-2.25 (m, 5H), 1.91-1.79 (m, 4H), 1.50-1.23 (m, 12H), 1.13-1.11 (m, 3H), 0.98-0.88 (m, 2H).

Compound 262: MS: m/z 884.3 (M⁺+1); ¹H NMR (CDCl₃) 10.49 (s, 1H), 8.08 (brs, 1H), 7.97 (d, 2H), 7.60 (d, 1H), 7.28 (s, 1H), 7.21 (dd, 1H), 7.01 (d, 2H), 6.51 (s, 1H), 5.68 (dt, 1H), 5.49 (brs, 1H), 5.03 (d, 1H), 4.98 (d, 1H), 4.93 (t, 1H), 4.82 (d, 1H), 4.34 (m, 1H), 4.15 (t, 1H), 3.99 (dd, 1H), 3.89 (s, 3H), 3.78 (s, 3H), 2.92 (m, 2H), 2.63 (m, 1H), 2.53 (m, 1H), 2.37 (dd, 1H), 1.94-1.75 (m, 8H), 1.58-1.22 (m, 12H), 1.06 (m, 2H), 0.89 (m, 1H).

Compound 263: MS: m/z 884.3 (M⁺+1); ¹H NMR (CDCl₃) 10.44 (s, 1H), 8.11 (d, 2H), 7.85 (s, 1H), 7.43 (t, 1H), 7.30 (d. 1H), 7.02 (d, 2H), 6.82 (s, 1H), 6.42 (dd, 1H), 5.65-5.63 (m, 2H), 5.08-4.80 (m, 4H), 4.34 (s, 1H), 4.18-4.03 (m, 2H), 4.03 (s, 3H), 3.89 (s, 3H), 3.57-3.55 (m, 1H), 2.91-2.54 (m, 4H), 2.33-1.89 (m, 8H), 1.49-0.70 (m, 15H).

Compound 264: MS: m/z 912.4 (M⁺+1); ¹H NMR (CDCl₃) 10.50 (s, 1H), 8.07 (d, 2H), 7.88 (s, 1H), 7.77 (d, 1H), 7.08-7.00 (m, 4H), 6.84-6.81 (m, 3H), 5.69 (m, 2H), 5.12-4.92 (m, 3H), 4.76 (d, 1H), 4.69-4.64 (m, 2H), 4.35 (s, 1H), 4.20 (t, 1H), 4.04 (s, 3H), 2.91 (m, 1H), 2.80-2.54 (m, 3H), 2.36 (q, 1H), 1.87-1.69 (m, 6H), 1.52-1.18 (m, 19H), 0.96-0.87 (m, 2H).

Compound 265: MS: m/z 912.3 (M⁺+1); ¹H NMR (CDCl₃) 10.45 (s, 1H), 8.07-7.91 (m, 7H), 7.02-7.00 (m, 7H), 8.80 (d, 2H), 6.70 (s, 1H), 5.70-536 (m, 4H), 5.07-4.96 (s, 7H), 4.80-4.64 (m, 4H). 4.34-3.62 (m, 4H), 2.907-2.35 (m, 8H), 2.04-0.874 (m, 12H).

Compound 266: MS: m/z 884.3 (M⁺+1); ¹H NMR (CDCl₃) 10.45 (s, 1H), 8.10-8.04 (m, 3H), 7.73 (s, 1H), 7.49 (s, 1H), 7.04 (d, 2H), 6.98 (d, 1H), 6.78 (s, 1H), 5.71 (dt, 1H), 5.64 (brs, 1H), 5.07-4.90 (m, 3H), 4.66 (dd, 1H), 4.32 (m, 1H), 4.19 (t, 1H), 4.03 (dd, 1H), 3.91 (s, 3H), 2.96-2.89 (m, 2H), 2.72-2.69 (m, 1H), 2.57-2.51 (m, 1H), 2.51 (s, 3H), 2.38 (q, 1H), 2.01-1.73 (m, 6H), 1.50-1.01 (m, 15H), 0.98-0.88 (m, 2H).

Compound 267: MS: m/z 868.4 (M⁺+1); ¹H NMR (CDCl₃) 10.48 (s, 1H), 8.05 (d, 2H), 7.91 (s, 1H), 7.87 (s, 1H), 7.62 (d, 1H), 7.37 (d, 1H), 7.04 (d, 2H), 6.64 (s, 1H), 5.70 (dt, 1H), 5.55 (brs, 1H), 5.02-4.92 (m, 3H), 4.82 (dd, 1H), 4.30 (s, 1H), 4.17 (t, 1H), 4.02 (dd, 1H), 3.92 (s, 3H), 2.96-2.91 (m, 2H), 2.75-2.45 (m, 2H), 2.43 (s, 3H), 2.40-2.37 (m, 1H), 1.91-1.88 (m, 3H), 1.72-1.23 (m, 13H), 1.16-1.09 (m, 3H), 0.98-0.91 (m, 2H).

Compound 268: MS: m/z 912.4 (M⁺+1); ¹H NMR (CDCl₃) 10.44 (s, 1H), 8.09 (d, 2H), 8.01 (d, 1H), 7.82 (s, 1H), 7.03 (d, 2H), 6.99 (s, 1H), 6.79-6.75 (m, 2H), 5.70-5.64 (m, 3H), 5.05 (d, 1H), 5.00-4.92 (m, 2H), 4.79 (d, 1H), 4.90-4.67 (m, 1H), 4.32 (s, 1H), 4.18 (t, 1H), 4.02 (dd, 1H), 3.89 (s, 3H), 2.93-2.90 (m, 2H), 2.69-53 (m, 2H), 2.33 (q, 1H), 1.99-1.68 (m, 9H), 1.99-0.85 (m, 19H).

Compound 269: MS: m/z 940.4 (M⁺+1); ¹H NMR (CDCl₃) 10.42 (s, 1H), 8.40 (m, 4H), 7.60 (s, 1H), 7.02 (d, 2H), 9.79-6.74 (m, 2H), 5.71 (m, 2H), 5.07-4.63 (m, 9H), 4.33 (s, 1H). 4.20 (t, 1H), 4.03 (d, 1H), 2.93-2.34 (m, 8H), 2.02-0.823 (m, 29H).

Compound 270: MS: m/z 825.4 (M⁺+1); ¹H NMR (CDCl₃) 10.46 (s, 1H), 8.67 (m, 2H), 8.12 (m, 1H), 7.89 (t, 2H), 7.74 (d, 1H), 7.65 (m, 1H), 7.51 (t, 1H), 7.38 (t, 1H), 7.09 (m, 1H), 5.70 (dt, 1H), 5.64 (m, 1H), 5.06-4.85 (m, 4H), 4.08 (m, 3H), 2.91 (m, 2H), 2.70 (m, 1H), 2.54 (m, 1H), 2.38 (dd, 1H), 1.98-1.64 (m, 6H), 1.56-1.02 (m, 16H), 0.89 (m, 1H).

Compound 271: MS: m/z 825.4 (M⁺+1).

Compound 272: MS: m/z 824.4 (M⁺+1); ¹H NMR (CDCl₃) 10.35 (s, 1H), 8.81 (brs, 1H), 8.23 (d, 1H), 8.07 (brs, 1H), 7.81 (d, 1H), 7.60 (t, 1H), 7.46 (m, 1H), 7.01 (s, 1H), 6.95 (m, 1H), 5.74 (m, 2H), 5.34 (m, 2H), 5.04-4.82 (m, 4H), 4.12 (m, 2H), 4.05 (m, 1H), 3.62 (m, 3H), 2.90 (m, 2H), 2.76 (m, 1H), 2.52 (m, 1H), 2.31 (m, 3H), 2.04-1.02 (m, 16H), 0.85 (m, 2H).

Compound 273: MS: m/z 826.3 (M⁺+1); ¹H NMR (CDCl₃) 10.46 (s, 1H), 9.84 (s, 1H), 8.66 (d, 1H), 8.61 (s, 1H), 8.10 (d, 1H), 7.94 (brs, 1H), 7.72 (d, 1H), 7.49 (t, 1H), 7.46 (s, 1H), 7.07 (t, 1H), 5.70 (dt, 1H), 5.60 (brs, 1H), 5.2-4.88 (m, 4H), 4.16-4.02 (m, 3H), 2.94 (m, 2H), 2.70 (m, 1H), 2.56 (m, 1H), 2.37 (dd, 1H), 1.90 (m, 2H), 1.80-1.64 (m, 2H), 1.58-1.04 (m, 18H), 0.90 (m, 1H).

Compound 274: MS: m/z 867.4 (M⁺+1); ¹H NMR (CDCl₃) 10.57 (s, 1H), 8.47 (d, 1H), 8.13-8.10 (m, 2H), 7.82 (t, 2H), 7.72 (s, 2H), 7.50 (t, 1H), 7.29 (d, 2H), 7.06 (t, 1H), 5.71-5.63 (m, 2H), 5.05-4.95 (m, 4H), 4.87 (dd, 1H), 4.12-4.07 (m, 3H), 3.21-3.14 (m, 1H), 2.98-2.92 (m, 2H), 2.68-2.55 (m, 2H), 2.40-2.37 (m, 1H), 2.00-1.77 (m, 6H), 1.42-0.71 (m, 20H).

Compound 275: MS: m/z 814.3 (M⁺+1); ¹H NMR (CDCl₃) 10.57 (s, 1H), 8.34 (s, 1H), 8.08 (d, 1H), 7.65 (t, 1H), 7.60 (s, 1H), 7.53 (t, 1H), 7.28 (d, 2H), 7.07 (t, 1H), 6.64-6.62 (m, 2H), 5.68 (dt, 1H). 5.49 (brs, 1H), 5.06 (d, 1H), 4.98 (m, 2H), 4.86 (d, 1H), 4.18-4.12 (m, 2H), 4.03 (d, 1H), 2.94 (m, 2H), 2.68-2.51 (m, 2H), 2.38 (q, 1H), 2.14-1.79 (m, 6H), 1.48-0.70 (m, 16H).

Compound 276: MS: m/z 814.3 (M⁺+1); ¹H NMR (CDCl₃) 10.49 (s, 1H), 8.12 (m, 4H), 7.71 (d, 1H), 7.57-7.52 (m, 3H), 7.14 (t, 1H), 7.02 (s, 1H), 6.51 (s, 1H), 5.68 (dt, 1H). 5.53 (brs, 1H), 5.08 (d, 1H), 5.00-4.94 (m, 2H), 4.79 (d, 1H), 4.28 (s, 1H), 4.16 (t, 1H), 3.98 (dd, 1H), 2.92-2.89 (m, 3H), 2.63-2.35 (m, 3H), 2.04-1.68 (m, 6H), 1.48-0.70 (m, 14H).

Compound 277: MS: m/z 830.3 (M⁺+1).

Compound 278: MS: m/z 829.1 (M⁺+1); ¹H NMR (CDCl₃) 10.48 (s, 1H), 8.16 (d, 2H), 7.76 (d, 1H), 7.56 (t, 1H), 7.20-7.15 (m, 2H), 6.81 (s, 1H), 5.70 (dt, 1H), 5.60 (brs, 1H), 4.97 (t, 2H), 4.91 (d, 2H), 4.09-3.94 (m, 3H), 2.97-2.88 (m, 2H), 2.69-2.61 (m, 2H), 2.56 (s, 3H), 2.38 (q, 1H), 2.03-1.07 (m, 20H), 0.91 (m, 3H).

Compound 279: MS: m/z 842.3 (M⁺+1); ¹H NMR (CDCl₃) 10.43 (s, 1H), 8.16 (d, 1H), 7.78 (s, 1H), 7.72 (d, 1H), 7.51 (t, 1H), 7.16 (s, 1H), 7.13 (t, 1H), 6.87 (s, 1H), 6.65 (dt, 1H), 5.61 (brs, 1H), 5.02 (d, 1H), 4.95 (t, 1H), 4.90 (t, 1H), 4.82 (d, 1H), 4.14 (m, 2H), 4.03 (dd, 1H), 3.86 (s, 3H), 2.90 (m, 2H), 2.68 (m, 1H), 2.52 (m, 1H), 2.35 (s, 3H), 2.2-1.67 (m, 7H), 1.56-1.02 (m, 16H), 0.90 (m, 1H).

Compound 280: MS: m/z 873.3 (M⁺+1); ¹H NMR (CDCl₃) 10.42 (s, 1H), 8.21 (d, 1H), 7.78 (d, 1H), 7.61-7.54 (dd, 2H), 7.39 (s, 1H), 7.18 (d, 1H), 7.14 (s, 1H), 5.70 (dt, 1H), 5.40 (s, 1H), 5.03-4.89 (m, 3H), 4.09-3.97 (m, 2H), 3.22 (m, 1H), 2.94 (m, 1H), 2.73 (m, 1H), 2.57-2.48 (m, 2H), 2.36 (m, 1H), 2.14 (q, 1H), 1.74 (m, 9H), 1.53-1.12 (m, 14H), 0.97-0.83 (m, 7H).

Compound 281: MS: m/z 788.3 (M⁺+1); ¹H NMR (CDCl₃) 10.54 (s, 1H), 8.46 (s, 1H), 8.09 (d, 1H), 7.63 (d, 1H), 7.51 (t, 1H), 7.11 (t, 1H), 6.02 (s, 1H), 5.63 (dt, 1H), 5.35 (brs, 1H), 5.10 (d, 1H), 4.93 (m, 2H), 4.77 (d, 1H), 4.19 (m, 1H), 4.13 (t, 1H), 3.96 (dd, 1H), 2.90 (m, 2H), 2.54 (m, 2H), 2.37 (dd, 1H), 2.21 (m, 5H), 1.90 (m, 2H), 1.80 (m, 3H), 1.58-1.04 (m, 16H), 0.88 (m, 2H).

Compound 282: MS: m/z 816.4 (M⁺+1); ¹H NMR (CDCl₃) 10.23 (s, 1H), 8.05 (m, 1H), 7.49 (m, 2H), 7.04 (m, 2H), 6.64 (d, 1H), 5.70 (dt, 1H), 5.37 (s, 1H), 5.31 (m, 2H), 4.96 (m, 2H), 4.76 (dd, 1H), 4.62 (d, 1H), 4.22 (m, 1H), 4.06 (m, 1H), 3.72 (m, 1H), 3.63 (m, 2H), 2.90 (m, 1H), 2.79 (m, 2H), 2.52 (m, 2H), 2.32-2.12 (m, 2H), 2.08-1.84 (m, 6H), 1.62-1.04 (m, 16H), 0.83 (m, 4H).

Compound 283: MS: m/z 816.1 (M⁺+1); ¹H NMR (CDCl₃) 10.30 (s, 1H), 8.32 (d, 1H), 7.90 (d, 1H), 7.67 (dd, 1H), 7.34 (dd, 1H), 7.17 (s, 1H), 6.92 (s, 1H), 5.72 (dt, 1H), 5.64 (s, 1H), 5.05-4.85 (m, 3H), 3.96-4.10 (m, 2H), 3.75 (m, 1H), 2.92 (m, 1H), 2.77 (m, 1H), 2.60-2.48 (m, 2H), 2.32 (m, 1H), 2.14 (q, 1H), 1.69 (m, 9H), 1.50-1.12 (m, 12H), 0.94-0.88 (m, 2H).

Compound 284: MS: m/z 792.3 (M⁺+1); ¹H NMR (CDCl₃) 10.17 (s, 1H), 8.12 (d, 1H), 7.72 (d, 1H), 7.51 (t, 1H), 7.21 (s, 1H), 7.17 (m, 1H), 6.85 (s, 1H), 6.79 (s, 1H), 5.99 (brs, 1H), 5.70 (dt, 1H), 5.38 (m, 1H), 5.15 (d, 1H), 5.06 (s, 2H), 4.98 (t, 1H), 4.89 (m, 1H), 4.60 (t, 1H), 4.33 (t, 2H), 4.08 (dd, 1H), 3.62 (s, 3H), 3.59 (m, 1H), 2.90 (m, 1H), 2.69 (m, 1H), 2.58 (m, 1H), 2.48 (m, 1H), 2.23 (dd, 2H), 2.08-1.84 (4H), 1.80-1.18 (m, 13H), 1.10 (m, 1H), 0.85 (m, 1H).

Compound 285: MS: m/z 790.4 (M⁺+1); ¹H NMR (CDCl₃) 10.51 (s, 1H), 8.27 (s, 1H), 8.20 (d, 1H), 7.71 (d, 1H), 7.55 (t, 1H), 7.18 (t, 1H), 6.26 (s, 1H), 5.65 (dt, 1H), 5.47 (s, 1H), 5.10 (d, 1H), 5.01-4.91 (m, 2H), 4.82 (d, 1H), 4.10-3.96 (m, 3H), 2.87-2.84 (m, 4H), 2.65-2.45 (m, 1H), 2.41-2.26 (m, 2H), 1.83-0.80 (m, 8H), 1.47-1.11 (m, 16H), 1.04-1.00 (t, 2H), 0.94-0.90 (m, 2H).

Compound 286: MS: m/z 856.4 (M⁺+1); ¹H NMR (CDCl₃) 10.37 (s, 1H), 8.30 (d, 1H), 7.89 (s, 1H), 7.81 (d, 1H), 7.15 (t, 1H), 6.44 (s, 1H), 5.67 (dt, 1H), 5.61 (s, 1H), 5.13 (d, 1H), 4.96 (t, 1H), 4.92 (dd, 1H), 4.75 (d, 1H), 4.51 (m, 1H), 4.22 (t, 1H), 4.03 (dd, 1H), 2.88-2.84 (m, 2H), 2.72-2.58 (m, 1H), 2.58-2.44 (m, 1H), 2.32 (dd, 1H), 2.03 (m, 1H), 1.86-1.72 (m, 11H), 1.62-10.4 (m, 14H), 0.91 (m, 2H).

Compound 287: MS: m/z 838.3 (M⁺+1); ¹H NMR (CDCl₃) 10.25 (s, 1H), 8.21 (d, 1H), 8.14 (d, 2H), 7.76 (d, 1H), 7.62-7.48 (m, 5H), 7.17 (t, 1H), 6.89 (s, 1H), 5.76-5.64 (m, 1H), 4.97 (dd, 1H), 4.88 (m, 1H), 4.14 (t, 1H), 4.03 (dd, 1H), 2.93 (dd, 1H), 2.75 (m, 1H), 2.53 (m, 1H), 2.34 (dd, 1H), 1.87 (t, 2H), 1.87 (d, 2H), 1.76-1.63 (m, 13H), 1.58-1.16 (m, 12H), 0.81 (m, 1H).

Compound 288: MS: m/z 868.0 (M⁺+1); ¹H NMR (CDCl₃) 10.30 (s, 1H), 8.15 (d, 1H), 8.08 (d, 2H), 7.80 (brs, 1H), 7.72 (d, 2H), 7.52 (t, 1H), 7.12 (t, 1H), 7.03 (d, 2H), 6.78 (s, 1H), 5.68 (dt, 1H), 5.61 (s, 1H), 5.01 (t, 2H), 4.93 (ddd, 1H), 4.81 (d, 1H), 4.23 (s, 1H), 4.15 (t, 1H), 4.03 (d, 1H), 3.89 (s, 3H), 2.90 (dd, 1H), 2.68 (m, 1H), 2.52 (m, 1H), 2.35 (dd, 1H), 1.96-1.73 (m, 11H), 1.60-1.18 (m, 13H), 0.82 (m. 1H).

Compound 289: MS: m/z 802.1 (M⁺+1); ¹H NMR (CDCl₃) 10.35 (s, 1H), 8.14 (brs, 1H), 8.10 (d, 1H), 7.66 (d, 1H), 7.51 (t, 1H), 7.12 (t, 1H), 6.15 (s, 1H), 5.65 (dt, 1H), 5.41 (s, 1H), 5.09 (d, 1H), 4.96 (t, 1H), 4.92 (dd, 1H), 4.77 (d, 1H), 4.19 (m, 1H), 4.16 (m, 1H), 4.99 (dd, 1H), 2.86 (dd, 1H), 2.66-2.43 (m, 2H), 2.36 (dd, 1H), 2.4-1.72 (m, 12H), 15.8-11.4 (m, 15H), 1.09 (d, 2H), 0.77 (s, 2H).

Compound 290: MS: m/z 812.3 (M⁺+1); ¹H NMR (CDCl₃) 10.62 (s, 1H), 8.55 (s, 1H), 8.11 (d, 1H), 8.03 (d, 2H), 7.70-7.68 (dd, 1H), 7.51 (m, 4H), 7.12-7.08 (dd, 1H), 6.68 (s, 1H), 5.64 (dt, 1H), 5.51 (s, 1H), 5.05-4.87 (m, 3H), 4.11-4.02 (m, 2H), 2.92 (m, 1H), 2.63 (m, 2H), 2.38 (m, 1H), 2.02-1.79 (m, 4H), 1.53-1.11 (m, 14H), 0.95-0.83 (m, 8H).

Compound 291: MS: m/z 842.4 (M⁺+1); ¹H NMR (CDCl₃) 10.60 (s, 1H), 8.40 (s, 1H), 8.07 (d, 1H), 8.00 (d, 2H), 7.67 (d, 1H), 7.51 (dd, 1H), 7.07-7.00 (m, 3H), 6.60 (s, 1H), 5.65 (dt, 1H), 5.47 (s, 1H), 5.11-4.83 (m, 3H), 4.15-4.02 (m, 2H), 3.90 (s, 3H), 2.96 (m, 1H), 2.61 (m, 2H), 2.38 (m, 1H), 2.03-1.82 (m, 4H), 1.53-1.07 (m, 14H), 0.99-0.82 (m, 8H).

Compound 292: MS: m/z 842.3 (M⁺+1); ¹H NMR (CDCl₃) 10.44 (s, 1H), 8.16 (d, 1H), 7.78 (s, 1H), 7.72 (d, 1H), 7.69 (s, 1H), 7.58 (d, 1H), 7.56 (t, 1H), 7.42 (t, 1H), 7.15 (t, 1H), 7.03 (dd, 1H), 6.73 (s, 1H), 5.67 (dt, 1H), 5.55 (brs, 1H), 4.94 (m, 3H), 4.10 (t, 1H), 4.03 (dd, 1H), 3.93 (s, 3H), 2.93 (m, 2H), 2.70 (m, 1H), 2.54 (m, 1H), 2.37 (dd, 1H), 1.87 (t, 2H), 1.73 (m, 6H), 1.56-1.20 (m, 8H), 1.10 (m, 2H), 0.92 (s, 6H), 0.90 (m, 1H).

Compound 293: MS: m/z 814.3 (M⁺+1); ¹H NMR (CDCl₃) 10.49 (s, 1H), 9.82 (s, 1H), 8.66 (d, 1H), 8.61 (t, 1H), 8.09 (m, 2H), 7.69 (d, 1H), 7.49 (t, 1H), 7.44 (s, 1H), 7.06 (t, 1H), 5.70 (dt, 1H), 5.58 (brs, 1H), 5.06-4.96 (m, 4H), 4.07 (dd, 2H), 2.97 (m, 2H), 2.74 (m, 1H), 2.56 (m, 1H), 2.37 (dd, 1H), 1.89 (t, 2H), 1.84-1.66 (m, 6H), 1.60-1.20 (m, 8H), 1.11 (m, 2H), 0.89 (s, 6H).

Compound 294: MS: m/z 802.3 (M⁺+1); ¹H NMR (CDCl₃) 10.49 (s, 1H), 8.10 (d, 1H), 8.00 (brs, 1H), 7.66 (d, 1H), 7.59 (s, 1H), 7.51 (t, 1H), 7.29 (d, 1H), 7.09 (t, 1H), 6.68 (s, 1H), 6.60 (t, 1H), 5.68 (dt, 1H), 5.50 (brs, 1H), 4.96 (m, 3H), 4.85 (d, 1H), 4.07 (m, 2H), 2.91 (m, 1H), 2.66 (m, 1H), 2.52 (m, 1H), 2.35 (dd, 1H), 2.03-1.71 (m, 8H), 1.58-1.20 (m, 9H), 1.11 (m, 2H), 0.94 (s, 6H).

Compound 295: MS: m/z 776.3 (M⁺+1); ¹H NMR (CDCl₃) 10.31 (s, 1H), 8.23 (s, 1H), 8.12 (d, 1H), 7.78 (d, 1H), 7.53 (dd, 1H), 7.16 (dd, 1H), 6.91 (s, 1H), 5.94 (s, 1H), 5.70 (dt, 1H), 5.25 (s, 1H), 4.97 (t, 1H), 4.63 (dd, 1H), 4.42-4.30 (m, 2H), 4.09-4.04 (m, 1H), 3.02 (m, 1H), 2.90 (m, 1H), 2.79 (m, 3H), 2.62 (m, 2H), 2.28 (m, 1H), 1.84 (m, 4H), 1.48-1.06 (m, 20H), 0.98-0.82 (m, 2H).

Compound 296: MS: m/z 697.2 (M⁺+1); ¹H NMR (CDCl₃) 10.48 (s, 1H), 8.12-8.05 (m, 4H), 7.76 (d, 1H), 7.59-7.52 (m, 4H), 7.15 (t, 1H), 6.68 (s, 1H), 5.70 (dt, 1H), 5.49 (brs, 1H), 4.96 (t, 1H), 4.79 (t, 1H), 4.12 (t, 2H), 2.89 (t, 1H), 2.77-2.67 (m, 2H), 2.52 (q, 2H), 2.31-1.09 (m, 14H), 0.91 (m, 3H).

Compound 297: MS: m/z 765.3 (M⁺+1); ¹H NMR (CDCl₃) 10.31 (s, 1H), 8.44 (d, 1H), 8.16 (d, 1H), 8.13 (d, 1H), 7.85 (d, 1H), 7.55 (m, 3H), 7.39 (s, 1H), 7.20 (d, 1H), 7.01 (s, 1H), 5.76 (m, 1H), 5.68 (dt, 1H), 4.96 (t, 1H), 4.80 (t, 1H), 4.29 (m, 2H), 4.91 (m, 1H), 2.77 (m, 2H), 2.58-2.37 (m, 3H), 2.26 (m, 1H), 1.96-1.64 (m, 3H), 1.58-1.22 (m, 9H), 1.10 (m, 2H), 0.88 (m, 1H).

Compound 298: MS: m/z 726.6 (M⁺+1); ¹H NMR (CDCl₃) 10.42 (s, 1H), 8.12 (d, 1H), 8.04 (d, 2H), 7.77 (d, 2H), 7.56 (t, 1H), 7.12 (t, 1H), 7.05 (d, 2H), 6.63 (s, 1H), 5.71 (dt, 1H), 5.47 (brs, 1H), 4.97 (t, 1H), 4.77 (t, 1H), 4.10 (m, 2H), 3.90 (s, 3H), 3.78 (m, 1H), 2.89 (m, 1H), 2.75-2.68 (m, 2H), 2.50 (q, 2H), 2.35-1.06 (m, 14H), 0.91 (m, 2H).

Compound 299: MS: m/z 727.3 (M⁺+1); ¹H NMR (CDCl₃) 10.32 (s, 1H), 8.15 (d, 1H), 7.77 (d, 1H), 7.71 (t, 1H), 7.64-7.50 (m, 2H), 7.43 (t, 1H), 7.18 (t, 1H), 7.05 (dd, 1H), 6.70 (s, 1H), 5.69 (dt, 1H), 5.52 (brs, 1H), 4.96 (t, 1H), 4.76 (t, 1H), 4.15 (m, 2H), 3.94 (s, 3H), 2.91 (m, 1H), 2.80 (m, 1H), 2.72 (m, 1H), 2.53 (m, 2H), 2.40-2.18 (m, 3H), 2.04-1.78 (m, 3H), 1.74-1.22 (m, 9H), 1.08 (m, 2H), 0.89 (m, 1H).

Compound 300: MS: m/z 699.2 (M⁺+1); ¹H NMR (CDCl₃) 10.49 (s, 1H), 9.85 (s, 1H), 8.68 (d, 2H), 8.12 (d, 1H), 7.79 (d, 1H), 7.58 (t, 1H), 7.51 (s, 1H), 7.48 (s, 1H), 7.17 (t, 1H), 5.71 (dt, 1H), 5.59 (brs, 1H), 4.97 (t, 1H), 4.77 (t, 1H), 4.19 (m, 2H), 3.70 (m, 2H), 3.14 (dd, 1H), 2.98-2.70 (m, 3H), 2.50 (dd, 2H), 2.42-2.18 (m, 3H), 2.06-1.82 (m, 2H), 1.78-1.24 (m, 6H), 1.09 (m, 2H), 0.89 (m, 1H).

Compound 301: MS: m/z 687.2 (M⁺+1); ¹H NMR (CDCl₃) 10.30 (s, 1H), 8.19 (d, 1H), 7.77 (d, 1H), 7.62 (d, 1H), 7.57 (m, 1H), 7.35 (d, 1H), 7.19 (t, 1H), 6.83 (s, 1H), 6.63 (dd, 1H), 5.71 (dt, 1H), 5.60 (brs, 1H), 4.97 (t, 1H), 4.73 (t, 1H), 4.20 (m, 2H), 3.62 (m, 1H), 2.93 (m, 1H), 2.77 (m, 2H), 2.52 (m, 2H), 2.29 (m, 1H), 2.24 (m, 1H), 1.93 (m, 1H), 1.90 (dd, 2H), 1.68-1.04 (m, 11H), 0.93 (m, 1H).

Compound 302: MS: m/z 660.6 (M⁺+1); ¹H NMR (CDCl₃) 10.29 (s, 1H), 8.13 (d, 1H), 7.79 (d, 1H), 7.53 (t, 1H), 7.16 (t, 1H), 6.98 (s, 1H), 6.18 (brs, 1H), 5.91 (s, 1H), 5.73 (dt, 1H), 4.98 (t, 1H), 4.56 (t, 1H), 4.12 (dd, 1H), 3.93 (d, 1H), 3.03 (m, 1H), 2.91 (m, 1H), 2.65-2.17 (m, 6H), 2.04-1.05 (m, 17H), 0.96-0.82 (m, 2H).

Compound 303: MS: m/z 787.6 (M⁺+1); ¹H NMR (CDCl₃) 10.54 (s, 1H), 8.20 (m, 1H), 8.10 (d, 2H), 7.72-7.43 (m, 6H), 7.07-7.02 (m, 2H), 6.93 (t, 1H), 6.66 (s, 1H), 6.55-6.41 (m, 3H), 5.65 (dt, 1H), 5.52 (brs, 1H), 5.39-5.26 (m, 2H), 4.94 (t, 1H), 3.71 (t, 1H), 4.24-4.10 (m, 3H), 2.90 (m, 1H), 2.60-2.44 (m, 3H), 2.21 (q, 1H), 2.07-0.99 (m, 12H), 0.87-0.75 (m, 2H).

### Example 15

### Synthesis of [4-Cyclopropanesulfonylaminocarbonyl-2,15-dioxo-18-(2-phenyl-benzo[4,5]imidazo[1,2-a]pyrimidin-4-yloxy)-3,16-diazatricyclo[14.3.0.04,6]nonadec-7-en-14-yl]-carbamic acid cyclopentyl ester (Compound 304)

Compound 304 was prepared by the following route:

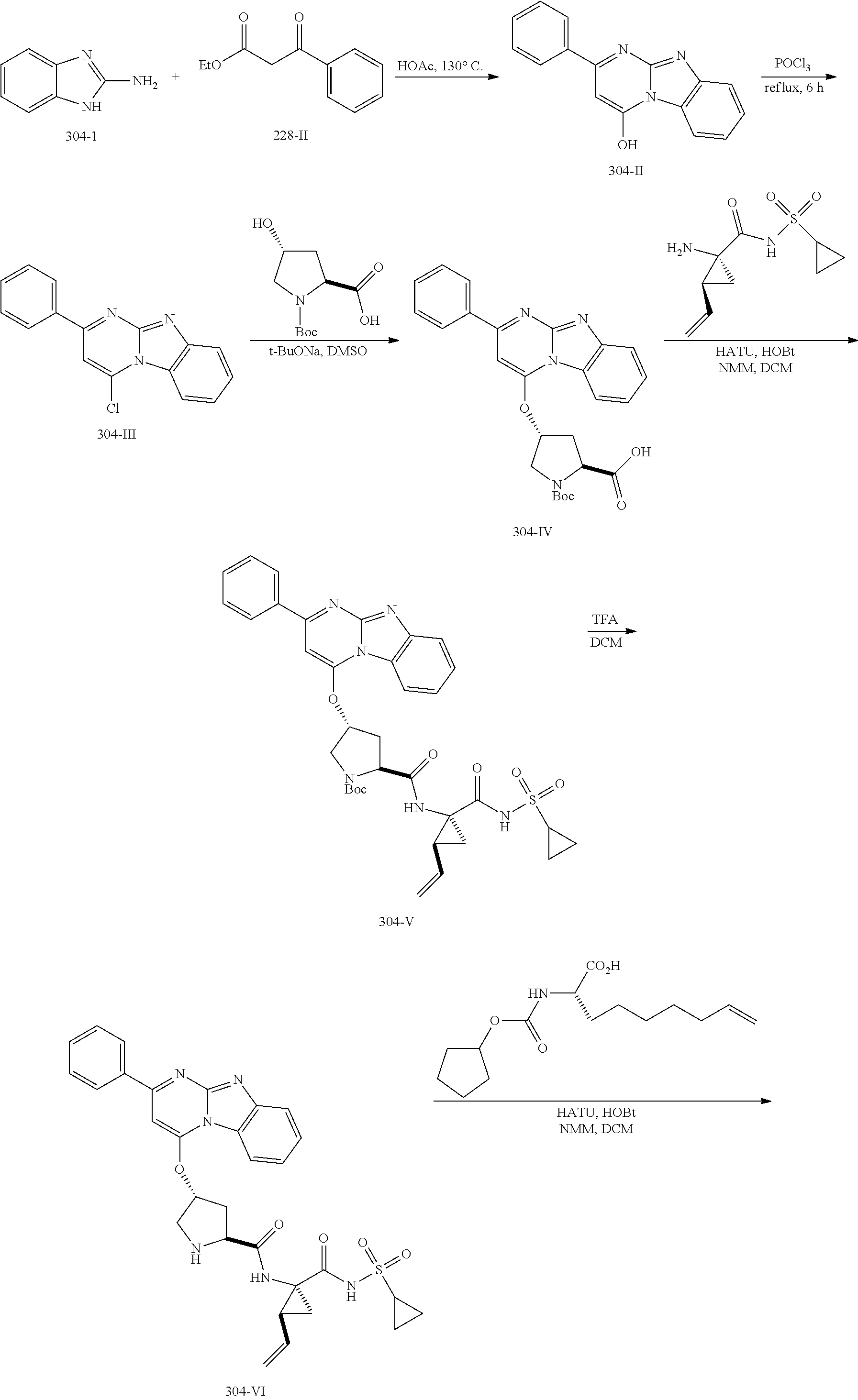

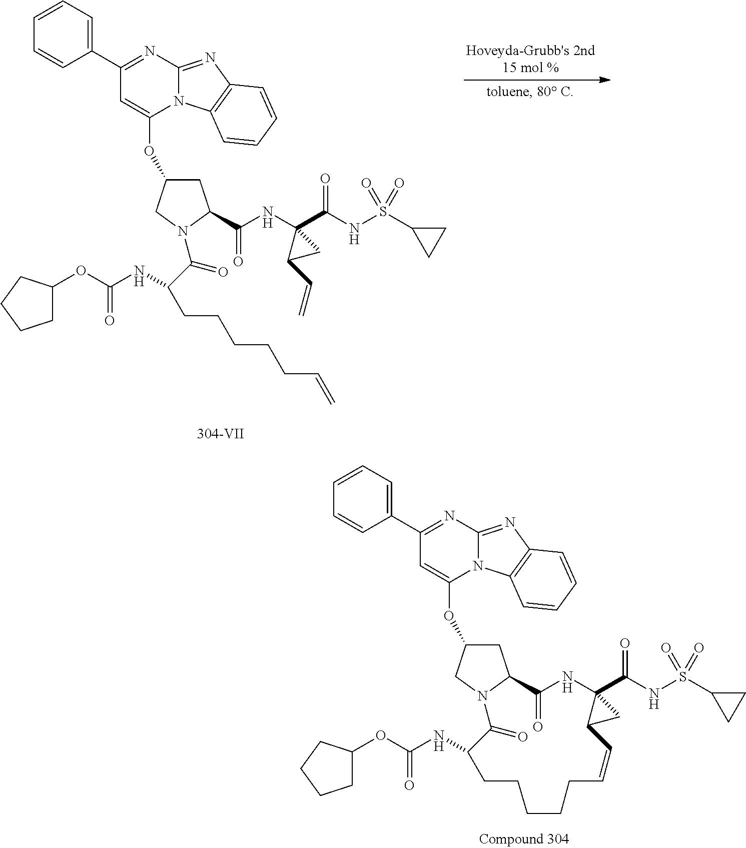

To a solution of ethyl benzoylacetate (228-II) (0.94 g, 4.9 mmol) in 10 mL of acetic acid was added 1H-Benzoimidazol-2-ylamine (304-I) (0.55 g, 4.1 mmol) at room temperature. The reaction mixture was then heated to 130° C. and stirred for 18 hours. After the reaction was completed, the mixture was concentrated under vacuum. The residue was washed with 20 mL of 50% ethyl acetate/hexanes solution to give yellow precipitate. The suspension was filtered to give compound 304-II as a pale yellow solid (0.96 g, yield=90%). MS m/z 262.1 (M⁺+1).

A solution of 304-II (1.65 g, 6.3 mmol) and excess phosphorus oxychloride (POCl₃) was refluxed for 2 hours. After cooled and thoroughly concentrated, the mixture was subjected to extraction with methylene chloride and 10% sodium hydroxide. The organic layer was dried over MgSO₄, concentrated, and crystallized from CH₂Cl₂ and n-hexane to give compound 304-III (1.50 g, yield=85%). MS m/z 280.1 (M⁺+1).

To a suspension of boc-trans-4-hydroxy-L-proline (1.36 g, 5.88 mmol) in DMSO (30 mL) was added t-BuONa (1.13 g, 11.76 mmol) at 0° C. After the mixture was warmed to room temperature and stirred for 30 min, compound 304-III (1.50 g, 5.36 mmol) was added slowly at 10° C. After stirred in a cold water bath for 30 min, the reaction mixture was neutralized to pH 6˜7 by 10% NaHSO₄ aqueous solution and diluted by 50 mL of water. The suspension was then filtered and air dried to afford compound 304-IV (1.78 g, yield=72%). MS m/z 475.2 (M⁺+1).

To a solution of HATU (1.68 g, 4.4 mmol), 1-hydroxybenzotriazole (HOBT, 0.60 g, 4.4 mmol), 304-IV (1.50 g, 3.16 mmol) and Cyclopropanesulfonic acid (1-amino-2-vinyl-cyclopropanecarbonyl)-amide (0.80 g, 3.47 mmol) in CH₂Cl₂ (30 mL) at room temperature was added N-methylmorpholine (NMM, 1.6 g, 15.8 mmol). After 18 hours of stirring, the reaction mixture was washed with saturated NaHCO₃ aqueous solution. Extraction with CH₂Cl₂ followed by concentration gave the crude compound 304-V. The residue was purified by silica gel column chromatography (EtOAc/CH₂Cl₂=4/1) to give compound 304-V (1.35 g, yield=62%). MS m/z 687.3 (M⁺+1).

To a solution of compound 304-V (1.35 g, 1.96 mmol) in CH₂Cl₂ (20 mL) was added 4 mL of TFA at room temperature. After 2 hours of stirring, the reaction mixture was concentrated under vacuum to give compound 304-VI. The resulting residue was used without further purification for the next step. MS: m/z 587.3 (M⁺+1).

NMM (0.99 g, 9.8 mmol) was added to a solution of 2-cyclopentyloxycarbonylamino-non-8-enoic acid (1.0 g, 2.16 mmol), HATU (1.04 g, 2.74 mmol), HOBT (0.37 g, 2.74 mmol) and crude compound 304-VI (1.15 g, 1.96 mmol) in CH₂Cl₂ (20 mL) at room temperature. After 18 hours of stirring, the reaction mixture was washed with saturated NaHCO₃ aqueous solution. Extraction with CH₂Cl₂ followed by concentration gave the crude compound 304-VII. The residue was purified by silica gel column chromatography (2% MeOH/CH₂Cl₂) to give compound 304-VII (1.16 g, yield=69%). MS m/z 852.4 (M⁺+1).

To a solution of compound 304-VII (0.85 g, 1.0 mmol) in toluene (200 mL) was added Hoveyda-Grubbs 2^{nd} (100 mg, 0.159 mmol) at room temperature under N₂. Then, the reaction mixture was stirred at 80° C. for 18 hours to carry out metathesis cyclization. The reaction was quenched and the reaction mixture was purified by silica gel column chromatography (5% MeOH/CH₂Cl₂) to give compound 304 (0.12 g, yield=15%). MS: m/z 824.4 (M⁺+1); ¹H NMR (CDCl₃) 9.75 (s, 1H), 7.79 (m, 3H), 7.55 (m, 2H), 7.44 (m, 2H), 7.15 (dd, 1H), 6.91 (dd, 1H), 6.21 (s, 1H), 5.70 (dt, 1H), 5.53 (s, 1H), 5.35 (m, 2H), 5.04-4.94 (m, 2H), 4.72 (m, 1H), 4.35 (m, 1H), 4.11 (m, 1H), 3.69-3.60 (m, 2H), 2.99 (m, 1H), 2.56 (m, 2H), 1.63-1.09 (m, 18H), 0.87-0.80 (m, 6H).

### Example 16

### Synthesis of Compound 305

Compound 305 was prepared according to the procedure described above.

Compound 305: MS: m/z 828.4 (M⁺+1); ¹H NMR (CDCl₃) 9.21 (s, 1H), 7.53 (d, 1H), 7.45 (s, 1H), 7.38 (d, 1H), 7.10 (dd, 1H), 6.90 (dd, 1H), 6.55 (d, 1H), 6.37 (s, 1H), 5.71 (dt, 1H), 5.54 (s, 1H), 5.30 (m, 1H), 5.21 (d, 1H), 5.06-5.00 (m, 2H), 4.70 (m, 1H), 4.39 (m, 1H), 4.17 (d, 1H), 3.79-3.70 (m, 2H), 3.48 (m, 1H), 2.58 (m, 2H), 1.99-1.49 (m, 18H), 1.25 (s, 3H), 0.88-0.79 (m, 6H).

### Example 17

### Inhibition of NS3/4A Protease Activity

### Protein Expression and Purification

A plasmid containing a gene encoding N-terminal His₆-tagged-NS4A₍₂₁₋₃₂₎-GSGS-NS3₍₃₋₁₈₁₎ was transformed into E. coli strain BL21(DE3) pLysS (Novagen) for protein expression. Single colony of transformed BL21 (DE3) pLysS was cultured in 200 mL of LB medium with Kanamycin and Chloramphenicol at 37° C. overnight. The bacterial culture was transferred to 6 L LB medium (Difco) containing antibiotics and incubated with shaking at 22° C. After the absorbance at 600 nm reached 0.6, the culture was induced with 1 mM isopropyl-1-thio-β-D-galactopyranoside (IPTG) at 22° C. for 5 hours. The culture was subsequently harvested by centrifugation (6,000×g for 15 minutes at 4° C.). Cell pellets were resuspended in 150 mL buffer A (50 mM HEPES, pH 7.4, 0.3 M NaCl, 0.1% (w/v) CHAPS, 10 mM imidazol, 10% (v/v) glycerol). After the mixture was disrupted by four passes through a Microfluidizer operated at 30 psi, the cell debris was removed by centrifugation (58,250×g for 30 minutes at 4° C.). The cell lysate containing His₆-tagged proteins was charged at 3 mL/min onto a 25 mL Ni-NTA (Qiagen) column in the presence of 10 mM imidazole using a gradiFrac system (Pharmacia). The column was washed with 10 column volumes of the lysis buffer. The bound NS4A₍₂₁₋₃₂₎-GSGS-NS3₍₃₋₁₈₁₎ was eluted with 8 column volumes of buffer A supplemented with 300 mM imidazole. The pooled fractions were further purified by Q-Sepharose column equilibrated with buffer B (50 mM HEPES, pH 7.4, 0.1% (w/v) CHAPS, 10% (v/v) glycerol, 5 mM dithiothreitol (DTT), and 1 M NaCl). The eluant containing NS4A₍₂₁₋₃₂₎-GSGS-NS3₍₃₋₁₈₁₎ was collected and further purified by size-exclusion chromatography at a flow rate of 0.5 mL/min using the sephacryl-75 column (16×100 cm, Pharmacia) pre-equilibrated with buffer C (50 mM HEPES, pH 7.4, 0.1% (w/v) CHAPS, 5 mM DTT, 10% (v/v) glycerol). The purified protein was frozen and stored at −80° C. for future use.

### HPLC Microbore Assay

A solution containing 50 mM Tris, pH 7.4, 100 mM NaCl, 20% glycerol, 0.012% CHAPS, 10 mM DTT, 5 μM substrate Ac-Asp-Glu-Asp(EDANS)-Glu-Glu-Abu-ψ-[COOAla]-Ser-Lys(DABCYL)-NH₂ (RET 51, ANASPEC), and 10 μM test compound was prepared. 80 μL of the solution was added to each well of a 96-well plate. Reaction was initiated by addition of 20 μL of 10 nM NS3/4A protease in a buffer containing 50 mM Tris buffer, pH 7.4, 100 mM NaCl, 20% glycerol, and 0.012% CHAPS. The final concentration of NS3/4A protease in assay was 2 nM.

The assay solution was incubated for 30 minutes at 30° C. The reaction was then quenched by addition of 100 μL of 1% TFA. 200 μL aliquot was transferred to each well of Agilent 96-well plates.

Reaction products were analyzed using reverse phase HPLC described below. The HPLC system included: Agilent 1100, Degasser G1379A, Binary pump G1312A, Autosampler G1367A, Column thermostated chamber G1316A, Diode array detector G1315B, Column: Agilent, ZORBAX Eclipse XDB-C18, 4.6 mm, 5 μm, P/N 993967-902, Column thermostat: room temperature, injection volume: 100 μL, Solvent A=HPLC grade water+0.09% TFA, Solvent B=HPLC grade acetonitrile+0.09% TFA. Total HPLC running time was 7.6 minutes with a linear gradient from 25 to 50% solvent B in 4 minutes, 50% solvent B for 30 seconds, and a gradient from 50 to 25% solvent B for additional 30 seconds. The column was re-equilibrated with 25% solvent B for 2.6 minutes before next sample was injected. The IC₅₀ value (the concentration at which 50% inhibition of NS3/4A activity was observed) was calculated for each test compound based on the HPLC results.

Compounds 1-305 were tested in the above inhibition assay. All of these compounds were active in inhibiting NS3/4A activity. Some compounds showed IC₅₀ values in the range of 0.1 to 100 nM.

In addition, certain compounds of this invention were found to unexpectedly inhibit in an effective manner HCV protease mutants resistant to one or more other HCV drugs.

### Example 18

### HCV Replicon Cell Assay Protocol

Cells containing HCV replicon were maintained in DMEM containing 10% fetal bovine serum (FBS), 1.0 mg/ml of G418, and appropriate supplements (media A).

On day 1, the replicon cell monolayer was treated with a trypsin/EDTA mixture, removed, and was diluted with media A to a final concentration of 48,000 cells/ml. The solution (1 ml) was added to each well of a 24-well tissue culture plate, and cultured overnight in a tissue culture incubator at 37° C. with 5% CO₂.

On day 2, a test compound (in 100% DMSO) was serially diluted by DMEM containing 10% FBS and appropriate supplements (media B). The final concentration of DMSO was maintained at 0.2% throughout the dilution series.

The media on the replicon cell monolayer was removed, and then media B containing various concentrations of compounds was added. Media B without any compound was added to other wells as compound-free controls.

The cells were incubated with a compound or 0.2% DMSO in media B for 72 hours in a tissue culture incubator with 5% CO₂ at 37° C. Then, the media was removed and the replicon cell monolayer was washed once with PBS. RNA extraction reagents from RNeasy kits or TRIZOL reagents were added to the cells immediately to avoid degradation of RNA. Total RNA was extracted according to the instruction provided by manufacturer with modification to improve extraction efficiency and consistency. Finally, total cellular RNA, including HCV replicon RNA, was eluted and stored at −80° C. until further processing.

A TaqMan® real-time RT-PCR quantification assay was set up with two sets of specific primers: one was for HCV and the other was for ACTB (beta-actin). The total RNA was added to the PCR reactions for quantification of both HCV and ACTB RNA in the same PCR well. Experimental failure was flagged and rejected based on the level of ACTB RNA in each well. The level of HCV RNA in each well was calculated according to a standard curve run in the same PCR plate. The percentage of inhibition of HCV RNA level by the compound treatment was calculated using the DMSO or compound-free control as 0% of inhibition. EC50 (concentration at which 50% inhibition of the HCV RNA level was achieved) was calculated from the titration curve of any given compound.

Compounds 1-305 were tested in the above inhibition assay. All of these compounds inhibited the HCV RNA level. Some compounds showed IC₅₀ values in the range of 0.1 to 100 nM.

### Example 19

### Pharmacokinetic Study

Male Sprague-Dawley rats (300-400 g) were surgically implanted with polyethylene cannula in the jugular vein for blood sampling while under pentobarbital anesthesia the day before the in-life phase. They were fasted overnight with water ad libitum, and then dosed the next day with a test compound by oral gavage. Serial blood samples were collected from animals until 48 hrs post-dose and heparinized plasma was recovered following centrifugation. The test compound in blood plasma was extracted and determined by liquid chromatography-mass spectrometry analysis (LC-MS/MS).

Standard pharmacokinetic parameters were assessed by non-compartmental analysis using WinNonlin (Version 4.0, Pharsight, Calif., USA). The maximum in the curve of the test compound concentration in blood plasma vs. time is denoted Cₘₐₓ. The apparent terminal-phase elimination (t_{1/2}) were calculated as ln(2)/λ_{z}, where λ_{z} is an elimination rate constant. The area under the concentration vs. time curve from the time of dosing to infinity (AUC_{(0-inf)}) was calculated according to the linear trapezoidal rule.

Certain compounds of this invention showed prolonged half-life and high AUC values.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of Formula (I): 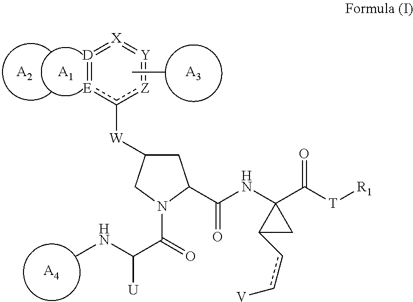
wherein
R₁ is H, or is a moiety selected from C₁₋₆ alkyl, C₃₋₁₀ cycloalkyl, C₁₋₁₀ heterocycloalkyl, aryl, and heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl;
T is —O—, —NH—, —NH(CO)—, —NHS(O)—, or —NHSO₂—;
each of U and V, independently, is H, C₁₋₆ alkyl, or C₃₋₁₀ cycloalkyl;
W is —O—, —S—, —NH—, —OCH₂—, or a bond;
each of D and E, independently, is C or N;
one of X, Y, and Z is C and bonded to A₃, and each of the others, independently, is CH, N, or deleted;
each of A₁, A₂, and A₃, independently, is a moiety selected from C₄₋₁₀ cycloalkyl, C₁₋₁₀ heterocycloalkyl, aryl, and heteroaryl, each of which is optionally mono-, di-, or tri-substituted with halo, nitro, cyano, amino, amideo, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl;
A₄ is a moiety selected from C₁₋₁₀ heterocycloalkyl, aryl, and heteroaryl, each of which is optionally mono-, di-, or tri-substituted with halo, cyano, amino, amido, carbonyloxy, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; each of C₁₋₆ alkyl, C₁₋₆ alkoxyl, C₂₋₆ alkenyl, and C₂₋₆ alkynyl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, and each of C₃₋₁₀ cycloalkyl, C₁₋₁₀ heterocycloalkyl, aryl, and heteroaryl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, or optionally fused with C₃₋₁₀ cycloalkyl, C₁₋₁₀ heterocycloalkyl, aryl, or heteroaryl; and [symbol]
is a single bond or a double bond.

2. The compound of claim 1, wherein 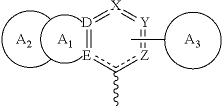
is 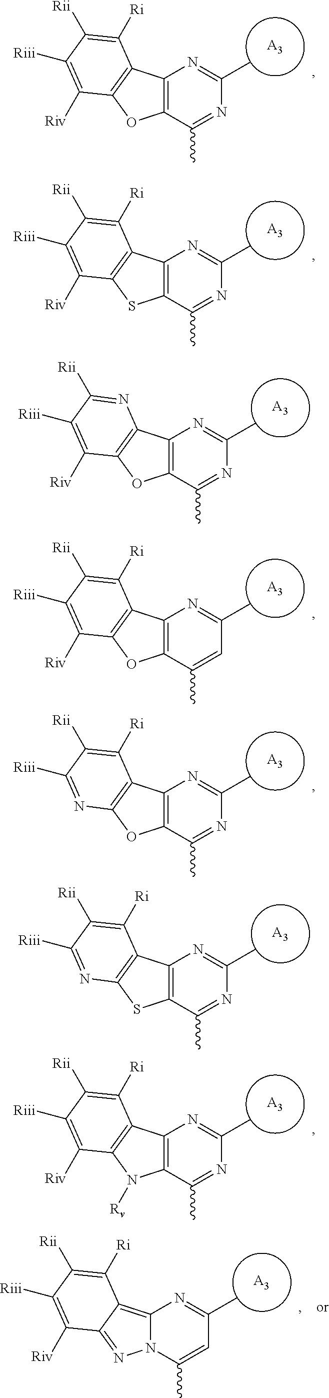 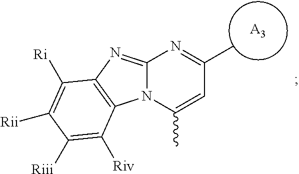
in which A₃ is aryl or heteroaryl optionally substituted with halo, amino, amido, C₁₋₆ alkyl, or C₁₋₆ alkoxyl; and each of Rᵢ, Rᵢᵢ, Rᵢᵢᵢ, Rᵢᵥ, and Rᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

3. The compound of claim 2, wherein A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl.

4. The compound of claim 1, 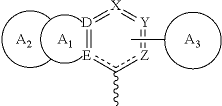
is a moiety selected from 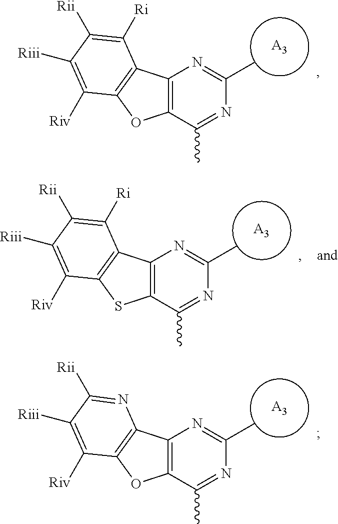
in which A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl; and each of Rᵢ, Rᵢᵢ, Rᵢᵢᵢ, Rᵢᵥ, and Rᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

5. The compound of claim 1, wherein R₁ is 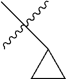
or 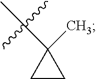
W is O; and T is —NHSO₂—.

6. The compound of claim 4, wherein
R₁ is 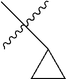
or 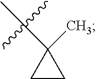
T is —NHSO₂—;
U is C₁₋₆ alkyl;
V is H; [symbol]
is a double bond; and 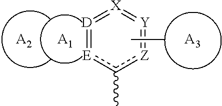
is a moiety selected from 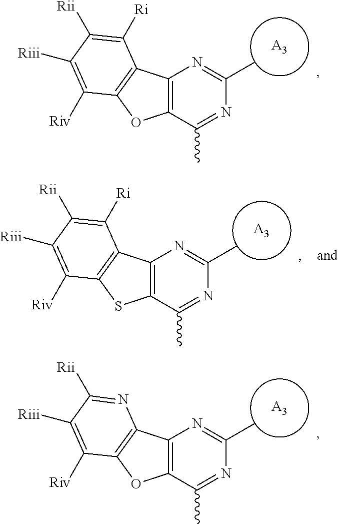
in which A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl; and each of Rᵢ, Rᵢᵢ, Rᵢᵢᵢ, Rᵢᵥ, and Rᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

7. The compound of claim 1, wherein the compound is one of compounds 1-183.

8. The compound of claim 1, wherein the compound is one of compounds 95, 102, 123, 139, 144, 150, 162, 164, 165, 180, and 183.

9. A compound of Formula (II): 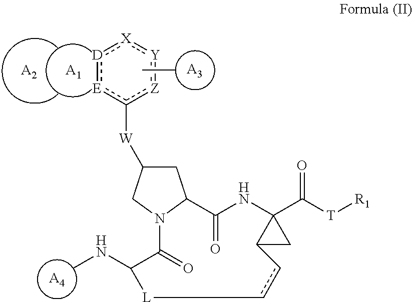
wherein
R₁ is H, or is a moiety selected from C₁₋₆ alkyl, C₃₋₁₀ cycloalkyl, C₁₋₁₀ heterocycloalkyl, aryl, and heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl;
T is —O—, —NH—, —NH(CO)—, —NHS(O)—, or —NHSO₂—;
L is C₁₋₆ alkylene, C₂₋₆ alkenylene, or C₃₋₁₀ cycloalkylene;
W is —O—, —S—, —NH—, —OCH₂—, or a bond;
each of D and E is C or N;
one of X, Y, and Z is C and bonded to A₃, and each of the others, independently, is CH, N, or deleted;
each of A₁, A₂, and A₃, independently, is a moiety selected from C₄₋₁₀ cycloalkyl, C₁₋₁₀ heterocycloalkyl, aryl, and heteroaryl, each of which is optionally mono-, di-, or tri-substituted with halo, nitro, cyano, amino, amideo, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl;
A₄ is a moiety selected from C₁₋₁₀ heterocycloalkyl, aryl, and heteroaryl, each of which is optionally mono-, di-, or tri-substituted with halo, cyano, amino, amido, carbonyloxy, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; each of C₁₋₆ alkyl, C₁₋₆ alkoxyl, C₂₋₆ alkenyl, and C₂₋₆ alkynyl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, and each of C₃₋₁₀ cycloalkyl, C₁₋₁₀ heterocycloalkyl, aryl, and heteroaryl being optionally mono-, di- or tri-substituted with halo, cyano, amino, amido, C₁₋₆ alkyl, C₁₋₆ alkoxyl, C₂₋₆ alkenyl, C₂₋₆ alkynyl, C₃₋₁₀ cycloalkyl, C₁₋₁₀ heterocycloalkyl, aryl, or heteroaryl, or optionally fused with C₃₋₁₀ cycloalkyl, C₁₋₁₀ heterocycloalkyl, aryl, or heteroaryl; and [symbol]
is a single bond or a double bond.

10. The compound of claim 9, wherein 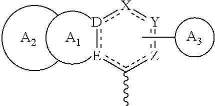
is 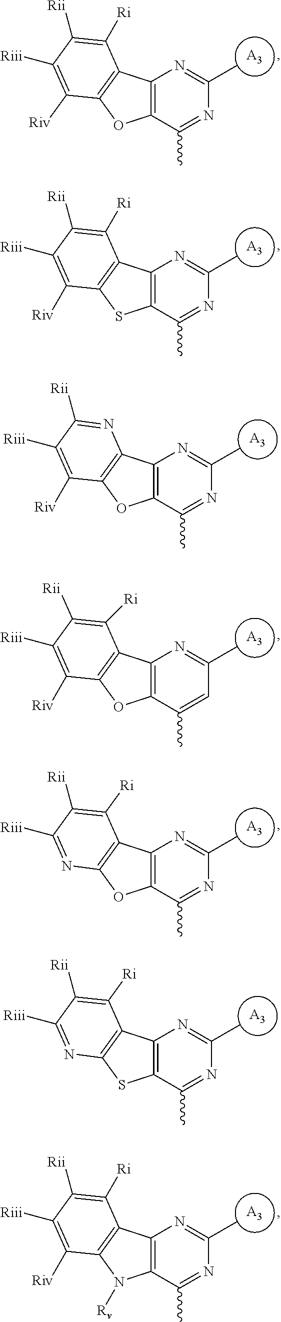 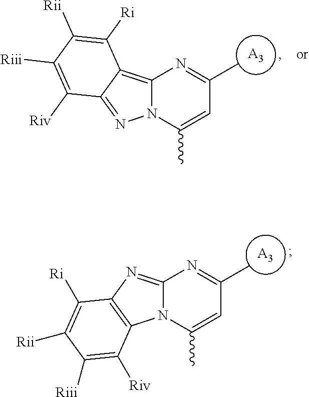
in which A₃ is aryl or heteroaryl optionally substituted with halo, amino, amido, C₁₋₆ alkyl, or C₁₋₆ alkoxyl; and each of Rᵢ, Rᵢᵢ, Rᵢᵢᵢ, Rᵢᵥ, and Rᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

11. The compound of claim 10, wherein A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl.

12. The compound of claim 9, 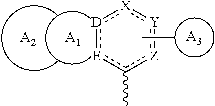
is a moiety selected from 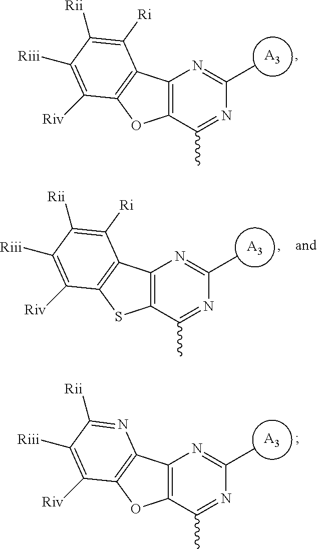
in which A₃ is phenyl optionally substituted with optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl.

13. The compound of claim 9, wherein R₁ is 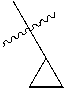
or

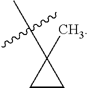

14. The compound of claim 9, wherein A₄ is phenyl, pyrimidyl, or thiazolyl which is optionally mono-, di-, or tri-substituted with halo, cyano, amido, carbonyloxy, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

15. The compound of claim 9, wherein
R₁ is 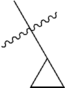
or 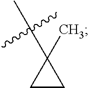
T is —NHSO₂—;
L is —(CH₂)ₘ—, m being 3, 4, or 5; [symbol]
is a double bond; and 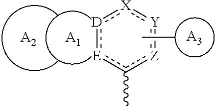
is a moiety selected from 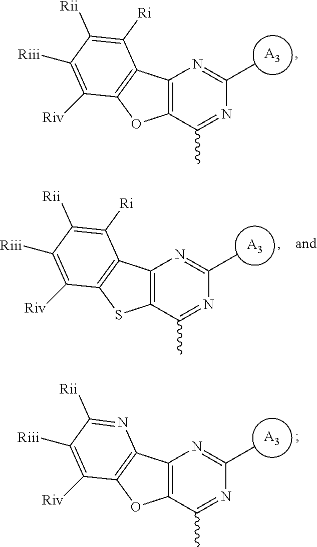
in which A₃ is aryl or heteroaryl optionally substituted with halo, amino, amido, C₁₋₆ alkyl, or C₁₋₆ alkoxyl; and each of Rᵢ, Rᵢᵢ, Rᵢᵢᵢ, and Rᵢᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl; and
A₄ is phenyl, pyrimidyl, or thiazolyl which is optionally mono-, di-, or tri-substituted with halo, cyano, amido, carbonyloxy, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

16. The compound of claim 9, wherein the compound is one of compounds 184-227.

17. The compound of claim 19, wherein the compound is one of compounds 190, 199, 209, 218, 222, 224, and 226.

18. A compound of Formula (III): 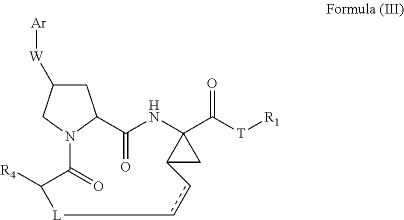
wherein
R₁ is H, OH, halo, nitro, cyano, amino, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, or heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl;
T is —O—, —NH—, —NH(CO)—, —NHS(O)—, or —NHSO₂—;
L is —(CH₂)ₘ—, m being 3, 4, or 5;
W is —O—, —S—, —NH—, —OCH₂—, or a bond;
R₄ is H, NHR, or NHZR; in which R is C₁₋₆ alkyl, C₃₋₁₀ cycloalkyl, C₁₋₁₀ heterocycloalkyl, aryl, or heteroaryl, each of which is optionally mono-, di-, or tri-substituted with halo, nitro, cyano, amino, C₁₋₆ alkyl, C₁₋₆ alkoxyl, C₂₋₆ alkenyl, C₂₋₆ alkynyl, aryl, or heteroaryl; Z is —(C═O)—, —(C═O)O—, —(C═O)(C═O)O—, —(C═O)(C═O)NH—, —(C═O)NHR′—, —(C═O)S—, (C═S)NHR, OR C(NH)O, R′ being H, C₁₋₆ alkyl, C₃₋₁₀ cycloalkyl, C₁₋₁₀ heterocycloalkyl, aryl, or heteroaryl; and
Ar is 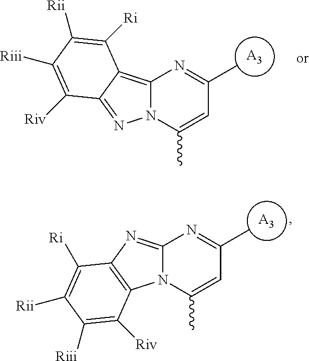
in which A₃ is C₁₋₆ alkyl, C₁₋₆ alkoxy, C₃₋₈ cycloalkyl, C₁₋₈ heterocycloalkyl, aryl, or heteroaryl optionally substituted with halo, amino, amido, C₁₋₆ alkyl, or C₁₋₆ alkoxyl; and each of Rᵢ, Rᵢᵢ, Rᵢᵢᵢ, and Rᵢᵥ, independently, is H, halo, cyano, amino, amido, C₁₋₆ alkyl, C₁₋₆ alkoxyl, C₂₋₆ alkenyl, or C₂₋₆ alkynyl, or is a moiety selected from C₃₋₁₀ cycloalkyl, C₁₋₁₀ heterocycloalkyl, aryl, and heteroaryl, each of which is optionally mono-, di-, or tri-substituted with halo, nitro, cyano, amino, amido, C₁₋₆ alkyl, C₁₋₆ alkoxyl, C₂₋₆ alkenyl, C₂₋₆ alkynyl, aryl, or heteroaryl, or optionally fused with C₃₋₁₀ cycloalkyl, C₁₋₁₀ heterocycloalkyl, aryl, or heteroaryl.

19. The compound of claim 18, wherein R₁ is 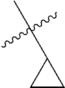
or 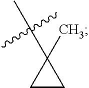
T is —NHSO₂—; and [symbol] is a double bond and L is —(CH₂)ₘ—, m being 3, 4, or 5.

20. The compound of claim 18, wherein the compound is one of compounds 228-305.

21. The compound of claim 18, wherein the compound is one of compounds 245, 274, 280, 281, 286, 288, and 289.

22. A method for treating hepatitis C virus infection, comprising administering to a subject in need thereof an effective amount of a compound of claim 1.

23. A method for treating hepatitis C virus infection, comprising administering to a subject in need thereof an effective amount of a compound of claim 9.

24. A method for treating hepatitis C virus infection, comprising administering to a subject in need thereof an effective amount of a compound of claim 18.
